(19) United States
(12) Patent Application Publication
(10) Pub. No.: US 2023/0407243 A1
(43) Pub. Date: Dec. 21, 2023

(54) GENETIC PLATFORM TO INVESTIGATE THE FUNCTIONS OF BACTERIAL DRUG EFFLUX PUMPS

(71) Applicant: University of Guelph, Guelph (CA)

(72) Inventors: Georgina Cox, Guelph (CA); Tanisha Teelucksingh, Brampton (CA); Laura Thompson, Guelph (CA)

(21) Appl. No.: 18/210,416

(22) Filed: Jun. 15, 2023

Related U.S. Application Data
(60) Provisional application No. 63/352,569, filed on Jun. 15, 2022.

(51) Int. Cl. C12N 1/20; C12N 15/01

(52) U.S. Cl. CPC C12N 1/205; C12N 15/01; C12R 2001/19

(57) ABSTRACT

The present disclosure provides an Escherichia coli strain comprising at least 20 of inactivated genes from acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. Also provided is a method for identifying a compound that is an antibacterial agent using an Escherichia coli strain disclosed herein. Further provided is a method for creating an Escherichia coli strain with one active efflux pump.

### CROSS REFERENCE TO RELATED APPLICATION

The present disclosure claims priority from U.S. provisional application No. 63/352,569 filed on Jun. 15, 2022, which is hereby incorporated by reference in its entirety.

### INCORPORATION OF SEQUENCE LISTING

A computer readable form of the Sequence Listing “6580-P68227US01_SequenceListing.xml” (5,017,998 bytes), submitted via Patent Center and created on Aug. 4, 2023, is herein incorporated by reference.

### FIELD

The present disclosure provides an Escherichia coli strain comprising inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. Also provided is a method for identifying a compound that is an antibacterial agent using an Escherichia coli strain disclosed herein. Further provided is a method for creating an Escherichia coli strain with one active efflux pump.

### BACKGROUND

Gram-negative bacteria represent a serious challenge for antibacterial drug discovery efforts. The outer membrane (OM) is a formidable barrier for the entry of large and hydrophobic compounds, and the inner membrane (IM) reduces the influx of hydrophilic drugs. These two membranes augment the next line of defense, membrane-spanning efflux pumps, which effectively reduce the intracellular and periplasmic concentrations of compounds that have penetrated the cell. Synergy between influx retardation and active efflux contributes considerably to the intrinsic antibiotic resistome of Gram-negative pathogens.

Multidrug-resistance (MDR) efflux pumps, which typically extrude a wide range of structurally unrelated substances, have been particularly well studied. However, bacterial species harbor large networks of additional and often poorly characterized drug efflux pumps. For example, sequence annotation of the Escherichia coli K-12 genome highlighted the presence of 36 known or putative drug efflux pumps, which span five protein families: the ATP-binding cassette (ABC) superfamily, the resistance-nodulation-cell division (RND) superfamily, the major facilitator superfamily (MFS), the small multidrug resistance family (SMR), and the multi-antimicrobial extrusion (MATE) family. E. coli efflux pumps within the RND and ABC superfamilies complex with periplasmic adaptor proteins and the OM channel TolC. These tripartite complexes span the entire cell envelope. Certain MFS pumps, such as EmrB and EmrY, also form tripartite complexes with TolC. However, a majority of MFS members are single component pumps that extrude substrates to the periplasm. Efflux pumps from the MATE and SMR families are also single component efflux pumps. In the case of antibiotics with cytoplasmic targets, synergistic relationships are thought to exist between tripartite systems and single component IM pumps. In this instance, single component pumps extrude substrates to the periplasm, and tripartite assemblies then efflux to the exterior of the cell, which is often referred to as functional ‘interplay’.

Since efflux pumps are a major contributor to antibiotic resistance, delineating the substrate specificities and functions of these membrane-spanning proteins is critical for the development of strategies to compromise and/or circumvent these ancient resistance elements. In addition, while efflux pumps have largely been studied for their ability to extrude most classes of clinically important antibiotics, they are also increasingly associated with physiological functions. Indeed, conservation of the E. coli efflux system further supports such physiological functions. However, important questions remain regarding the physiological roles of these proteins. Overall, a major limitation hindering the study of bacterial efflux pumps has been the lack of a suitable genetic background. It is difficult to delineate the substrate specificities and functions of each pump due to the sheer number encoded within the genome, the differential expression of efflux pump-encoding genes, and functional redundancies. For example, in terms of functional redundancy, E. coli K-12 strains harbor six pumps that efflux tetracycline, which is proposed to provide a more robust and flexible defense mechanism.

### SUMMARY

To address these limitations, inventors generated and thoroughly characterized an extensively efflux-deficient mutant strain of E. coli. This strain provides a simplified genetic background free of the masking effects and redundancies of promiscuous efflux pumps. While a growing body of literature associates drug efflux pumps with important physiological processes, which suggests their removal could be detrimental or infeasible, inventors successfully inactivated 35 IM efflux pumps comprising the E. coli drug efflux network, generating Efflux KnockOut-35 (EKO-35). Phenotypic profiling of this strain revealed the E. coli drug efflux network is dispensable under optimal growth conditions, with little impact on the cellular proteome in nutrient-rich conditions. Importantly, when EKO-35 is propagated under diverse growth conditions, inventors reveal distinct patterns of dispensability, which opens the way for future studies to investigate the efflux pumps responsible for these conditionally essential phenotypes. To the best of inventors' knowledge, EKO-35 represents the most efflux-deficient bacterial mutant to be reported.

In addition to the important biological insight gained through generation of EKO-35, this strain can also be used as a well-characterized simplified genetic background to study the functions of efflux pumps of interest. To demonstrate the utility of EKO-35, inventors constructed an efflux platform consisting of EKO-35 genomic integrations of genes encoding E. coli efflux pumps forming tripartite complexes with the OM channel TolC. Each strain was profiled against a curated collection of physicochemically diverse compounds, which enabled the inventors to summarize molecular properties contributing to transport in each of these proteins. Inventors also profiled the MexCD pump from Pseudomonas aeruginosa, showing that the platform can be used to study efflux pumps from other organisms. Through the introduction of a large non-selective pore into the OM of EKO-35 (EKO-35-Pore), inventors demonstrate the efflux platform can be additionally utilized to study the specificity of efflux pump inhibitors, and to explore efflux pump interplay. Overall, EKO-35, the developed efflux platform, and the important insight gained into physicochemical substrate specificities and efflux essentiality, will have widespread application for the study of bacterial drug efflux pumps.

Accordingly, provided herein is an Escherichia coli strain comprising at least 20 of inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA.

In some embodiments, the strain comprises at least 25, at least 30 or more of the inactivated genes. In some embodiments, the strain comprises at least 34 of the inactivated genes. In some embodiments, strain comprises all 35 inactivated genes. In some embodiments, the Escherichia coli strain is deposited under International Depositary Authority of Canada (IDAC) accession number 310522-01 deposited on May 31, 2022, or IDAC accession number 070623-01 deposited on Jun. 7, 2023. In some embodiments, the Escherichia coli comprises a nucleic acid having the sequence as shown in SEQ ID NO: 255. In some embodiments, the strain further comprises an open variant of outer membrane ferric siderophore transporter FhuA. In some embodiments, the strain comprises at least one of reactivated acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, optionally under the control of a constitutive promoter. In some embodiments, one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, is reactivated. In some embodiments, the inactivation comprises deletion of the gene, or introducing a mutation into the gene to ablate expression of the gene or eliminate efflux pump activity of the protein expressed by the gene. In some embodiments, acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA are genes encoding for efflux pumps. In some embodiments, the strain comprising at least 20 of inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA is an efflux pump deficient strain. In some embodiments, the strain comprises all 35 inactivated genes is EKO-35.

Also provided is a method for identifying a compound that is an antibacterial agent, comprising
(a) i) contacting the compound with the Escherichia coli strain described herein and with wild-type Escherichia coli; and/or
ii) contacting the compound with the Escherichia coli strain described herein having reactivated genes and EKO-35; and/or
iii) contacting the compound with the Escherichia coli strain described herein having reactivated genes and an efflux pump deficiency strain described herein; and
(b) detecting viability of each of the Escherichia coli;
wherein the compound is identified as an antibacterial agent if the compound decreases viability of wild-type less than the Escherichia coli strain with efflux pump deficiency described herein;
optionally wherein the compound is identified as an antibacterial agent if the compound decreases viability of the Escherichia coli strain having reactivated efflux pump genes less than the Escherichia coli strain having at least 20 inactivated efflux pump genes;
optionally wherein the compound is identified as an antibacterial agent if the wild-type Escherichia coli or the Escherichia coli strain having reactivated efflux pump genes is resistant to the compound, and the compound decreases the viability of the Escherichia coli strain having at least 20 inactivated efflux pump genes.

In some embodiments, the decrease in viability of wild-type or the Escherichia coli strain described herein after contacting the compound is at most 10%, 20%, 30%, 40%, 50%, 60%, 70%, or 80%, or at least 20%, 30%, 40%, 50%, 60%, 70%, 80%, 90%, or 100%.

In some embodiments, the compound is identified as an antibacterial agent if the compound decreases the viability of efflux pump deficient Escherichia coli strain described herein at a faster rate than the decrease in viability of wild-type or the Escherichia coli strain comprising reactivated efflux pump genes.

In some embodiments, the compound decreases the viability of the Escherichia coli strain with one reactivated gene in EKO-35 is less than an efflux deficient strain disclosed herein, optionally Escherichia coli strain EKO-35, thereby identifying specificity of the compound for an efflux pump encoded by the reactivated gene, optionally the compound has a lower minimum inhibitory concentration (MIC) in the Escherichia coli strain of EKO-35 than EKO-35 with a reactivated efflux pump.

In some embodiments, the contacting comprises the Escherichia coli in a suitable culturing media for optimal Escherichia coli growth. In some embodiments, the contacting comprises incubating the Escherichia coli in nutrient-limiting culturing media. In some embodiments, the contacting comprises incubating the Escherichia coli for at least 24 h, 48 h, 72 h, or 96 h. In some embodiments, the culturing media is a media having a pH of about 2, about 3, about 4, or about 5.

Also provided is a method for creating an Escherichia coli strain producing one or more efflux pump, comprising reactivating one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA in an efflux deficient strain disclosed herein, optionally Escherichia coli strain EKO-35.

In some embodiments, the reactivation comprises introducing one or more of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, optionally by reversing the inactivation, optionally by re-introducing the gene back in genome with the same promoter or a different promoter, at the same locus or a different locus, optionally by introducing a knock down-resistant version of the gene.

Other features and advantages of the present disclosure will become apparent from the following detailed description. It should be understood, however, that the detailed description and the specific Examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments are described below in relation to the drawings in which:

FIG. 1A shows characterization of EKO-35 in Lysogeny broth (LB) at 37° C. FIG. 1A shows Western blot analysis of AcrB in the membranes of the E. coli K-12 wild-type and EKO-35 strains. AcrB and the AcrB_{D408A} mutant were produced in equivalent amounts when the genes were integrated into the arabinose operon (araC) of EKO-35, with gene expression under the control of the constitutive P_{LacI} promoter. AcrB band intensity was normalized to total protein using stain-free gels. Data represent mean values±s.d. of three independent biological replicates. P-values were calculated using a two-tailed Student's t-test (***P<0.001 and ****P<0.0001).

FIG. 1B shows characterization of EKO-35 in Lysogeny broth (LB) at 37° C. Measurement of EKO-35 growth kinetics revealed a marginal increase in the lag phase and generation time relative to the wild-type strain, which was assessed using three biological replicates (see Table 1).

FIG. 1C shows characterization of EKO-35 in Lysogeny broth (LB) at 37° C. Measuring the length of 50 cells of the wild-type K-12 and EKO-35 strains revealed no significant changes in cell length or morphology.

FIG. 1D shows characterization of the wild-type strain in Lysogeny broth (LB) at 37° C. FIG. 1D shows scanning electron microscopy of the wild-type strain during the mid-exponential phase of growth.

FIG. 1E shows characterization of EKO-35 in Lysogeny broth (LB) at 37° C. FIG. 1E shows scanning electron microscopy of EKO-35 during the mid-exponential phase of growth.

FIG. 1F shows characterization of EKO-35 in Lysogeny broth (LB) at 37° C. Principal component analysis revealed separation between the proteomes of EKO-35 (gray) and the wild-type strain (black) (Component 1, 40.6%), and slight biological variation (Component 2, 17.2%).

FIG. 1G shows characterization of EKO-35 in Lysogeny broth (LB) at 37° C. FIG. 1G shows volcano plot depicting significant changes in protein abundance in EKO-35 (gray), relative to the wild-type (black) strain's proteome. Statistical analysis was performed with four biological replicates using a Student's t-test (P-value 0.05, false discovery rate=0.05, S0=1).

FIG. 1H shows characterization of EKO-35 in Lysogeny broth (LB) at 37° C. FIG. 1H shows 1D-annotation enrichment of Uniprot Keywords enriched in EKO-35, relative to wild-type K-12 for proteins that were significantly (Student t-test, P-value false discovery rate=0.05) differentially abundant between wild-type and EKO-35.

FIG. 2A shows characterization of EKO-35 under nutrient-limitation at 37° C. The growth of EKO-35 was marginally impacted in M9 minimal glucose medium (see Table 1 for growth kinetics), which was assessed using three biological replicates. The strain entered the exponential phase of growth ˜5 h later than the wild-type strain.

FIG. 2B shows characterization of EKO-35 under nutrient-limitation at 37° C. Measuring the length of 50 cells of two biological replicates revealed EKO-35 cells were significantly longer under nutrient-limitation. P-values were calculated using a two-tailed Student's t-test (****P<0.0001).

FIG. 2C shows characterization of EKO-35 under nutrient-limitation at 37° C. FIG. 2C shows scanning electron microscopy of the wild-type strain during the mid-exponential phase of growth.

FIG. 2D shows characterization of EKO-35 under nutrient-limitation at 37° C. FIG. 2D shows scanning electron microscopy of EKO-35 during the mid-exponential phase of growth.

FIG. 2E shows characterization of EKO-35 under nutrient-limitation at 37° C. The growth of EKO-35 was not restored in amino acid-limited MOPS medium supplemented with iron, as determined using three biological replicates (see Table 1 for growth kinetics).

FIG. 2F shows characterization of EKO-35 under nutrient-limitation at 37° C. Principal component analysis separated EKO-35 (gray) from the wild-type (black) proteome (Component 1, 53.2%), and revealed slight biological variation (Component 2, 14%).

FIG. 2G shows characterization of EKO-35 under nutrient-limitation at 37° C. FIG. 2G shows 1 D-annotation enrichment of Uniprot Keywords enriched in EKO-35, relative to wild-type K-12 for proteins that exhibited significantly (Student t-test, P-value 0.05, false discovery rate=0.05) changes in abundance between wild-type and EKO-35.

FIG. 2H shows characterization of EKO-35 under nutrient-limitation at 37° C. FIG. 2H shows volcano plot illustrating significant changes in protein abundance of EKO-35 (gray), relative to the wild-type (black) strain. Statistical analysis was performed with four biological replicates using a Student's t-test (P-value 0.05, false discovery rate=0.05, S0=1).

FIG. 3A shows that the E. coli efflux system is contextually essential. FIG. 3A shows growth of the wild-type (K-12), EKO-35, ΔtolC, and EKO-35 araC::acrB strains in nutrient-rich medium under extreme acid and alkaline conditions. Data represent mean values±s.d. of three independent biological end-point readings after 18 h of growth. P-values were calculated by a two-tailed Student's t-test (****P<0.0001).

FIG. 3B shows that the E. coli efflux system is contextually essential. EKO-35 and EKO-35 araC::acrB_{D408A} show a significant increase in biofilm formation in nutrient-rich (P=3.61×10⁻⁵ and P=3.52×10⁻⁸, respectively) conditions. Data represents mean values±the s.d. for six biological replicates (nutrient-rich) and three biological replicates (nutrient-limited).

FIG. 3C shows that the E. coli efflux system is contextually essential. EKO-35 and EKO-35 araC::acrB_{D408A} show a significant increase in biofilm formation in nutrient-limited conditions (P=8.88×10⁻⁷ and P=6.23×10⁻⁷, respectively). Data represents mean values±the s.d. for six biological replicates (nutrient-rich) and three biological replicates (nutrient-limited).

FIG. 3D shows that the E. coli efflux system is contextually essential. Measurement of EKO-35 growth kinetics (Table 1) revealed a marginally extended lag phase in nutrient-rich medium (P=1.16×10⁻⁶).

FIG. 3E shows that the E. coli efflux system is contextually essential. Measurement of EKO-35 growth kinetics (Table 1) revealed no significant differences in nutrient-limited medium (P=0.187) at 25° C.

FIG. 3F shows that the E. coli efflux system is contextually essential. Under low oxygen (1%) conditions, in nutrient-rich medium, EKO-35 growth kinetics revealed significant changes (P=1.51×10⁻⁴) compared to the wild-type strain.

FIG. 3G shows that the E. coli efflux system is contextually essential. Supplementation of the nutrient-rich medium with 10 mM KNO₃ further impacted EKO-fitness (P=2.02×10⁻⁴) compared to the wild-type strain, which was partially restored through expression of mdtEF (EKO-35 araC::mdtEF) (P=5.09×10⁻⁴).

FIG. 3H shows that the E. coli efflux system is contextually essential. The E. coli drug efflux system is essential for growth in nutrient-limited, low oxygen (5%) conditions. Expression of mdtEF in EKO-35 (EKO-35 araC::mdtEF) marginally restored fitness, exhibiting a significantly improved generation time (P=6.09×10⁻⁴) compared to EKO-35. All P-values were calculated using a two-tailed Student's t-test (*P<0.05, **P<0.01, ***P<0.001, ****P<0.0001). Growth kinetic statistics are shown in Table 1.

FIG. 4A shows EKO-35 and the efflux platform enabled summation of efflux substrate physicochemical properties. Minimum inhibitory concentration (MIC) assays were conducted for 52 compounds against non-porinated (−) and porinated (+) wild-type K-12, ΔtolC, and EKO-35 (see Table 10). Compounds that increased the resistance of at least one EKO-35 integrated strain by ≥4-fold are shown in the heat map. Strains were assessed in technical duplicate. MIC values of wild-type K-12, ΔtolC, and EKO-35+/−the pore were log 2 transformed and normalized to 100% for each compound tested, where dark gray on the heat map represents the highest MIC value and white represents the lowest MIC value (see key).

FIG. 4B shows EKO-35 and the efflux platform enabled summation of efflux substrate physicochemical properties. FIG. 4B shows MIC assays for 52 compounds against 10 strains of non-porinated (−) and porinated (+) EKO-35 with chromosomally integrated efflux genes (see Tables 13A and 13B, and 14). Fold changes in MIC values of each strain compared to EKO-35+/−the pore were log 2 transformed and normalized to 100% for each compound (see Tables 15A, 15B, 16A, and 16B). Dark gray on the heat map indicates the greatest fold change, and white indicates the lowest fold change (see key). Slashes represent 2-fold increases in MIC value, which falls within the acceptable error range and were not considered significant. Box plots show individual data points representing substrate compounds and the corresponding property value, with the center line indicating the median, and whiskers the minimum and maximum values. Physicochemical substrate ranges are also summarized in Table 17. Abbreviations: RIF, rifampicin; VAN, vancomycin; FOF, fosfomycin; AMP, ampicillin; OXA, oxacillin; CHL, chloramphenicol; PURO, puromycin; AZM, azithromycin; ERY, erythromycin; TET, tetracycline; LZD, linezolid; MIN, minocycline; FA, fusidic acid; CIP, ciprofloxacin; NOR, norfloxacin; NAL, nalidixic acid; NOV, novobiocin; TMP, trimethoprim; DXR, doxorubicins DNR, daunorubicin; EtBr, ethidium bromide; ACF, acriflavine; BZK, benzalkonium chloride; DC, deoxycholate; STDC, sodium taurodeoxycholate.

FIG. 4C shows EKO-35 and the efflux platform enabled summation of efflux substrate physicochemical properties. Molecular weight (MW) for each compound was calculated using DataWarrior (Version 5.5.0). Box plots show individual data points representing substrate compounds and the corresponding property value, with the center line indicating the median, and whiskers the minimum and maximum values. Physicochemical substrate ranges are also summarized in Table 17.

FIG. 4D shows EKO-35 and the efflux platform enabled summation of efflux substrate physicochemical properties. Lipophilicity (log P) for each compound was calculated using DataWarrior (Version 5.5.0). Box plots show individual data points representing substrate compounds and the corresponding property value, with the center line indicating the median, and whiskers the minimum and maximum values. Physicochemical substrate ranges are also summarized in Table 17.

FIG. 4E shows EKO-35 and the efflux platform enabled summation of efflux substrate physicochemical properties. Aqueous solubility (log S) for each compound was calculated using DataWarrior (Version 5.5.0). Box plots show individual data points representing substrate compounds and the corresponding property value, with the center line indicating the median, and whiskers the minimum and maximum values. Physicochemical substrate ranges are also summarized in Table 17.

FIG. 4F shows EKO-35 and the efflux platform enabled summation of efflux substrate physicochemical properties. Polar Surface Area (PSA) for each compound was calculated using DataWarrior (Version 5.5.0). Box plots show individual data points representing substrate compounds and the corresponding property value, with the center line indicating the median, and whiskers the minimum and maximum values. Physicochemical substrate ranges are also summarized in Table 17.

FIG. 5A shows EKO-35 and the efflux platform can be used to assess efflux pump inhibitor specificities and efflux pump interplay. FIG. 5A shows bar charts depicting fractional inhibitory concentration index (FICI) values of phenylalanine-arginine-β-naphthylamide (PAβN) in combination with oxacillin and novobiocin. The FICI represents the ΣFIC of each drug. The FIC for each drug was determined by dividing the MIC of each drug in combination, by the MIC of each drug alone. ΣFIC=FIC_{A}+FIC_{B}=(C_{A}/MIC_{A})+(C_{B}/MIC_{B}). Synergy (FICI<0.5), additive (FICI>0.5-1.0), indifferent (FICI>1.0-2.0), and antagonistic (FICI>2.0), as highlighted on each bar chart. Related to Tables 18A, 18B, 19A and 19B.

FIG. 5B shows EKO-35 and the efflux platform can be used to assess efflux pump inhibitor specificities and efflux pump interplay. FIG. 5B shows bar charts depicting fractional inhibitory concentration index (FICI) values of phenylalanine-arginine-8-naphthylamide (PAβN) in combination with fusidic acid and ciprofloxacin. The FICI represents the ΣFIC of each drug. The FIC for each drug was determined by dividing the MIC of each drug in combination, by the MIC of each drug alone. ΣFIC=FIC_{A}+FIC_{B}=(C_{A}/MIC_{A})+(C_{B}/MIC_{B}). Synergy (FICI<0.5), additive (FICI>0.5-1.0), indifferent (FICI>1.0-2.0), and antagonistic (FICI>2.0), as highlighted on each bar chart. Related to Tables 18A, 18B, 19A and 19B.

FIG. 5C shows EKO-35 and the efflux platform can be used to assess efflux pump inhibitor specificities and efflux pump interplay. FIG. 5C shows bar charts depicting fractional inhibitory concentration index (FICI) values of phenylalanine-arginine-β-naphthylamide (PAM) in combination with erythromycin and linezolid. The FICI represents the ΣFIC of each drug. The FIC for each drug was determined by dividing the MIC of each drug in combination, by the MIC of each drug alone. ΣFIC=FIC_{A}+FIC_{B}=(C_{A}/MIC_{A})+(C_{B}/MIC_{B}). Synergy (FICI<0.5), additive (FICI>0.5-1.0), indifferent (FICI>1.0-2.0), and antagonistic (FICI>2.0), as highlighted on each bar chart. Related to Tables 18A, 18B, 19A and 19B.

FIG. 5D shows EKO-35 and the efflux platform can be used to assess efflux pump inhibitor specificities and efflux pump interplay. Bar charts depicting fractional inhibitory concentration index (FICI) values of 1-(1-naphthylmethyl)-piperazine (NMP) in combination with oxacillin and ethidium bromide. The FICI represents the ΣFIC of each drug. The FIC for each drug was determined by dividing the MIC of each drug in combination, by the MIC of each drug alone. ΣFIC=FIC_{A}+FIC_{B}=(C_{A}/MIC_{A})+(C_{B}/MIC_{B}). Synergy (FICI<0.5), additive (FICI>0.5-1.0), indifferent (FICI>1.0-2.0), and antagonistic (FICI>2.0), as highlighted on each bar chart. Related to Tables 18A, 18B, 19A and 19B.

FIG. 5E shows EKO-35 and the efflux platform can be used to assess efflux pump inhibitor specificities and efflux pump interplay. FIG. 5E shows bar charts depicting fractional inhibitory concentration index (FICI) values of 1-(1-naphthylmethyl)-piperazine (NMP) in combination with fusidic acid and ciprofloxacin. The FICI represents the ΣFIC of each drug. The FIC for each drug was determined by dividing the MIC of each drug in combination, by the MIC of each drug alone. ΣFIC=FIC_{A}+FIC_{B}=(C_{A}/MIC_{A})+(C_{B}/MIC_{B}). Synergy (FICI<0.5), additive (FICI>0.5-1.0), indifferent (FICI>1.0-2.0), and antagonistic (FICI>2.0), as highlighted on each bar chart. Related to Tables 18A, 18B, 19A and 19B.

FIG. 5F shows EKO-35 and the efflux platform can be used to assess efflux pump inhibitor specificities and efflux pump interplay. Bar charts depicting fractional inhibitory concentration index (FICI) values of 1-(1-naphthylmethyl)-piperazine (NMP) in combination with erythromycin and linezolid. The FICI represents the ΣFIC of each drug. The FIC for each drug was determined by dividing the MIC of each drug in combination, by the MIC of each drug alone. ΣFIC=FIC_{A}+FIC_{B}=(C_{A}/MIC_{A})+(C_{B}/MIC_{B}). Synergy (FICI<0.5), additive (FICI>0.5-1.0), indifferent (FICI>1.0-2.0), and antagonistic (FICI>2.0), as highlighted on each bar chart. Related to Tables 18A, 18B, 19A and 19B.

FIG. 5G shows EKO-35 and the efflux platform can be used to assess efflux pump inhibitor specificities and efflux pump interplay. The efflux pump platform identified instances of interplay between pumps with acriflavine (EKO-35). All integrated strains were transformed with the empty vector (pGDP-2). Genes encoding AcrB, AcrEF, AcrD, and MdtEF were integrated into the arabinose operon (araC) of EKO-35. Related to Table 20. Data represent mean MIC values for which the OD₆₀₀ₙₘ value >0.100, ±s.d. of three independent biological replicates. P-values were calculated by a two-tailed Student's t-test (*P<0.05, **P<0.01, ***P<0.001, ****P<0.0001).

FIG. 5H shows EKO-35 and the efflux platform can be used to assess efflux pump inhibitor specificities and efflux pump interplay. Interplay was maintained in porinated EKO-35 (EKO-35-Pore) with acriflavine, pGDP-2 harboring emrE is denoted as pEmrE. All integrated strains were transformed with the empty vector (pGDP-2). Genes encoding AcrB, AcrEF, AcrD, and MdtEF were integrated into the arabinose operon (araC) of EKO-35. Related to Table 20. Data represent mean MIC values for which the OD₆₀₀ₙₘ value >0.100, ±s.d. of three independent biological replicates. P-values were calculated by a two-tailed Student's t-test (*P<0.05, **P<0.01, ***P<0.001, ****p<0.0001).

FIG. 5I shows EKO-35 and the efflux platform can be used to assess efflux pump inhibitor specificities and efflux pump interplay. The efflux pump platform identified instances of interplay between pumps with ethidium bromide (EKO-35). All integrated strains were transformed with the empty vector (pGDP-2). Genes encoding AcrB, AcrEF, AcrD, and MdtEF were integrated into the arabinose operon (araC) of EKO-35. Related to Table 20. Data represent mean MIC values for which the OD₆₀₀ₙₘ value >0.100, ±s.d. of three independent biological replicates. P-values were calculated by a two-tailed Student's t-test (*P<0.05, **P<0.01, ***P<0.001, ****p<0.0001).

FIG. 5J shows EKO-35 and the efflux platform can be used to assess efflux pump inhibitor specificities and efflux pump interplay. Interplay was maintained in porinated EKO-35 (EKO-35-Pore) with ethidium bromide, pGDP-2 harboring emrE is denoted as pEmrE All integrated strains were transformed with the empty vector (pGDP-2). Genes encoding AcrB, AcrEF, AcrD, and MdtEF were integrated into the arabinose operon (araC) of EKO-35. Related to Table 20. Data represent mean MIC values for which the OD₆₀₀ₙₘ value >0.100, ±s.d. of three independent biological replicates. P-values were calculated by a two-tailed Student's t-test (*P<0.05, **P<0.01, ***P<0.001, ****P<0.0001).

FIG. 6 shows genotype of EKO-35 as determined by next-generation Illumine sequencing. Deleted and/or inactivated efflux-encoding genes are shown as black arrows. Secondary genomic mutations incurred in non-efflux encoding genes are shown as white arrows.

FIG. 7 shows susceptibility testing to confirm the AcrB_{D408A} mutant (EKO-35 araC::acrB_{D408A}) is inactive despite being produced in equivalent amounts to AcrB (FIG. 1A) when the genes were integrated into the arabinose operon (araC gene) of EKO-35, with gene expression under the control of the constitutive P_{LacI} promoter. Data points represent the mean of four technical replicates ±s.d.

FIG. 8A shows profiling EKO-35 with ASKA constructs expressing pitA in nutrient-rich Lysogeny broth (left) and M9 minimal glucose medium (right). Solid symbols represent empty vector controls while open symbols represent ASKA expressing constructs. Plasmids were induced with 0.1 mM IPTG and plasmid selection for EKO-35 was achieved using 1 μg/mL in M9 minimal glucose medium and 4 μg/mL in Lysogeny broth. Data represent mean values of three independent biological replicates.

FIG. 8B shows profiling EKO-35 with ASKA constructs expressing yjfC in nutrient-rich Lysogeny broth (left) and M9 minimal glucose medium (right). Solid symbols represent empty vector controls while open symbols represent ASKA expressing constructs. Plasmids were induced with 0.1 mM IPTG and plasmid selection for EKO-35 was achieved using 1 μg/mL in M9 minimal glucose medium and 4 μg/mL in Lysogeny broth. Data represent mean values of three independent biological replicates.

FIG. 8C shows profiling EKO-35 with ASKA constructs expressing tufA in nutrient-rich Lysogeny broth (left) and M9 minimal glucose medium (right). Solid symbols represent empty vector controls while open symbols represent ASKA expressing constructs. Plasmids were induced with 0.1 mM IPTG and plasmid selection for EKO-35 was achieved using 1 μg/mL in M9 minimal glucose medium and 4 μg/mL in Lysogeny broth. Data represent mean values of three independent biological replicates.

FIG. 8D shows profiling EKO-35 with ASKA constructs expressing rspA in nutrient-rich Lysogeny broth (left) and M9 minimal glucose medium (right). Solid symbols represent empty vector controls while open symbols represent ASKA expressing constructs. Plasmids were induced with 0.1 mM IPTG and plasmid selection for EKO-35 was achieved using 1 μg/mL in M9 minimal glucose medium and 4 μg/mL in Lysogeny broth. Data represent mean values of three independent biological replicates.

FIG. 8E shows profiling EKO-35 with ASKA constructs expressing wcaC in nutrient-rich Lysogeny broth (left) and M9 minimal glucose medium (right). Solid symbols represent empty vector controls while open symbols represent ASKA expressing constructs. Plasmids were induced with 0.1 mM IPTG and plasmid selection for EKO-35 was achieved using 1 μg/mL in M9 minimal glucose medium and 4 μg/mL in Lysogeny broth. Data represent mean values of three independent biological replicates.

FIG. 8F shows profiling EKO-35 with ASKA constructs expressing gyrB in nutrient-rich Lysogeny broth (left) and M9 minimal glucose medium (right). Solid symbols represent empty vector controls while open symbols represent ASKA expressing constructs. Plasmids were induced with 0.1 mM IPTG and plasmid selection for EKO-35 was achieved using 1 μg/mL in M9 minimal glucose medium and 4 μg/mL in Lysogeny broth. Data represent mean values of three independent biological replicates.

FIG. 9A shows the growth of EKO-35 at acidic and alkaline pH is not restored through plasmid-based complementation of genes in EKO-35 harboring nonsynonymous genomic mutations. EKO-35 growth is significantly reduced at pH 5.0.

FIG. 9B shows the growth of EKO-35 at acidic and alkaline pH is not restored through plasmid-based complementation of genes in EKO-35 harboring nonsynonymous genomic mutations. EKO-35 growth is significantly reduced at pH 5.5. ASKA plasmids were induced with 0.1 mM IPTG and plasmid selection for EKO-35 was achieved using 1 μg/mL chloramphenicol. Data represent mean values±s.d. of three independent biological end-point readings after 18 h of growth. P-values were calculated by a two-tailed Student's t-test (****P<0.0001) relative to EKO-35 pCA24N,

FIG. 9C shows the growth of EKO-35 at acidic and alkaline pH is not restored through plasmid-based complementation of genes in EKO-35 harboring nonsynonymous genomic mutations. EKO-35 growth is significantly reduced at pH 8.5. ASKA plasmids were induced with 0.1 mM IPTG and plasmid selection for EKO-35 was achieved using 1 μg/mL chloramphenicol. Data represent mean values±s.d. of three independent biological end-point readings after 18 h of growth. P-values were calculated by a two-tailed Student's t-test (****P<0.0001) relative to EKO-35 pCA24N,

FIG. 9D shows the growth of EKO-35 at acidic and alkaline pH is not restored through plasmid-based complementation of genes in EKO-35 harboring nonsynonymous genomic mutations. EKO-35 growth is significantly reduced at pH 9.0. ASKA plasmids were induced with 0.1 mM IPTG and plasmid selection for EKO-35 was achieved using 1 μg/mL chloramphenicol. Data represent mean values±s.d. of three independent biological end-point readings after 18 h of growth. P-values were calculated by a two-tailed Student's t-test (****P<0.0001) relative to EKO-35 pCA24N,

FIG. 10A shows plasmid-based complementation of genes in EKO-35 harboring genomic mutations alters biofilm formation in wild-type E. coli. Expression of rspA significantly increased biofilm formation in wild-type E. coli, whilst expression of pitA, tufA, gyrB, and yjfC significantly lowers biofilm formation. ASKA plasmids were induced using 0.1 mM IPTG and plasmid selection for EKO-35 was achieved using 1 μg/mL chloramphenicol in M9 minimal glucose medium and 4 μg/mL in Lysogeny broth. Data represent mean values±s.d. of three independent biological end-point readings after 24 h and 48 h of growth in nutrient-rich and -limited media, respectively. P-values were calculated by a two-tailed Student's t-test (****P<0.0001).

FIG. 10B shows plasmid-based complementation of genes in EKO-35 harboring genomic mutations alters biofilm formation in EKO-35. Expression of rspA significantly increased biofilm formation in EKO-35 in nutrient-rich Lysogeny broth, whilst expression of pitA, tufA, gyrB, and yjfC significantly lowers biofilm formation. ASKA plasmids were induced using 0.1 mM IPTG and plasmid selection for EKO-35 was achieved using 1 μg/mL chloramphenicol in M9 minimal glucose medium and 4 μg/mL in Lysogeny broth. Data represent mean values±s.d. of three independent biological end-point readings after 24 h and 48 h of growth in nutrient-rich and -limited media, respectively. P-values were calculated by a two-tailed Student's t-test (****P<0.0001).

FIG. 11A shows profiling of EKO-35 in low oxygen environments with ASKA constructs expressing pitA in nutrient-rich Lysogeny broth with 10 mM KNO₃ (1% oxygen) (left) and M9 minimal glucose medium (5% oxygen) (right). Solid symbols represent empty vector controls while open symbols represent ASKA expressing constructs. Plasmids were induced with 0.1 mM IPTG and plasmid maintenance for EKO-35 was achieved using 1 μg/mL chloramphenicol in M9 minimal glucose medium and 4 μg/mL chloramphenicol in lysogeny broth. Data represent mean values of three independent biological replicates.

FIG. 11B shows profiling of EKO-35 in low oxygen environments with ASKA constructs expressing yjfC in nutrient-rich Lysogeny broth with 10 mM KNO₃ (1% oxygen) (left) and M9 minimal glucose medium (5% oxygen) (right). Solid symbols represent empty vector controls while open symbols represent ASKA expressing constructs. Plasmids were induced with 0.1 mM IPTG and plasmid maintenance for EKO-35 was achieved using 1 μg/mL chloramphenicol in M9 minimal glucose medium and 4 μg/mL chloramphenicol in lysogeny broth. Data represent mean values of three independent biological replicates.

FIG. 11C shows profiling of EKO-35 in low oxygen environments with ASKA constructs expressing tufA in nutrient-rich Lysogeny broth with 10 mM KNO₃ (1% oxygen) (left) and M9 minimal glucose medium (5% oxygen) (right). Solid symbols represent empty vector controls while open symbols represent ASKA expressing constructs. Plasmids were induced with 0.1 mM IPTG and plasmid maintenance for EKO-35 was achieved using 1 μg/mL chloramphenicol in M9 minimal glucose medium and 4 μg/mL chloramphenicol in lysogeny broth. Data represent mean values of three independent biological replicates.

FIG. 11D shows profiling of EKO-35 in low oxygen environments with ASKA constructs expressing rspA in nutrient-rich Lysogeny broth with 10 mM KNO₃ (1% oxygen) (left) and M9 minimal glucose medium (5% oxygen) (right). Solid symbols represent empty vector controls while open symbols represent ASKA expressing constructs. Plasmids were induced with 0.1 mM IPTG and plasmid maintenance for EKO-35 was achieved using 1 μg/mL chloramphenicol in M9 minimal glucose medium and 4 μg/mL chloramphenicol in lysogeny broth. Data represent mean values of three independent biological replicates.

FIG. 11E shows profiling of EKO-35 in low oxygen environments with ASKA constructs expressing wcaC in nutrient-rich Lysogeny broth with 10 mM KNO₃ (1% oxygen) (left) and M9 minimal glucose medium (5% oxygen) (right). Solid symbols represent empty vector controls while open symbols represent ASKA expressing constructs. Plasmids were induced with 0.1 mM IPTG and plasmid maintenance for EKO-35 was achieved using 1 μg/mL chloramphenicol in M9 minimal glucose medium and 4 μg/mL chloramphenicol in lysogeny broth. Data represent mean values of three independent biological replicates.

FIG. 11F shows profiling of EKO-35 in low oxygen environments with ASKA constructs expressing gyrB in nutrient-rich Lysogeny broth with 10 mM KNO₃ (1% oxygen) (left) and M9 minimal glucose medium (5% oxygen) (right). Solid symbols represent empty vector controls while open symbols represent ASKA expressing constructs. Plasmids were induced with 0.1 mM IPTG and plasmid maintenance for EKO-35 was achieved using 1 μg/mL chloramphenicol in M9 minimal glucose medium and 4 μg/mL chloramphenicol in lysogeny broth. Data represent mean values of three independent biological replicates.

FIG. 12 shows structures of synthetic compounds determined to be substrates for efflux. Compounds were visualized using Chem Prime 20.1 (Version 20.1.0.112). EKO-35 was susceptible to compounds 4, 5, 11, 13, 16, and 18.

FIG. 13A shows results from assessing the susceptibility of EKO-35 harboring ASKA plasmids expressing rspA, tufA, pitA, wcaC, gyrB, and yjfC. Strains were profiled against benzalkonium chloride. MICs were determined i, without (left) and with chloramphenicol (right) to maintain ASKA plasmids. All plasmids were induced with 0.1 mM IPTG. 25 μg/mL and 4 μg/mL chloramphenicol was used to maintain ASKA plasmids in wild-type E. coli and EKO-35, respectively. Strains were assessed in technical duplicate.

FIG. 13B shows results from assessing the susceptibility of EKO-35 harboring ASKA plasmids expressing rspA, tufA, pitA, wcaC, gyrB, and yjfC. Strains were profiled against novobiocin. MICs were determined without (left) and with chloramphenicol (right) to maintain ASKA plasmids. All plasmids were induced with mM IPTG. 25 μg/mL and 4 μg/mL chloramphenicol was used to maintain ASKA plasmids in wild-type E. coli and EKO-35, respectively. Strains were assessed in technical duplicate.

FIG. 13C shows results from assessing the susceptibility of EKO-35 harboring ASKA plasmids expressing rspA, tufA, pitA, wcaC, gyrB, and yjfC. Strains were profiled against trimethoprim. MICs were determined without (left) and with chloramphenicol to maintain ASKA plasmids. All plasmids were induced with 0.1 mM IPTG. 25 μg/mL and 4 μg/mL chloramphenicol was used to maintain ASKA plasmids in wild-type E. coli and EKO-35, respectively. Strains were assessed in technical duplicate.

FIG. 13D shows results from assessing the susceptibility of EKO-35 harboring ASKA plasmids expressing rspA, tufA, pitA, wcaC, gyrB, and yjfC. Strains were profiled against synthetic 2. MICs were determined without (left) and with chloramphenicol (right) to maintain ASKA plasmids. All plasmids were induced with mM IPTG. 25 μg/mL and 4 μg/mL chloramphenicol was used to maintain ASKA plasmids in wild-type E. coli and EKO-35, respectively. Strains were assessed in technical duplicate.

FIG. 13E shows results from assessing the susceptibility of EKO-35 harboring ASKA plasmids expressing rspA, tufA, pitA, wcaC, gyrB, and yjfC. Strains were profiled against synthetic 14. MICs were determined without (left) and with chloramphenicol (right) to maintain ASKA plasmids. All plasmids were induced with mM IPTG. 25 μg/mL and 4 μg/mL chloramphenicol was used to maintain ASKA plasmids in wild-type E. coli and EKO-35, respectively. Strains were assessed in technical duplicate.

FIG. 13F shows results from assessing the susceptibility of EKO-35 harboring ASKA plasmids expressing rspA, tufA, pitA, wcaC, gyrB, and yjfC. Strains were profiled against synthetic 19. MICs were determined without (left) and with chloramphenicol (right) to maintain ASKA plasmids. All plasmids were induced with mM IPTG. 25 μg/mL and 4 μg/mL chloramphenicol was used to maintain ASKA plasmids in wild-type E. coli and EKO-35, respectively. Strains were assessed in technical duplicate.

FIG. 14A shows heat maps depicting susceptibility levels of strains expressing efflux pump-encoding genes. Each strain was tested in technical duplicate and MIC values were normalized to 100% for each compound tested, where dark gray represents the highest MIC value, and white represents the lowest value (see key). Genes encoding AcrEF, were chromosomally integrated into the wild-type strain, a single gene deletion mutant, and EKO-35. Susceptibility testing revealed no changes in the resistance levels of the wild-type or single gene deletion backgrounds, except for ΔacrB (FIG. 14C). EKO-35 was highly susceptible to all compounds tested, revealing susceptibility profiles similar to ΔtolC, and integration of efflux genes into this strain conferred resistance to the known substrates.

FIG. 14B shows heat maps depicting susceptibility levels of strains expressing efflux pump-encoding genes. Each strain was tested in technical duplicate and MIC values were normalized to 100% for each compound tested, where dark gray represents the highest MIC value, and white represents the lowest value (see key). Genes encoding AcrD were chromosomally integrated into the wild-type strain, a single gene deletion mutant, and EKO-35. Susceptibility testing revealed no changes in the resistance levels of the wild-type or single gene deletion backgrounds, except for ΔacrB (FIG. 14C). EKO-35 was highly susceptible to all compounds tested, revealing susceptibility profiles similar to ΔtolC, and integration of efflux genes into this strain conferred resistance to the known substrates.

FIG. 14C shows heat maps depicting susceptibility levels of strains expressing efflux pump-encoding genes. Each strain was tested in technical duplicate and MIC values were normalized to 100% for each compound tested, where dark gray represents the highest MIC value, and white represents the lowest value (see key). Genes encoding AcrB were chromosomally integrated into the wild-type strain, a single gene deletion mutant, and EKO-35. EKO-35 was highly susceptible to all compounds tested, revealing susceptibility profiles similar to ΔtolC, and integration of efflux genes into this strain conferred resistance to the known substrates.

FIG. 14D shows heat maps depicting susceptibility levels of strains expressing efflux pump-encoding genes. Each strain was tested in technical duplicate and MIC values were normalized to 100% for each compound tested, where dark gray represents the highest MIC value, and white represents the lowest value (see key). Genes encoding MdtEF were chromosomally integrated into the wild-type strain, a single gene deletion mutant, and EKO-35. Susceptibility testing revealed no changes in the resistance levels of the wild-type or single gene deletion backgrounds, except for ΔacrB (FIG. 14C). EKO-35 was highly susceptible to all compounds tested, revealing susceptibility profiles similar to ΔtolC, and integration of efflux genes into this strain conferred resistance to the known substrates.

FIG. 14E shows heat maps depicting susceptibility levels of strains expressing efflux pump-encoding genes. Each strain was tested in technical duplicate and MIC values were normalized to 100% for each compound tested, where dark gray represents the highest MIC value, and white represents the lowest value (see key). Genes encoding MdtBC were chromosomally integrated into the wild-type strain, a single gene deletion mutant, and EKO-35. Susceptibility testing revealed no changes in the resistance levels of the wild-type or single gene deletion backgrounds, except for ΔacrB (FIG. 14C). EKO-35 was highly susceptible to all compounds tested, revealing susceptibility profiles similar to ΔtolC, and integration of efflux genes into this strain conferred resistance to the known substrates.

FIG. 14F shows heat maps depicting susceptibility levels of strains expressing efflux pump-encoding genes. Each strain was tested in technical duplicate and MIC values were normalized to 100% for each compound tested, where dark gray represents the highest MIC value, and white represents the lowest value (see key). Genes encoding MacAB were chromosomally integrated into the wild-type strain, a single gene deletion mutant, and EKO-35. Susceptibility testing revealed no changes in the resistance levels of the wild-type or single gene deletion backgrounds, except for ΔacrB (FIG. 14C). EKO-35 was highly susceptible to all compounds tested, revealing susceptibility profiles similar to ΔtolC, and integration of efflux genes into this strain conferred resistance to the known substrates.

FIG. 14G shows heat maps depicting susceptibility levels of strains expressing efflux pump-encoding genes. Each strain was tested in technical duplicate and MIC values were normalized to 100% for each compound tested, where dark gray represents the highest MIC value, and white represents the lowest value (see key). Genes encoding MacAB were chromosomally integrated into the wild-type strain, a single gene deletion mutant, and EKO-35. Susceptibility testing revealed no changes in the resistance levels of the wild-type or single gene deletion backgrounds, except for ΔacrB (FIG. 14C). EKO-35 was highly susceptible to all compounds tested, revealing susceptibility profiles similar to ΔtolC, and integration of efflux genes into this strain conferred resistance to the known substrates.

FIG. 14H shows heat maps depicting susceptibility levels of strains expressing efflux pump-encoding genes. Each strain was tested in technical duplicate and MIC values were normalized to 100% for each compound tested, where dark gray represents the highest MIC value, and white represents the lowest value (see key). Genes encoding MexCD were chromosomally integrated into the wild-type strain, a single gene deletion mutant, and EKO-35. Susceptibility testing revealed no changes in the resistance levels of the wild-type or single gene deletion backgrounds, except for ΔacrB (FIG. 14C). EKO-35 was highly susceptible to all compounds tested, revealing susceptibility profiles similar to ΔtolC, and integration of efflux genes into this strain conferred resistance to the known substrates.

FIG. 15A shows susceptibility and growth profiling of porinated wild-type (WT) K-12, ΔtolC, and EKO-35 strains. FIG. 15A shows heatmap depicting vancomycin susceptibility of the porinated strains. Each strain was tested in technical duplicate and MIC values were normalized to 100%, where dark gray represents the highest MIC value, and white represents the lowest value (see key). Susceptibility of the WT K-12, ΔtolC, and EKO-35 strains+/−the pore was assessed for 52 compounds.

FIG. 15B shows susceptibility and growth profiling of porinated wild-type (WT) K-12, ΔtolC, and EKO-35 strains. FIG. 15B shows growth profiling of WT K-12-Pore and EKO-35-Pore in LB at 37° C. Measurement of growth kinetics revealed the pore did not significantly impact the length of the lag phase (P>0.05) or generation time (P>0.05) compared to the parental strains, which was assessed using three biological replicates. Susceptibility of the WT K-12, ΔtolC, and EKO-35 strains+/−the pore was assessed for 52 compounds.

FIG. 15C shows molecular weight for each compound that resulted in a 4-fold or greater increase in susceptibility compared to the wild-type strain presented as individual data points in the box plots, the line through the center of each box indicates the median, and whiskers the minimum and maximum values. Molecular weights (MW) were calculated using DataWarrior (Version 5.5.0).

FIG. 15D shows lipophilicity for each compound that resulted in a 4-fold or greater increase in susceptibility compared to the wild-type strain presented as individual data points in the box plots, the line through the center of each box indicates the median, and whiskers the minimum and maximum values. Lipophilicities (log P) were calculated using DataWarrior (Version 5.5.0).

FIG. 15E shows aqueous solubility for each compound that resulted in a 4-fold or greater increase in susceptibility compared to the wild-type strain presented as individual data points in the box plots, the line through the center of each box indicates the median, and whiskers the minimum and maximum values. Aqueous solubilities (log S) were calculated using DataWarrior (Version 5.5.0).

FIG. 15F shows polar surface area for each compound that resulted in a 4-fold or greater increase in susceptibility compared to the wild-type strain presented as individual data points in the box plots, the line through the center of each box indicates the median, and whiskers the minimum and maximum values. Polar Surface Areas (PSA) were calculated using DataWarrior (Version 5.5.0).

FIG. 15G shows physicochemical properties calculated using DataWarrior (Version 5.5.0). FIG. 15G shows summary of the physicochemical properties ranges for each strain. Medians are indicated in parentheses.

FIG. 16A shows heat maps depicting aminoglycoside susceptibility of the wild-type K-12 strain and an ΔacrD mutant expressing the chromosomally integrated acrD gene (araC::acrD) using kanamycin in cation-adjusted Mueller Hinton II Broth (MHB II). The cell inoculum used was 10⁴ cells/mL to replicate susceptibility testing conditions used in previous studies. Each strain was assessed using three technical replicates and MIC values were normalized to 100% for each compound tested, where dark gray represents the highest MIC value, and white represents the lowest value (see key).

FIG. 16B shows heat maps depicting aminoglycoside susceptibility of the wild-type K-12 strain and an ΔacrD mutant expressing the chromosomally integrated acrD gene (araC::acrD) using kanamycin in Lysogeny broth (LB). The cell inoculum used was 10⁴ cells/mL to replicate susceptibility testing conditions used in previous studies. Each strain was assessed using three technical replicates and MIC values were normalized to 100% for each compound tested, where dark gray represents the highest MIC value, and white represents the lowest value (see key).

FIG. 16C shows heat maps depicting aminoglycoside susceptibility of the wild-type K-12 strain and an ΔacrD mutant expressing the chromosomally integrated acrD gene (araC::acrD) using gentamicin in MHB II. The cell inoculum used was 10⁴ cells/mL to replicate susceptibility testing conditions used in previous studies. Each strain was assessed using three technical replicates and MIC values were normalized to 100% for each compound tested, where dark gray represents the highest MIC value, and white represents the lowest value (see key).

FIG. 16D shows heat maps depicting aminoglycoside susceptibility of the wild-type K-12 strain and an ΔacrD mutant expressing the chromosomally integrated acrD gene (araC::acrD) using gentamicin in LB. The cell inoculum used was 10⁴ cells/mL to replicate susceptibility testing conditions used in previous studies. Each strain was assessed using three technical replicates and MIC values were normalized to 100% for each compound tested, where dark gray represents the highest MIC value, and white represents the lowest value (see key).

FIG. 17A shows EKO-35 and the efflux platform can be used to assess efflux pump interplay. Interplay was not observed for novobiocin in EKO-35. pGDP-2 harboring emrE is denoted as pEmrE. All integrated strains were transformed with the empty vector (pGDP-2). Genes encoding AcrB, AcrEF, AcrD, and MdtEF were integrated into the arabinose operon (araC) of EKO-35. Related to Table 20. Data represent mean MIC values for which the OD₆₀₀ₙₘ value >0.100, ±s.d. of three independent biological replicates. P-values were calculated by a two-tailed Student's t-test (*P<0.05, **P<0.01, ***P<0.001, ****P<0.0001).

FIG. 17B shows EKO-35 and the efflux platform can be used to assess efflux pump interplay. Interplay was not observed for novobiocin in EKO-35-Pore pGDP-2 harboring emrE is denoted as pEmrE. All integrated strains were transformed with the empty vector (pGDP-2). Genes encoding AcrB, AcrEF, AcrD, and MdtEF were integrated into the arabinose operon (araC) of EKO-35. Related to Table 20. Data represent mean MIC values for which the OD₆₀₀ₙₘ value >0.100, ±s.d. of three independent biological replicates. P-values were calculated by a two-tailed Student's t-test (*P<0.05, **P<0.01, ***P<0.001, ****P<0.0001).

FIG. 17C shows EKO-35 and the efflux platform can be used to assess efflux pump interplay. Interplay was not observed for novobiocin in EKO-35. pGDP-2 harboring emrE is denoted as pEmrE. All integrated strains were transformed with the empty vector (pGDP-2). Genes encoding AcrB, AcrEF, AcrD, and MdtEF were integrated into the arabinose operon (araC) of EKO-35. Related to Table 20. Data represent mean MIC values for which the OD₆₀₀ₙₘ value >0.100, ±s.d. of three independent biological replicates. P-values were calculated by a two-tailed Student's t-test (*P<0.05, **P<0.01, ***P<0.001, ****P<0.0001).

FIG. 17D shows EKO-35 and the efflux platform can be used to assess efflux pump interplay. Interplay was not observed for novobiocin in EKO-35-Pore. pGDP-2 harboring emrE is denoted as pEmrE. All integrated strains were transformed with the empty vector (pGDP-2). Genes encoding AcrB, AcrEF, AcrD, and MdtEF were integrated into the arabinose operon (araC) of EKO-35. Related to Table 20. Data represent mean MIC values for which the OD₆₀₀ₙₘ value >0.100, ±s.d. of three independent biological replicates. P-values were calculated by a two-tailed Student's t-test (*P<0.05, **P<0.01, ***P<0.001, ****P<0.0001).

FIG. 18 shows a graphical depiction of K-12, EKO-35, EKO-35 Pore, EKO-35 araC::X, and EKO-35 araC::X Y:pGDP of the present disclosure.

FIG. 19 show phenotypic analysis of EKO-35v2 in nutrient-rich conditions. Measurement of growth kinetics of EKO-35v1 (Example 1) and EKO-35v2 was assessed using n=3 biological replicates (Table 25).

### DETAILED DESCRIPTION

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

As used herein, the singular forms “a”, “an” and “the” include plural references unless the content clearly dictates otherwise.

The term “nucleic acid”, “nucleic acid molecule” or its derivatives, as used herein, is intended to include unmodified DNA or RNA or modified DNA or RNA. For example, the nucleic acid molecules of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

As used herein, the term “inactivation of a gene”, or a derivative thereof, refers to reduction or elimination in the activity of the protein encoded by a gene due to the reduction or elimination of the gene expression via mutation induced by one or more methods selected from the group consisting of deletion of all or a part of the corresponding gene, substitution of a part of the nucleotide sequence, or deletion or insertion of one or more base pairs into the nucleotide sequence.

As used herein, the term “reactivation”, or a derivative thereof, when relating to a gene, refers to increase or reintroduction in the activity of the protein encoded by a gene due to the increase or reintroduction of the gene expression via mutation induced by one or more methods selected from the group consisting of insertion of all or a part of the corresponding gene, substitution of a part of the nucleotide sequence, or deletion or insertion of one or more base pairs into the nucleotide sequence. Reactivation or reactivated includes restoring the gene as prior to inactivation, with previous promoter or with a different promoter, whether it a constitutive or conditional promoter. Reactivation can include tunable expression to control the level of the restored gene, including overexpression, returning to previous level or under-expressing as compared to previous levels. The reactivation, including reintroduction, can be at the same locus or at a different locus of the bacterial strain's genome. The reactivation, including reintroduction, of gene can include introduction of mutation that affect the function of the gene, for example, efflux pump function, including interaction with compounds or other genes. In some embodiments, reactivation of a gene comprises reintroduction of a gene. In some embodiments, reactivation of a gene occurs at the same locus, or at a different locus in a bacterial strain's genome. In some embodiments, reintroduction of a gene occurs at the same locus, or at a different locus in a bacterial strain's genome.

As used herein, the term “polypeptide” encompasses both peptides and proteins, and fragments thereof of peptides and proteins, unless indicated otherwise. In one embodiment, the therapeutic agent is a polypeptide.

The term “promoter,” as used herein, refers to a nucleotide sequence that directs the transcription of a gene or coding sequence to which it is operably linked.

The term “operably linked”, as used herein, refers to an arrangement of two or more components, wherein the components so described are in a relationship permitting them to function in a coordinated manner. For example, a transcriptional regulatory sequence or a promoter is operably linked to a coding sequence if the transcriptional regulatory sequence or promoter facilitates aspects of the transcription of the coding sequence. The skilled person can readily recognize aspects of the transcription process, which include, but not limited to, initiation, elongation, attenuation and termination. In general, an operably linked transcriptional regulatory sequence is joined in cis with the coding sequence, but it is not necessarily directly adjacent to it.

A “segment” of a nucleotide sequence is a sequence of contiguous nucleotides. A segment can be at least about 5, 10, 15, 20, 25, 30, 35, 40, 45, 50, 60, 85, 100, 110, 120, 130, 145, 150, 160, 175, 200, 250, 300, 350, 400, 450, 500 or more contiguous nucleotides.

A “fragment” of an amino acid sequence is a sequence of contiguous amino acids. A segment can be at least about 5, 10, 15, 20, 25, 30, 35, 40, 45, 50, 60, 85, 100, 110, 120, 130, 145, 150, 160, 175, 200, 250, 300, 350, 400, 450, 500 or more contiguous amino acids.

The term “antibacterial agent” as used herein refers to a microbial inhibiting agent, including anything that reduces virulence or modifies efflux pump activity, with or without the action of other compounds and adjuvants. For example, an antibacterial agent can be an efflux pump inhibitor.

The term “viability” as used herein refers to measurement known to the skilled person in assessing health of bacteria. Methods known in the art can be used to determine viability. Viability can be determined as a percentage over a control or as a minimal inhibitory concentration (MIC) when it is being affected by a compound, for example, an antibacterial agent such as an efflux pump inhibitor. Viability can also be determined relatively, for example, by comparing the rate of killing or inhibition of growth of the bacterial strain or wild-type bacteria described herein.

### Composition and Method of the Disclosure

The present disclosure provides an Escherichia coli strain that is deficient in efflux pump activity. Accordingly, provided herein is an Escherichia coli strain comprising at least 20, 21, 22, 23, 24, 25, 26, 27, 28, 29, 30, 31, 32, 33, 34, or of inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the strain comprises at least 20 of the inactivated genes. In some embodiments, the strain comprises at least 21 of the inactivated genes. In some embodiments, the strain comprises at least 22 of the inactivated genes. In some embodiments, the strain comprises at least 23 of the inactivated genes. In some embodiments, the strain comprises at least 24 of the inactivated genes. In some embodiments, the strain comprises at least 25 of the inactivated genes. In some embodiments, the strain comprises at least 26 of the inactivated genes. In some embodiments, the strain comprises at least 27 of the inactivated genes. In some embodiments, the strain comprises at least 28 of the inactivated genes. In some embodiments, the strain comprises at least 29 of the inactivated genes. In some embodiments, the strain comprises at least 30 of the inactivated genes. In some embodiments, the strain comprises at least 31 of the inactivated genes. In some embodiments, the strain comprises at least 32 of the inactivated genes. In some embodiments, the strain comprises at least 33 of the inactivated genes. In some embodiments, the strain comprises at least 34 of the inactivated genes. In some embodiments, strain comprises all 35 inactivated genes. EKO-35v1 and EKO-35v2 are examples of EKO-35. In some embodiments, the Escherichia coli strain is EKO-35v1. In some embodiments, the Escherichia coli strain is EKO-35v2. In some embodiments, the Escherichia coli strain is deposited under IDAC accession number 310522-01 or IDAC accession number 070623-01. In some embodiments, the Escherichia coli strain is deposited under IDAC accession number 310522-01. In some embodiments, the Escherichia coli strain is deposited under IDAC accession number 070623-01. In some embodiments, the strain further comprises an open variant of outer membrane ferric siderophore transporter FhuA. In some embodiments, the strain further comprises deletion of tolC gene. In some embodiments, the strain comprises at least one of reactivated acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, optionally under the control of a constitutive promoter. In some embodiments, one of reactivated acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the inactivation comprises deletion of the gene, or introducing a mutation into the gene to ablate expression of the gene or eliminate efflux pump activity of the protein expressed by the gene. In some embodiments, acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA are genes encoding for efflux pumps. In some embodiments, the strain comprising at least 20 of inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA is an efflux pump deficient strain. In some embodiments, the strain comprises all 35 inactivated genes is EKO-35. In some embodiments, the strain comprises a nucleic acid comprising at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.91%, 99.92%, 99.93%, 99.94%, 99.95%, 99.96%, 99.97%, 99.98%, 99.99%, 99.999%, or 99.9999% sequence identity as any nucleotide sequence described herein. In some embodiments, the strain comprises a nucleic acid comprising 100% sequence identity as any nucleotide sequence described herein. In some embodiments, the strain comprises a nucleic acid comprising at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.91%, 99.92%, 99.93%, 99.94%, 99.95%, 99.96%, 99.97%, 99.98%, 99.99%, 99.999%, or 99.9999% sequence identity as the nucleotide sequence as shown in SEQ ID NO: 255. In some embodiments, the strain comprises a nucleic acid comprising at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.91%, 99.92%, 99.93%, 99.94%, 99.95%, 99.96%, 99.97%, 99.98%, 99.99%, 99.999%, or 99.9999% sequence identity as the nucleotide sequence as shown in SEQ ID NO: 255, and having least 20, 21, 22, 23, 24, 25, 26, 27, 28, 29, 30, 31, 32, 33, 34, or 35 of inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the strain comprises a nucleic acid comprising 100% sequence identity as the nucleotide sequence as shown in SEQ ID NO: 255.

The molecular tools for inactivating and reactivating genes are known in the art. In some embodiments, the reactivation of gene comprises reintroducing gene under control of a constitutive promoter.

Also provided is a method for identifying a compound that is an antibacterial agent, comprising
(a) i) contacting the compound with the Escherichia coli strain described herein and with wild-type Escherichia coli; and/or
ii) contacting the compound with the Escherichia coli strain described herein having reactivated genes and EKO-35; and/or
iii) contacting the compound with the Escherichia coli strain described herein having reactivated genes and an efflux pump deficiency strain described herein; and
(b) detecting viability of each of the Escherichia coli;
wherein the compound is identified as an antibacterial agent if the compound decreases viability of wild-type less than the Escherichia coli strain with efflux pump deficiency described herein;
optionally wherein the compound is identified as an antibacterial agent if the compound decreases viability of the Escherichia coli strain having reactivated efflux pump genes less than the Escherichia coli strain having at least 20 inactivated efflux pump genes;
optionally wherein the compound is identified as an antibacterial agent if the wild-type Escherichia coli or the Escherichia coli strain having reactivated efflux pump genes is resistant to the compound, and the compound decreases the viability of the Escherichia coli strain having at least 20 inactivated efflux pump genes, or EKO-35.

In some embodiments, the antibacterial agent is a microbial inhibiting agent that reduces virulence or modifies efflux pump activity, with or without the action of other compounds and adjuvants. In some embodiments, the antibacterial agent is an efflux pump inhibitor.

In some embodiments, the decrease in viability of wild-type or the Escherichia coli strain described herein after contacting the compound is at most 10%, 20%, 30%, 40%, 50%, 60%, 70%, or 80%, or at least 20%, 30%, 40%, 50%, 60%, 70%, 80%, 90%, or 100%.

In some embodiments, the compound is identified as an antibacterial agent if the compound decreases the viability of efflux pump deficient Escherichia coli strain described herein at a faster rate than the decrease in viability of wild-type or the Escherichia coli strain comprising reactivated efflux pump genes.

In some embodiments, the compound decreases the viability of the Escherichia coli strain with one reactivated gene in an efflux deficient strain disclosed herein, optionally Escherichia coli strain EKO-35, is less than the efflux deficient strain disclosed herein, optionally Escherichia coli strain EKO-35, identifying specificity of the compound for an efflux pump encoded by the reactivated gene, optionally the compound has a lower minimum inhibitory concentration (MIC) in the Escherichia coli strain of EKO-35 than EKO-35 with a reactivated efflux pump.

The skilled person recognizes optimal conditions to carry out methods for identifying antibacterial agent or growth condition for the E. coli strain described herein. For example, optimal aeration can include broth cultures grown with aeration at 220 rpm. For example, for growth profiling, microtiter plates can be incubated at 37° C. or 25° C. with continuous linear shaking at 600 rpm. Other optimal conditions, as well as nutrient-limited conditions, are described in the Example.

In some embodiments, the contacting comprises the Escherichia coli in a suitable culturing media for optimal Escherichia coli growth. In some embodiments, the contacting comprises incubating the Escherichia coli in nutrient-limiting culturing media. In some embodiments, the contacting comprises incubating the Escherichia coli for at least 24 h, 48 h, 72 h, or 96 h. In some embodiments, the culturing media is a media having a pH of about 2, about 3, about 4, or about 5.

Also provided is a method for identifying a compound that is an antibacterial agent, comprising
(a) i) contacting the compound with an Escherichia coli strain of comprising at least 20 of inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA (Strain A), or the Escherichia coli strain deposited under IDAC accession number 310522-01 or IDAC accession number 070623-01, or an Escherichia coli comprising a nucleic acid having the sequence as shown in SEQ ID NO: 255, and with wild-type Escherichia coli; and/or
ii) contacting the compound with Strain A or the Escherichia coli strain deposited under IDAC accession number 310522-01 or IDAC accession number 070623-01, or an Escherichia coli comprising a nucleic acid having the sequence as shown in SEQ ID NO: 255, and an Escherichia coli strain comprising inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, wherein at least one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, is reactivated (Strain B); and/or
iii) contacting the compound with Strain A or the Escherichia coli strain deposited under IDAC accession number 310522-01 or IDAC accession number 070623-01, or an Escherichia coli comprising a nucleic acid having the sequence as shown in SEQ ID NO: 255, and an Escherichia coli strain comprising inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, wherein one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, is reactivated (Strain C); and
(b) detecting viability of each of the Escherichia coli;
wherein the compound is identified as an antibacterial agent if the compound decreases viability of wild-type less than Strain A or the Escherichia coli strain deposited under IDAC accession number 310522-01 or IDAC accession number 070623-01, or comprising or an Escherichia coli a nucleic acid having the sequence as shown in SEQ ID NO: 255;
optionally wherein the compound is identified as an antibacterial agent if the compound decreases viability of Strain B, less than Strain A or the Escherichia coli strain deposited under IDAC accession number 310522-01 or IDAC accession number 070623-01, or an Escherichia coli comprising a nucleic acid having the sequence as shown in SEQ ID NO: 255;
optionally wherein the compound is identified as an antibacterial agent if the wild-type Escherichia coli or Strain B is resistant to the compound, and the compound decreases the viability of Strain A or the Escherichia coli strain deposited under IDAC accession number 310522-01 or IDAC accession number 070623-01, or an Escherichia coli comprising a nucleic acid having the sequence as shown in SEQ ID NO: 255.

Also provided is a method for creating an Escherichia coli strain producing one or more efflux pump, comprising reactivating one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA in the Escherichia coli strain EKO-35.

In some embodiments, the reactivation comprises introducing one or more of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, optionally by reversing the inactivation, optionally by re-introducing the gene back in genome with the same promoter or a different promoter, at the same locus or a different locus, optionally by introducing a knock down-resistant version of the gene.

Also provided is a method for creating an efflux pump deficiency E. coli strain, the method comprises inactivating at least 20, 21, 22, 23, 24, 25, 26, 27, 28, 29, 30, 31, 32, 33, 34, or 35 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. mdlA, emrY, mdfA, fsr, mdtG, mdtH, yieO, mdlB, mdtO, yojI, yajR, ydhC, and cusA. In some embodiments, the method comprises inactivating at least 20 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 21 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 22 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 23 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 24 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 25 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 26 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 27 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 28 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 29 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 30 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 31 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 32 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 33 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating at least 34 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating all 35 genes of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA. In some embodiments, the method comprises inactivating genes in the following order: ΔyajR; mdtO; ydhC; emrE; yojI; mdtD, sugE; ynfM, emrD, ydeF; mdlA, emrY; mdtK, bcr; mdtG; mdtH, mdlB, macB, yddA; fsr; ydiM; yieO; mdfA; mdtM; mdtJ; emrB, mdtB, mdtL; yebQ, cusA; mdtF; ydeA; acrF; acrD, and acrB.

### EXAMPLES

The following non-limiting Examples are illustrative of the present disclosure:

### Example 1. Development and Utilization of EKO-35

### Materials and Methods

### Strains, Plasmids, and Growth Conditions

Bacterial strains and plasmids used in this disclosure are provided in Table 2. E. coli K-12 str. BW25113, the parental strain of the Keio Collection (Baba, T. et al., 2006) was used as the background for generation of EKO-35. Specifically, an ΔacrB mutant from the Keio Collection was used as the first deletion mutant. E. coli TOP10 or E. coli DH5a strains were used as routine cloning hosts. E. coli strains for resistance cassette amplification were obtained from the Keio Collection, P. aeruginosa PAO1 was provided by Dr. Cezar Khursigara (University of Guelph). An E. coli K-12 str. BW25113 harboring the fhuA ΔC/Δ4L gene under the control of the constitutive synthetic promoter BBa_J23104 was used as a source for the ‘Pore’ (Johnson, J. W. et al., 2022). Plasmids for CRISPR-Cas9 mediated counterselection, pCas and pTargetF, were purchased from Addgene (Jiang, Y. et al., 2015). Plasmids for the λ-Red recombinase system, pKD46 and pCP20 (Datsenko, K. A. & Wanner, B. L, 2000), and expression of efflux genes, pINT2 and pGDP2 were used (Cox, G. et al., 2017). Strains were routinely grown in Lysogeny broth (LB) (Bioshop) at 37° C. or For optimal aeration, broth cultures were grown with aeration at 220 rpm. For growth profiling, microtiter plates were incubated at 37° C. or 25° C. with continuous linear shaking at 600 rpm. For susceptibility testing, microtiter plates were grown at 37° C. with continuous linear shaking at 900 rpm. Ampicillin (100 μg/mL) (Bioshop), kanamycin (50 μg/mL) (Sigma-Aldrich), spectinomycin (50 μg/mL) (Bioshop), and gentamicin (10 μg/mL) (BioBasic) were used at the listed concentrations for selection of resistance markers. For nutrient-limited conditions, strains were grown in M9 (Bioshop) supplemented with 2 mM MgSO4 (Bioshop), 0.1 mM CaCl₂) (Bioshop) and glucose (w/v) (Bioshop). To profile growth in amino acid-limited medium supplemented with iron, MOPS medium (TEKNOVA) was utilized. Buffered “low-salt” medium was prepared (Lewinson, O. et al., 2014) with 100 mM of the appropriate buffer [pH 5.0, homopiperazine-N,N=-bis-2-(ethanesulfonic acid) (HOMOPIPES), pH 5.5 to 6.5, 2-(N-morpholino)ethanesulfonic acid (MES); pH 7.0 to 7.5, 3-(N-morpholino)propanesulfonic acid (MOPS)]. For profiling growth at pH 8.0 to 9.0, 1,3-bis(tris(hydroxymethyl)methylamino)propane (Bis-tris propane) was used at 50 mM due to toxicity at higher concentrations. Susceptibility testing was conducted in cation-adjusted Mueller Hinton II Broth (MHB II) (BD Difco).

TABLE 1
Measurement of growth kinetics revealed statistically significant
differences between EKO-35 and the wild-type strain. Generation
time and the duration of the lag phase for each strain are
shown in minutes. The measurements represent the mean +
standard deviation for three biological replicates.

| | | | |
|---|---|---|---|
| | Generation | Lag | |
| Strain | time (min) | phase (min) | Final OD600nm |

Nutrient-rich medium at 37° C.

| | | | |
|---|---|---|---|
| K-12 | 26.54 ± 0.454 | 204.8 ± 2.969 | 0.741 ± 0.066 |
| EKO-35 | 28.33 ± 0.911 | 265.8 ± 3.285 | 0.725 ± 0.048 |
| P-value | 4.00 × 10⁻² | 1.83 × 10⁻⁵ | 0.178* |

Nutrient-rich medium at 25° C.

| | | | |
|---|---|---|---|
| K-12 | 83.06 ± 6.06 | 592.3 ± 6.536 | 0.979 ± 0.0172 |
| EKO-35 | 171.51 ± 13.08 | 809.5 ± 4.40 | 1.082 ± 0.0029 |
| P-value | 4.44 × 10⁻⁴ | 1.16 × 10⁻⁶ | 5.13 × 10⁻⁴ |

Nutrient-rich medium at 37° C. with 1% O₂

| | | | |
|---|---|---|---|
| K-12 | 24.85 ± 1.263 | 185.0 ± 5.122 | 0.525 ± 0.009 |
| EKO-35 | 37.15 ± 0.849 | 279.7 ± 9.516 | 0.452 ± 0.008 |
| P-value | 1.51 × 10⁻⁴ | 1.10 × 10⁻⁴ | 4.98 × 10⁻⁴ |

Nutrient-rich medium with KNO₃ supplementation at 37° C. with 1% O₂

| | | | |
|---|---|---|---|
| K-12 | 26.87 ± 1.035 | 209.0 ± 2.165 | 0.502 ± 0.007 |
| EKO-35 | 43.52 ± 1.962 | 315.4 ± 4.048 | 0.293 ± 0.004 |
| EKO-35 | 30.10 ± 1.137 | 276.0 ± 6.187 | 0.331 ± 0.006 |
| araC::MdtEF | | | |
| P-value | 2.02 × 10⁻⁴ | 2.30 × 10⁻⁶ | 1.83 × 10⁻⁶ |
| ^{a}P-value | 5.09 × 10⁻⁴ | 7.70 × 10⁻⁴ | 6.47 × 10⁻⁴ |

Nutrient-limited medium at 37° C.

| | | | |
|---|---|---|---|
| K-12 | 56.5 ± 3.020 | 841.1 ± 7.401 | 0.585 ± 0.030 |
| EKO-35 | 52.05 ± 1.058 | 1137.8 ± 9.803 | 0.492 ± 0.004 |
| P-value | 0.074* | 1.95 × 10⁻⁶ | 6.13 × 10⁻³ |

Amino acid-limited medium supplemented with iron at 37° C.

| | | | |
|---|---|---|---|
| K-12 | 58.40 ± 1.779 | 848.6 ± 17.19 | 0.688 ± 0.118 |
| EKO-35 | 70.45 ± 1.875 | 1242.9 ± 29.68 | 0.818 ± 0.006 |
| P-value | 1.27 × 10⁻³ | 0.913* | 0.130* |

Nutrient-limited medium at 25° C.

| | | | |
|---|---|---|---|
| K-12 | 219.42 ± 26.09 | 2603.55 ± 64.90 | 0.591 ± 0.056 |
| EKO-35 | 231.27 ± 18.49 | 2539.27 ± 82.34 | 0.666 ± 0.173 |
| P-value | 9.18 × 10⁻² | 1.87 × 10⁻¹ | 3.41 × 10⁻¹ |

Nutrient-limited medium at 37° C. with 5% O₂

| | | | |
|---|---|---|---|
| K-12 | 80.79 ± 3.885 | 1541.2 ± 26.27 | 0.321 ± 0.002 |
| EKO-35 | 134.70 ± 2.331 | 2420.9 ± 36.30 | 0.314 ± 0.010 |
| EKO-35 | 114.94 ± 2.603 | 2133.09 ± 71.84 | 0.375 ± 0.006 |
| araC::MdtEF | | | |
| P-value | 3.27 × 10⁻⁵ | 5.09 × 10⁻⁶ | 0.29* |
| ^{a}P-value | 6.09 × 10⁻⁴ | 5.06 × 10⁻³ | 8.40 × 10⁻⁴ |

* non-significant P-values.
Statistical significance was assessed using a two-tailed Student's t-test (P-value ≤ 0.05).
^{a}P-value for EKO-35 compared to EKO-35 araC::MdtEF

TABLE 2
Strains and plasmids used in this Example.

| | |
|---|---|
| Genotype or Description | Source |

Strains

| | | |
|---|---|---|
| E. coli K-12 str. | The parental strain of the KEIO collection | Baba, T. et |
| BW25113 | | al. (2006) |
| E. coli TOP10 | Cloning host, mcrA deficient for increased | Thermo |
| | efficiency in foreign DNA uptake | Fisher |
| | | Scientific |
| E. coli DH5a | Cloning host, endA deficient for high quality | Thermo |
| | DNA preparations | Fisher |
| | | Scientific |
| E. coli EKO-35 | BW25113 efflux deficient derivative (AacrB; | This |
| | acrD; acrF; mdtF; macB; emrB; mdtL; mdtK; | disclosure |
| | bcr; ydeA; mdtM; yddA; yebQ; emrE; mdtD; | |
| | sugE; ynfM; emrD; ydeF; mdtJ; ydiM; mdtB; | |
| | mdIA; emrY; mdfA; fsr; mdtG; mdtH; yieO; | |
| | mdlB, mdtO, yojI, yajR, ydhC; cusA) | |
| E. coli δtolC | BW25113 derivative from the KEIO collection | Baba, T. et |
| | | al. (2006) |
| E. coli pore | BW25115 attTn7::mini-Tn7T (Gm^{r}-PBBa_J23104- | Johnson, J. |
| | fhuA AC/A4L) | W. et al. |
| | | (2022) |
| P. aeruginosa PAO1 | Derivative of the Australian PAO isolate | Stover, C. |
| | | K. et al. |
| | | (2000) |

Plasmids

| | | |
|---|---|---|
| pKD46 | Amp^{r}, temperature sensitive, arabinose- | Datsenko, |
| | induced expression of the A-Red recombinase | K. A. & |
| | for homologous recombination | Wanner, B. |
| | | L. (2000) |
| pCP20 | Amp^{r}, temperature sensitive: permissive | Cherepanov |
| | (30° C.), non-permissive (42° C.). Contains flp | P. P. & |
| | from Saccharomyces cerevisiae for resistance | Wackernag |
| | cassette removal | el, W. |
| | | (1995) |
| pCas | Kan^{r}, temperature sensitive permissive (30° C.), | Jiang, Y. et |
| | non-permissive (37° C.), arabinose-induced | al. (2015) |
| | expression of the λ-Red recombinase for | |
| | homologous recombination. Constitutive | |
| | expression of Cas-9 | |
| pTargetF | Spec^{r}, modifiable by PCR to contain N20 | Jiang, Y. et |
| | sequence recognizable by Cas-9 | al. (2015) |
| pTargetF-emrE | Spec^{r}, modified by PCR to contain an N20 | This |
| | sequence recognizable by Cas-9 to target a | disclosure |
| | PAM site within emrE | |
| pTargetF-mdtD | Spec^{r}, modified by PCR to contain an N20 | This |
| | sequence recognizable by Cas-9 to target a | disclosure |
| | PAM site within mdtD | |
| pTargetF-sugE | Spec^{r}, modified by PCR to contain an N20 | This |
| | sequence recognizable by Cas-9 to target a | disclosure |
| | PAM site within sugE | |
| pTargetF-ynfM | Spec^{r}, modified by PCR to contain an N20 | This |
| | sequence recognizable by Cas-9 to target a | disclosure |
| | PAM site within ynfM | |
| pTargetF-emrD | Spec^{r}, modified by PCR to contain an N20 | This |
| | sequence recognizable by Cas-9 to target a | disclosure |
| | PAM site within emrD | |
| pTargetF-ydeF | Spec^{r}, modified by PCR to contain an N20 | This |
| | sequence recognizable by Cas-9 to target a | disclosure |
| | PAM site within ydeF | |
| pTargetF-mdtJ | Spec^{r}, modified by PCR to contain an N20 | This |
| | sequence recognizable by Cas-9 to target a | disclosure |
| | PAM site within mdtIJ | |
| pTargetF-ydiM | Spec^{r}, modified by PCR to contain an N20 | This |
| | sequence recognizable by Cas-9 to target a | disclosure |
| | PAM site within ydiM | |
| pTargetF-mdtB | Spec^{r}, modified by PCR to contain an N20 | This |
| | sequence recognizable by Cas-9 to target a | disclosure |
| | PAM site within mdtB | |
| pTargetF-mdIA | Spec^{r}, modified by PCR to contain an N20 | This |
| | sequence recognizable by Cas-9 to target a | disclosure |
| | PAM site within mdlA | |
| pTargetF-emrY | Spec^{r}, modified by PCR to contain an N20 | This |
| | sequence recognizable by Cas-9 to target a | disclosure |
| | PAM site within emrY | |
| pTargetF-mdfA | Spec^{r}, modified by PCR to contain an N20 | This |
| | sequence recognizable by Cas-9 to target a | disclosure |
| | PAM site within mdfA | |
| pTargetF-fsr | Spec^{r}, modified by PCR to contain an N20 | This |
| | sequence recognizable by Cas-9 to target a | disclosure |
| | PAM site within fsr | |
| pTargetF-mdtG | Spec^{r}, modified by PCR to contain an N20 | This |
| | sequence recognizable by Cas-9 to target a | disclosure |
| | PAM site within mdtG | |
| pTargetF-mdtH | Spec^{r}, modified by PCR to contain an N20 | This |
| | sequence recognizable by Cas-9 to target a | disclosure |
| | PAM site within mdtH | |
| pTargetF-yieO | Spec^{r}, modified by PCR to contain an N20 | This |
| | sequence recognizable by Cas-9 to target a | disclosure |
| | PAM site within yieO | |
| pTargetF-mdlB | Spec^{r}, modified by PCR to contain an N20 | This |
| | sequence recognizable by Cas-9 to target a | disclosure |
| | PAM site within mdlB | |
| pTargetF-mdtO | Spec^{r}, modified by PCR to contain an N20 | This |
| | sequence recognizable by Cas-9 to target a | disclosure |
| | PAM site within mdtO | |
| pTargetF-yojH | Spec^{r}, modified by PCR to contain an N20 | This |
| | sequence recognizable by Cas-9 to target a | disclosure |
| | PAM site within yojH | |
| pTargetF-yojI | Spec^{r}, modified by PCR to contain an N20 | This |
| | sequence recognizable by Cas-9 to target a | disclosure |
| | PAM site within yojI | |
| pTargetF-yajR | Spec^{r}, modified by PCR to contain an N20 | This |
| | sequence recognizable by Cas-9 to target a | disclosure |
| | PAM site within yajR | |
| pTargetF-ydhC | Spec^{r}, modified by PCR to contain an N20 | This |
| | sequence recognizable by Cas-9 to target a | disclosure |
| | PAM site within ydhC | |
| pTargetF-cusA | Spec^{r}, modified by PCR to contain an N20 | This |
| | sequence recognizable by Cas-9 to target a | disclosure |
| | PAM site within cusA | |
| PINT2 | Amp^{r}, enables chromosomal integration into | Cox, G. et |
| | the arabinose operon via homologous | al. (2017) |
| | recombination. Genes of interest are ligated | |
| | into the MCS, downstream of a Kan^{r} resistance | |
| | cassette | |
| pINT2-acrB | Amp^{r}, contains acrB for chromosomal | This |
| | integration | disclosure |
| pINT2-acrB D408A | Ampr, contains acrBD408A for chromosomal | This |
| | integration | disclosure |
| PINT2-acrD | Amp^{r}, contains acrD for chromosomal | This |
| | integration | disclosure |
| PINT2-acrEF | Amp^{r}, contains acrEF for chromosomal | This |
| | integration | disclosure |
| PINT2-mdtEF | Amp^{r}, contains mdtEF for chromosomal | This |
| | integration | disclosure |
| PINT2-emrKY | Amp^{r}, contains emrKY for chromosomal | This |
| | integration | disclosure |
| pINT2-mdtBC | Amp^{r}, contains mdtBC for chromosomal | This |
| | integration | disclosure |
| PINT2-macAB | Ampr, contains macAB for chromosomal | This |
| | integration | disclosure |
| pINT2-emrAB | Amp^{r}, contains emrAB for chromosomal | This |
| | integration | disclosure |
| PINT2-mexCD | Amp^{r}, contains mexCD from P. aeruginosa | This |
| | PAO1 for chromosomal integration | disclosure |
| pGDP2 | KanR, low-copy number plasmid that enables | Cox, G. et |
| | constitutive expression from P_{Lacl} constitutive | al. (2017) |
| | promoter. Genes of interest are ligated into the | |
| | MCS, downstream of a Kan^{r} resistance | |
| | cassette | |
| pGDP2-emrE | KanR, low-copy number plasmid contains emrE | This |
| | for constitutive expression | disclosure |

### Generation of an Efflux Deficient Strain

Generation of EKO-35 was achieved using a combination of the λ-Red recombinase system (Datsenko, K. A. & Wanner, B. L., 2000) and CRISPR-Cas9 counter-selection (Jiang, Y. et al., 2015). The efflux genes were inactivated in the order denoted in Table 3. All PCR reactions and restriction enzyme digests were prepared according to manufacturers' guidelines. Amplicons were purified using a GeneJET PCR purification kit (Thermo Fisher Scientific) according to manufacturer's guidelines. The 2×GB-AMP™ high-fidelity PaCeR™ polymerase Master Mix (GeneBio Systems Inc) and Taq 2× polymerase Master Mix (FroggaBio) were used according to the manufacturer's suggested guidelines.

For λ-Red recombineering, electrocompetent cells of the mutant strain of interest were transformed with 50 ng of pKD46 (Datsenko, K. A. & Wanner, B. L., 2003). A broth culture was grown to the mid exponential phase (OD600 nm˜0.5) in the presence of 2 mM arabinose and ampicillin to induce recombinase expression. Utilizing the PaCeR™ high-fidelity polymerase, and primers annealing 50 base pairs (bp) upstream and downstream of the gene of interest (Table 4), kanamycin resistance cassettes were amplified from the respective Keio strain. Amplicon size (1500 bp) was verified via gel electrophoresis and the remaining PCR product was purified. Recombinase induced electrocompetent cells were transformed with 250 ng of each amplicon (Bio-Rad MicroPulser, Ec1 setting, 1 mm electroporation cuvette (Fisher)). The cells were recovered and grown overnight on selective agar (LB with kanamycin) to identify gene disruptions. Successful gene knockouts were transformed with pCP20 (Table 2 “Strains and Plasmid)). A single colony was then inoculated into 3 mL of LB containing ampicillin and incubated at 30° C. to induce removal of the resistance cassette. Cassette removal was confirmed using PCR and primers annealing 250 bp upstream and downstream of the gene of interest (Table 4). Amplicon size (500 bp) for successful cassette removal was verified via gel electrophoresis. Efflux genes inactivated using the λ-Red recombinase system are indicated in Table 3.

For CRISPR-Cas9-mediated counterselection, the methodology described by Jiang et al. was modified for high-throughput screening of mutants (Jiang, Y. et al., 2015). CRISPR guide software (Benchling) was employed for selection of appropriate N20 sequences. pTargetF was modified via PCR to introduce an N20 for the gene of interest (Table 4). Amplicon size (2100 bp) was verified via gel electrophoresis, and the remaining PCR product was purified. To enable rapid screening of positive mutants and to disrupt the target gene, ssDNA repair oligos (˜100 bp in length) were designed to contain an AseI restriction site and three tandem stop codons (Table 4). All ssDNA repair oligos were purchased through Integrated DNA Technologies (IDT). Electrocompetent cells of the mutant strain of interest were transformed with 50 ng of pCas. A broth culture of each strain was grown to the mid exponential phase (OD600 nm˜0.5) in the presence of kanamycin and 10 mM arabinose to induce recombinase expression. To recombinase induced electrocompetent cells, 100 ng of pTargetF that was modified to contain the desired N20 sequence, and 2000 ng of repair ssDNA targeting the gene of interest were electroporated (Bio-Rad MicroPulser, Ec1 setting, 1 mm electroporation cuvette (Fisher)). The cultures were recovered in LB at 30° C. and propagated on selective agar (LB with kanamycin and spectinomycin) to identify successful gene disruptions. For high-throughput screening of colonies, Taq polymerase was used with primers annealing to the target region of each gene (Table 4). The amplicons were digested with AseI and successfully inactivated genes were identified via gel electrophoresis by digestion relative to a wild-type negative control. Insertion of the three tandem stop codons into the gene of interest was verified using Sanger sequencing at the Advanced Analysis Centre (AAC) (University of Guelph). Genes disrupted using CRISPR-Cas9-mediated counter selection are indicated in Table 3.

TABLE 3
Gene inactivation during the generation of EKO-35. For genes
inactivated using CRISPR-Cas9 mediated counter selection,
the location of the inserted stop codons are noted.

| | Gene | | Stop Codon |
|---|---|---|---|
| | Size | | Placement |
| Gene | (bp) | KO Method | (bp) |
| acrB | 3150 | λ-Red | N/A |
| acrD | 3114 | λ-Red | N/A |
| acrF | 3105 | λ-Red | N/A |
| mdtF | 3114 | λ-Red | N/A |
| macB | 1947 | λ-Red | N/A |
| emrB | 1539 | λ-Red | N/A |
| mdtL | 1176 | λ-Red | N/A |
| mdtK | 1374 | λ-Red | N/A |
| bcr | 1191 | λ-Red | N/A |
| ydeA | 1191 | λ-Red | N/A |
| mdtM | 1233 | λ-Red | N/A |
| yddA | 1686 | λ-Red | N/A |
| yebQ | 1374 | λ-Red | N/A |
| emrE | 333 | CRISPR-Cas9 | 93 |
| mdtD | 1416 | CRISPR-Cas9 | 26 |
| sugE | 318 | CRISPR-Cas9 | 93 |
| ynfM | 1254 | CRISPR-Cas9 | 115 |
| emrD | 1185 | CRISPR-Cas9 | 206 |
| ydeF | 1188 | CRISPR-Cas9 | 43 |
| mdtJ | 366 | CRISPR-Cas9 | 98 |
| ydiM | 1215 | CRISPR-Cas9 | 124 |
| mdtB | 3123 | CRISPR-Cas9 | 160 |
| mdlA | 1773 | CRISPR-Cas9 | 69 |
| emrY | 1539 | CRISPR-Cas9 | 41 |
| mdfA | 1233 | CRISPR-Cas9 | 116 |
| fsr | 1221 | CRISPR-Cas9 | 242 |
| mdtG | 1227 | CRISPR-Cas9 | 276 |
| mdtH | 1209 | CRISPR-Cas9 | 136 |
| yieO | 1428 | CRISPR-Cas9 | 177 |
| mdlB | 1782 | CRISPR-Cas9 | 72 |
| mdtO | 2052 | CRISPR-Cas9 | 75 |
| yojH* | 1647 | CRISPR-Cas9 | 187 |
| yojI | 1644 | CRISPR-Cas9 | 25 |
| yajR | 1365 | CRISPR-Cas9 | 47 |
| ydhC | 1212 | CRISPR-Cas9 | 90 |
| cusA | 3144 | CRISPR-Cas9 | 400 |

*YojH was repaired due to lack of supporting evidence of efflux pump activity.

TABLE 4
Primers and oligonucleotides used in this Example.

| | | SEQ ID |
|---|---|---|
| Primer | Sequence (5′-3′) | NO: |
| AcrB Seq Up | CTGAAACAAGAGAACGGCAAAGGC | 1 |
| AcrB Seq | CTTACTGACCTGGACTTGCCCTCTCG | 2 |
| Low | | |
| AcrD 50 Up | CGCTACAGTGAAGCAAGTCAAGC | 3 |
| AcrD 50 Low | CCGCTGAGCAGGTTCTTAATCG | 4 |
| AcrD Seq Up | CGCTGACTTTTTCACAACCTTCCG | 5 |
| AcrD Seq | GTCCCCCGGAGGTAGTCATCGCAGC | 6 |
| Low | | |
| AcrF 50 Up | GGTTAAAGCCACTACCGATACCCC | 7 |
| AcrF 50 Low | GAAGGGAGCGGGAACTATAGAAAGC | 8 |
| AcrF Seq Up | GAGGCTGAAGCAATCCGTAGAGC | 9 |
| AcrD Seq | GATACTGTATCGTTAAAAAGAGCGCG | 10 |
| Low | | |
| MdtF KO Up | GCACGAGCAATTTCCTCCAGCCAGG | 11 |
| MdtF KO | GGTTGTTGAGTGGTGAATGGTTAGC | 12 |
| Low | | |
| MdtF 200 Up | GATGTCGTGCAGCTACGCGAAAT | 13 |
| MdtF 200 | ACGAATGGCTGGAGTGGTTTC | 14 |
| Low | | |
| MacB KO Up | CGATACCGATGTTGAGATTGTCAAAGG | 15 |
| MacB KO | GCATAGCTCTTCTGTCTCATTGTGTAC | 16 |
| Low | | |
| MacB 200 Up | ATGTGCTGACGATCCCTCTGTC | 17 |
| MacB 200 | TTCGCTCATTATTCCACCATTCAG | 18 |
| Low | | |
| EmrB KO Up | GGCACCTGTCAATAAACTGATCG | 19 |
| EmrB KO | GCACATCTAGTCAGTAAACTATCTTCAC | 20 |
| Low | | |
| EmrB 200 Up | CGTTCAGCGTCTGCCTGTGCG | 21 |
| EmrB 200 | GCGGCAATGGAAGACGTGCTG | 22 |
| Low | | |
| MdtL KO Up | GGCAACTCGCCTGATCCTCCTTC | 23 |
| MdtL KO | ATCCGCTTCGCCGCTTTGGTTAC | 24 |
| Low | | |
| MdtL 200 Up | CATTCTCTTTGGTATAACCGTG | 25 |
| MdtL 200 | TTTGCTCCATGCTGACCAT | 26 |
| Low | | |
| MdtK KO Up | TTCACTGGAGAATTAATAAATCC | 27 |
| MdtK KO | GACGGGATTGGCGCAACGCTCC | 28 |
| Low | | |
| MdtK 200 Up | GAAATCAGTTAAGACATTCTGTTC | 29 |
| MdtK 200 | CATGTGCAACTGAAAGTGAAAC | 30 |
| Low | | |
| Bcr KO Up | GGAATGATAGATTTGTGGTTGAC | 31 |
| Bcr KO Low | CGGTGAATATCGTCCGTTAACTG | 32 |
| Bcr 200 Up | CCTCTATGGCTCTGATTTAAGTA | 33 |
| Bcr 200 Low | GTTATCATCAGGTGAAACGCAT | 34 |
| YdeA KO Up | GAAAGAATCAGCGTCAGAGAAA | 35 |
| YdeA KO | ATTAACGGCATCCTGGAAATC | 36 |
| Low | | |
| YdeA 200 Up | CACGAAGACCCTGGTGAAA | 37 |
| YdeA 200 | CGCCGCCGCTGATCTTT | 38 |
| Low | | |
| MdtM KO Up | GCCCTTCTCACCTGCCT | 39 |
| MdtM KO | GCGTAACGACAAAGGTAGCAG | 40 |
| Low | | |
| MdtM 250 Up | CAGCGTAACGACAAAGGTAGCAG | 41 |
| MdtM 200 | ACCACCGCAAACCAGTC | 42 |
| Low | | |
| YddA KO Up | CATCAGTAAATCATTGCCATAG | 43 |
| YddA KO | GACGCCAGGAATAAGAAC | 44 |
| Low | | |
| YddA 250 Up | TGTCGGGTGTTTCGTCAT | 45 |
| YddA 250 | TCGCTGATATTGCCATTC | 46 |
| Low | | |
| YebQ KO Up | TAGTTACAATTCTGCGACATCC | 47 |
| YebQ KO | CGCTCAGTGAGTTTGTTCAT | 48 |
| Low | | |
| YebQ 200 Up | CACGGAAGATACAGAATCAGG | 49 |
| YebQ 200 | CAGCTATGAACCGCAAGAA | 50 |
| Low | | |
| EmrE N20 Up | AGTTTTCAGAAGGTTTTACAGTTTTAGAGCTAGAAATAGCA | 51 |
| | AG | |
| EmrE N20 | TGTAAAACCTTCTGAAAACTACTAGTATTATACCTAGGACT | 52 |
| Low | GAG | |
| EmrE ssDNA | ATA ACA AAT AAT TGT ACC AAC AGA TGG CCA | 53 |
| Repair | TAA TTA TTA TTA ATT TGT AAA ACC TTC TGA | |
| | AAA CTT CAT TAA GGT TGT ACC AAT GAC CTT | |
| | TAT TAT TAC TGC | |
| EmrE 200 Up | CGGTTCGCTACCAGAGAAGAATG | 54 |
| EmrE 200 | CATGGTGACACCTGCTAACGTATGC | 55 |
| Low | | |
| MdtD N20 Up | CCACAATCCACAATTGCCAAGTTTTAGAGCTAGAAATAGCA | 56 |
| | AG | |
| MdtD N20 | TTGGCAATTGTGGATTGTGGACTAGTATTATACCTAGGACT | 57 |
| Low | GAG | |
| MdtD ssDNA | TG TCC AGC GAC TGC ATA AAG AAG CCG AAA GCC | 58 |
| Repair | ACA ATC CAC AAT TGC CAA TTA TTA TTA ATT | |
| | GGT GCT GTC GGG AAG ATC TGT CAT TTA CTC | |
| | GGT TAC CGT TTG TTT AGG TT | |
| MdtD 200 Up | CGCCCGATTATGATGACTAC | 59 |
| MdtD 200 | CTGAAAGACAAAGCGATCATTG | 60 |
| Low | | |
| SugE N20 Up | CGTCAAACGACTAAAGCCGTGTTTTAGAGCTAGAAATAGCA | 61 |
| | AG | |
| SugE N20 | CGTCAAACGACTAAAGCCGTACTAGTATTATACCTAGGACT | 62 |
| Low | GAG | |
| SugE ssDNA | GAC AAT CAT CGC CGT CAC AGT AAT AAC ACT | 63 |
| Repair | CGG CGT CAA ACG ACT AAA GCC GTG TTA TTA | |
| | TTA ATT ATA TTT CAG GCC AAC GGC CCA TAC | |
| | CAC TTC CAG CAG ACC AGC AAT AAC TAA GAT | |
| SugE 200 Up | CGCAGCAACGAAAGCGCA | 64 |
| YnfM N20 Up | TCCGGCAGAGAACAGCGCCAGTTTTAGAGCTAGAAATAGCA | 65 |
| | AG | |
| YnfM N20 | TGGCGCTGTTCTCTGCCGGAACTAGTATTATACCTAGGACT | 66 |
| Low | GAG | |
| YnfM ssDNA | CTG CAC ACA ATA GAG AAG TGC AAA TGT TGC | 67 |
| Repair | CAG TCC GGC AGA GAA CAG CGC CAG TTA TTA | |
| | TTA ATT GAC GCG CAT AAA TTG CGG CGT ACC | |
| | GCG TTT AAT AAA TTG ATT TGG CTG AGA AAT | |
| YnfM 200 Up | GTTGCGAAATATTCAGGC | 68 |
| YnfM 200 | AAAGCAGTAGAATAACTGC | 69 |
| Low | | |
| EmrD N20 | TCACCGGTCGGCGGCCCACGGTTTTAGAGCTAGAAATAGCA | 70 |
| Up | AG | |
| EmrD N20 | CGTGGGCCGCCGACCGGTGAACTAGTATTATACCTAGGACT | 71 |
| Low | GAG | |
| EmrD ssDNA | CGT TGC CAG CAT AAA AAT GGA CAT TCC GAC | 72 |
| Repair | GAG GAT CAC CGG TCG GCG GCC CAC GCG TTA | |
| | TTA TTA ATT GGA AAT CGG GCC ATA AAA CAG | |
| | CTG TGA GAC ACC GTA AGT CAG CAG ATA AGC | |
| | GCC | |
| EmrD 200 Up | CGATGCTGACGCATCTTATCCGCCC | 73 |
| EmrD 200 | GGTGCGGGCAGATATCAGTCGTATC | 74 |
| Low | | |
| YdeF N20 Up | GATGGTTAATAACAACGACGGTTTTAGAGCTAGAAATAGCA | 75 |
| | AG | |
| YdeF N20 | CGTCGTTGTTATTAACCATCACTAGTATTATACCTAGGACT | 76 |
| Low | GAG | |
| YdeF ssDNA | AAT GGT CAT AAA TGG CAG CGT AGC GCC GCG | 77 |
| Repair | TCC GAT GGT TAA TAA CAA CGA CGA TTA TTA | |
| | TTA ATT AAG AAG GGC GCT GGT AGA GCG TCG | |
| | TAG GGA TAA GTT CAT | |
| YdeF 250 Up | CTGATGGTTAATCCATACCCCAGC | 78 |
| YdeF Check | GATGCTCTGCATTACCAACAGCGTG | 79 |
| Internal | | |
| MdtJ N20 Up | AGCGTCAGTGAGGGAAATGGGTTTTAGAGCTAGAAATAGCA | 80 |
| | AG | |
| MdtJ N20 | CCATTTCCCTCACTGACGCTACTAGTATTATACCTAGGACT | 81 |
| Low | GAG | |
| MdtJ ssDNA | CGA CAG AGA AAT CAT CAC CAG CAT TAA AAT | 82 |
| Repair | AAA TTA TTA TTA ATT GCC ATT TCC CTC ACT | |
| | GAC GCT CGC CCA TTT CAT TGA CAG CGT ACC | |
| | GGT | |
| MdtJ 200 Up | CAATGCATAAGCGACAGACAAGTCG | 83 |
| MdtJ 200 | CATCCGCGATGACGAGAAGCAACAC | 84 |
| Low | | |
| YdiM N20 Up | GATAACTATCGAGACACCCGGTTTTAGAGCTAGAAATAGCA | 85 |
| | AG | |
| YdiM N20 Up | CGGGTGTCTCGATAGTTATCACTAGTATTATACCTAGGACT | 86 |
| | GAG | |
| YdiM ssDNA | CAA GAC ACT TAA TCG ACC AAT GCC CAG CGA | 87 |
| Repair | TGA GAT AAC TAT CGA GAC ACC CGC TTA TTA | |
| | TTA ATT ATT AGT CTG CCA AAG TGT CTC CAG | |
| | CGA GGC CAT ATT CAG | |
| YdiM Check | CAAGTGTGCCATTCCTGATCGTG | 88 |
| Up | | |
| YdiM Check | GAACCCACGGTGTAGATACTGAG | 89 |
| Up | | |
| MdtB N20 Up | GTAGAGCGTGACCACCTGAAGTTTTAGAGCTAGAAATAGCA | 90 |
| | AG | |
| MdtB N20 | TTCAGGTGGTCACGCTCTACACTAGTATTATACCTAGGACT | 91 |
| Low | GAG | |
| MdtB ssDNA | AAC GGC AGA GGT CAT GAC ATC CGG GCT GGC | 92 |
| Repair | ACC TGG GTA GAG CGT GAC CAC CTG AAT TTA | |
| | TTA TTA ATT CGG ATA GTC CAC TTC CGG CAG | |
| | CGC CGA AAC GGG CAG | |
| MdtB 200 Up | GTCAGAAAGTGGTGATCCGTGCAG | 93 |
| MdtB Check | GATCGCTCGGCAACAAGTTGGTCG | 94 |
| Low | | |
| MdIA N20 Up | CATCGCGATAATGACAAGCAGTTTTAGAGCTAGAAATAGCA | 95 |
| | AG | |
| MdIA N20 | GCTTGTCATTATCGCGATGACTAGTATTATACCTAGGACTG | 96 |
| Low | AGT | |
| MdIA ssDNA | ACC AAC CAC TTT TGG CGG AAC CAG TTG CAG | 97 |
| Repair | CAT CGC GAT AAT GAC AAG CAA TTA TTA TTA | |
| | ATT GAC AGC CCC GAG ATA GCG ACG CCA TTC | |
| | CCG ACG GAA ATA CCA GCT | |
| MdIA 300 Up | GTCACGGTGGTTACCGAAATGCCAG | 98 |
| MdIA Check | GCGTTAAACTGCCCTGCACCAC | 99 |
| Internal | | |
| EmrY N20 Up | ACTCCGGCACCATTAACCGGGTTTTAGAGCTAGAAATAGCA | 100 |
| | AG | |
| EmrY N20 | CCGGTTAATGGTGCCGGAGTACTAGTATTATACCTAGGACT | 101 |
| Low | GAG | |
| EmrY ssDNA | TTG CAT AAA TGT CGC TAA TGA CAA TGC AAT | 102 |
| Repair | AGT GAC GCA CCA TAA CGT TTA TTA TTA ATT | |
| | ACC GGT TAA TGG TGC CGG AGT TGA TTT AGT | |
| | GAT TGC CAT | |
| EmrY 200 Up | AGCGCAGAACAACTGCGTAATA | 103 |
| EmrY 200 | GTACGGGTTGAAGTTTCTCTTG | 104 |
| Low | | |
| MdfA N20 Up | GGCAACGATATGATTCAACCGTTTTAGAGCTAGAAATAGCA | 105 |
| | AG | |
| MdfA N20 | GGTTGAATCATATCGTTGCCACTAGTATTATACCTAGGACT | 106 |
| Low | GAG | |
| MdfA ssDNA | AAT GCC CGC CTG ATA TTG TTC CAC CAC GGC | 107 |
| Repair | CAA CAT TTA TTA TTA ATT GGG TTG AAT CAT | |
| | ATC GTT GCC GAT ATA GGT TGA AAA TTC GTA | |
| | AAG CAC CAG ACA | |
| MdfA_200_U | ATCGTCTTATTTCCCTCAAGC | 108 |
| p | | |
| MdfA_200_L | ATGTGCCGAGTGGATACAAAGT | 109 |
| OW | | |
| Fsr N20 Up | TCGCTACTGCAACCAGTGGTGTTTTAGAGCTAGAAATAGCA | 110 |
| | AG | |
| Fsr N20 Up | ACCACTGGTTGCAGTAGCGAACTAGTATTATACCTAGGACT | 111 |
| | GAG | |
| Fsr ssDNA | CGA CCA TGG CAT CGG ATA TTT ATC GGT CCA | 112 |
| Repair | GTA TTA TTA TTA ATT GAC CAC TGG TTG CAG | |
| | TAG CGA AGA GGC GAG CTG GAA GGT GAG GGT | |
| | TAT CAT GCC AAT CTG | |
| Fsr Check | GGTTAACAGCGCTAACGCCACG | 113 |
| Up | | |
| Fsr Check | GTGGCGTGATGCATTCCGTCTC | 114 |
| Low | | |
| MdtG N20 Up | ATGCTATTACGCTCTGCCCTGTTTTAGAGCTAGAAATAGCA | 115 |
| | AG | |
| MdtG N20 | AGGGCAGAGCGTAATAGCATACTAGTATTATACCTAGGACT | 116 |
| Low | GAG | |
| MdtG ssDNA | GAT ATT TTG TGC CAG CCC CAT CAA CAC CAT | 117 |
| Repair | CAC GAT GCC CAT TTA TTA TTA ATT GAG GGC | |
| | AGA GCG TAA TAG CAT GAG TTT TCG GCC TTT | |
| | ACG GTC GGC GAG TCC ACC CCA AAA CGG | |
| MdtG Check | GGCATTGAACTGTTGCACATTCGC | 118 |
| Up | | |
| MdtG Check | CATGATGGCACCAGAGCAGTATATG | 119 |
| Low | | |
| MdtH N20 Up | GAGAGCAATACCGACCATGAGTTTTAGAGCTAGAAATAGCA | 120 |
| | AG | |
| MdtH N20 | TCATGGTCGGTATTGCTCTCACTAGTATTATACCTAGGACT | 121 |
| Low | GAG | |
| MdtH ssDNA | GAA AAT ACC CAG ACC TTG CTG AAT AAA TTG | 122 |
| Repair | GCG TAG ACC GAG AGC AAT ACC GAC CAT GAC | |
| | TTA TTA TTA ATT GGC CCA GCC CAT TTG ATC | |
| | AAC GAA GCG GAT AGA | |
| MdtH Check | GCGTCGTCGTTGAGCAGAACATG | 123 |
| Up | | |
| MdtH Check | GTCGGTCTGTGGTTAAGCGCAC | 124 |
| Low | | |
| YieO N20 Up | CATCAGTTATACGCTGACGGGTTTTAGAGCTAGAAATAGCA | 125 |
| | AG | |
| YieO N20 | CCGTCAGCGTATAACTGATGACTAGTATTATACCTAGGACT | 126 |
| Low | GAG | |
| YieO ssDNA | GCG ATC GGC TAG CCA TCC GCT TAC CGG AAT | 127 |
| Repair | AAG CAT TTA TTA TTA ATT CAC CGT CAG CGT | |
| | ATA ACT GAT GAT GGC TGA TTG CAT CGC GAG | |
| | AGG AGA ACG ATT AAG | |
| YieO Check | CGTCAATTACCAGCGACACAGTG | 128 |
| Up | | |
| YieO Check | CGTGCATGGAGAATATAGAGAAGC | 129 |
| Internal | | |
| MdIB N20 Up | ATCAGGACCGCAATCCCCAGGTTTTAGAGCTAGAAATAGCA | 130 |
| | AG | |
| MdIB N20 | CTGGGGATTGCGGTCCTGATACTAGTATTATACCTAGGACT | 131 |
| Low | GAG | |
| MdIB ssDNA | ACT GAC TTC TGC CGC CGC CGC AAC CCA CAT | 132 |
| Repair | CAT CAG GAC CGC AAT CCC CAG TTA TTA TTA | |
| | ATT TTT ACG CCA CGG CGA ACC GTA CGC TAA | |
| | CAG GCG CTT GAG AGT | |
| MdIB Check | CTTGATGATGCGCTTTCGGCGGTG | 133 |
| Up | | |
| MdIB Check | CGCCATATAGTGTGAACGACTGGCC | 134 |
| Internal | | |
| MdtO N20 Up | TTCATGAAGAGTTAAGCGAGGTTTTAGAGCTAGAAATAGCA | 135 |
| | AG | |
| MdtO N20 | CTCGCTTAACTCTTCATGAAACTAGTATTATACCTAGGACT | 136 |
| Low | GAG | |
| MdtO | GAG TTG CAC GGT CTG CGG CAC GCG ACC TGG | 137 |
| SSDNA | TCG TTA TTA TTA ATT CTC GCT TAA CTC TTC | |
| Repair | ATG AAA GAA CGC CAG CAG CCT GAC CAC CGG | |
| | TAA TGG CAG GGA GTT | |
| MdtO Check | CGTAGCGCATATAGTCTGGATTGG | 138 |
| Internal | | |
| MdtO Check | GAGGGTAAAGTGGATTCGATTGGC | 139 |
| Up | | |
| YojH N20 Up | TGAATGGTCGATGACCATGGGTTTTAGAGCTAGAAATAGCA | 140 |
| | AG | |
| YojH N20 | CCATGGTCATCGACCATTCAACTAGTATTATACCTAGGACT | 141 |
| Low | GAG | |
| YojH ssDNA | GCC GTT CGA ACT CTC CTG CGC GAC ACC CTC | 142 |
| Repair | CAG GCG TTA TTA TTA ATT CAC CAT GGT CAT | |
| | CGA CCA TTC AGG CTC CAG CTC GCG TAA ATA | |
| | GGT CCC CAA CGT | |
| YojH Check | CACTGACGACTTCAGTACCCAGACG | 143 |
| Internal | | |
| YojH Check | CTTAAGTGTTACCGTTGATGCCGC | 144 |
| Up | | |
| YojI N20 Up | AACTTCTTGTACTTGTCTGGGTTTTAGAGCTAGAAATAGCA | 145 |
| | AG | |
| YojI N20 Low | GCCAGACAAGTACAAGAAGTTACTAGTATTATACCTAGGAC | 146 |
| | TGAG | |
| YojI ssDNA | TAG CGC CAT CAC ACT GAT AAA TGG CCA GCG | 147 |
| Repair | ATA CTG TTA TTA TTA ATT CCA GAC AAG TAC | |
| | AAG AAG TTC CAT GCA GAA AAC CCG GAC AAT | |
| | GAA TTA CAG CCC GCA GTT | |
| YojI Check | GTCGCGGCAACGTTGGTATCAG | 148 |
| Internal | | |
| YojI Check | GCTGCATCAGGATAAAGACGAACCG | 149 |
| Up | | |
| YajR N20 Up | GGTGAGAGGCGCGCGACCTGGTTTTAGAGCTAGAAATAGCA | 150 |
| | AG | |
| YajR N20 | CAGGTCGCGCGCCTCTCACCACTAGTATTATACCTAGGACT | 151 |
| Low | GAG | |
| YajR ssDNA | GCC CAG CAT GCG CAA CGA GAA TAC GGT CCC | 152 |
| Repair | TTA TTA TTA ATT CCA GGT CGC GCG CCT CTC | |
| | ACC TGG CGT CAT TTT ATA ATC GTT cat TAC | |
| | CAC CTC TGT TTT AAA TTC | |
| YajR Check | GTTGCTGATGACAGAATCTGGGCGC | 153 |
| Up | | |
| YajR Check | CCATTCCGGACTCACGATTAAGTACG | 154 |
| Internal | | |
| YdhC N20 Up | TTGCAGGTCGGCCTGTATGGGTTTTAGAGCTAGAAATAGCA | 155 |
| | AG | |
| YdhC N20 | CCATACAGGCCGACCTGCAAACTAGTATTATACCTAGGACT | 156 |
| Low | GAG | |
| YdhC | AAG GAA CAG ACT AAG GCT GGC ACT GAC AGC | 157 |
| SSDNA | AGA | |
| Repair | CGC AGG CGT TTG CAG GTC GGC CTG TAT GGC | |
| | TTA TTA TTA ATT GAA AGC AGG CAG ATA CAT | |
| | ATC GGT TGC CAG AAA | |
| YdhC Check | CACATCACGGTGCCGTCGTTCAAAG | 158 |
| Up | | |
| YdhC Check | CCGGTTTACGACCATAACGGTCGG | 159 |
| Internal | | |
| CusA N20 Up | ATAGATCCAGCCAACACCCGGTTTTAGAGCTAGAAATAGCA | 160 |
| | AG | |
| CusA N20 | CGGGTGTTGGCTGGATCTATACTAGTATTATACCTAGGACT | 161 |
| Low | GAG | |
| CusA ssDNA | CAG ATC GTG CTT ACC GCT GCG ATC CAC CAG | 162 |
| Repair | TGC ATA TTC ATA GAT CCA GCC AAC ACC CGT | |
| | TTA TTA TTA ATT ATC TGG CCC CAG CTC GGC | |
| | GCT GAC TCC | |
| CusA 200 Up | GGTGATTACCGTTGATGCCGAC | 163 |
| CusA Check | GAGAAACCAGTCCTGTAATGAGCG | 164 |
| Internal | | |
| fhuA 40 Up | TGTCACATGGAGTTGGCAGG | 165 |
| glmS 3680 | CTTACCATGTCGCGCTGATC | 166 |
| Low | | |
| pstS 520 Up | CAGGTAGCTGGTGAAGACGAAG | 167 |
| F1B | CAGGCGCTTTTCGTAATTCATC | 168 |
| F4B | CCCACCATTCAGAGAAGAAACC | 169 |
| F1C | CCATCAAAAAACCAGGCTTGAG | 170 |
| F4C | GCATTCTGTAACAAAGCGGGAC | 171 |
| pLac-Fwd | GCTCAACGGCCTCAACCTAC | 172 |
| pINT-Rev | CCAACTCAGCTTCCTTTCGG | 173 |
| PolB-Fwd | CAGTTCGAAATCAAGCGAGGAG | 174 |
| AcrB Ndel | GGAATTCCATATGAACAAAAACAGAGGGTTTACG | 175 |
| Up | | |
| AcrB XhoI | CCGCTCGAGTCAATGATGATCGACAGTATG | 176 |
| Low | | |
| AcrBD408A | CCTGTTGGTGGATGCCGCCATCGTTGT- | 177 |
| Fwd | | |
| AcrBD408A | CCACAACGATGGCGGCATCCACCAACAGG | 178 |
| Rev | | |
| AcrD Ndel | GCTCATATGGCGAATTTCTTTATTGATCG | 179 |
| Up | | |
| AcrD XhoI | TATCTCGAGTTATTCCGGGCGCGGCTTCA | 180 |
| Low | | |
| AcrE Ndel | GGAATTCCATATGACGAAACATGCCAGGTTTTTCC | 181 |
| Up | | |
| AcrF XhoI | CCGCTCGAGTTATCCTTTAAAGCAACGGCG | 182 |
| Low | | |
| EmrA Ndel | GGAATTCCATATGAGCGCAAATGCGGAGAC | 183 |
| Up | | |
| EmrB XhoI | CCGCTCGAGTTAGTGCGCACCGCCTC | 184 |
| Low | | |
| EmrK NdeI | GGAATTCCATATGGAACAGATTAATTCAAATAAAAAACATT | 185 |
| Up | C | |
| EmrY BamHI | CCGGGATCCTCACCCAACGCCTTTCGCT | 186 |
| Low | | |
| MdtE Ndel | GACCATATGAACAGAAGAAGAAAGCT | 187 |
| Up | | |
| MdtF XhoI | ATACTCGAGTTACGCTTTTTTAAAGCGG | 188 |
| Low | | |
| MdtB Ndel | GTCCATATGCAGGTGTTACCCCCGAGCAGC | 189 |
| Up | | |
| MdtC XhoI | GTTCTCGAGTTACTCGGTTACCGTTTGTTTAG | 190 |
| Low | | |
| MacA Ndel | GGAATTCCATATGAAAAAGCGGAAAACCG | 191 |
| Up | | |
| MacB XhoI | CCGCTCGAGTTACTCTCGTGCCAGAGC | 192 |
| Low | | |
| EmrE Ndel | GTACCATATGAACCCTTATATTTATC | 193 |
| Up | | |
| EmrE XhoI | GTACCTCGAGTTAATGTGGTGTGCTTCG | 194 |
| Low | | |
| MdtK Ndel | GTCCATATGGTGCAGAAGTATATCAGTGAAG | 195 |
| Up | | |
| MdtK XhoI | GTCCTCGAGTTAGCGGGATGCTCGTTGCAG | 196 |
| Low | | |
| MexC Ndel | GGAATTCCATATGGCTGATTTGCGTGCAATA | 197 |
| Up | | |
| MexD HindIII | CTATAAGCTTTCACTCCCCGGCCGAA | 198 |
| Low | | |

### Whole Genome Sequencing of EKO-35 and EKO-35-Pore

Genomic DNA was extracted using the One-4-All Genomic DNA Miniprep Kit (BioBasic), according to the manufacturer's guidelines. Quality of the extracted gDNA was assessed using gel electrophoresis. Illumina DNA library preparation was performed using an Illumina Nextera kit by the Microbial Genome Sequencing Center (Pennsylvania, USA), which was followed by Illumina sequencing on a NextSeq 2000 platform. Analysis of the raw reads was performed using Geneious Prime 2021.0.2 (Kearse, M. et al., 2012). Low quality reads were trimmed using an in-suite BBDuk plug-in. Raw wild-type reads were assembled to an NCBI reference genome (Accession No. CP009273.1) with bowtie2. The resulting assembly was used as a reference to assemble the EKO-35 mutant reads. Differences between the wild-type BW25113 and EKO-35 strains were identified by searching for single nucleotide polymorphisms (SNPs) and deletions using the following thresholds: minimum variant frequency of 0.75, maximum variant P-value of 10⁻⁶, and minimum variant P-value of 10⁻⁵. 11 mutations were identified, including a mutation in hdfR (T806C, L269P). CRISPR-Cas9-mediated counter selection was utilized to repair the mutation in hdfR, which introduced two intentional silent mutations (hdfR C722G and A838G) to remove the adjacent PAM site and AseI-guided screening purposes as described above. For the EKO-35-Pore strain, genomic DNA extraction, sequencing, analysis, genome assembly, and mutation identification was performed as described above. Whole genome sequencing was deposited in the GenBank database (BioProject ID PRJNA838981).

Construction of the pINT2 Efflux Gene Library

Inventors first attempted to generate marker less integrations of efflux-encoding genes into araC using the pINT1 plasmid (Cox, G. et al., 2022), under the constitutive control of the strong PBIa promoter. However, inventors observed numerous deleterious mutations following ligation into this vector and genomic integration, indicating high expression levels were not feasible. Consequently, the pINT2 plasmid was selected for the expression of efflux pump encoding genes from the same constitutive PLacI promoter. This plasmid enables single-copy genomic integration of a selected gene through integration into the nonessential araC gene within the arabinose operon (Cox, G. et al., 2022). All E. coli genes were amplified from E. coli BW25113 genomic DNA, and mexCD was amplified from P. aeruginosa PAO1, using a high-fidelity polymerase, followed by ligation into pINT2. Successful clones were verified using PCR (Primers: pLac-Fwd/pINT-Rev, Table 4) and confirmed via Sanger sequencing at The Centre for Applied Genomics (TCAG) (The Hospital for Sick Children) and the AAC (University of Guelph). For the interplay studies, this process was repeated using the pGDP2 plasmid (Cox, G. et al., 2022).

### Construction of the Efflux Platform

For genomic integration, genes of interest and the adjacent kanamycin resistance cassette were amplified from pINT2 using a high-fidelity polymerase and the F1B-Fwd/F4B-Rev or F1C-Fwd/F4C-Rev primers (Table 4). For the integration of efflux pump-encoding genes, electrocompetent EKO-35 cells were transformed with ng of pKD46. Electrocompetent recombinase-induced cells were transformed with 500 ng of each amplicon (BioRad MicroPulser, Ec1 setting, 1 mm electroporation cuvette (Fisher). Genomic integrations were verified via PCR (Primers: PolB-Fwd/pINT-Rev, Table 4). Successful integrations were transformed with pCP20 to remove the kanamycin resistance cassette. Prior to phenotypic profiling, all integrated genes, including their promoters, were verified using Sanger sequencing (TCAG, The Hospital for Sick Children).

For genomic integration of the pore, the fhuA ΔC/Δ4L gene and the adjacent gentamicin resistance cassette were amplified using a high-fidelity polymerase and the fhuA_40_Up/gImS_3680_Low primers (Table 4). Integration of the pore gene was performed as described above. The pore gene was introduced into the intergenic region between the glmS and pstS genes, with gene expression under the control of a constitutive promoter (Kearse, M. et al., 2012). Genomic integrations were verified using PCR (Primers: pstS_520_Up/glmS_3680_Low, Table 4). Successful integrations were transformed with pCP20 to remove the gentamicin resistance cassette as previously described. Activity of the pore was confirmed through susceptibility testing with the large antibiotic vancomycin prior to phenotypic profiling and further susceptibility testing.

### Assessing the Utility of the EKO-35 Efflux Platform

To enable comparison between efflux production in different genetic backgrounds, each efflux pump-encoding gene was also integrated into the genome of the wild-type BW25113 strain, the single gene deletion mutant from the Keio Collection, and EKO-35. Strains of interest were propagated on LB agar at 37° C. Cell inoculum was prepared using the colony resuspension method and applied to a 96-well well plate (VWR) in technical duplicate, and the minimum inhibitory concentrations were determined according to Clinical & Laboratory Standards Institute (CLSI) protocols in MHB II (Patel J. B. et al, 2015). The plates were incubated for 18 h (Multitron Shaker, Infors HT) at 37° C. with aeration at 900 rpm. The plates were equilibrated to room temperature before the OD600 nm was measured using a BioTek Synergy H1 microplate reader. For interplay profiling, blank subtracted OD600 nm values >0.1 were considered to represent growth.

### Phenotypic Profiling of EKO-35

For growth profiling in nutrient-rich conditions, strains were propagated on LB agar for 18 h at 37° C. Single colonies were inoculated into LB and grown at 37° C. until the mid-exponential phase (OD600 nm˜0.6) was reached. All strains were assessed with at least three biological replicates. The cultures were standardized to an OD600 nm˜0.1 in sterile 0.85% saline (w/v). Standardized cultures were diluted 1/200 into LB and 100 μL of the resulting dilution were applied to round-bottom 96-well microtiter plates (VWR). To prevent evaporation, the microtiter plates were sealed (labeling tape, Fisher Scientific). The OD600 nm was measured every 15 minutes over the course of 24 h using a BioTek Synergy H1 microplate reader. Growth was assessed at both 37° C. and 25° C.

For growth profiling in nutrient-limited conditions, strains were propagated in biological triplicate on LB agar at 37° C. Multiple single colonies were suspended, using three separately prepared inoculums per strain, in sterile 0.85% saline (w/v) to an OD600 nm˜0.1. Standardized cultures were diluted 1/200 in fresh M9 and 100 μL of the resulting dilution was applied to round-bottom 96-well microtiter plates (VWR). The plate was sealed (labeling tape, Fisher Scientific) to prevent evaporation. Growth was assessed as described above, at both 37° C. and 25° C., for 48 h.

For physiological profiling in low-oxygen conditions, strains were prepared in LB or M9 as described above. To a round-bottom 96-well microtiter plate (VWR, tissue culture treated), 100 μL of the diluted cultures were applied in triplicate. Prior to incubation, the sample plate was placed into an anaerobic jar (Oxoid™ AnaeroJar, Thermo Fisher Scientific) with an anaerobic gas generator (AnaeroPack™ Thermo Fisher Scientific) for 30 min. The plates were incubated in a BioTek Synergy H1 plate reader, equipped with an 02 gas controller. Using nitrogen gas, the oxygen levels in the plate reader were maintained at 1% (LB) and 5% (M9). Growth was assessed as described above.

To assess fitness of EKO-35 expressing wild-type copies of the genes identified to harbor nonsynonymous mutations, Inventors obtained clones from the E. coli ASKA library (Kitagawa, M. et al., 2005) with the rspA, tufA, pitA, wcaC, gyrB, and yjfC genes carried on plasmids, with gene expression under the control of an IPTG-inducible promoter. The ASKA plasmids were verified using Sanger sequencing and gene expression was induced with 0.1 mM IPTG. In the wild-type E. coli K-12 strain, the plasmids were maintained with 25 μg/mL chloramphenicol. Due to the changes in susceptibility of the efflux-deficient strains, in EKO-35 the plasmids were maintained with 4 μg/mL and 1 μg/mL chloramphenicol in Lysogeny broth and M9 minimal glucose medium, respectively.

### Assessing Biofilm Formation

Overnight cultures were propagated in LB at 37° C. for 18 h. Saturated cultures were diluted 1/100 into LB or M9. To a flat-bottom 96-well microtiter plate (CoStar, untreated polystyrene), 150 uL of diluted culture was applied in triplicate. The plates were sealed to reduce evaporation and incubated statically at 37° C. for 24 h (nutrient-rich media) or 48 h (nutrient-limited media). To assess the effect of the nonsynonymous mutations on biofilm formation, this procedure was repeated with the addition of 0.1 mM IPTG for plasmid induction and chloramphenicol for plasmid maintenance at the concentrations specified above.

Following incubation, the optical density (OD600 nm) of the samples were measured using a BioTek Synergy H1 microplate reader. The cultures were aspirated, and the plates were washed in triplicate with 300 uL of deionized water using a BioTek ELx405 microtiter plate washer. Each well was stained with 175 uL of (w/v) crystal violet (CV), followed by static incubation at room temperature for 20 min. The CV was removed, and the plates were washed with deionized water until no excess stain was present. The plates were allowed to air dry before 175 uL of 0.7% acetic acid was applied to each well, followed by incubation at room temperature for min to solubilize the CV. The absorbance of each sample was measured at 595 nm using a BioTek Synergy H1 microplate reader.

### Susceptibility Profiling of the EKO-35 Platform to Determine Physicochemical Substrate Profiles

EKO-35 strains containing the chromosomally integrated genes of interest were propagated on LB agar at 37° C. for 18 h. Single colonies were used to inoculate 3 mL of LB followed by incubation at 37° C. for 18 h with aeration at 220 rpm. The bacterial cells were harvested by centrifugation and resuspended in phosphate buffered saline (PBS) ([pH 7.4], 137 mM NaCl (Bioshop), 2.7 mM KCl (Bioshop), 10 mM Na2PO4 (Bioshop) and 1.8 mM K2PO4) to an OD600 nm˜1.0, followed by 1/2000 dilution into MHB II.

To a 384-well microtiter plate (Corning, untreated polystyrene), 500 nL of compound was dispensed, in duplicate, and serially titrated 2-fold using an Echo 550 acoustic liquid dispenser (Labcyte Inc.). For compounds dissolved in DMSO, the concentration of DMSO did not exceed 1% of the final well volume. As a solvent control, 500 nL of DMSO was applied to wells that contained no compound. Using a multichannel pipette, 50 μL of the prepared bacterial inoculum was applied to each well. To enable background correction post-incubation, the OD600 nm was measured (Biotek Synergy Neo2 microplate reader) prior to incubation. The plates were incubated at 37° C. with aeration at 900 rpm for 18 h. The plates were equilibrated to room temperature and the OD600 nm measured using a BioTek Synergy Neo2 microplate reader. Data was analyzed in both Prism (GraphPad, Version 9.2.0) and Microsoft Excel (Version 16.53). Raw data were input into Microsoft Excel and the pre-incubation OD600 nm measurements were subtracted from the post-incubation OD600 nm measurements. These corrected measurements were input into Prism 9 as a grouped analysis. MIC values for each compound and the corresponding strain were normalized using Prism 9, where the highest MIC value per compound represented 100%. All other MIC values were adjusted accordingly and visualized using the single-color scale grouped heat map function. Identification of compound chemical properties was achieved using DataWarrior (Version 5.5.0). Using PubChem, the SMILES chemical notation for each compound was obtained and compiled in Microsoft Excel. The resulting spreadsheet was input into DataWarrior and properties were calculated from the compound chemical structures.

### Measuring PAβN and NMP Antibiotic Synergy Using the EKO-35 Efflux Platform

Synergy measurement using checkerboard analysis was performed in 96-well microtiter plates using the microdilution broth method, according to CLSI guidelines (Patel J. B., 2015). The cell inocula were prepared using the colony resuspension method. The minimum inhibitory concentrations (MICs) were defined as the lowest concentration that provided no growth, as determined by measurement of the OD₆₀₀ₙₘ using a Synergy H1 microplate reader (BioTek). Susceptibility testing was performed in MHB II with a final volume of 100 μL. PAI3N (Bachem Americas) was solubilized in MHB II. When assessing PAI3N synergy, linezolid, oxacillin, fusidic acid, and erythromycin were solubilized in 100% DMSO. Ciprofloxacin and novobiocin were solubilized in distilled water. NMP, linezolid, oxacillin, fusidic acid were solubilized in 50% DMSO (v/v), and erythromycin in 50% ethanol (v/v). Ethidium bromide and ciprofloxacin were solubilized in distilled water. Solvent controls were included in each plate.

The Fractional Inhibitory Concentration Index (FICI) was used to assess the synergy of PAI3N in combination with different antibiotics. The FICI represents the ΣFIC of each drug. The FICI for each drug was calculated using the following formula: FICI=FIC_{A}+FIC_{B}=(C_{A}/MIC_{A})+(C_{B}/MIC_{B}). Where MIC_{A} and MIC_{B} are the MICs of drugs A (PAI3N/NMP) and B (antibiotic) alone, and CA and CB are the MICs of the drugs in combination. The effects of PAβN or NMP in combination with antibiotics were classified as: synergistic (FICI<0.5), additive (FICI>0.5-1.0), indifferent (FICI>1.0-2.0), and antagonistic (FICI>2.0). Fold increases in resistance provided by efflux pumps were calculated by dividing the MIC values of strains expressing efflux pumps by the MIC value of EKO-35.

### AcrB Western Blot Analysis

Overnight cultures of EKO-35, EKO-35 araC::acrB, and EKO-35 araC::acrBD408A were subcultured (1:100) into 200 mL LB and incubated at 37° C. with 220 rpm shaking until an optical density (OD600 nm) of 0.6 was reached. The cells were harvested by centrifugation (5,000×g) and resuspended in 100 mM Tris HCl [pH 7.0] with 150 mM NaCl. The cells were lysed by sonication and the sonicate was cleared at 4,000×g for 10 min, followed by removal of occlusion bodies at 20,000×g for 20 min. Crude membranes were isolated by ultracentrifugation at 100,000×g for 1 h. Membrane fractions were resuspended in 100 mM Tris HCl [pH 7.0] with 150 mM NaCl and 2% sodium dodecyl sulphate (SDS). Fractions were quantified using a BCA assay (Thermo Fisher). 10 μg of each sample was resolved by SDS-PAGE and transferred to Amersham™ Protran™ 0.45 μM nitrocellulose membrane (GE) for Western blot analysis using a polyclonal rabbit anti-AcrB (Hazel, A. J. et al., 2019) and an anti-Rabbit IgG(H+L) horseradish peroxidase (HRP) conjugated secondary antibody (Invitrogen). Visualization was performed using the Luminata Crescendo Western chemiluminescent HRP substrate (Millipore) and a Bio-Rad ChemiDocXRS+ system. Total protein normalization was achieved using Bio-Rad stain-free gel imaging and ImageLab (version 6.1).

### Sample Preparation for Proteomic Analysis

Saturated LB overnight cultures of the wild-type and EKO-35 strains were inoculated (1/100 dilution) into 50 mL of LB until the mid-exponential phase of growth was reached (OD600 nm=0.5). 3×10 9 cells were harvested by centrifugation (4000×g), washed twice in phosphate-buffered saline (PBS), and the cell pellet was flash frozen in liquid nitrogen. Whole-cell proteome samples were generated using a modified total proteome extraction protocol (Rappsilber, J. et al., 2007). The cell pellets were stored at −80° C. until the experiment was performed. Briefly, the cell pellet was resuspended in ice cold 100 mM Tris-HCl [pH 8.5]. The cells were lysed using a probe sonicator (three cycles of 30 sec on/off, 30% amplitude) and the samples were treated with 2% SDS and 10 mM dithiothreitol (DTT). To enrich membrane proteins, bacterial membranes were isolated as escribed above. To minimize contamination with cytoplasmic proteins, the membrane pellet was washed in ice cold 100 mM Tris-HCl [pH 8.5], followed by ultracentrifugation. The membrane pellet was resuspended in 100 mM Tris-HCl [pH 8.5] with 2% SDS and 10 mM dithiothreitol (DTT). Both the whole-cell and membrane samples were heated to 95° C. prior to treatment with 55 mM iodoacetamide (IAA). The samples were then incubated with 100% acetone overnight at −20° C. Precipitated protein from both the whole-cell and membrane fractions were combined by centrifugation at 10,000×g at 4° C. and washed twice with 80% acetone. The protein pellets were then solubilized in 40 mM HEPES [pH 5.0] with 8 M urea and quantified using a bovine serum albumin (BSA) tryptophan assay. The samples were digested overnight at room temperature with LysC and trypsin proteases (Promega, protein/enzyme ratio 50:1). 10% v/v trifluoroacetic acid (TFA) was then added and 50 μm acidified peptides were purified and desalted using a STop And Go Extraction (STAGE) tip with 3 layers of C18 resin using the described protocol (Rappsilber, J. et al., 2007).

### Mass Spectrometry and Bioinformatic Analysis

Dried peptides were suspended in Buffer A (2% (v/v) acetonitrile, 0.1% (v/v) trifluoroacetic acid, 0.5% (v/v) acetic acid) and 25 ng of peptides were analyzed on a Q Exactive™ HF-X hybrid quadrupole-orbitrap mass spectrometer (ThermoFisher Scientific) coupled to an EasynLC™ 1200 High-Performance Liquid Chromatography (ThermoFisher Scientific). The samples were loaded onto an in-line 75 μm×50 cm PepMap RSLC EASY-Spray column filled with 2 μm C18 reverse-phase silica beads (ThermoFisher Scientific). Peptides were separated and directly electrosprayed into the mass spectrometer using a linear gradient from 3 to 20% Buffer B (80% (v/v) acetonitrile, 0.5% (v/v) acetic acid) over 18 min, from 20 to 35% Buffer B over 31 mins, followed by a steep 2 min ramp to 100% Buffer B for 9 min in 0.1% formic acid at a constant flow of 250 nL/min. The mass spectrometer was operated in data-dependent mode, switching automatically between one fill scan and subsequent MS/MS scans of 30 most abundant peaks, with full-scans (m/z 400-1600) acquired in the Orbitrap analyzer with a resolution of 60,000 at 400 m/z.

Raw mass spectrometry files were analyzed using MaxQuant (ver. 1.6.14.0 (Cox, J. & Mann, M., 2008). The spectra were searched using the Andromeda search engine with the E. coli K-12 proteome as reference (Accession No. P000000625, accessed June 2021 with 4391 sequences). A minimum of two distinct peptides were required for protein identification and the FDR was set to 1%. The ‘match between runs’ feature of MaxQuant was enabled. Quantification was performed by label-free quantification (LFQ) using the MaxLFQ algorithm (Cox, J. et al., 2014). The mass spectrometry proteomics data have been deposited to the ProteomeXchange Consortium via the PRIDE partner repository with the dataset identifier PXD033975. All mass spectrometry experiments were performed in biological quadruplicate.

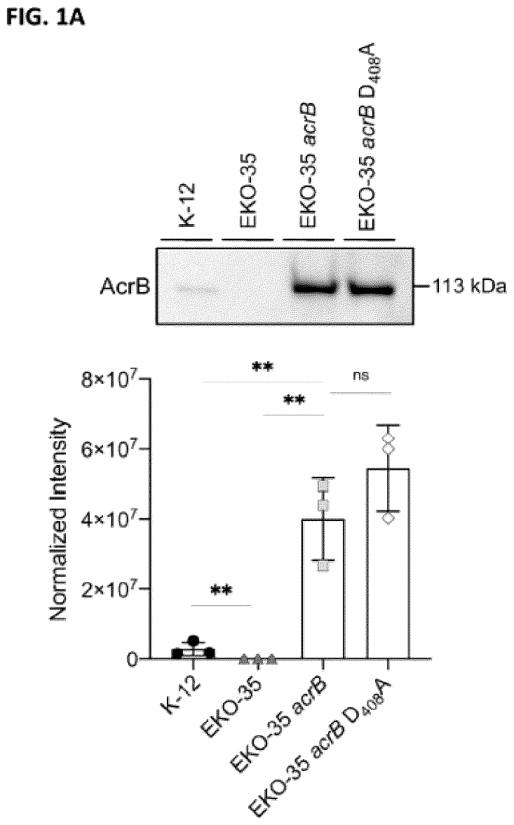

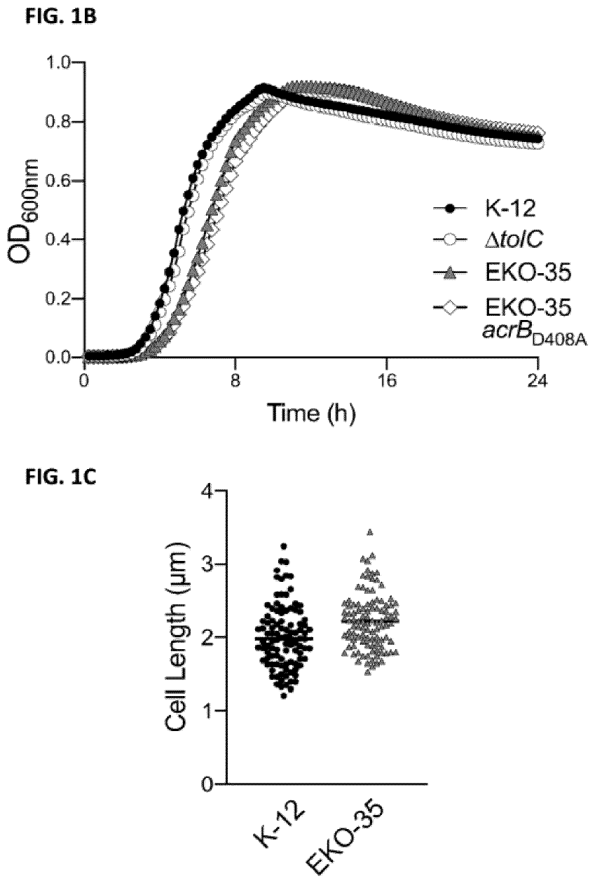

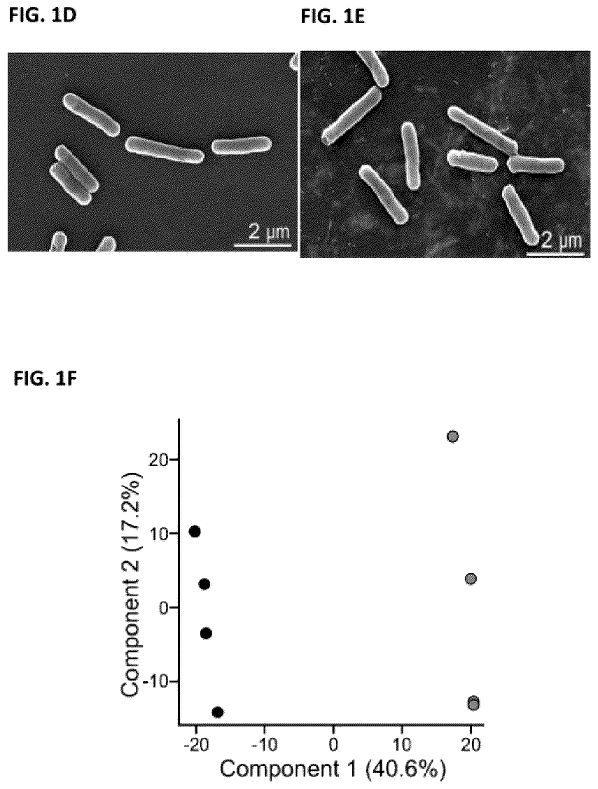

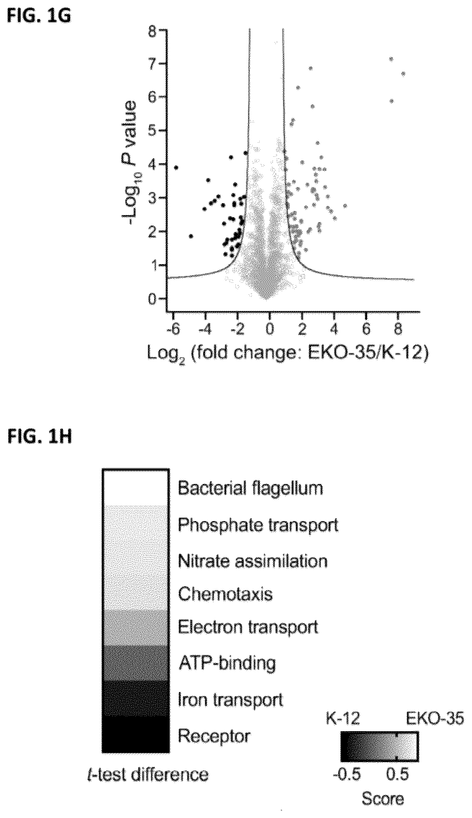

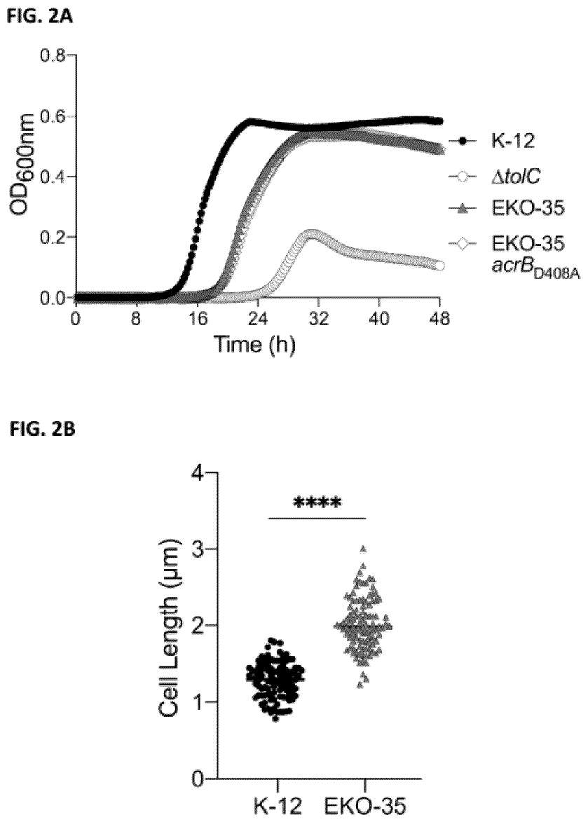

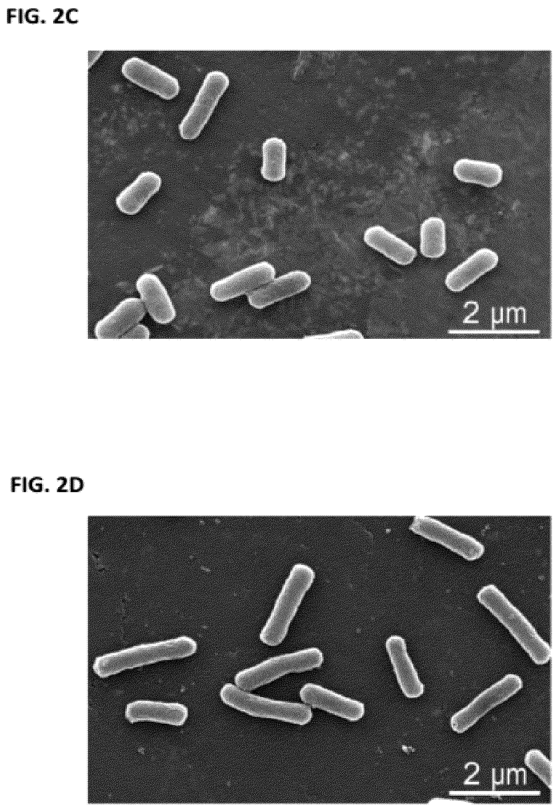

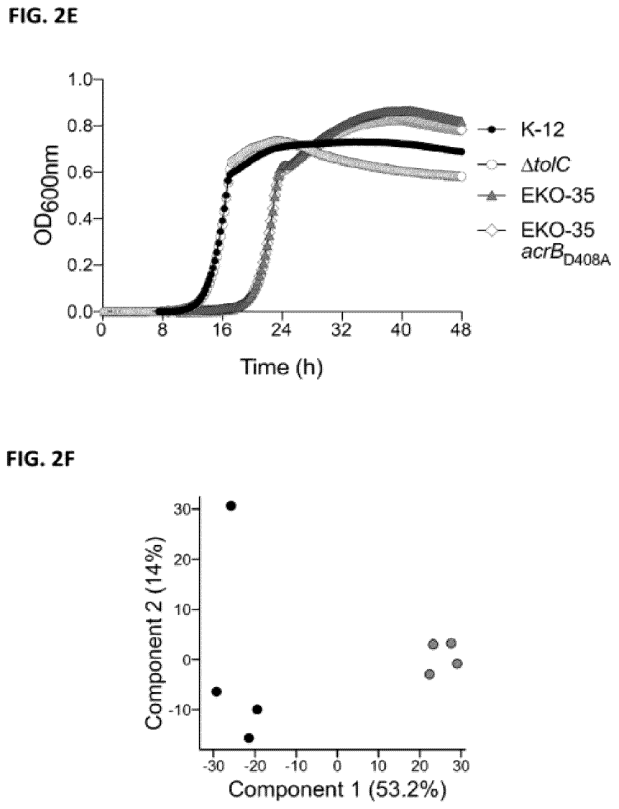

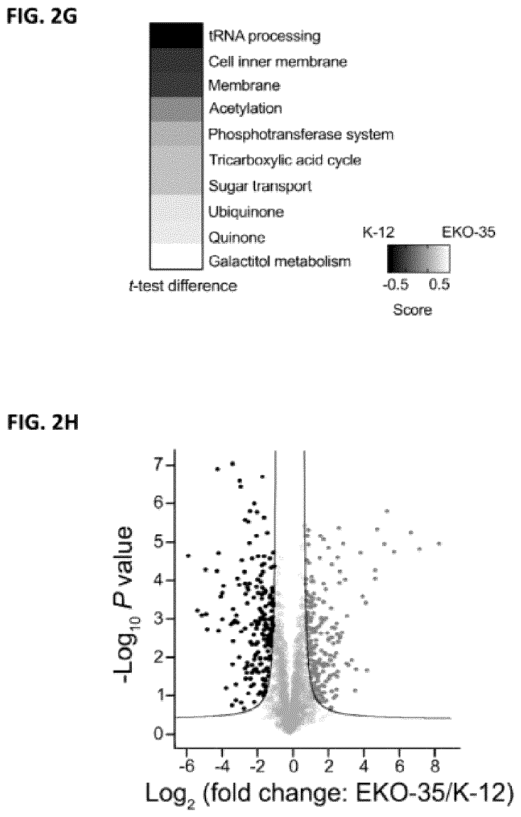

In nutrient-rich conditions, a total of 1,979 proteins were identified from whole cell extracts of the wild-type strain, which represented 45% of the predicted proteome. Principal component analysis (PCA) separated EKO-35 from the wild-type strain (Component 1, 40.6%), with slight variation observed between the biological replicates (Component 2, 17.2%) (FIG. 1F). In nutrient-rich conditions, biological replicate reproducibility was 96.9% and 96.6% between wild-type and EKO-35 replicates, respectively. In nutrient-limited conditions, Inventors identified a total of 2,019 proteins from whole cell extracts of the wild-type strain, which represented 46% of the predicted proteome. PCA defined significant separation between EKO-35 and the wild-type strain (Component 1, 53.2%), and biological variation (Component 2, 14%) (FIG. 2F). In nutrient-limited conditions, biological replicate reproducibility was 94.8% and 96.6% between wild-type and EKO-35 replicates, respectively. Comparative analysis was performed with proteins identified in at least three biological replicates (1,880 proteins in nutrient-rich, and 1,819 proteins in nutrient-limited).

Statistical analysis and data visualization was performed using Perseus (version 1.6.2.2) (Cox, J. & Mann, M. 2012). Further analysis was only performed on proteins present in at least three out of four biological replicates within each strain. Upon filtering, missing values were imputed from a normal distribution. Significant changes in abundance of proteins between the two proteomes were defined with a false discovery rate (FDR)-correct Student's t-test (p-value 0.05) and Benjamini-Hochberg multiple hypothesis correction testing (FDR=0.05) was applied. 1D-annotation enrichment analysis of UniProt keywords enabled global assessment of changes in protein abundance (Tyanova, S. et al., 2016) using two-sample t-tests with P-value 0.05, FDR=0.05 and score <−0.5, <0.5. Data visualization was performed using GraphPad (version 8).

### Results

Generation of EKO-35: Inactivation of E. coli Drug Efflux Pumps

Inventors' first goal was to generate a simplified genetic background to overcome the challenges associated with the complexities of intact drug efflux networks. Using a combination of λ-Red recombineering and CRISPR Cas9-mediated counter selection. Inventors sequentially inactivated 36 genes encoding IM pumps from the genome of E. coli K-12 strain BW25113. Inventors started with an ΔacrB mutant from the Keio Collection, and then removed a further 12 pumps using the λ-Red system. One limitation of this approach is the possibility of unintended genomic deletions due to the introduction of adjacent Flp recognition target sites following the removal of numerous genes. Thus, the remaining genes were inactivated using CRISPR Cas9-mediated counter selection, and the introduction of three tandem stop codons into the beginning of each gene (Table 3).

The efflux genes were inactivated in the following order: ΔacrB; acrD; acrF; mdtF; macB; emrB; mdtL; mdtK; bcr; ydeA; mdtM; yddA; yebQ; emrE; mdtD; sugE; ynfM; emrD; ydeF; mdtJ; ydiM; mdtB, mdlA; emrY; mdfA; fsr; mdtG; mdtH; yieO; mdlB, mdtO, yojH, yojI, yajR, ydhC; cusA. While generating the strain, inventors identified a missense mutation (L269P) within the gene encoding the Hns-dependent flhDC regulator (HdfR), which inventors deduced was present in the acrB mutant used as the starting background of EKO-35. To ascertain whether the hdfR mutation occurred in response to loss of acrB, inventors removed acrB from the genome of the wild-type E. coli BW25113 strain using the λ-Red system. Sequencing of the hdfR gene revealed the mutation was not present (data not shown). Since HdfR is a transcriptional regulator of important physiological processes, and the mutation did not appear to be induced by loss of acrB, inventors repaired this mutation using CRISPR Cas9-mediated counter selection.

Since many of the efflux pump-encoding genes have predicted start codons and are poorly characterized, it is possible that alternative start codons located downstream from the inserted tandem stop codons (Table 3) could be utilized for a subset of the CRISPR-inactivated pumps. To investigate, inventors profiled the genome of the efflux-deficient strain, which included the inserted stop codons, using the Prodigal prokaryotic gene recognition and translation initiation site identifier. Overall, the program did not predict production of any potentially functional efflux pumps, which supports the notion that the tandem stop codons were sufficient to prematurely terminate translation, and that alternate start codons would not be utilized (Tables 26A and 26B). Indeed, Prodigal analysis of the wild-type strain's genome predicted production of all pumps (Tables 26A and 26B).

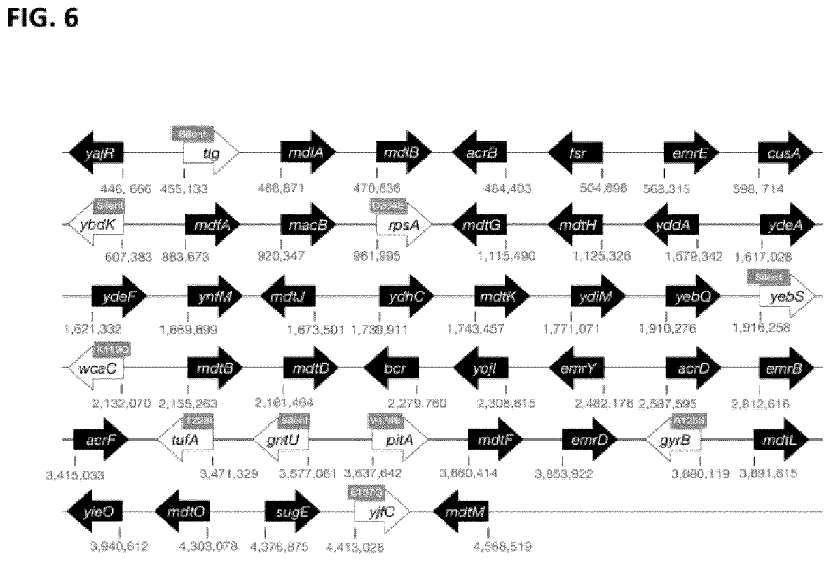

In addition, inventors also took advantage of new developments in protein structure predictions, and carefully analyzed AlphaFold-generated models of each efflux pump to ascertain whether the predicted start codons were correct based on the structural features of the proteins. Such an analysis indicated the inserted stop codons were sufficient to inactivate the different genes; however, the modeled structure of a predicted ABC efflux pump (YojH) indicated that the yojH gene does not encode an efflux pump. While yojH is located adjacent to another ABC efflux pump, YojI, which does structurally resemble an ABC transporter, the predicted YojH structure lacks the structural features of an ABC-type efflux pump, including transmembrane helices. Investigating further, inventors observed that YojH is described as malate:quinone oxidoreductase, a membrane-associated enzyme involved in the citric acid cycle/glyoxylate cycle. However, the function of this protein in E. coli is poorly described and the enzyme has not been shown to catalyze oxidation of malate in E. coli. Since the gene does not encode an efflux pump, inventors repaired the inactivated yojH gene by removing the inserted stop codons using CRISPR Cas9-mediated counter selection. This strain was subsequently designated Efflux KnockOut-35 (EKO-35), and was used for phenotypic characterization and construction of an efflux platform, as described below. The EKO-35 genome sequence confirmed successful disruption of the 35 efflux-encoding genes, including successful repair of hdfR and yojH, and also revealed ten additional secondary mutations (FIG. 6), six of which encoded missense mutations and four silent substitutions. Inventors mapped the occurrence of these missense mutations to the disruption of specific genes (Table 5 and Table 6).

### Characterizing EKO-35: Phenotypic Analysis

With the intent of using EKO-35 as a simplified genetic background to study the functions of drug efflux pumps, inventors' second goal was to fully characterize EKO-35 prior to further investigations. Other efflux-deficient backgrounds (e.g., tolC mutants) display pleiotropic phenotypes, including severe growth defects in minimal M9 medium with glucose supplied as the carbon source. Indeed, tolC inactivation causes periplasmic accumulation of enterobactin in this low-iron growth medium, which underlies this conditionally essential phenotype. In addition, tolC mutants exhibit membrane stress and depletion of essential metabolites, resulting in ‘metabolic shutdown’ in minimal glucose medium. Thus, inventors were curious to ascertain whether loss of 35 IM efflux pumps also affected the fitness of E. coli.

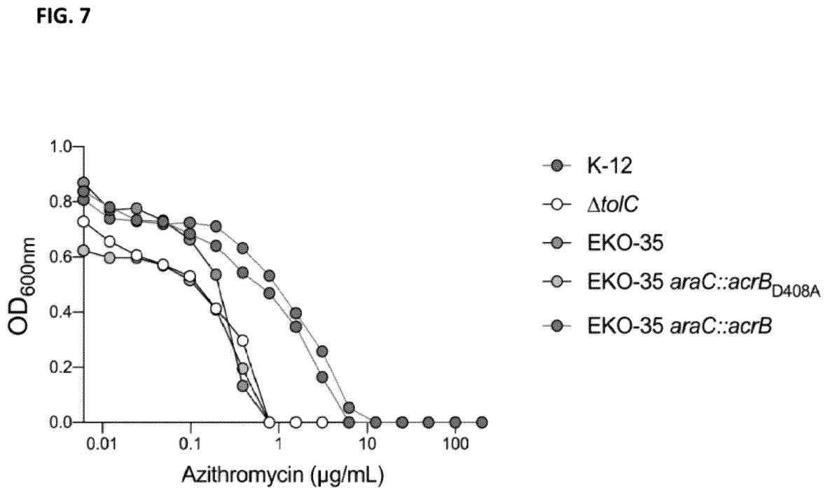

First, inventors generated a control strain that was used as a comparator during phenotypic profiling of EKO-35. RND efflux pumps, such as AcrB, are trimeric complexes spanning the IM up to 36 times. It has been suggested that caution should be taken when interpreting phenotypes of gene deletion mutants since such phenotypes could be due to loss of integral membrane proteins, rather than loss of efflux function. Based on previous studies, inventors introduced a D₄₀₈A substitution into AcrB to enable the production of an efflux inactive variant in EKO-35. Using the pINT2 plasmid, the mutated acrB gene was integrated into the arabinose operon of EKO-35 (EKO-35 araC::acrB_{D408A}), with gene expression under the control of the constitutive P_{LacI} promoter. Inventors confirmed the AcrB_{D408A} protein was inactive and present at comparable levels to the wild-type protein within the membrane of EKO-35 (FIGS. 1A and 7).

Next, while the successful generation of EKO-35 showed the E. coli drug efflux system is dispensable for growth, inventors measured the growth kinetics and assessed the cellular morphology of EKO-35 under commonly used laboratory conditions: nutrient-rich (Lysogeny broth) and nutrient-limited (M9 defined minimal medium with glucose) medium at 37° C. (FIGS. 1B and 2A, Table 1). Compared to the wild-type strain, EKO-35 exhibited a 1 h extended lag phase in the nutrient-rich medium (FIG. 1B and Table 1), and phenotypic analysis revealed no significant changes in morphology (FIGS. 1C and 1E). Under nutrient-limitation at 37° C., EKO-35 entered the exponential stage of growth ˜5 h later than the parental strain, and exhibited longer cell length (FIG. 2B and Table 1). As a comparison, inventors also profiled a ΔtolC mutant from the Keio Collection, which revealed there were distinct differences between the strains under nutrient-limited conditions (FIGS. 1B and 2A). As mentioned above, loss of TolC results in severe growth defects in nutrient-limited conditions due to compounding pleiotropic effects, including the accumulation of enterobactin due to iron limitation. However, inventors reveal deletion of all IM efflux pumps, including those that form complexes with TolC, does not elicit the same fitness cost (FIG. 2A). Inventors also measured the growth kinetics of EKO-35 in a defined MOPS medium, which included supplemental iron, revealing that the ˜5 h delay in the EKO-35 lag phase was not restored (FIG. 2E).

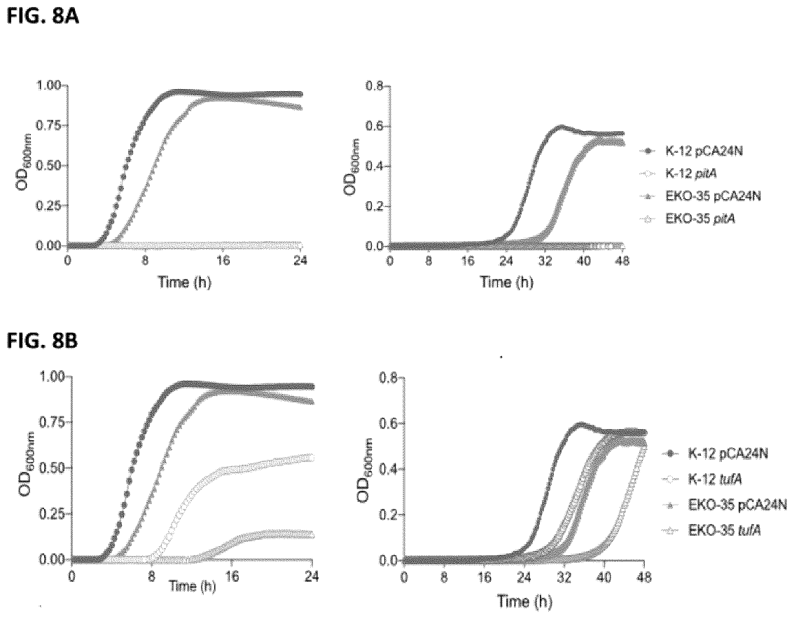

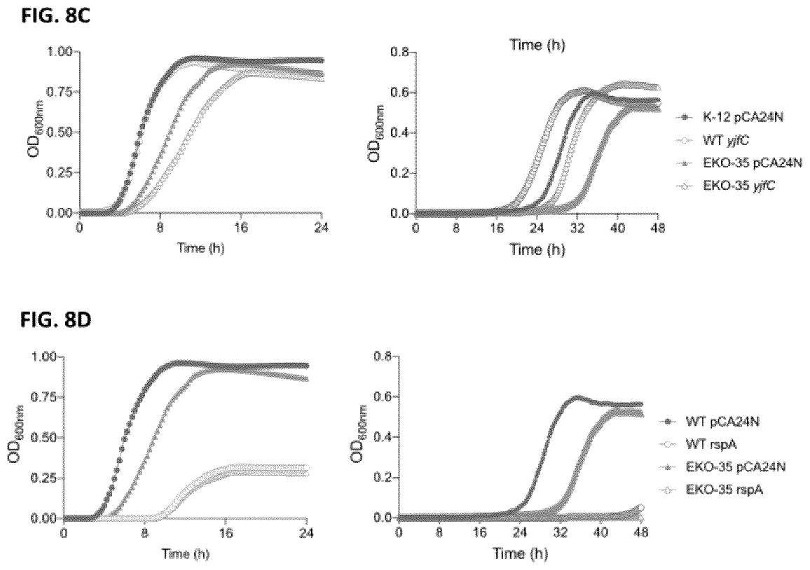

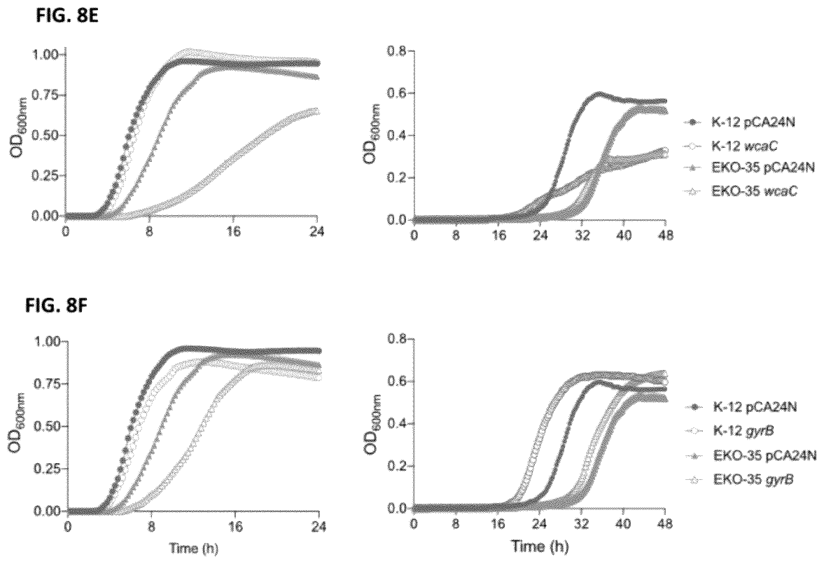

Next, to explore whether the nonsynonymous EKO-35 mutations (Table 5 and Table 6) were compensatory, and to ascertain if they impacted any of the observed phenotypes detailed in this disclosure, inventors obtained clones from the E. coli ASKA library harboring the rspA, tufA, pitA, wcaC, gyrB, and yjfC genes on plasmids with gene expression under the control of an IPTG-inducible promoter. Growth profiling revealed pitA overexpression conferred a lethal phenotype, and the overexpression of the remaining genes induced a fitness cost in EKO-35 under nutrient-rich optimal growth conditions, indicating that the mutations could be compensatory in nature (FIG. 8A (right)). A lethal phenotype was also observed for pitA in the wild-type strain, and tufA and rspA also decreased fitness (FIGS. 8A (right), 8C (right), and 8D (right)). Under nutrient-limited conditions, pitA and rspA overexpression induced a lethal phenotype and wcaC reduced the fitness of both strains (FIGS. 8E (left), 8A (left), and 8D (left)). Finally, gyrB and yjfC marginally increased the fitness of both strains in nutrient-limited conditions (FIG. 8F (left), FIG. 8B (left)). Overall, findings of this disclosure show the nonsynonymous mutations could alleviate fitness costs induced by loss-of-efflux in EKO-35.

### Characterizing EKO-35: Phenotypic Analysis

With the intent of using EKO-35 as a simplified genetic background to study the functions of drug efflux pumps, inventors' second goal was to fully characterize EKO-35 prior to further investigations. Other efflux-deficient backgrounds (e.g., tolC mutants) display pleiotropic phenotypes, including severe growth defects in minimal M9 medium with glucose supplied as the carbon source. Indeed, tolC inactivation causes periplasmic accumulation of enterobactin in this low-iron growth medium, which underlies this conditionally essential phenotype. In addition, tolC mutants exhibit membrane stress and depletion of essential metabolites, resulting in ‘metabolic shutdown’ in minimal glucose medium. Thus, inventors were curious to ascertain whether loss of 35 IM efflux pumps also affected the fitness of E. coli.

First, inventors generated a control strain that was used as a comparator during phenotypic profiling of EKO-35. RND efflux pumps, such as AcrB, are trimeric complexes spanning the IM up to 36 times. It has been suggested that caution should be taken when interpreting phenotypes of gene deletion mutants since such phenotypes could be due to loss of integral membrane proteins, rather than loss of efflux function. Based on previous studies, inventors introduced a D₄₀₈A substitution into AcrB to enable the production of an efflux inactive variant in EKO-35. Using the pINT2 plasmid, the mutated acrB gene was integrated into the arabinose operon of EKO-35 (EKO-35 araC::acrB_{D408A}), with gene expression under the control of the constitutive P_{LacI} promoter. Inventors confirmed the AcrB_{D408A} protein was inactive and present at comparable levels to the wild-type protein within the membrane of EKO-35 (FIGS. 1A and 7).

Next, while the successful generation of EKO-35 shows the E. coli drug efflux system is dispensable for growth, inventors measured the growth kinetics and assessed the cellular morphology of EKO-35 under commonly used laboratory conditions: nutrient-rich (Lysogeny broth) and nutrient-limited (M9 defined minimal medium with glucose) medium at 37° C. (FIGS. 1B and 2A, Table 1). Compared to the wild-type strain, EKO-35 exhibited a 1 h extended lag phase in the nutrient-rich medium (FIG. 1B and Table 1), and phenotypic analysis revealed no significant changes in morphology (FIG. 1C). Under nutrient-limitation at 37° C., EKO-35 entered the exponential stage of growth ˜5 h later than the parental strain, and exhibited longer cell length (FIG. 2B and Table 1). As a comparison, inventors also profiled a ΔtolC mutant from the Keio Collection, which revealed there were distinct differences between the strains under nutrient-limited conditions (FIGS. 1B and 2A). As mentioned above, loss of TolC results in severe growth defects in nutrient-limited conditions due to compounding pleiotropic effects, including the accumulation of enterobactin due to iron limitation. However, inventors reveal deletion of all IM efflux pumps, including those that form complexes with TolC, does not elicit the same fitness cost (FIG. 2A). Inventors also measured the growth kinetics of EKO-35 in a defined MOPS medium, which included supplemental iron, revealing that the ˜5 h delay in the EKO-35 lag phase was not restored (FIG. 2E).

Next, to explore whether the nonsynonymous EKO-35 mutations (Table and Table 6) were compensatory, and to ascertain if they impacted any of the observed phenotypes detailed in this disclosure, inventors obtained clones from the E. coli ASKA library harboring the rspA, tufA, pitA, wcaC, gyrB, and yjfC genes on plasmids with gene expression under the control of an IPTG-inducible promoter. Growth profiling revealed pitA overexpression conferred a lethal phenotype, and the overexpression of the remaining genes induced a fitness cost in EKO-35 under nutrient-rich optimal growth conditions, indicating the mutations could be compensatory in nature (FIG. 8A (left)). A lethal phenotype was also observed for pitA in the wild-type strain, and tufA and rspA also decreased fitness (FIGS. 8A (left), 8C (left), and 8E (left)). Under nutrient-limited conditions, pitA and rspA overexpression induced a lethal phenotype and wcaC reduced the fitness of both strains (FIGS. 8A (right), 8D (right), 8E (right)). Finally, gyrB and yjfC marginally increased the fitness of both strains in nutrient-limited conditions (FIGS. 8F (right) and 8B (right)). Overall, findings from this disclosure show that the nonsynonymous mutations could alleviate fitness costs induced by loss-of-efflux in EKO-35.

### Characterizing EKO-35: Quantitative Proteomic Profiling

To further characterize EKO-35, and to gain fundamental insight into how E. coli responds to loss-of-efflux, inventors assessed fluctuations in the membrane-enriched cellular proteome of cells sampled at the mid-exponential phase of growth (FIGS. 1F and 2D). In nutrient-rich conditions, a total of 1,979 proteins were identified from whole cell extracts of the wild-type strain, which represented 45% of the predicted proteome. Principal component analysis (PCA) separated EKO-35 from the wild-type strain (Component 1, 40.6%), with slight variation observed between the biological replicates (Component 2, 17.2%) (FIG. 1F).

Comparative proteomics showed significant changes in the abundance of 111 proteins in response to loss of the E. coli efflux system: 38 proteins were significantly increased in the wild-type proteome, and 73 proteins were significantly increased in the proteome of EKO-35 (FIG. 1G). The most significant differences in protein abundance were identified for two distantly related Fus homologs, YddB and PqqL (Table 7), which were >7-fold more abundant in EKO-35. YddB is an OM protein with structural homology to the OM ferredoxin transporter FusA. PqqL is a periplasmic metalloprotease analogous to the ferrodoxin processing protease FusC. Along with YddA, these proteins are proposed to function as a poorly characterized iron-uptake system. YddA is a member of the ABC efflux pump superfamily, which was disrupted during the generation of EKO-35. YddA phylogenetically clusters with other putative drug efflux pumps—YojI, MdlA, and MdlB. Other proteins highly abundant in EKO-35 included components of the flagellar apparatus (FliC), chemotaxis-associated proteins (Tsr), periplasmic chaperones (Spy), and metabolism-associated proteins, SrlA and SrlB (Table 7). In the wild-type strain, the most differentially abundant proteins (>3-fold) included components of the ferric citrate transport system⁴⁰ (FecA, FecB, and FecE), heat-shock molecular chaperone proteins (IbpA, IbpB), and the peptidase component of the HslVU protease (HslV) (Table 7).

Using a false discovery rate (FDR) of 2%, annotation enrichment 42 of the EKO-35 cellular proteome revealed few differences compared to the wild-type proteome. Increasing the FDR to 5% identified three categories enriched in the wild-type strain, including iron transport. These categories correlated to the reduced abundance of proteins with annotated functions associated with iron-transport in EKO-Drug efflux pumps are well-characterized for their role in siderophore extrusion. Therefore, EKO-35 can downregulate iron acquisition systems to alleviate the fitness cost associated with accumulation of these molecules. However, it is important to note that these experiments were performed in relatively nutrient-rich conditions. In addition, five categories were enriched in EKO-35, including bacterial flagellum, phosphate transport, nitrate assimilation, chemotaxis, and electron transport (FIG. 1H).

Next, comparative proteomics was applied to assess fluctuations in the proteome of EKO-35 in nutrient-limited conditions. Inventors identified a total of 2,019 proteins from whole cell extracts of the wild-type strain, which represented 46% of the predicted proteome. PCA defined significant separation between EKO-35 and the wild-type strain (Component 1, 53.2%), and biological variation (Component 2, 14%) (FIG. 2F). Compared to nutrient-rich conditions, a greater number of significant changes in protein abundances were identified: 188 proteins were significantly increased in the wild-type proteome, and 190 proteins were significantly increased in the EKO-35 proteome (FIGS. 2G and 2H). Similar to nutrient-rich conditions, EKO-35 displayed increased abundance of YddB and PqqL (>7-fold). However, inventors also observed increased abundance of nickel ABC transport component NikD, metabolic proteins (SdaA, GarL, DmlA), periplasmic chaperones (Spy and CpxP), and the efflux pump membrane fusion protein MdtA (Table 8). The abundance of Spy, CpxP, and MdtA is tightly controlled at the transcriptional level by the Cpx envelope stress response regulon, which is induced by extracellular stress or the disruption of periplasmic homeostasis. This shows that loss-of-efflux disrupted periplasmic homeostasis under nutrient-limitation. In the wild-type strain, AcrB was observed under both nutrient-limited and nutrient-rich conditions, reinforcing that AcrB is constitutively produced under standard laboratory conditions (Tables 7 and 8).

Similar to nutrient-rich conditions, annotation enrichment revealed few differences between the wild-type and EKO-35 proteomes using an FDR of 2%. When the FDR was increased to 5%, the wild-type proteome was enriched with categories including acetylation, tricarboxylic acid cycle, sugar transport, ubiquinone, and quinones (FIG. 2H). In the EKO-35 proteome, proteins with annotated subcellular localization in the cell inner membrane were enriched (FIG. 2H), further indicating membrane stress in EKO-35, which can be compensated by increased abundance of membrane-associated proteins to alleviate changes in membrane fluidity due to lack of efflux pumps, or the accumulation of endogenously produced physiological substrates. In addition, the increased abundance of proteins with annotated functions associated with metabolism in the wild-type strain shows the metabolism of EKO-35 was impacted due to loss-of-efflux under these conditions.

Finally, two proteins associated with the EKO-35 nonsynonymous mutations were detected in the proteomes of wild-type E. coli and EKO-35; PitA was significantly increased in the wild-type strain, supporting the notion that PitA can negatively impact EKO-35 fitness. Additionally, inventors also detected a higher abundance of RspB in the wild-type proteome, which is encoded in an operon with rspA, further supporting that the nonsynonymous mutation in rspA may mitigate an associated fitness cost. Indeed, EKO-35 can have both reduced protein abundance and mutated the corresponding genes to alleviate the fitness cost associated with loss-of-efflux.

### Characterizing EKO-35: Investigating Conditional Essentiality

E. coli is a versatile organism withstanding diverse and challenging environments. It is suggested the majority of efflux pumps are not constitutively produced under optimal growth conditions, which can underpin the observed dispensability of the drug efflux system (FIGS. 1B and 2A). Indeed, in nutrient-rich conditions inventors identified AcrB, YojI, and MdtK in the proteome of the wild-type strain, and AcrB, YojI, MdtK, and CusA in nutrient-limited conditions (Table 9). A significant number of efflux pumps were not identified in these conditions, indicating they can be differentially expressed, or that they are produced at levels that inventors were unable to detect.

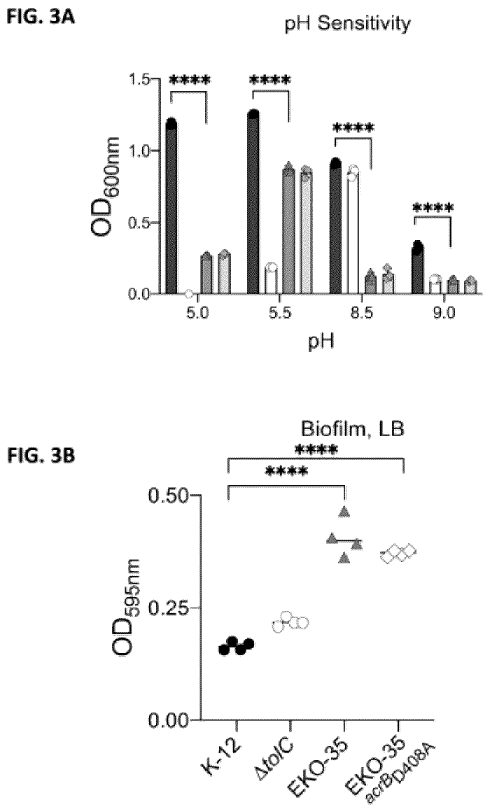

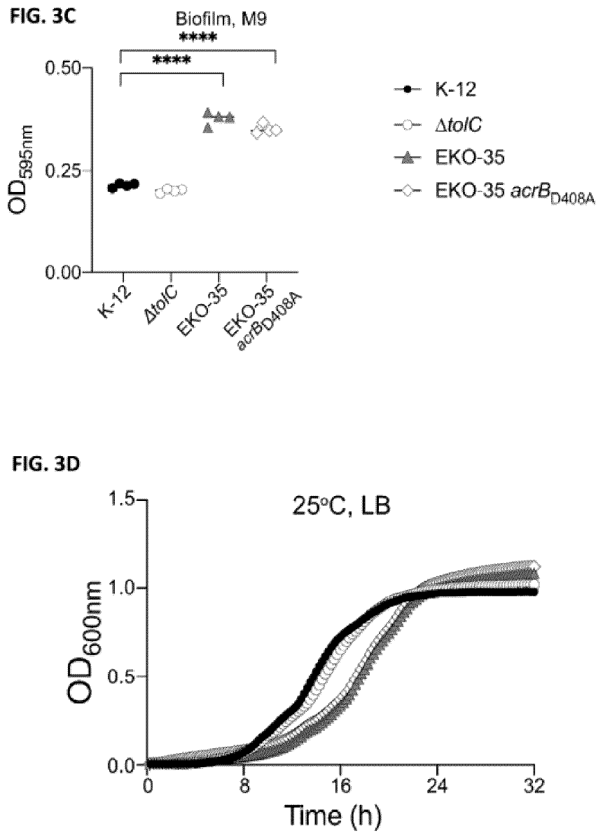

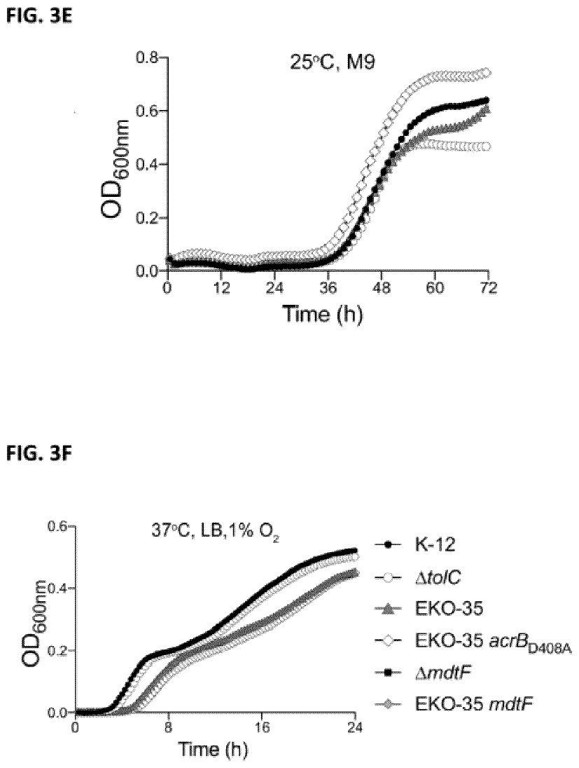

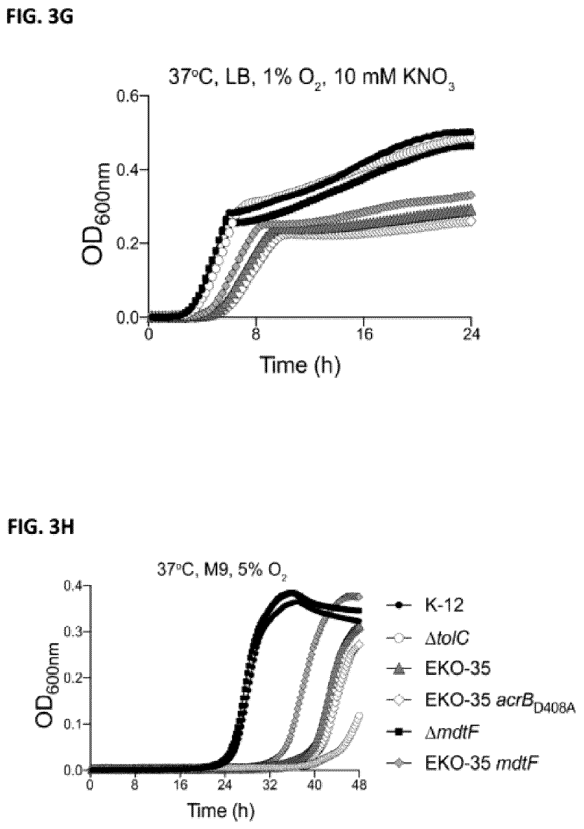

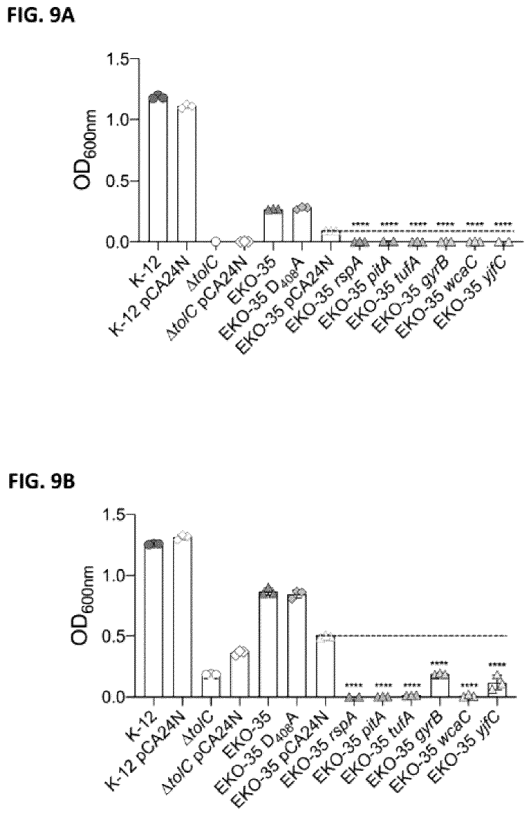

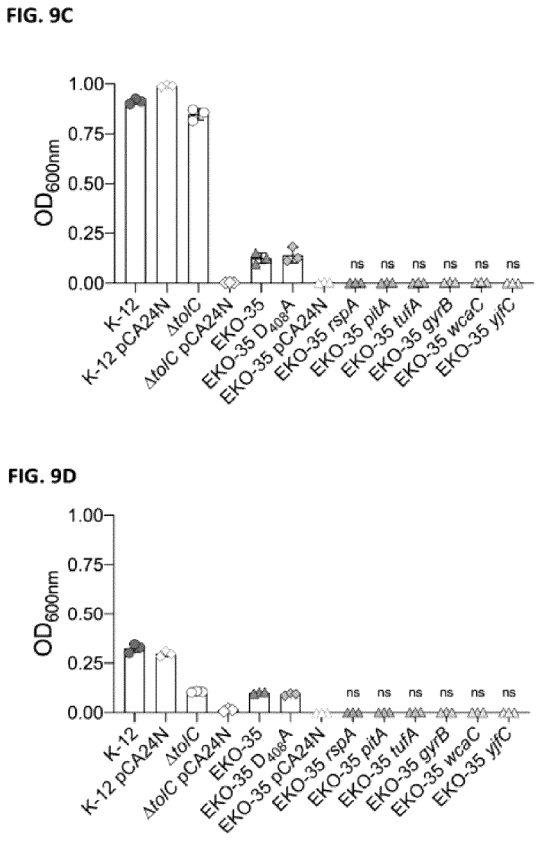

To ascertain whether the E. coli efflux system becomes conditionally essential, inventors profiled EKO-35 growth under a range of different conditions. First, inventors confirmed the essentiality of drug efflux pumps for survival under extreme acid and alkaline conditions (FIG. 3A), Efflux pumps are frequently associated with pH homeostasis; for example, moderate acidic conditions increase the expression of mdtEF, mdtG, mdtlJ, and mdtL efflux genes, and the loss of MdtB and EmrB drug efflux pumps impacts fitness at extreme acidic pH. In addition, ΔtolC mutants show decreased fitness during extreme acid exposure. Efflux pumps (e.g., MdfA and MdtM) also contribute to alkalitolerance via Na⁺/(K⁺)/H⁺ antiport-based mechanisms, decreasing the cytoplasmic pH. As such, here inventors show that at pH values ≤5.5 and >8, the E coil efflux system is essential for growth (FIG. 3A). In addition, due to the abovementioned contribution of single component IM pumps to alkaline tolerance, EKO-35 shows pH dispensability patterns that differ from the ΔtolC mutant (FIG. 3A). To ensure the increased pH sensitivity was not associated with the nonsynonymous EKO-35 genomic mutations, growth of EKO-35 harboring the different ASKA clones—rspA, tufA, pitA, wcaC, gyrB, and yjfC—was assessed, revealing these genes were unable to restore the fitness of EKO-35 under acidic and alkaline conditions (FIGS. 9A-9D).

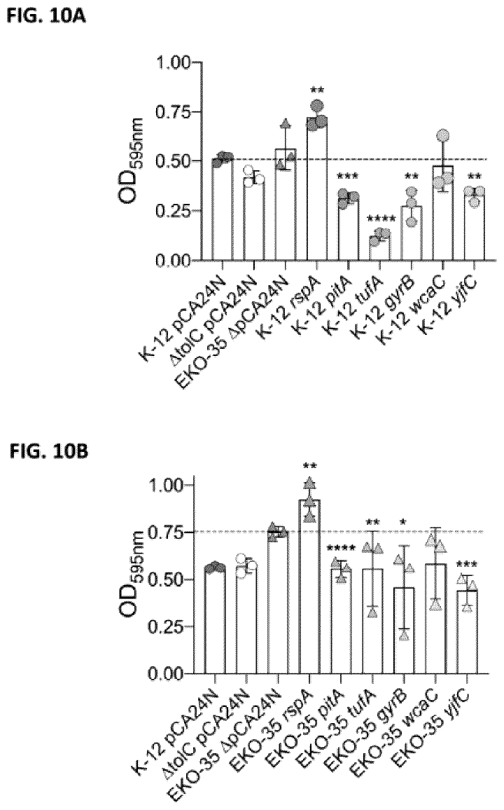

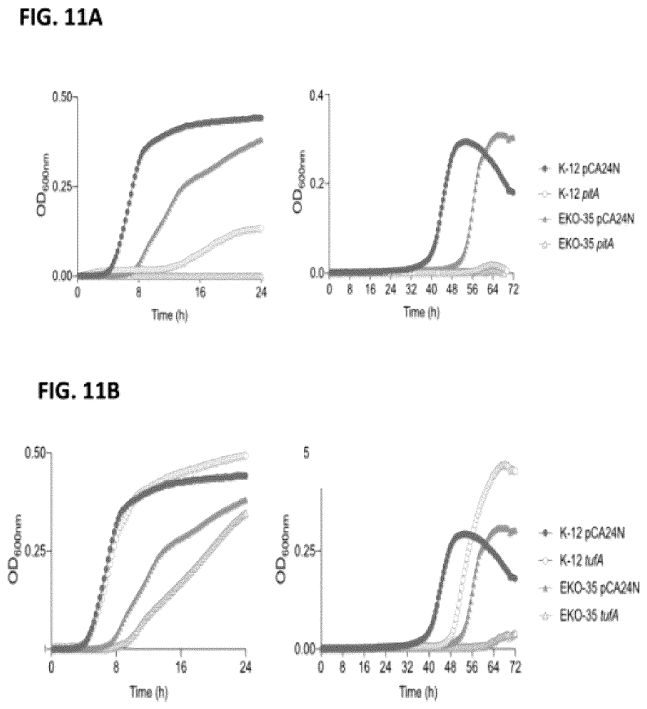

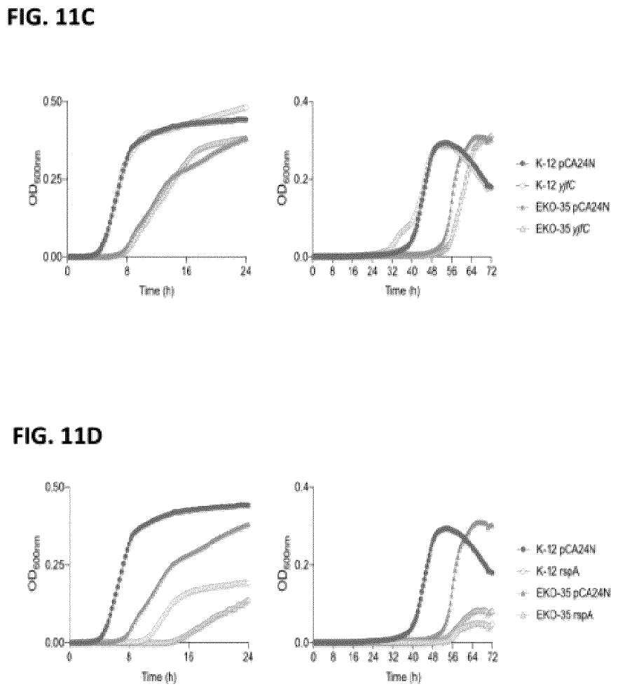

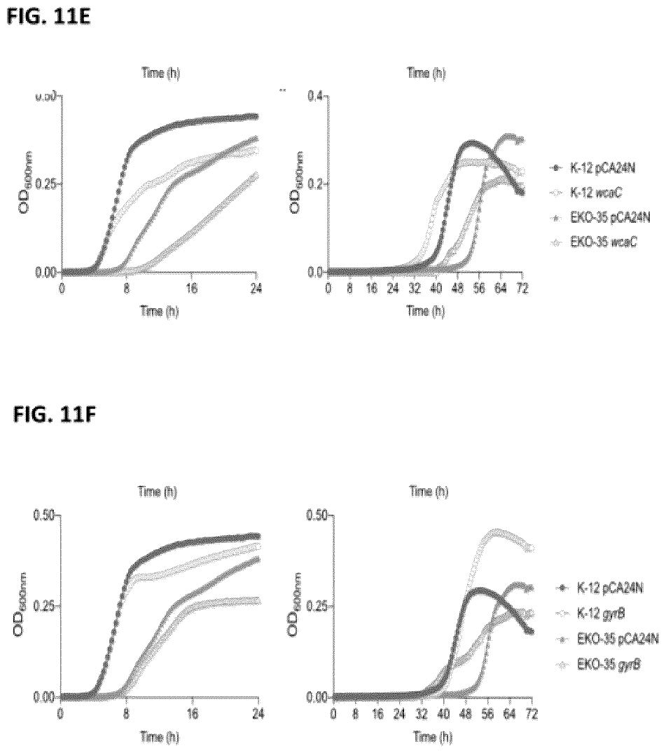

Since efflux pumps have also been associated with biofilm formation, inventors next explored whether loss-of-efflux impacted biofilm formation in E. coli. EKO-35 was propagated statically under both nutrient-rich and nutrient-limited conditions (FIGS. 3B and 3C), revealing loss-of-efflux enhanced biofilm formation compared to the wild-type strain in both conditions. In addition, the ΔtolC mutant exhibited biofilm phenotypes distinct to EKO-35. To investigate whether the phenotype was associated with the nonsynonymous mutations in EKO-35, the biofilms of EKO- and wild-type E, coil expressing rspA, tufA, pitA, wcaC, gyrB, and yjfC were characterized in nutrient-rich conditions. Expression of rspA significantly increased biofilm formation in both EKO-35 and the wild-type strain, whilst the remaining genes significantly lowered biofilm formation in both strains (FIGS. 10A and 10B) Overall, findings of this disclosure highlight that rspA, tufA, pitA, wcaC, gyrB, and yjfC are associated with biofilm formation in E. coli. Future studies should investigate whether the increase in EKO-35 biofilm formation is directly induced by loss-of-efflux, or is indirectly associated with efflux due to these apparently compensatory nonsynonymous mutations.

Next, inventors measured the growth kinetics of EKO-35 at 25° C., and under reduced oxygen concentrations, in both nutrient-rich and -limited conditions (Table 1). The lag-phase was significantly extended in nutrient-rich medium; however, no significant differences were identified in nutrient-limited medium (FIGS. 3D and 3E, and Table 1). Under low oxygen conditions, inventors observed the fitness of EKO-35 was impaired in the nutrient-rich medium (1% 02) at 37° C. (FIG. 3F and Table 1). Growth profiling of EKO-35 harboring the ASKA plasmids did not restore the observed phenotype (FIGS. 9A-9D). The addition of KNOB, which emulates the presence of nitrosative indole derivatives, further impacted EKO-35 fitness (FIG. 3G and Table 1). The MdtEF tripartite efflux assembly has been reported to contribute to fitness under anaerobic conditions, through the removal of toxic by-products produced during anaerobic respiration. However, an MdtF mutant (ΔmdtF) from the Keio Collection did not show any differences in growth (FIG. 3H). In contrast, expression of mdtEF in EKO-35 (EKO-35 araC::mdtEF strain) partially restored the growth of EKO-35 (Table 1), which supports the contribution of MdtEF to E. coli fitness under these conditions, and also shows that additional efflux genes can contribute to the observed phenotype.

Most notably, inventors observed that the E. coli drug efflux system is essential for growth in a nutrient-limited low-oxygen environment (5% O₂) (FIG. 3H). Measurement of EKO-35 growth kinetics (Table 1) revealed the strain remained in the lag phase for ˜18 h longer than the wild-type strain and was unable to enter the stationary phase during the duration of the experiment. This phenotype was not restored through the expression of genes harboring nonsynonymous mutations in EKO-35; in fact, the overexpression of pitA, tufA, and rspA decreased fitness further (FIGS. 9A-9D). Expression of mdtEF partially restored fitness (FIG. 3H). In addition, inventors also observed significant differences between the growth of EKO-35 and ΔtolC (FIG. 3H).

In summary, by profiling diverse growth conditions, inventors reveal instances where the fitness of EKO-35 is impacted due to loss-of-efflux. EKO-35 also exhibited phenotypes distinct to the ΔtolC mutant, which provides important biological insight into the physiological functions of drug efflux pumps. Indeed, EKO-35 is an important tool to further characterize the role of drug efflux pumps in physiological processes. The present findings highlight potentially compensatory nonsynonymous mutations that require further investigation.

EKO-35 Displays Differing Susceptibility Levels to a ΔtolC Mutant

Due to the observed phenotypic differences observed between EKO-35 and the ΔtolC mutant, inventors explored whether the strains also exhibited differences in susceptibility to growth inhibitors. Since inventors disrupted all drug efflux pumps known to form complexes with TolC, in addition to single component IM pumps, inventors posited it was likely EKO-35 would exhibit comparable—if not increased susceptibility—to antimicrobial agents.

Inventors curated and profiled a collection of compounds (n=52) with diverse physicochemical properties, including molecular weight (138.059 g/mol to 1449.27 g/mol), lipophilicity (log P −7.8597 to 5.823), aqueous solubility (log S −9.422 to and total polar surface area (PSA) (0 to 530.49 A2) (Table 10). This collection included well-described antibiotics, dyes, detergents, antiseptics, bile acids, and a subset of poorly characterized synthetic compounds (FIG. 12; compounds 1-19) recently identified as efflux substrates in E. coli. Inventors also included vancomycin as a permeability control, which is ordinarily unable to cross the OM, and is not a substrate for efflux 22.

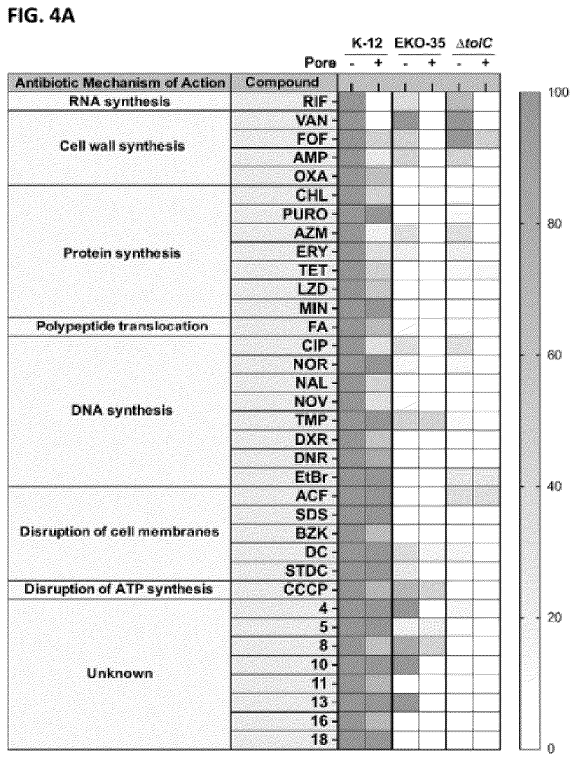

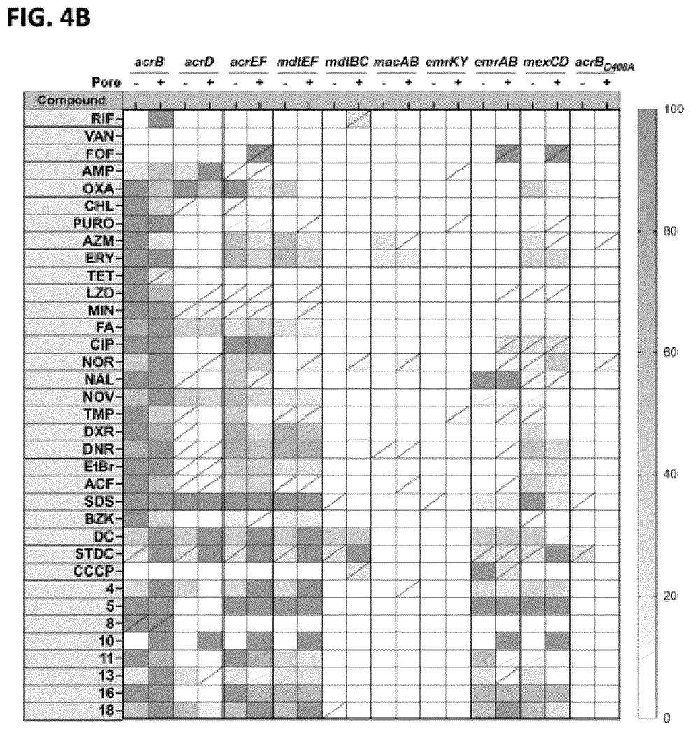

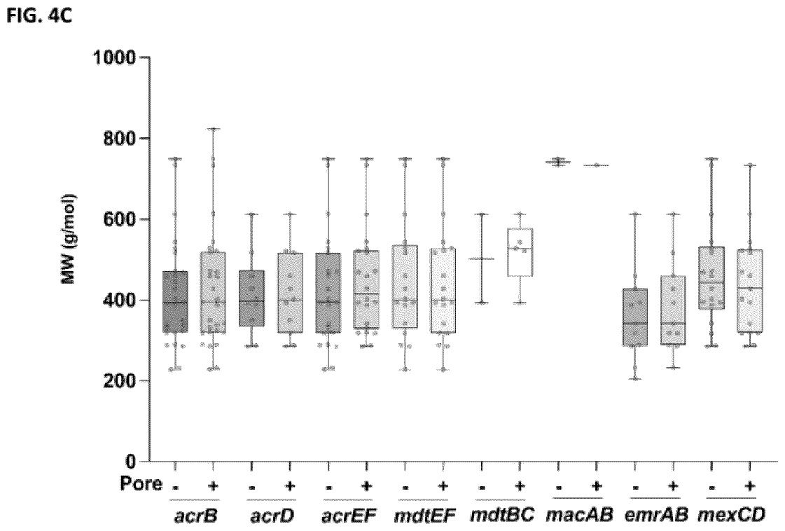

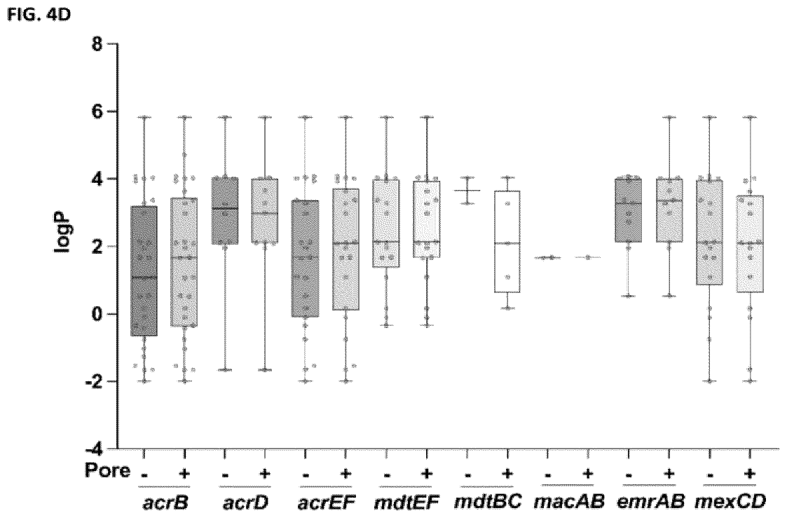

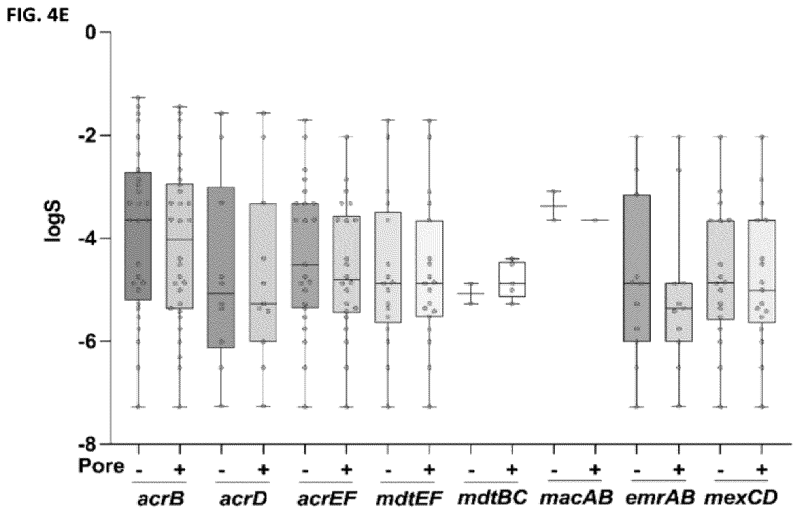

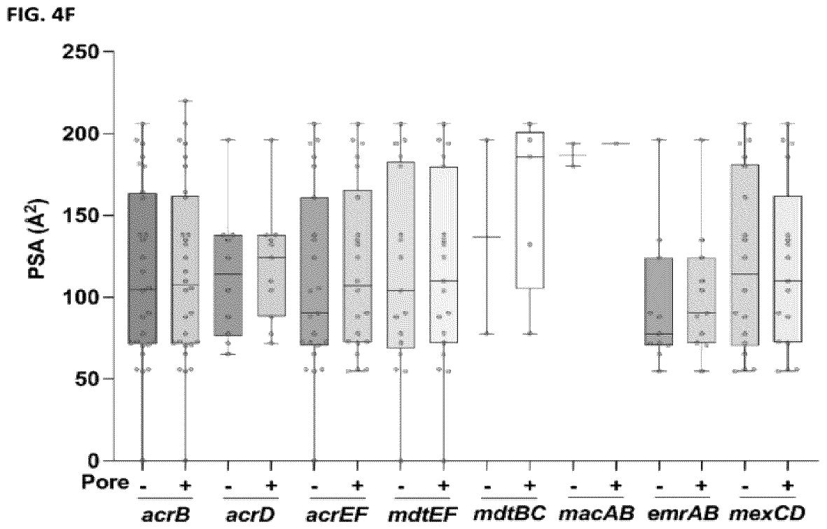

Overall, EKO-35 and the ΔtolC mutant had similar susceptibility profiles for ˜65% (n=34) of the compounds, which were largely well-characterized antibiotics. However, the ΔtolC mutant was more susceptible to 31% of the profiled compounds, and a majority of these were the synthetic and poorly-characterized compounds (Table 10). These compounds also included novobiocin (4-fold difference) (Table 10), which inventors predicted could be due to the EKO-35 nonsynonymous mutation in gyrB (Table 5 and Table 6). Finally, EKO-35 was more susceptible to two compounds (acriflavine and ethidium bromide), which display relatively lower log P values (−1.9857 and −0.102, respectively) and a narrow molecular weight range (˜390 to 460 g/mol) (FIG. 4A and Table 10).

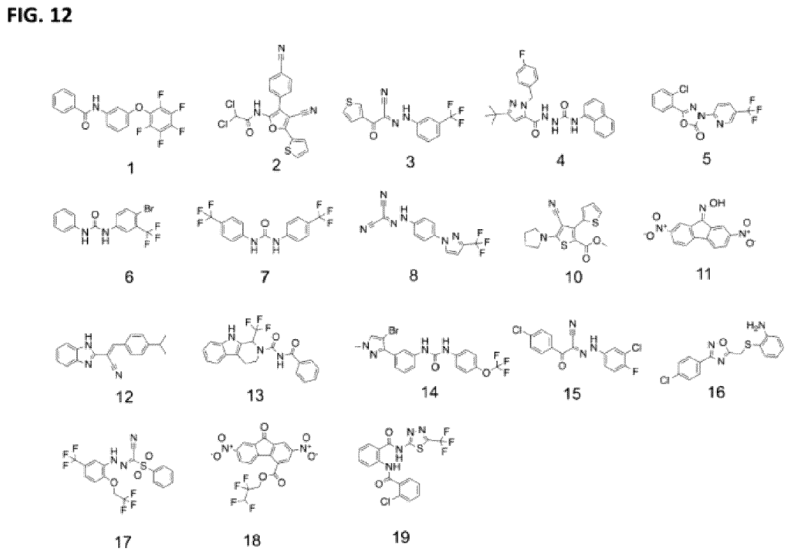

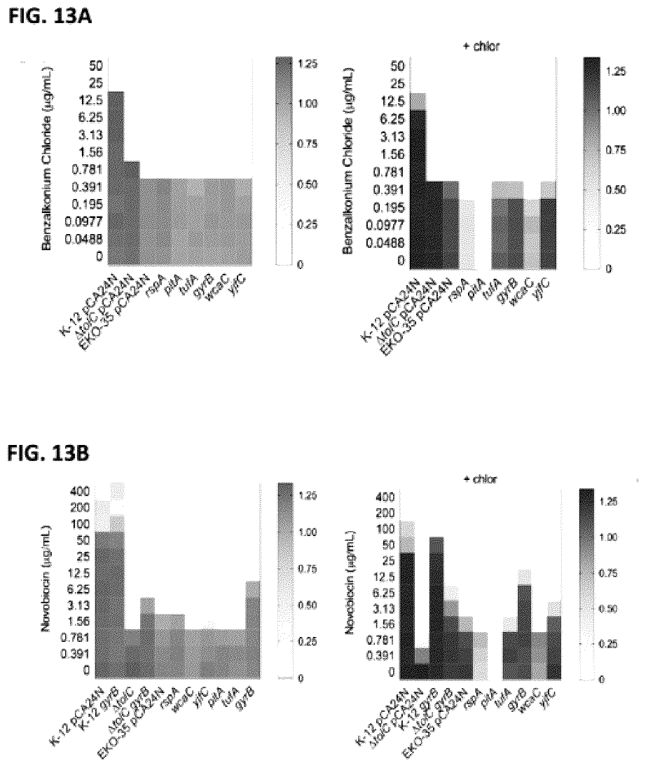

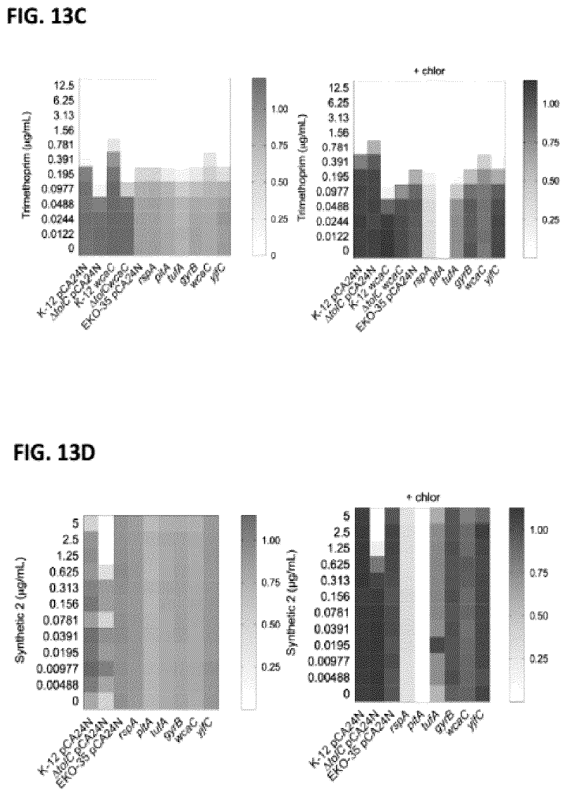

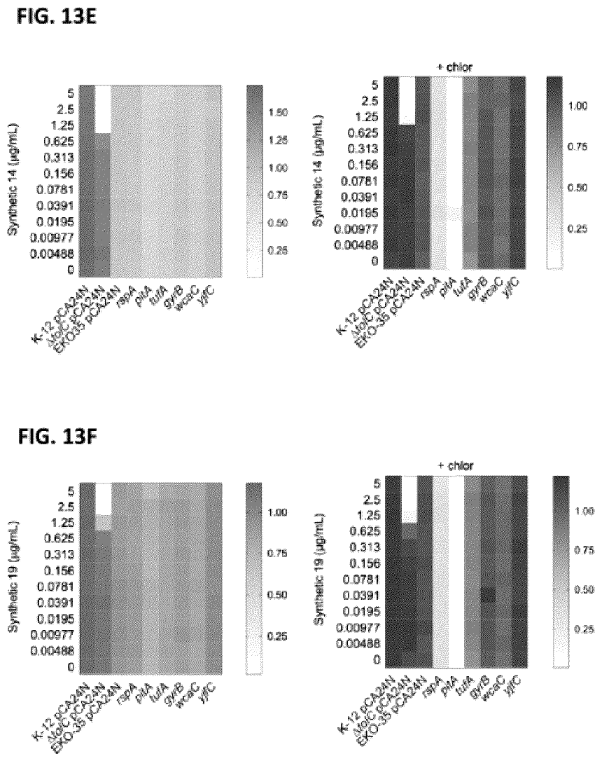

To investigate whether the differences in susceptibility between EKO-35 and ΔtolC were associated with the six missense mutations identified within the EKO-genome (FIGS. 13A-13D), inventors assessed the susceptibility of EKO-35 harboring the ASKA plasmids expressing rspA, tufA, pitA, wcaC, gyrB, and yjfC. It was not possible to assess the contribution of pitA to susceptibility since overexpression induced a lethal phenotype. Overall, the remaining genes did not impact the susceptibility of EKO-35 to these compounds, with the exception of gyrB overexpression, which increased novobiocin resistance due to target over production 57 (Tables 11A and 11B). Indeed, this phenotype was also observed in the ΔtolC strain (FIGS. 13A-13D). In addition, while profiling the ASKA EKO-35 strains, inventors only observed a 2-fold difference in novobiocin susceptibility between EKO-35 and ΔtolC, which is within the acceptable error range for these assays.

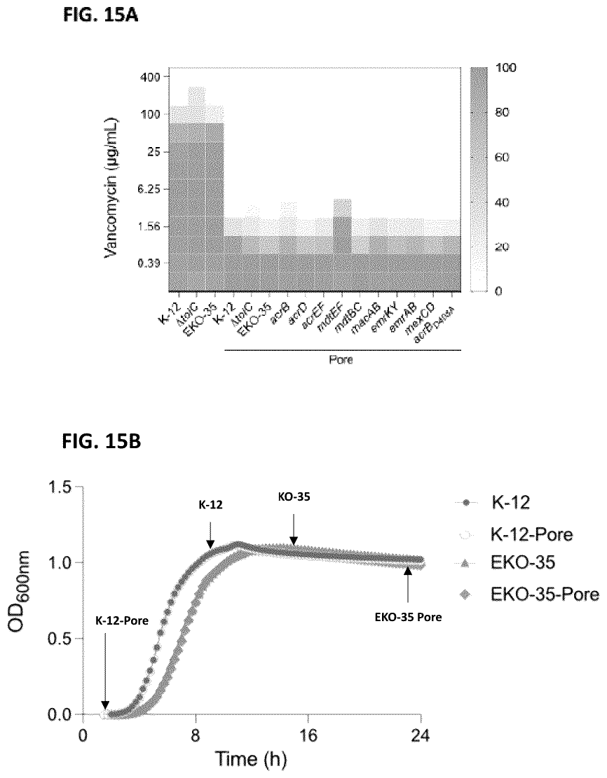

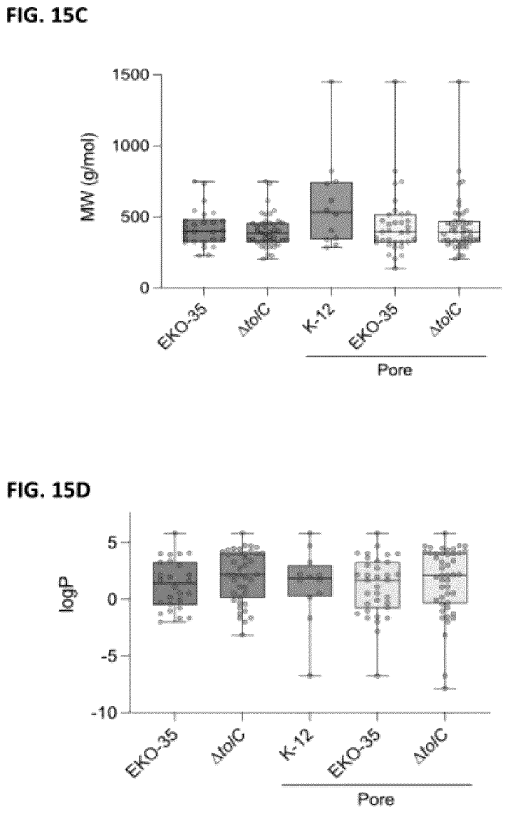

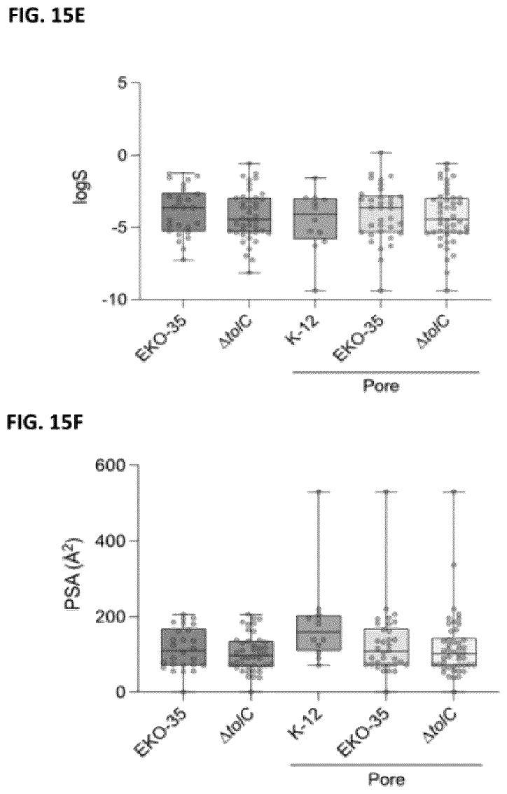

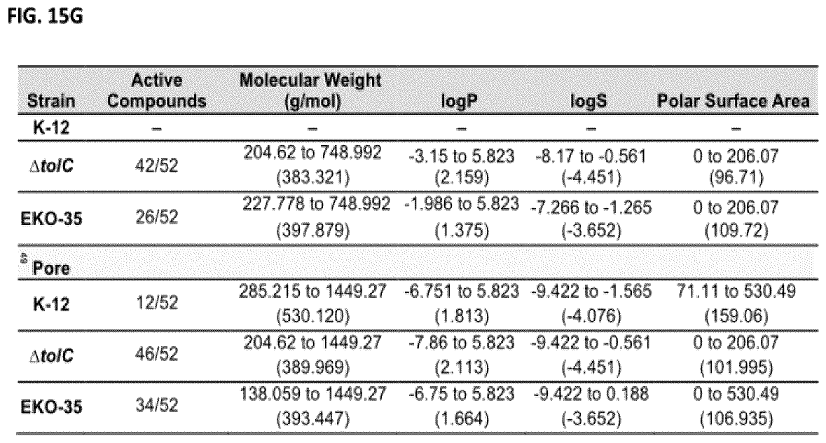

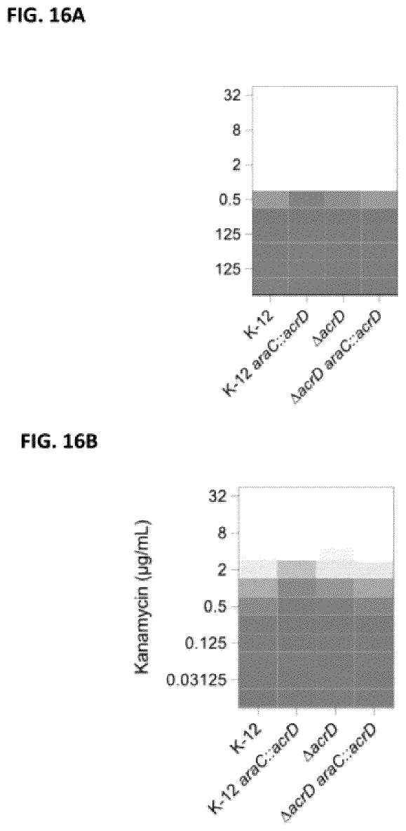

Finally, inventors also generated an EKO-35 porinated strain (EKO-35-Pore), through introduction of an open variant of the OM siderophore transporter FhuA, which herein will be denoted as a ‘pore’. The FhuA transporter is rendered non-selective through removal of a terminal plug domain and four large external loops, which enables production of a ‘pore’ with an internal diameter of ˜2.4 nm. This non-selective pore increases the influx of both hydrophilic and hydrophobic compounds, without affecting efflux. As described previously, inventors introduced the pore into the intergenic region between the glmS and pstS genes in the genome of EKO-35 and the wild-type strain, with gene expression under the control of a constitutive promoter. Growth of the EKO-35-Pore strain was comparable to EKO-35 under optimal conditions (FIG. 15B), and the genome sequence of this strain did not highlight any secondary mutations induced upon introduction of the pore. The pore was also introduced into the wild-type and ΔtolC strains. Inventors confirmed activity of the pore through susceptibility testing with the large antibiotic vancomycin (FIG. 15A).

Next, inventors profiled the wild-type, EKO-35 and ΔtolC porinated strains against the same curated collection of physicochemically diverse compounds (n=52), to gain insight into the physicochemical properties of compounds retarded by the OM and/or those that are susceptible to efflux (FIG. 4 and Tables 13A, 13B, 14A, and 14B). In regard to OM permeability, compared to the wild-type strain, the porinated wild-type strain displayed increased susceptibility to ˜23% of the compounds; the physicochemical properties of these compounds were quite diverse, including compounds with larger molecular weights (˜286 to 1449 g/mol), lower hydrophobicity (−6.751 to 5.823), and larger PSAs (71.11 to 530.49 Å²) (FIGS. 4A-4F and 15A-15G). The pore did not sensitize E. coli to a large number of compounds, showing that efflux plays a more significant role in intrinsic resistance. Indeed, EKO-35 was susceptible to 50% of the compounds, and the ΔtolC strain to ˜80%, demonstrating that active efflux contributes more significantly than OM retardation for the majority of the compounds (FIGS. 4A and 15A). These compounds were relatively smaller (˜204 to 823 g/mol), more hydrophobic (−3.15 to 5.823), exhibiting a narrower range of relatively lower PSAs (0 to 220 Å²) (FIGS. 15B-15G). Compromising the OM of EKO-35 (EKO-rendered the strain susceptible to 65% of the compounds, and the ΔtolC (ΔtolC-Pore) strain to ˜88% (FIGS. 4A and 15A). This observation supports the well-known synergistic relationship between OM diffusion and efflux, which significantly expanded the physicochemical properties of compounds exhibiting activity against E. coli (FIGS. 4C and 15C).

In summary, the porinated strains provide an additional tool kit to study the transport of compounds across the E. coli cell envelope, enabling dissociation between permeation across the OM and active efflux. Indeed, with an intact OM, the physicochemical properties of compounds that could be assessed using the presently disclosed platform would be limited to those that can permeate the OM; for example, the exclusion limit for porins is considered to be ˜600 g/mol. The pore overcomes the selectivity barrier of the OM, widening the physicochemical properties that could be profiled in this disclosure. In addition, the differing susceptibility levels of EKO-35 and the ΔtolC mutant emphasizes that while both strains can be considered efflux-deficient, inactivation of IM efflux pumps impacts susceptibility differently than inactivation of an OM channel. Therefore, functions distinct from these IM efflux pumps could be attributed to the TolC-associated resistance phenotypes. This observation highlights the utility of EKO-35 for the study of drug efflux across the cell envelope.

### Construction of an Efflux Pump Expression Platform

To demonstrate the use of EKO-35 as a tool to investigate the physicochemical substrate specificities of efflux pumps, inventors constructed an efflux platform consisting of strains individually expressing efflux pump-encoding genes. Inventors selected genes encoding pumps that form tripartite complexes with TolC (AcrB, AcrD, AcrEF, MdtEF, MdtBC, EmrAB, EmrKY, and MacAB). In addition, inventors included mexCD from P. aeruginosa—which can function with TolC⁶²—to show that EKO-35 can be used as a broad-spectrum tool to study efflux pumps from other bacterial species. Overall, the intent is for expansion of the platform to include additional efflux pumps of interest as needed.

First, inventors attempted to integrate each gene into the genome of EKO-35 using the pINT1 plasmid, which enables markerless integration of genes into araC, with gene expression under the control of the strong and constitutive P_{Bla} promoter. Inventors reasoned that integrated genes with constitutive expression would provide increased stability and circumvent the need for selective markers and inducers. However, inventors observed numerous deleterious mutations in a subset of the genes following ligation into this vector, and also following integration into the genome (data not shown). Consequently, inventors posited that the expression level could be too high and inventors instead integrated each gene using the pINT2 plasmid, which utilizes the relatively weaker and constitutive P_{LacI} promoter. Following genomic integration into araC and removal of the resistance cassette, each gene integration was confirmed using Sanger sequencing. The EKO-35 integrated strains will herein be referred to as EKO-35 araC::X, where X represents the gene of interest. As a proof of principle, comparison of AcrB levels between the wild-type, EKO-35, and EKO-35 araC::acrB strains confirmed the complementation system was functional and the protein was produced at higher levels than when the gene is expressed at the basal level (FIG. 1A).

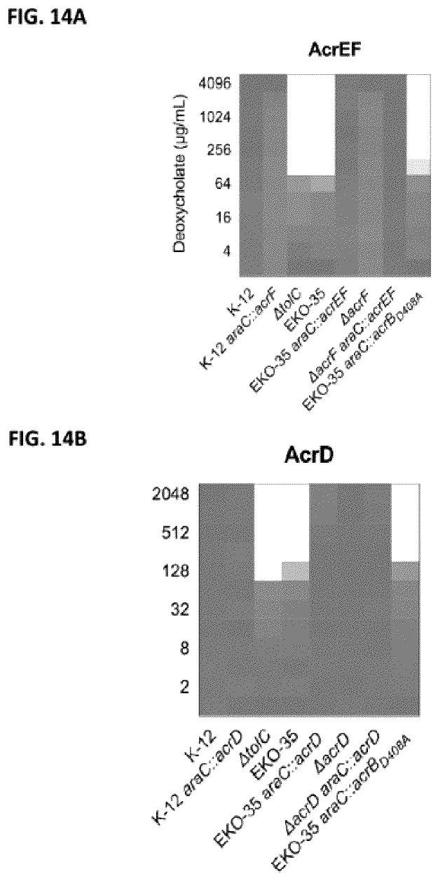

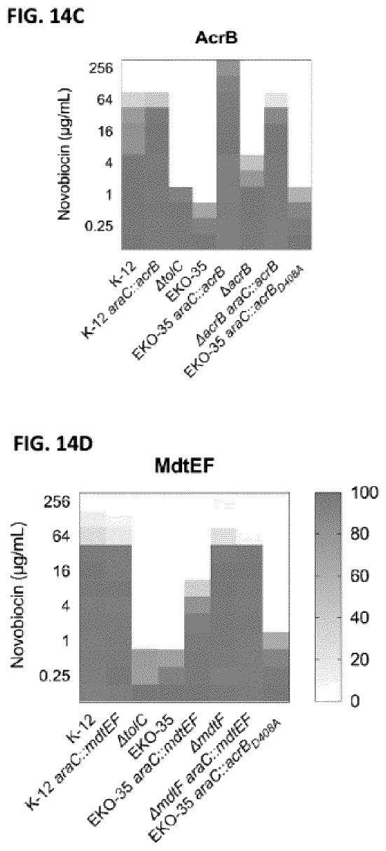

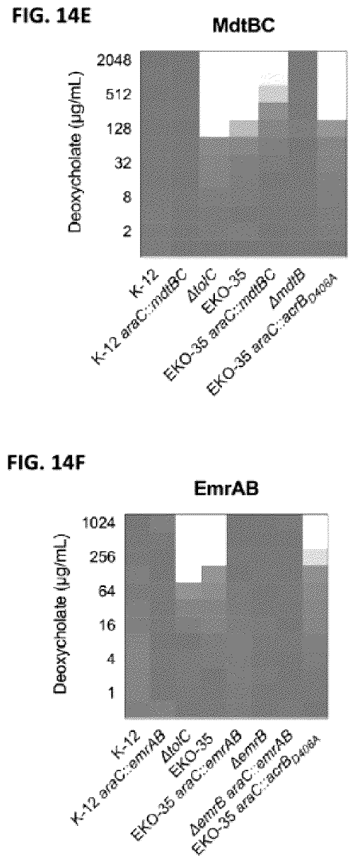

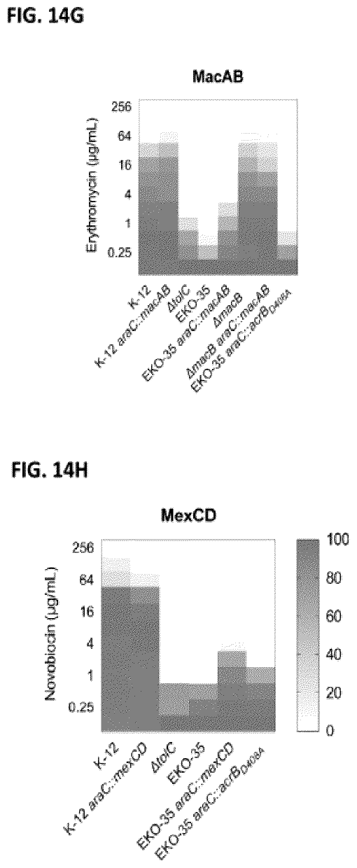

To highlight the utility of EKO-35 for the investigation of efflux pump substrate profiles, the efflux genes mentioned above were also integrated into the genome of the wild-type E. coli BW25113 strain, and single deletion mutants corresponding to the pumps of interest. These strains, and the EKO-35 integrated strains, were profiled against known substrates for each respective efflux pump (Table 12). Since the wild-type strain harbors an intact drug efflux network, there were no differences in susceptibility when the efflux pump-encoding genes were expressed (FIGS. 14A and 14D-14H). Similarly, only one single efflux deletion strain—ΔacrB—displayed susceptibility profiles that differed from the parental strain (FIG. 14C). Indeed, the single efflux deletion mutants still harbor a relatively intact efflux network, which can mask the effects of other efflux pumps, emphasizing the need for a strain such as EKO-35. With the exception of EmrKY, for which inventors were unable to identify a known substrate, all of the integrated genes increased the resistance levels of EKO-35, confirming that each pump was functional (FIGS. 14A-14H).

In summary, due to the well-described differences in efflux pump basal expression levels, the developed efflux platform enables the study of efflux pumps in isogenic background(s) free of the masking effects of promiscuous pumps, with gene expression under the control of the same constitutive promoter. As such, this platform allows for the comparison of efflux pump substrate profiles, profiling of efflux pump inhibitors (EPIs), efflux pump interplay, and delineation of the molecular properties governing efflux, as described below.

### Defining the Physicochemical Substrate Profiles of Drug Efflux Pumps

To investigate the tripartite efflux pump substrate profiles, inventors determined the minimum inhibitory concentrations (MICs) of each compound in the curated collection against each strain within the efflux platform, for both the EKO-35 and EKO-35-Pore strains producing efflux pumps (FIGS. 4A, 4B and Tables 13A, 13B, 14A, and 14B). As described, this compound collection covered a diverse physicochemical space that enabled us to summarize the molecular properties that contribute to efflux in each pump (FIGS. 4C-4E and Tables 13A, 13B, 14A, 14B, 15A, 16A, 16B, and 17).

As anticipated, AcrB was associated with resistance to a significant subset (˜85%) of the compounds (FIG. 4B), supporting the described promiscuous nature of this tripartite pump. The introduction of the pore increased AcrB-mediated resistance to rifampicin, which is a large antibiotic significantly hindered by the OM²² (Tables 14A and 14B). The tripartite complexes AcrEF and MdtEF also conferred broad-spectrum resistance (70% and 52% of the compounds, respectively) (FIG. 4B). Expression of the pore in combination with all three of these pumps (AcrB, AcrEF, and MdtEF) increased resistance to sodium taurodeoxycholate and a poorly-characterized synthetic compound (compound 10), which are both hydrophobic molecules (log P values of 2.091 and 3.638, respectively) (FIG. 4B and Tables 14A and 14B). These changes did not alter the overall physicochemical properties of AcrB and MdtEF (FIGS. 4C-4E and Table 17). In addition, OM porination impacted the ability of AcrEF to confer resistance to nalidixic acid, trimethoprim, and benzalkonium chloride, which are relatively small molecules (FIG. 4B and Tables 14A, 14B and 17). Overall, these three pumps were polyspecific, demonstrating little preference for molecular weight or lipophilicity when compared to the other pumps profiled (FIG. 4C-4E and Tables 13A, 13B, 14A, 14B, and 17).

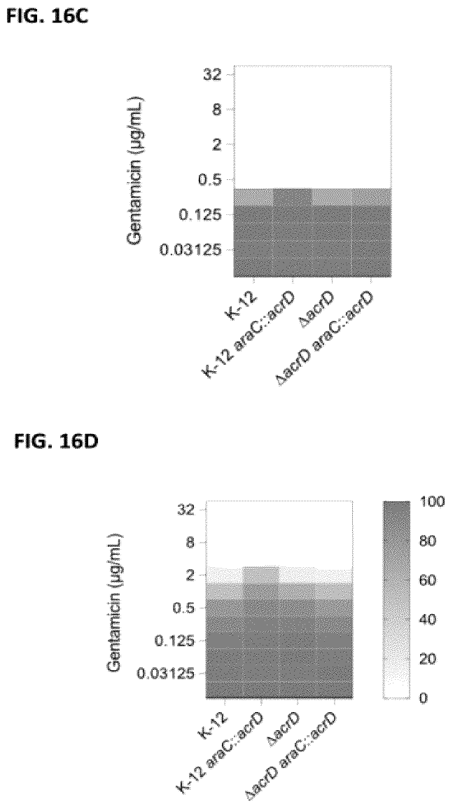

In contrast, AcrD and EmrAB decreased the susceptibility of EKO-35 to a smaller subset of compounds (30% and 33%, respectively) (Table 17). AcrD was associated with resistance to compounds with a narrower molecular weight range and PSA (FIGS. 4C and 4F), including non-polar compounds with positive log P values, contrary to previous reports that AcrD does not extrude hydrophobic molecules. Several known substrates for AcrD were confirmed (e.g., oxacillin, novobiocin, SDS, and deoxycholate); however, resistance to the aminoglycosides was not observed (FIG. 4B and Tables 13A and 13B). Indeed, despite previous studies showing increased susceptibility of E. coli to various aminoglycosides upon inactivation of acrD, inventors did not observe changes in susceptibility when acrD was inactivated (ΔacrD) in the wild-type K-12 strain, or when acrD was expressed in the ΔacrD or wild-type K-12 strains (FIGS. 16A-16D). Since aminoglycoside susceptibility can be affected by the growth medium, susceptibility testing was performed in both cation-adjusted Mueller Hinton II Broth (MHB II) and LB, showing similar results. These results are in agreement with a previous study reporting that AcrD in Salmonella enterica Serovar Typhimurium does not confer resistance to aminoglycosides. Finally, compromising the OM expanded the substrate profile of AcrD, however, the overall physicochemical profile was unaffected, and aminoglycoside resistance was not observed (FIGS. 4C-4E and Tables 13A, 13B, and 17).

In contrast to the broad substrate profiles of the RND efflux pumps, the remaining pumps were associated with resistance to a much smaller range of compounds. For example, EmrAB primarily conferred resistance to non-polar compounds spanning a small range of lipophilicity, including the uncoupler CCCP, which is consistent with previous studies. EmrAB no longer provided resistance to CCCP in the porinated strain, and was instead associated with resistance to fusidic acid and synthetic compound 10 (FIG. 4B, Tables 14A, 14B, and 17). MacAB and MdtBC conferred resistance to only 6% of the compounds (FIG. 4B and Tables 13A and 13B); consistent with previous reports, MacAB was specific for macrolides (erythromycin and azithromycin). However, MacAB with a porinated OM reduced the MIC of azithromycin to only 2-fold greater than EKO-35, which is within the acceptable error range for these assays (FIG. 4B and Tables 14A and 14B). MdtBC conferred resistance to novobiocin and deoxycholate, as previously reported (FIG. 4B, Tables 13A and 13B). Introduction of the pore additionally associated MdtBC with resistance to sodium taurodeoxycholate (FIG. 4B, Tables 14A and 14B), supporting a role in bile salt tolerance. Production of MdtBC in combination with the pore was also associated with resistance to daunorubicin and doxorubicin, substantially expanding the lipophilicity range of this pump (FIG. 4D and Table 17). EmrKY did not increase EKO-35 resistance to any of the compounds profiled, even when the OM was compromised (FIG. 4B and Tables 13A, 13B, 14A, and 14B).

Finally, inventors also profiled the MexCD pump from P. aeruginosa to demonstrate the use of EKO-35 as a tool for the study of bacterial efflux pumps from other bacterial species. MexCD conferred resistance to ˜55% of compounds profiled, many of which were known substrates, in addition to six of the poorly-characterized compounds (FIG. 4B and Tables 13A and 13B). These compounds were chemically unrelated, which was consistent with the polyspecific substrate profiles of the other multidrug-resistant RND pumps profiled (AcrB, AcrEF, and MdtEF) (FIG. 4B). Furthermore, compromising the OM no longer associated MexCD with resistance to puromycin, azithromycin, deoxycholate, and synthetic compound 13, and instead increased resistance to norfloxacin, sodium taurodeoxycholate, and synthetic compound 10 (FIG. 4B). However, these changes did not affect the overall physicochemical substrate profile, and therefore the polyspecificity of MexCD (FIGS. 4C-4E).

Overall, porinating the OM of the EKO-35 efflux-integrated strains did not substantially affect the activity of the efflux pumps, including their overall physicochemical substrate profiles. However, inventors did identify additional substrates when the OM was compromised, revealing that efflux pumps have expanded substrate profiles in environments that increase OM permeation. In the few instances (e.g., AcrEF, MacAB, and MexCD) where inventors observed decreased efflux-mediated resistance following introduction of the pore, it is possible that increased permeation overwhelmed the pump. However, the findings of the present disclosure indicate that efflux pumps can function robustly even when the OM is compromised.

### EKO-35 and the Efflux Platform can be Used to Evaluate the Specificity of Efflux Pump Inhibitors

Due to their role in antibiotic resistance, efflux pumps are attractive antibacterial targets. Indeed, an inhibitor of a polyspecific efflux pump, such as AcrAB, could simultaneously enhance the activity of numerous antibiotics against resistant strains. Phenylalanine-Arginine 8-Naphthylamide (PAβN) is one of the best-studied efflux pump inhibitors (EPIs), which inhibits AcrAB and its homologues, including numerous P. aeruginosa pumps (e.g., MexAB-OprM, MexCD-OprJ, MexXY-OprM, and MexEF-OprN). In addition, several arylpiperazines have been associated with E. coli efflux inhibition, including 1-(1-naphthylmethyl)-piperazine (NMP). Here, using PAβN and NMP as a proof of concept, inventors show EKO-35 and the developed efflux platform can be used to assess the specificity of EPIs.

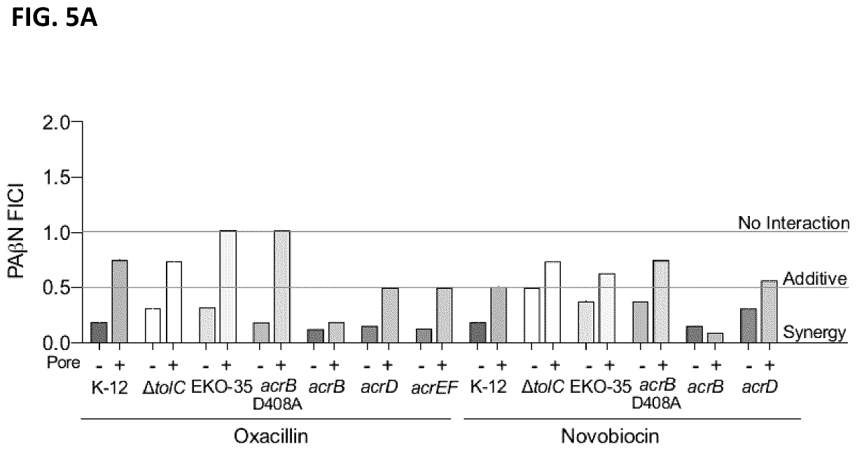

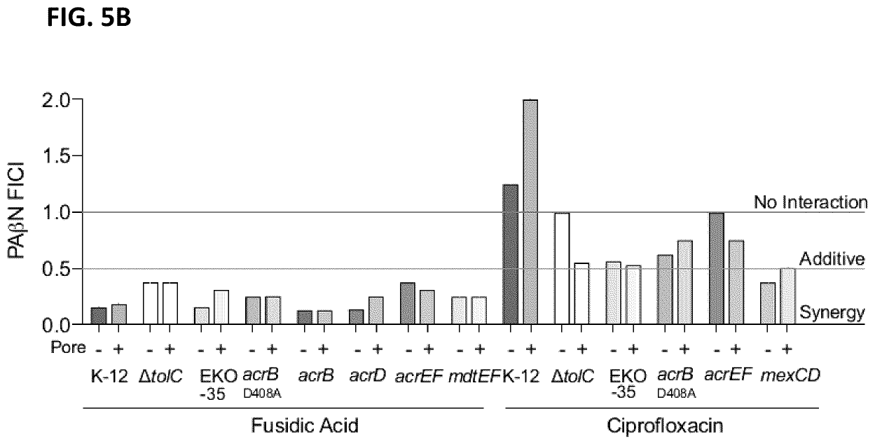

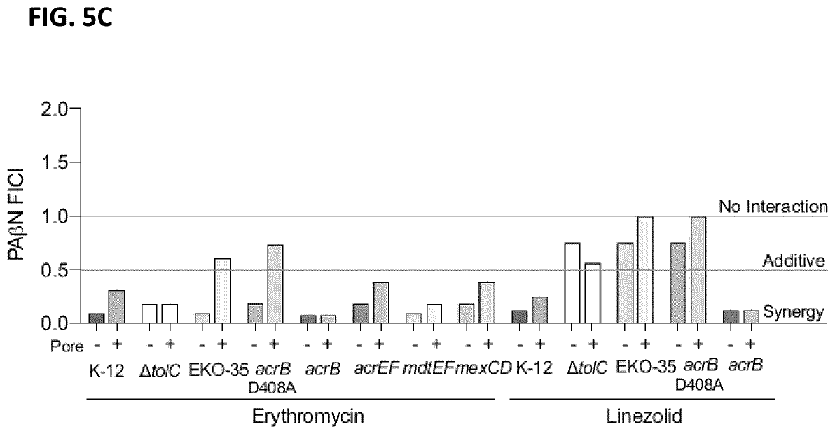

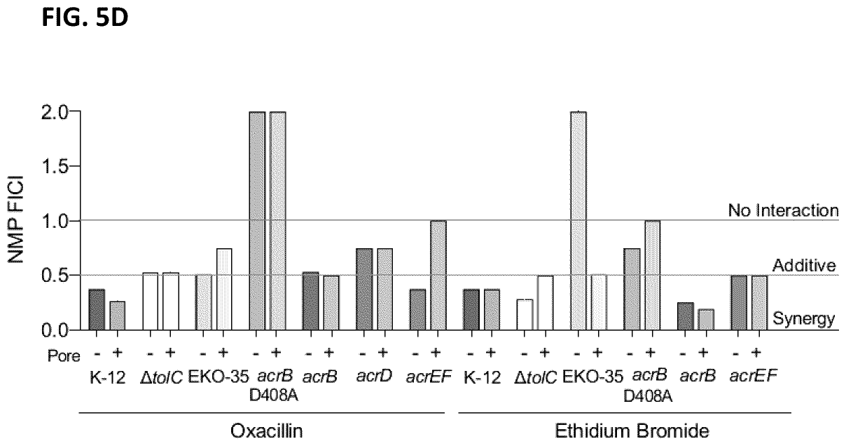

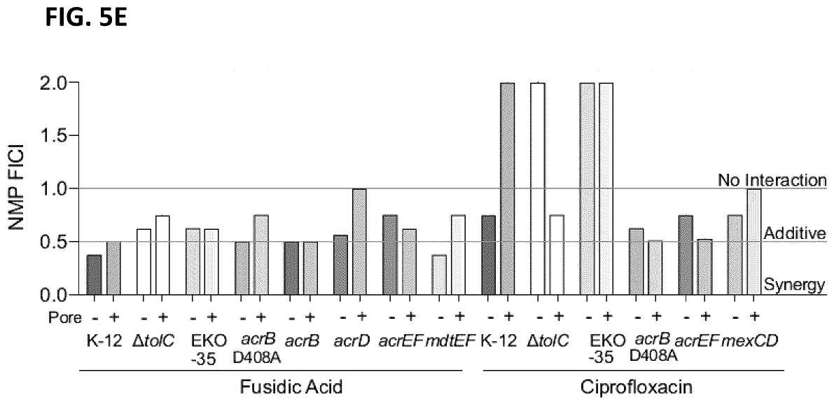

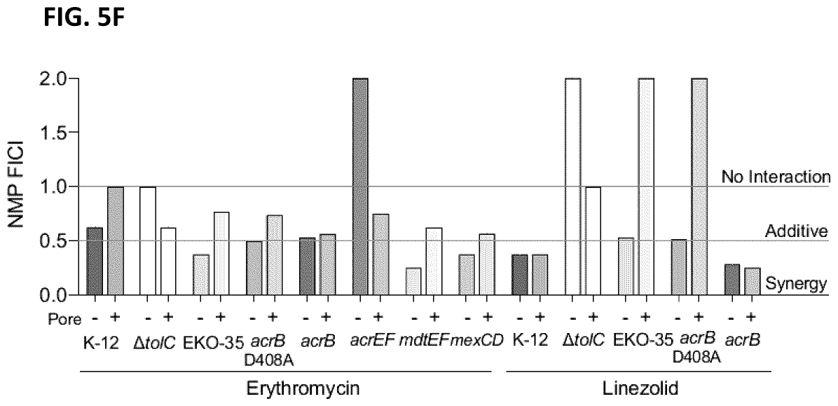

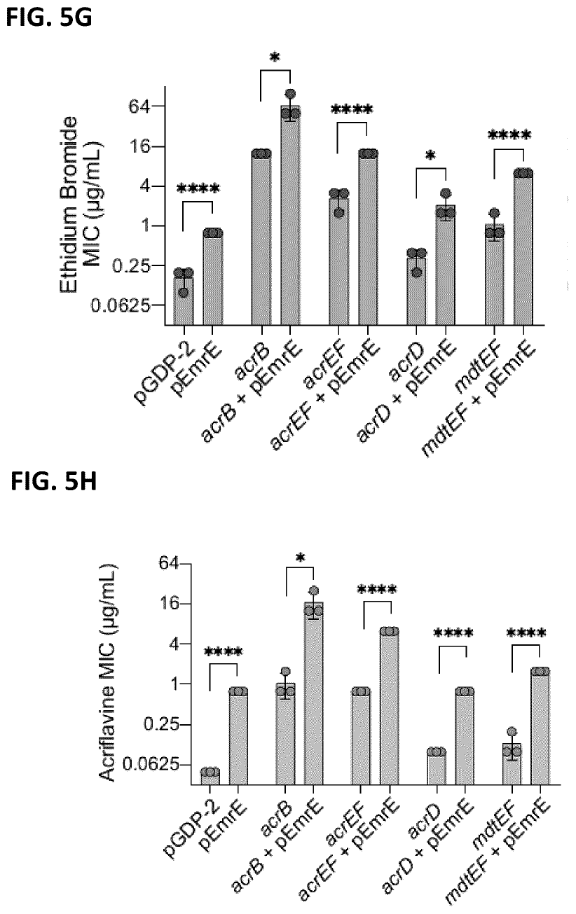

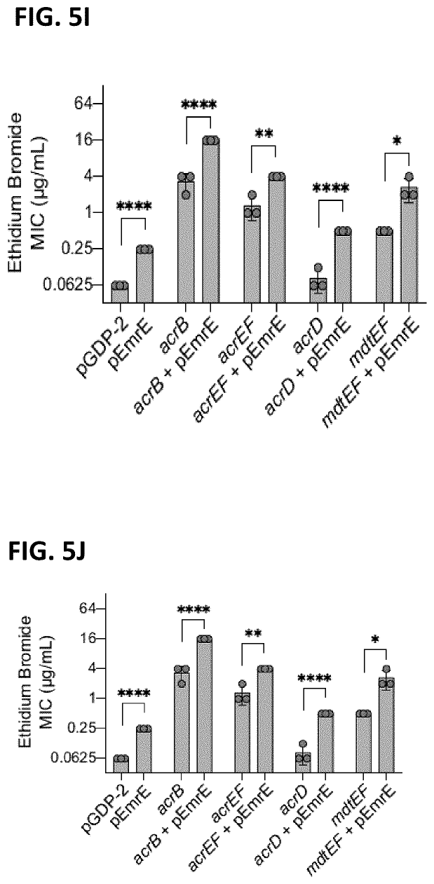

First, inventors assessed PAM and NMP synergy with various antibiotics by checkerboard analysis against the efflux-deficient strains, EKO-35, EKO-35 acrB_{D408A}, and ΔtolC, in addition to the wild-type strain (FIGS. 5A-5J and Tables 18A, 18B, 19A, and 19B). Both EPIs synergized with several antibiotics in the wild-type strain (FIG. 5). PAM is an efflux substrate, albeit a poor substrate that reduces the extrusion of other substrates. The compound also exhibits antibacterial activity, which is enhanced upon efflux inactivation (Tables 18A and 18B). In contrast, the susceptibility of the efflux-deficient strains was comparable to the wild-type strain for NMP, indicating the compound inhibits efflux in a different way, or does not exhibit antibacterial properties (Tables 19A and 19B). Consistent with the observation that PAM permeabilizes membranes in a concentration-dependent manner, the EPI synergized with various antibiotics in the efflux-deficient strains, including oxacillin, novobiocin, fusidic acid, and erythromycin (FIGS. 5A-5C and Tables 18A and 18B). However, PAM was not synergistic in these strains in combination with linezolid, which can more readily penetrate the OM (FIG. 5C and Table 18A and 18B). NMP did not exhibit synergy with the majority of the antibiotics against EKO-35 and EKO-35 acrB_{D408A} (FIG. 5D-5F and Tables 19A and 19B). However, inventors did observe low-level synergy with erythromycin, and also synergy with ethidium bromide against ΔtolC (FIGS. 5D and 5F and Tables 19A and 19B). Overall, the findings show NMP does not exhibit the same OM permeability permeabilizing properties as PaβN, consistent with previous findings indicating NMP only destabilizes membranes at concentrations exceeding 250 μg/mL (Tables 19A and 19B).

Acknowledging that distinguishing EPI permeabilizing properties from efflux inhibition is a challenge, inventors next utilized the porinated strains to assess synergy, predicting that synergy would be lost in the porinated strains. Indeed, inventors observed that PaβN was no longer synergistic in combination with erythromycin, novobiocin, and oxacillin against the porinated efflux-deficient strains (FIGS. 5A, 5D and 5F, and Table 18A and 18B). However, synergy was maintained for fusidic acid (FIG. 5B and Tables 18A and 18B). Similarly, NMP was no longer synergistic in combination with erythromycin, indicating the compound exhibits weak OM permeabilizing properties (Tables 19A and 19B). Overall, the results demonstrate that profiling EPIs against efflux-deficient strains like EKO-35 and the EKO-35-Pore derivative is an important step during the characterization of such compounds.

Next, inventors investigated EPI specificity against both EKO-35 and the EKO-35-Pore strain producing different efflux pumps, by assessing synergy with identified antibiotic substrates for each pump (FIGS. 5G-5J, Tables 18A, 18B and Tables 19A and 19B). Use of the porinated strains enabled isolation of efflux pump inhibition from OM permeabilizing properties, and inventors observed distinct phenotypes for different efflux pumps and antibiotic combinations (FIGS. 5A-5F, Tables 18A, 18B, 19A, and 19B). For example, PAM was synergistic in combination with all of the antibiotics profiled against the EKO-35 porinated strain expressing acrB and acrF, and no synergy was observed for the functionally inactive AcrB variant (FIG. and Tables 18A and 18B). In contrast, when acrD was expressed in the porinated strain, synergy was lost for novobiocin, yet was still observed for oxacillin and erythromycin. Similarly, synergy was lost for ciprofloxacin in EKO-35-Pore expressing the P. aeruginosa pump mexCD. Overall, the findings show that PAM exhibits different levels of inhibition and specificity against the various efflux pumps and their antibiotic substrates (Tables 18A and 18B).

Overall, NMP is not as potent as PAβN, providing relatively higher fractional inhibitory concentration index values (FICIs) (FIGS. 5A-5F). Additionally, inventors demonstrate that, with the exception of potentiating ethidium bromide against the strain producing AcrEF, NMP is specific for AcrB, enhancing the activity of ethidium bromide, fusidic acid, linezolid and oxacillin in the porinated strain producing AcrB (FIGS. 5A-5F and Tables 19A and 19B).

In summary, EKO-35 and the efflux platform are important tools for the assessment of candidate EPIs, enabling differentiation between compounds that permeabilize membranes, which increases the influx of antibiotics, and those that solely act as EPIs. In addition, there are major limitations associated with investigating EPI specificity in strains harboring intact drug efflux networks. EKO-35 and the efflux platform overcome these limitations, enabling users to systematically assess inhibition of each efflux pump within the platform.

### Investigating Efflux Pump Functional Interplay Using EKO-35 as a Simplified Genetic Background

Previous studies revealed the combination of structurally distinct efflux pumps—single component inner membrane efflux pumps and tripartite systems, which span the cell envelope—can confer multiplicative effects on resistance. Specifically, the combination of these two pump types confers a fold increase—or multiplicative effect—on resistance that is equal to or greater than the product of the fold increases conferred by the individual efflux pumps alone. An additive effect is observed when the fold increase in resistance equates to the sum of the individual efflux pumps alone. Interplay is considered to be the result of single component inner membrane pumps effluxing compounds to the periplasm, where tripartite systems can then access these compounds, extruding to the outside of the cell. Indeed, AcrB was shown to only provide robust resistance to ethidium bromide and acriflavine when single component pumps are present, since AcrB is considered to only access substrates from the outer leaflet of the inner membrane and the periplasm. Therefore, for compounds with cytoplasmic targets, such as acriflavine and ethidium bromide, it is possible that tripartite efflux systems rely on single component inner membrane pumps to first efflux substrates to the periplasm. However, inventors observed that with the exception of ethidium bromide and acriflavine, the overexpression of acrB alone restored the sensitivity of EKO-35 to levels comparable, if not greater, than those observed in the wild-type strain for the majority of compounds profiled in this disclosure (Tables 13A and 13B). Thus, AcrB provides robust efflux independent of single component inner membrane pumps for a wide range of compounds with diverse physicochemical properties. Indeed, findings of this disclosure show that permeation across the cell envelope is sufficiently rate limiting, since the tripartite pumps can access the drugs from the periplasm and the inner-membrane outer leaflet as they are diffusing.

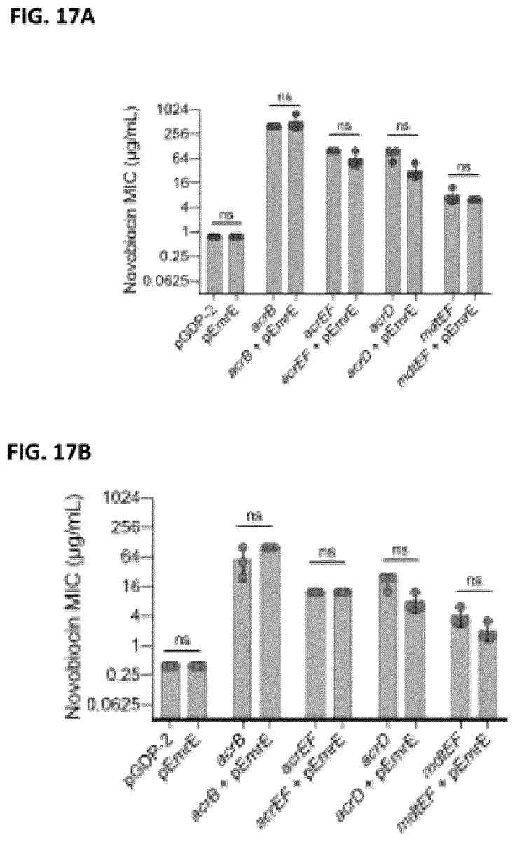

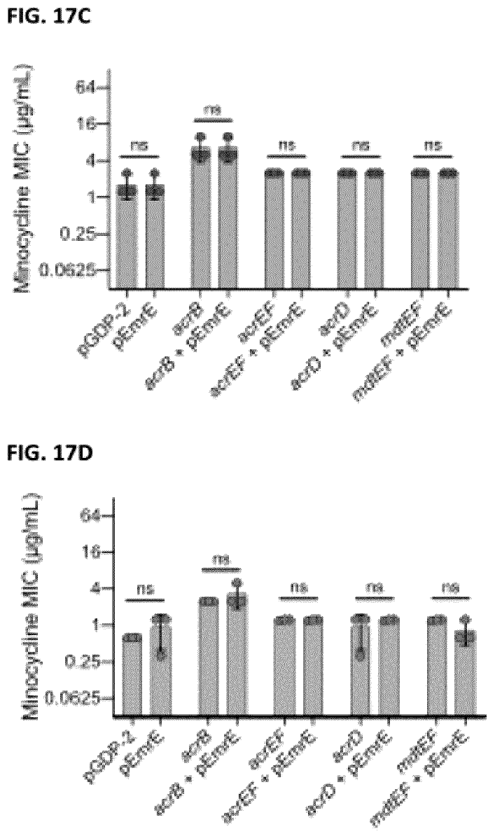

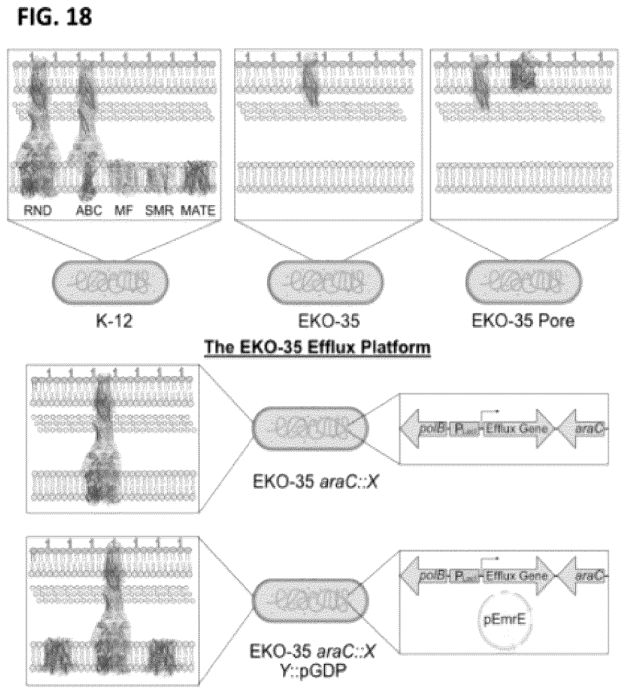

To demonstrate the use of EKO-35 to study efflux pump interplay, the inner membrane efflux pump EmrE was introduced into EKO-35 strains with integrated RND tripartite efflux pumps (AcrB, AcrEF, AcrD, and MdtEF) using the pGDP2 plasmid (pGDP2:emrE), which features the constitutive P_{LacI} promoter. Inventors then assessed resistance to ethidium bromide and acriflavine, since AcrB alone did not restore resistance to wild-type levels. Overall, inventors observed that the combination of EmrE with all three tripartite systems conferred multiplicative effects for both ethidium bromide and acriflavine (FIG. 5G-5J and Table 20). AcrB and AcrEF conferred a higher-level of resistance alone to these compounds; as such, the multiplicative effects of these pumps with EmrE was substantially greater than AcrD and MdtEF. Inventors also included novobiocin and minocycline as negative controls that were not extruded by EmrE, and inventors did not observe synergy (FIGS. 17A-17D and Table 20).

Next, inventors predicted that interplay can be more apparent when the OM is compromised, causing a greater influx of the compounds into the cell. To investigate, the EKO-35-Pore strains were profiled using the same approach. Overall, the multiplicative effects were maintained for the majority of the pump combinations, with the exception of AcrD with acriflavine, where the multiplicative effect was lost, and AcrD did not increase resistance to acriflavine when produced alone or in combination with EmrE (FIGS. 5G-5J and Table 20).

### Discussion

Bacterial drug efflux networks are expansive and poorly characterized, extending beyond archetypal pumps (e.g., AcrB) that are well-studied due to their polyspecific transporting capabilities. However, the substrate specificity and functions of many efflux pumps remain poorly understood, which can be attributed to the complexities of these systems, including differential gene expression and a high degree of functional redundancy. Here, inventors describe the generation of EKO-35, a simplified genetic background to address the described limitations of the efflux field. This strain can be used to study the functions and physicochemical substrate specificities of individually introduced efflux pumps, to assess the mechanism of action and efficacy of EPIs, and to study efflux pump interplay.

Inventors' ability to inactivate such a significant number of efflux pumps within the E. coli genome has provided important biological insight. Despite the high degree of functional redundancy, at least in terms of antibiotic detoxification, the E. coli drug efflux system is highly conserved. Conservation shows these proteins could contribute to physiologically essential roles, in addition to their well-described ability to extrude clinically important antibiotics. Indeed, numerous physiological functions have been associated with drug efflux pumps. For example, the TolC OM channel is broadly implicated in enterobacterial physiology. Inactivation causes pleiotropic phenotypes, including severe growth defects in nutrient-limited conditions. As described, MdtEF-TolC has been associated with the detoxification of nitrosative derivatives produced during anaerobic respiration. In addition, many E. coli efflux pumps have been associated with the extrusion of enterobactin, MdtJI exports the polyamine spermidine, and SugE is a guanidinium ion efflux pump.

Despite the contribution of these proteins to the maintenance of cellular homeostasis, here inventors show the E. coli drug efflux system is dispensable under optimal growth conditions (FIG. 1B), including during nutrient-limitation (FIG. 2A). Membrane-enriched comparative proteomics revealed only minor changes to the proteome of EKO-35 in nutrient-rich medium; however, numerous adaptations were observed in nutrient-limited medium (FIGS. 1A and 2B). While it was possible to inactivate such a significant number of IM efflux pumps, inventors did identify six nonsynonymous mutations during the generation of EKO-35 (FIG. 6). The overexpression of wild-type unmutagenized copies of these genes, using plasmids isolated from ASKA clones harboring the rspA, tufA, pitA, wcaC, gyrB, and yjfC genes, revealed a significant reduction in EKO-35 fitness under nutrient-rich optimal growth conditions for all of these genes. The fitness of the wild-type strain was also reduced by the overexpression of pitA, tufA and rspA. In addition, pitA and rspA overexpression was lethal, and wcaC negatively impacted fitness, in both EKO-35 and the wild-type strain in nutrient-limited conditions (FIGS. 8A (left) and 8C-8E (left)). EKO-35 was generated in a nutrient-rich medium, and the observation that the nonsynonymous mutations occurred in genes that are associated with EKO-35 fitness show they could be compensatory in nature (FIGS. 8A-8F (left)). Indeed, the findings show that the mutations could have been induced in response to loss-of-efflux, to enhance EKO-35 fitness, which could explain why the strain only exhibits minor changes in growth kinetics in nutrient-rich and nutrient-limited conditions (FIGS. 1B and 2A).

In support of efflux-associated physiological functions, inventors show the E. coli efflux network is conditionally essential. Indeed, the introduction of wild-type copies of the above mentioned genes did not restore the conditionally essential phenotypes described in this disclosure, showing these phenotypes correlate with loss-of-efflux pumps directly. The fitness of EKO-35 was significantly impacted under acid and alkali stress (FIG. 3A), in accordance with previous studies associating efflux pumps with pH homeostasis. Most striking was the observation that efflux is essential for growth in a nutrient-limited low oxygen environment (FIG. 3F), and findings of this disclosure highlight that other pumps can be associated with this phenotype, in addition to MdtEF-mediated extrusion of toxic by-products during anaerobic respiration. Finally, during the phenotypic characterization of EKO-35, inventors were surprised to observe differing growth phenotypes in comparison to the ΔtolC mutant. Indeed, while it is clear efflux pumps contribute to physiological processes, TolC inactivation induces a different and apparently pleiotropic cellular response that is not observed in EKO-35. Overall, these findings open the way for future studies to investigate the efflux pumps and underlying mechanisms associated with the identified conditionally essential phenotypes; the described efflux platform is ideal for such studies. In addition, by performing an in-depth assessment of EKO-35, inventors provide a well-characterized strain for the study of efflux pump functions and physicochemical substrate profiles.

To the best of inventors' knowledge, EKO-35 is the most efflux-deficient mutant to be reported. As such, this strain is highly susceptible to numerous antimicrobial agents (Tables 13A and 13B). Similar to the observed phenotypic differences, EKO-35 exhibited differing susceptibility levels to the ΔtolC mutant. Importantly, most of the well-characterized antibiotics inhibited the growth of both strains comparably, indicating tripartite efflux pumps are primarily responsible for extruding these compounds, and are the major contributors to resistance in the efflux network. However, numerous compounds exhibited increased activity against the ΔtolC mutant, despite the lack of tripartite efflux pumps in EKO-35. Most of these were the poorly characterized synthetic compounds (Tables 10A and 10B, compounds 1-19). Inventors have shown these differences were not associated with the nonsynonymous mutations, since the introduction of wild-type copies of these genes did not increase the susceptibility of EKO-35 (FIGS. 13A-F). Porination of the OM only increased the susceptibility of EKO-35 to two of the synthetic compounds that were originally inactive against EKO-35 (FIGS. 15A and 15B and Table 10), indicating that the OM of ΔtolC could be more permeable than EKO-35 in these instances. Indeed, the inactivation of tolC leads to membrane stress, which has been suggested to contribute significantly to its increased susceptibility to multiple antibiotics. However, the remaining synthetic compounds did not impact the susceptibility of the EKO-35-Pore strain, showing that permeability did not underlie the differences observed between ΔtolC and EKO-35. Clearly, loss of IM efflux pumps impacts susceptibility differently than deletion of an OM channel, which shows that TolC is associated with other functions related to these resistance phenotypes. As such, this should be taken into account when using ΔtolC mutants to study drug efflux. Indeed, EKO-35 is an important addition to the arsenal of tools to explore the movement of compounds across the cell envelope.

With an intact OM, the physicochemical properties that can be assessed using EKO-35 and the efflux integration platform would be limited to those that can penetrate the OM, which would restrict the molecular weight range to compounds <600 g/mol. To overcome this limitation, a pore was introduced into the wild-type, ΔtolC, EKO-35, and EKO-35 efflux pump-integrated strains, which enabled uncoupling of the contributions of influx and efflux. Inventors show that efflux contributed more significantly than OM permeability to the intrinsic resistance of E. coli, and compromising both the OM and efflux increased susceptibility to a wide range of compounds (FIGS. 4A, 4B, and 15A). Overall, compromising the OM of EKO-35 significantly broadened the physicochemical properties that could be profiled with the efflux platform, while maintaining efflux pump activity.

Previously, different approaches have been taken to investigate the E. coli drug efflux system. Sulavik et al., individually inactivated 16 efflux pumps and profiled these strains against a panel of 20 toxic molecules. Due to efflux pump functional redundancies, and differential expression levels, there are limitations associated with this approach. Nishino & Yamaguchi individually expressed 37 efflux pump-encoding genes in an AcrAB-devoid host, and subsequently profiled 26 toxic molecules. Both studies substantiated AcrAB-TolC as the major contributor to intrinsic resistance. A subset of additional efflux pumps were associated with resistance to a proportion of the compounds, but a large number did not provide resistance phenotypes. Additional studies have also investigated E. coli efflux using tolC inactivated mutants, to ascertain molecular features of compounds amenable to efflux. Since TolC is the predominant gate keeper for extrusion across the OM in E. coli, the study of tolC mutants represents loss of efflux as a whole, and provides important information relating to the properties of compounds that are susceptible to efflux by tripartite systems. However, little insight is provided into the physicochemical substrate specificities of the IM pump components. In addition, as described above, tolC inactivation causes pleiotropic effects that appear to be distinct from loss of drug efflux pump functions. Therefore, there are limitations associated with the use of tolC mutants to study drug efflux, which are overcome by EKO-35.

Construction of the developed efflux platform enabled us to assess the physicochemical substrate profiles of E. coli efflux pumps forming tripartite complexes with TolC. Inventors also included MexCD from P. aeruginosa, showing that the platform can be used to study pumps from other organisms. The efflux platform was built upon an isogenic and highly susceptible background (EKO-35), with gene expression under the control of the same constitutive promoter. Such an approach enabled direct comparisons to be made between strains and inventors were able to summarize molecular properties that contribute to efflux in each pump (FIGS. 4C-4F). Inventors revealed that AcrB, AcrEF, MdtEF, and MexCD exhibited polyspecific redundant substrate profiles, with little specificity for molecular weight, aqueous solubility or polar surface area (FIG. 4C-4F). AcrB and AcrEF mediated resistance to a wide array of hydrophilic and lipophilic compounds, and MdtEF was associated with resistance to more lipophilic substrates. AcrD provided resistance to a smaller subset of compounds that were mostly lipophilic and fell within a narrower molecular weight range (˜285 to 613 g/mol). EmrAB also exhibited resistance to a smaller fraction of the compounds, which displayed more specific criteria, including lipophilicity and a molecular weight ranging from 205-613 g/mol. MacAB was specific for macrolides, MdtBC for bile salts, and inventors were unable to identify any substrates of EmrKY. While EmrKY is a homologue of EmrAB, the pump has only been associated with resistance to deoxycholate, which inventors could not substantiate. Overall, despite a subset of the pumps appearing to have evolved to extrude specific substrates (e.g., MacAB and MdtBC), a significant number of the pumps were polyspecific and thus functionally redundant. When examined phylogenetically, these RND pumps (AcrB, AcrF, and MdtF) cluster together, which can be indicative of their multidrug-resistant functions. The clustering of AcrF and MdtF can explain the similarities observed in the physicochemical substrate profiles of these homologous proteins (Teelucksingh & Thompson et al., 2020) (FIGS. 4C-4F). In addition, the P. aeruginosa MexCD pump exhibited comparable resistance profiles to these two pumps. In the case of AcrB, this polyspecific phenotype appears to be an ancient and conserved trait. It is unclear why all three polyspecific pumps have been conserved across the E. coli species. Combined, the redundant polyspecific nature of these efflux pumps highlight the challenges of designing new antibacterial agents that overcome efflux.

Previous studies indicate that molecular weight and hydrophobicity are key factors impacting compound susceptibility to efflux; specifically, compounds exhibiting molecular weights between 300 and 700 g/mol are more susceptible to efflux. Findings from this disclosure were consistent with these observations, yet inventors observed a slightly larger molecular weight range through EKO-35 susceptibility testing, identifying compounds ranging from −227 to 750 g/mol as being susceptible to efflux (FIG. 15C). Indeed, numerous efflux pumps (AcrB, AcrEF, MdtEF, and MacAB) were associated with resistance to compounds >700 g/mol (FIG. 4C, Table 17). In addition, when the OM was compromised, AcrB provided resistance to rifampicin (MW=822.95 g/mol). Hydrophobic molecules have also been reported to be more susceptible to efflux than hydrophilic molecules. In this disclosure, efflux activity was only observed for compounds with log P≥−1.986; however, ˜33% of compounds active against EKO-35 were hydrophilic (Tables 10 and 17). Additionally, several hydrophobic compounds were inactive against EKO-35 (Table 10). Inventors also identified a subset of compounds that meet these molecular weight and hydrophobicity criteria, but were inactive against EKO-35 (Table 10). Altogether, findings of this disclosure show that molecular weight and hydrophobicity are not a prerequisite for efflux; the charge, globularity, flexibility, and stability of compounds are also important physicochemical properties governing the influx and efflux of compounds.

It is possible that efflux pump interplay could impact the substrate profiles of tripartite efflux pumps; as described, interplay refers to synergistic relationships between tripartite systems and single component IM pumps. However, inventors show the production of AcrB alone can restore the susceptibility of EKO-35 to wild-type levels for most compounds profiled. Indeed, acriflavine and ethidium bromide were the only compounds that tripartite pumps appeared to rely on contributions from single component inner membrane pumps to provide robust resistance (FIGS. 5G-5J). Inventors show the efflux platform is ideal for studying efflux pump interplay, which would otherwise be complicated by the complexities of the E. coli drug efflux network. Future studies could harness EKO-35 and the platform to investigate further instances of efflux pump interplay.

In addition to investigating efflux pump physicochemical substrate profiles, the efflux platform can also be used to evaluate EPIs. As a proof of principle, inventors assessed synergy of the EPIs PAβN and NMP with various antibiotics (Table 12 and FIGS. 5A-5F). Since decreased permeability and active efflux synergistically contribute to intrinsic resistance, it is difficult to ascertain the mechanism of action of antibiotic adjuvants such as EPIs. Uncoupling influx from efflux inhibition, through the use of the EKO-35-Pore strain and the integrated efflux pump library, can provide important insight into EPI mechanism(s) of action. Indeed, inventors propose that candidate EPIs are routinely profiled against the efflux-deficient strains, EKO-35 and EKO-35-Pore, providing insight into whether an EPI confers pleiotropic effects, including membrane destabilization, as exemplified by PAβN. In addition, profiling EPIs against the EKO-35-Pore strain producing individual efflux pumps provides insight into the pump specificity of EPIs. Indeed, findings of this disclosure highlight that both PAβN and NMP exhibit efflux pump inhibition; however, each inhibitor exhibits differing efflux pump specificities, including potentiation of different antibiotic substrates. There is precedent underlying this observation in the literature; PAβN is predicted to bind both the binding pocket groove and cave regions of AcrB, which enables the EPI to potentiate a diverse range of compounds with affinity for either binding site (e.g., novobiocin and oxacillin) (FIGS. 5A-5C). In addition, PAβN has been shown to inhibit AcrB primarily through perturbation of drug-binding structural dynamics, rather than competitive binding, which could underlie the compound's ability to potentiate such a wide range of antibiotics. In addition, inventors show that PAβN is a broad-spectrum inhibitor both in terms of antibiotic potentiation and efflux pump specificity (FIG. 5A-5C). In contrast, NMP is predicted to bind solely to the binding pocket cave and, as such, NMP largely potentiates cave binding compounds (e.g., ethidium bromide) (FIGS. 5D-5F). Finally, inventors show that for the most part NMP exhibits specificity for AcrB (FIGS. 5D-5F). In summary, the efflux platform provides an essential tool to deconvolute EPI mechanism(s) of action.

In conclusion, efflux pumps are a major contributor to the intrinsic antibiotic resistome of Gram-negative pathogenic bacteria. Understanding the molecular properties that influence efflux is key for overcoming these resistance mechanisms. It is important that all efflux pumps are considered since inventors have a poor understanding of the fitness advantage conferred by each pump during the bacterial lifecycle. The efflux platform represents an innovative tool kit to fully dissect the movement of compounds across the cell envelope, providing a unique opportunity for users to introduce desired combinations of efflux pumps, and to also probe the relationship and balance between permeation and active efflux. Overall, EKO-35 and the developed platform will be an important resource to the efflux field, which can be used to profile efflux pumps of interest, to assess physiological functions and substrate specificities, and ultimately assist the design of new antibacterial agents and EPIs.

TABLE 5
EKO-35 genomic mutations. Single nucleotide polymorphisms
were identified relative to the parent E. coli BW25113
K-12 (Grenier et al. 2014) genome (List #1).

| | | | | Efflux |
|---|---|---|---|---|
| | | | | gene |
| | Base | | | associated |
| | pair | | | with the |
| Gene | mutation | Mutation | Gene Function | mutation |
| gntU | G489A | Silent | Gluconate transporter | mdtF |
| pitA | T1433A | V478E | Metal phosphate:H⁺ sympoter | mdtF |
| yjfC | A470G | E157G | Putative acid-amine ligase | mdtF |
| tufA | C677T | T2261 | Elongation factor Tu | ydeA |
| tig | C225T | Silent | Chaperone protein known as | mdtB |
| | | | Trigger Factor F. Specifically | |
| | | | interacts with nascent proteins | |

TABLE 6
EKO-35 genomic mutations. Single nucleotide polymorphisms
were identified relative to the parent E. coli BW25113
K-12 (Grenier et al. 2014) genome (List #2).

| | Base | | | Efflux gene |
|---|---|---|---|---|
| | pair | | | associated |
| | muta- | Muta- | | with the |
| Gene | tion | tion | Gene Function | mutation |
| rpsA | T795A | D265E | 30S ribosomal subunit protein | mdtB |
| | | | S. The largest of the ribosomal | |
| | | | proteins | |
| wcaC | A355C | K119Q | Galactosyltransferase | mdtB |
| | | | predicted to be involved in | |
| | | | colonic acid biosynthesis | |
| ybdK | G834A | Silent | Carboxylate-amine ligase | mdlB |
| gyrB | G373T | A125S | Type II topoisomerase that | cusA |
| | | | negatively supercoils circular | |
| | | | double stranded DNA | |
| yebS | G639A | Silent | Inner membrane protein | cusA |
| | | | predicted to contribute to | |
| | | | membrane integrity | |

TABLE 7
Quantitative comparative proteomic analysis revealed statistically
significant differentially abundant proteins between EKO-35 and
the wild-type E. coli K-12 strain in nutrient-rich conditions.
Comparative analysis was performed with proteins identified
in at least three biological replicates (1,979 proteins), statistical
analysis was performed using a Student's t-test (P-value ≤0.05,
FDR = 0.05, SO = 1).

| | | Fold | | | |
|---|---|---|---|---|---|
| | Protein | differ- | | | |
| Strain | IDs | ence | P-value | Gene | Protein |
| EKO- | P31828 | 8.63 | 2.09E−07 | pqqL | Putative TonB- |
| 35 | | | | | dependent |
| | | | | | receptor YddB |
| EKO- | P31827 | 7.92 | 1.39E−06 | yddB | Periplasmic |
| 35 | | | | | metalloprotease PqqL |
| EKO- | P76520 | 7.87 | 7.75E−08 | yfdX | Uncharacterized |
| 35 | | | | | protein YfdX |
| EKO- | P04949 | 4.96 | 1.84E−03 | fliC | Flagellin filament |
| 35 | | | | | protein FliC |
| EKO- | P02942 | 4.31 | 4.40E−03 | tsr | Methyl-accepting |
| 35 | | | | | chemotaxis protein I |
| EKO- | P77754 | 4.09 | 2.69E−03 | spy | Periplasmic chaperone |
| 35 | | | | | Spy |
| EKO- | P05706 | 3.88 | 2.06E−03 | srlB | Sorbitol-specific |
| 35 | | | | | phosphotransferase |
| | | | | | enzyme IIA component |
| | | | | | SrlB |
| EKO- | P56579 | 3.87 | 9.81E−04 | srlA | Sorbitol permease |
| 35 | | | | | phosphotransferase |
| | | | | | enzyme IIC2 component |
| | | | | | SrlA |
| EKO- | P00634 | 3.69 | 5.03E−04 | phoA | Alkaline phosphatase |
| 35 | | | | | PhoA |
| EKO- | P28861 | 3.66 | 1.49E−04 | fpr | Ferredoxin--NADP⁺ |
| 35 | | | | | reductase Fpr |
| K-12 | P13036 | −2.64 | 2.59E−02 | fecA | Ferric citrate outer |
| | | | | | membrane transporter |
| | | | | | FecA |
| K-12 | POAAEO | −2.66 | 6.34E−03 | cycA | D-serine/D- |
| | | | | | alanine/glycine/:H⁺ |
| | | | | | symporter CycA |
| K-12 | P15028 | −2.74 | 1.76E−03 | fecB | Ferric citrate ABC |
| | | | | | transporter periplasmic |
| | | | | | binding protein FecB |
| K-12 | P33941 | −3.04 | 9.63E−04 | yojl | ABC efflux pump Yojl |
| K-12 | P38105 | −3.25 | 1.30E−03 | rspB | Putative zinc-binding |
| | | | | | dehydrogenase RspB |
| K-12 | POC058 | −3.48 | 1.57E−03 | ibpB | Small heat shock protein |
| | | | | | IbpB |
| K-12 | POA7B8 | −3.69 | 3,18E−04 | hsIV | ATP-dependent protease |
| | | | | | subunit HsIV |
| K-12 | POC054 | −3.90 | 2.31E−03 | ibpA | Small heat shock protein |
| | | | | | lbpA |
| K-12 | P15031 | −4.76 | 1.49E−02 | fecE | Ferric citrate ABC |
| | | | | | transporter ATP binding |
| | | | | | subunit FecE |
| K-12 | P31224 | −5.69* | 1.31E−04 | acrB | Multidrug efflux pump |
| | | | | | subunit AcrB |

*AcrB fold difference was calculated through imputation using a normal distribution.

TABLE 8
Quantitative comparative proteomic analysis revealed statistically
significant differentially abundant proteins between EKO-35 and
the wild-type E. coli K-12 strain in nutrient-limited conditions.
Comparative analysis was performed with proteins identified
in at least three biological replicates (2,019 proteins), statistical
analysis was performed using a Student's t-test (P-value ≤0.05,
FDR = 0.05, SO = 1).

| | | Fold | | | |
|---|---|---|---|---|---|
| | Protein | differ- | | | |
| Strain | IDs | ence | P-value | Gene | Protein |
| EKO- | P31827 | 8.70 | 1.05E−05 | yddB | Putative TonB- |
| 35 | | | | | dependent receptor |
| | | | | | YddB |
| EKO- | P77754 | 7.56 | 1.47E−05 | spy | Periplasmic chaperone |
| 35 | | | | | Spy |
| EKO- | P31828 | 7.04 | 5.63E−06 | pqqL | Periplasmic |
| 35 | | | | | metalloprotease PqqL |
| EKO- | P33593 | 6.07 | 1.73E−05 | nikD | Nickel ABC transporter |
| 35 | | | | | ATP binding subunit |
| | | | | | NikD |
| EKO- | P76397 | 5.66 | 1.52E−06 | mdtA | Multidrug efflux pump |
| 35 | | | | | membrane fusion |
| | | | | | protein MdtA |
| EKO- | P21865 | 5.50 | 1.10E−05 | kdpD | Sensor histidine kinase |
| 35 | | | | | KdpD |
| EKO- | POAE85 | 5.09 | 4.59E−06 | cpxP | Periplasmic protein |
| 35 | | | | | CpxP |
| EKO- | P16095 | 4.98 | 5.29E−05 | sdaA | L-serine dehydratase 1 |
| 35 | | | | | SdaA |
| EKO- | P23522 | 4.96 | 8.72E−05 | garL | 5-keto-4-deoxy-D- |
| 35 | | | | | glucarate aldolase GarL |
| EKO- | P76251 | 2.28 | 2.28E−02 | dmlA | D-malate |
| 35 | | | | | dehydrogenase DmlA |
| K-12 | P13036 | −4.15 | 2.07E−03 | agp | Glucose-1-phosphatase |
| | | | | | Agp |
| K-12 | POAAEO | −4.16 | 1.89E−05 | pitA | Low-affinity inorganic |
| | | | | | phosphate transporter 1 |
| | | | | | PitA |
| K-12 | P15028 | −4.22 | 1.17E−07 | cusA | Copper/silver RND |
| | | | | | permease CusA |
| K-12 | P33941 | −4.25 | 5.67E−05 | ggt | Gamma- |
| | | | | | glutamyltranspeptidase |
| | | | | | Ggt |
| K-12 | P38105 | −4.82 | 1.90E−03 | otsB | Trehalose-6-phosphate |
| | | | | | phosphatase OtsB |
| K-12 | POC058 | −4.89* | 7.41E−04 | acrB | Multidrug RND |
| | | | | | permease AcrB |
| K-12 | POA7B8 | −4.91 | 5.11E−05 | gcd | Quinoprotein glucose |
| | | | | | dehydrogenase Qcd |
| K-12 | POC054 | −5.16 | 8.20E−04 | lamB | Maltose outer |
| | | | | | membrane channel |
| | | | | | LamB |
| K-12 | P15031 | −5.40 | 6.01E−04 | tnaA | Tryptophanase TnaA |
| K-12 | P31224 | −5.93 | 2.21E−05 | puuE | 4-aminobutyrate |
| | | | | | aminotransferase PuuE |

*AcrB fold difference was calculated through imputation using a normal distribution

TABLE 9
Efflux peptides detected in LC-MS/MS comparative proteomics from
cultures grown in Lysogeny broth and minimal M9 medium with a glucose
carbon source, in the absence of antibiotic selection. Peptides were
considered significant if they were identified in at least three biological
replicates. Additionally, a minimum of two peptides detected for each
efflux pump was required.

| Efflux | |
|---|---|
| Pump | Peptides Observed in K-12 |
| AcrB | AADGQMVPFSAFSSSR (SEQ ID NO: 256), |
| | AQALGVSINDINTTLGAAWGGSYVNDFIDR (SEQ ID NO: 257), |
| | AQNAQVAAGQLGGTPPVK (SEQ ID NO: 258), DWADRPGEENKVEAITMR |
| | (SEQ ID NO: 259), FQLTPVDVITAIK (SEQ ID NO: 260), |
| | GFFGWENR (SEQ ID NO: 261), GLIEATLDAVR (SEQ ID NO: |
| | 262), GQNTGIAFVSLK (SEQ ID NO: 263), |
| | HPDMLTSVRPNGLEDTPQFK (SEQ ID NO: 264), IVYPYDTTPFVK |
| | (SEQ ID NO: 265), IWMNPNELNK (SEQ ID NO: 266), |
| | LATGANALDTAAAIR (SEQ ID NO: 267), LPTGVGYDWTGMSYQER |
| | (SEQ ID NO: 268), LQLAMPLLPQEVQQQGVSVEK (SEQ ID NO: |
| | 269), MEPFFPSGLK (SEQ ID NO: 270), MLPDDIGDWYVR (SEQ ID |
| | NO: 271), NAILIVEFAK (SEQ ID NO: 272), |
| | NNVESVFAVNGFGFAGR (SEQ ID NO: 273), STGEAMELMEQLASK |
| | (SEQ ID NO: 274), TSGVGDVQLFGSQYAMR (SEQ ID NO: 275), |
| | VEAITMR (SEQ ID NO: 276), VLNEVTHYYLTK (SEQ ID NO: |
| | 277), VMAEEGLPPK (SEQ ID NO: 278), VYVMSEAK (SEQ ID NO: |
| | 279), WEYGSPR (SEQ ID NO: 280), YNGLPSMEILGQAAPGK (SEQ |
| | ID NO: 281) |
| CusA | ASGYLQTLDDENHIVLK (SEQ ID NO: 282), DRDMVSVVHDLQK (SEQ |
| | ID NO: 283), HDLADLR (SEQ ID NO: 284), IIEELDNTVR (SEQ |
| | ID NO: 285), LAQYGISLAEVK (SEQ ID NO: 286), |
| | LDEALYHGAVLR (SEQ ID NO: 287), LFGPLAFTK (SEQ ID NO: |
| | 288), LPGLANLWVPPIR (SEQ ID NO: 289), QLPILTPMK (SEQ ID |
| | NO: 290), QQITLADVADIK (SEQ ID NO: 291), SLQDWELK (SEQ |
| | ID NO: 292), TIPDVAEVASVGGVVK (SEQ ID NO: 293), |
| | TVPGVASALAER (SEQ ID NO: 294), VLEYLNQVQGK (SEQ ID NO: |
| | 295) |
| MdtK | GTAKPDPAVMK (SEQ ID NO: 296), LPSAIILQR (SEQ ID NO: |
| | 297) |
| Yojl | AEFPRPQAFPNWQTLELR (SEQ ID NO: 298), |
| | EFYQVLLPLMQEMGK (SEQ ID NO: 299), ILDTHVER (SEQ ID NO: |
| | 300), LFSAVFTDVWLFDQLLGPEGKPANPQLVEK (SEQ ID NO: 301) |

TABLE 10
Susceptibility of ΔtolC and EKO-35 +/− the pore to a diverse
panel of antimicrobial agents. Strains were assessed in technical duplicate
and instances where the MIC value differed (4-fold or greater) between
EKO-35 and ΔtolC are bolded. Values with a 4-fold or greater change
in MIC are in bold font to indicate increased susceptibility in EKO-35
compared to ΔtolC. STDC: Sodium taurodeoxycholate.

| | Minimum inhibitory concentration (MIC) (μg/mL) | | | | | |
|---|---|---|---|---|---|---|
| | | | | Pore | | |
| Compound | K-12 | ΔtolC | EKO-35 | K-12 | ΔtolC | EKO-35 |
| Rifampicin | 12.5 | 6.25 | 6.25 | 0.781 | 0.391 | 0.391 |
| Vancomycin | 200 | 200 | 200 | 3.125 | 6.25 | 6.25 |
| Fosfomycin | 6.25 | 6.25 | 3.125 | 3.125 | 3.125 | 0.781 |
| Ampicillin | 100 | 25 | 25 | 12.5 | 1.563 | 1.563 |
| Oxacillin | 160 | 0.625 | 0.625 | 40 | 0.313 | 0.313 |
| Chloramphenicol | 6.5 | 1.563 | 0.781 | 3.125 | 0.781 | 0.781 |
| Puromycin | 50 | 3.125 | 1.563 | 50 | 1.563 | 0.781 |
| Azithromycin | 6.25 | 0.781 | 0.781 | 0.391 | 0.049 | 0.049 |
| Erythromycin | 100 | 3.125 | 3.125 | 6.25 | 0.195 | 0.195 |
| Spectinomycin | 25 | 6.25 | 25 | 12.5 | 6.25 | 12.5 |
| Tetracycline | 1.563 | 0.391 | 0.195 | 0.781 | 0.391 | 0.195 |
| Linezolid | 500 | 7.813 | 7.813 | 125 | 3.906 | 3.906 |
| Kanamycin | 3.125 | 1.563 | 3.125 | 3.125 | 1.563 | 1.563 |
| Streptomycin | 6.25 | 3.125 | 3.125 | 12.5 | 1.563 | 3.125 |
| Minocycline | 0.781 | 0.098 | 0.049 | 0.781 | 0.098 | 0.049 |
| Fusidic acid | 400 | 3.125 | 3.125 | 100 | 0.391 | 0.391 |
| Ciprofloxacin | 0.010 | 0.005 | 0.005 | 0.005 | 0.002 | 0.002 |
| Norfloxacin | 0.078 | 0.020 | 0.020 | 0.078 | 0.010 | 0.010 |
| Nalidixic acid | 10 | 1.25 | 1.25 | 5 | 1.25 | 1.25 |
| Novobiocin | 200 | 0.781 | 3.125 | 12.5 | 0.391 | 0.781 |
| Trimethoprim | 0.391 | 0.098 | 0.195 | 0.391 | 0.049 | 0.195 |
| Doxorubicin | 200 | 1.563 | 1.563 | 50 | 0.781 | 0.781 |
| Daunorubicin | 200 | 1.563 | 3.125 | 100 | 0.781 | 0.781 |
| Ethidium | 100 | 3.125 | 0.195 | 100 | 3.125 | 0.098 |
| bromide | | | | | | |
| Bicyclomycin | 200 | 200 | 200 | 200 | 200 | 200 |
| Sulfathiazole | 5 | 5 | 10 | 5 | 2.5 | 5 |
| Acriflavine | 50 | 3.125 | 0.098 | 50 | 3.125 | 0.098 |
| SDS | 1000 | 15.625 | 31.25 | 1000 | 15.625 | 31.25 |
| Benzalkonium | 12.5 | 0.391 | 0.391 | 6.25 | 0.391 | 0.391 |
| chloride | | | | | | |
| Deoxycholate | 1500 | 187.5 | 375 | 1500 | 46.875 | 187.5 |
| STDC | 1000 | 250 | 500 | 1000 | 250 | 250 |
| Chlorhexidine | 0.781 | 0.391 | 0.391 | 0.781 | 0.391 | 0.391 |
| CCCP | 25 | 0.781 | 12.5 | 12.5 | 0.391 | 6.25 |
| Spermine | 2000 | 2000 | 2000 | 2000 | 2000 | 2000 |
| Compound 1 | 320 | 5 | 320 | 320 | 10 | 320 |
| Compound 2 | 320 | 1.25 | 320 | 320 | 1.25 | 320 |
| Compound 3 | 320 | 2.5 | 320 | 320 | 1.25 | 320 |
| Compound 4 | 320 | 10 | 20 | 320 | 1.25 | 2.5 |
| Compound 5 | 320 | 10 | 20 | 320 | 2.5 | 20 |
| Compound 6 | 320 | 0.625 | 320 | 320 | 0.625 | 320 |
| Compound 7 | 320 | 0.313 | 320 | 320 | 0.313 | 320 |
| Compound 8 | 640 | 1.25 | 320 | 160 | 1.25 | 80 |
| Compound 10 | 160 | 10 | 160 | 160 | 5 | 10 |
| Compound 11 | 320 | 0.156 | 0.078 | 80 | 0.156 | 0.078 |
| Compound 12 | 320 | 1.25 | 320 | 320 | 0.625 | 160 |
| Compound 13 | 320 | 1.25 | 40 | 320 | 1.25 | 5 |
| Compound 14 | 320 | 2.5 | 320 | 320 | 1.25 | 320 |
| Compound 15 | 320 | 1.25 | 320 | 320 | 0.625 | 320 |
| Compound 16 | 320 | 10 | 10 | 160 | 5 | 5 |
| Compound 17 | 320 | 1.25 | 320 | 320 | 0.313 | 320 |
| Compound 18 | 320 | 5 | 5 | 320 | 1.25 | 1.25 |
| Compound 19 | 320 | 2.5 | 320 | 320 | 0.625 | 320 |

TABLE 11A
Assessing the susceptibility of EKO-35 harboring ASKA
plasmids expressing rspA, tufA, pitA, wcaC, gyrB, and yjfC.
Strains were assessed in technical duplicate and instances
where the MIC value differed >2-fold between EKO-35 and
EKO-35 harboring ASKA plasmids are bolded. Gene expression
was induced with 0.1 mM IPTG. Control strains harbored
empty pCA24N. ND: not determined. NV: not viable.
Susceptibility testing was performed both in the presence
and absence of plasmid selection (25 μg/mL and 4 μg/mL
chloramphenicol for the wild-type E. coli
and EKO-35 strains, respectively).

| | Strain (pCA24N) | | | | | |
|---|---|---|---|---|---|---|
| | Benzalkonium | | Novobiocin | | Trimethoprim | |
| | chloride (μg/mL) | | (μg/mL) | | (μg/mL) | |
| Chlor | − | + | − | + | − | + |
| K-12 | 25 | 25 | 400 | 200 | 0.781 | 0.781 |
| K-12 gyrB | ND | ND | 400 | 100 | ND | ND |
| K-12 | ND | ND | ND | ND | 1.56 | 0.195 |
| wcaC | | | | | | |
| ΔtolC | 1.56 | 0.781 | 3.13 | 1.56 | 0.195 | 1.56 |
| ΔtolC | ND | ND | 12.5 | 12.5 | ND | ND |
| gyrB | | | | | | |
| ΔtolC | ND | ND | ND | ND | 0.195 | 0.195 |
| wcaC | | | | | | |
| EKO-35 | 0.781 | 0.781 | 3.13 | 3.13 | 0.391 | 0.391 |
| EKO-35 | 0.781 | 0.781 | 12.5 | 25 | 0.391 | 0.391 |
| gyrB | | | | | | |
| EKO-35 | 0.781 | 0.391 | 1.56 | 1.56 | 0.781 | 0.781 |
| wcaC | | | | | | |
| EKO-35 | 0.781 | 0.781 | 3.13 | 1.56 | 0.391 | 0.391 |
| rspA | | | | | | |
| EKO-35 | 0.781 | NV | 1.56 | NV | 0.391 | NV |
| pitA | | | | | | |
| EKO-35 | 0.781 | 0.781 | 1.56 | 3.13 | 0.391 | 0.391 |
| tufA | | | | | | |
| EKO-35 | 0.781 | 0.781 | 3.13 | 3.13 | 0.391 | 0.391 |
| yjfC | | | | | | |

TABLE 11B
Assessing the susceptibility of EKO-35 harboring ASKA
plasmids expressing rspA, tufA, pitA, wcaC, gyrB, and yjfC.
Strains were assessed in technical duplicate and instances
where the MIC value differed >2-fold between EKO-35 and
EKO-35 harboring ASKA plasmids are bolded. Gene expression
was induced with 0.1 mM IPTG. Control strains harbored
empty pCA24N. ND: not determined. NV: not viable.
Susceptibility testing was performed both in the presence
and absence of plasmid selection (25 μg/mL and 4 μg/mL
chloramphenicol for the wild-type E. coli
and EKO-35 strains, respectively).

| Strain (pCA24N) | | |
|---|---|---|
| Synthetic 2 | Synthetic 14 | Synthetic 19 |
| (μg/mL) | (μg/mL) | (μg/mL) |

| Chlor | − | + | − | + | − | + |
|---|---|---|---|---|---|---|
| K-12 | >5 | >5 | >5 | >5 | >5 | >5 |
| K-12 | ND | ND | ND | ND | ND | ND |
| gyrB | | | | | | |
| K-12 | ND | ND | ND | ND | ND | ND |
| wcaC | | | | | | |
| ΔtolC | 1.25 | 1.25 | 1.25 | 1.25 | 2.5 | 2.5 |
| ΔtolC | ND | ND | ND | ND | ND | ND |
| gyrB | | | | | | |
| ΔtolC | ND | ND | ND | ND | ND | ND |
| wcaC | | | | | | |
| EKO-35 | >5 | >5 | >5 | >5 | >5 | >5 |
| EKO-35 | >5 | >5 | >5 | >5 | >5 | >5 |
| gyrB | | | | | | |
| EKO-35 | >5 | >5 | >5 | >5 | >5 | >5 |
| wcaC | | | | | | |
| EKO-35 | >5 | >5 | >5 | >5 | >5 | >5 |
| rspA | | | | | | |
| EKO-35 | >5 | NV | >5 | NV | >5 | NV |
| pitA | | | | | | |
| EKO-35 | >5 | >5 | >5 | >5 | >5 | >5 |
| tufA | | | | | | |
| EKO-35 | >5 | >5 | >5 | >5 | >5 | >5 |
| yjfC | | | | | | |

TABLE 12
Efflux genes and reported substrates.

| Gene | Known Substrates |
|---|---|
| acrB | Ampicillin, oxacillin, meropenem, chloramphenicol, puromycin, |
| | erythromycin, ciprofloxacin, norfloxacin, nalidixic acid, linezolid, fusidic |
| | acid, tetracycline, novobiocin, trimethoprim, acriflavine, ethidium |
| | bromide, SDS, deoxycholate, sodium cholate, rifampin |
| acrD | Kanamycin, amikacin, gentamicin, tobramycin, neomycin, novobiocin, |
| | SDS, deoxycholate, oxacillin, carbenicillin puromycin |
| acrF | Chloramphenicol, erythromycin, nalidixic acid, tetracycline, acriflavine, |
| | trimethoprim, doxorubicin, novobiocin, norfloxacin, SDS, deoxycholate, |
| | benzalkonium chloride, oxacillin |
| emrB | CCCP, SDS, deoxycholate, TSA, nalidixate |
| emrY | Deoxycholate |
| mdtF | Erythromycin, telithromycin, azithromycin, ethidium bromide, |
| | doxorubicin, crystal violet, TTP, SDS, novobiocin, ciprofloxacin, |
| | deoxycholate, cholate, taurocholate, oxacillin |
| mdtBC | Norfloxacin, nalidixic acid, novobiocin, SDS, deoxycholate, fosfomycin, |
| | benzalkonium chloride |
| macB | Erythromycin, azithromycin, additional macrolides |
| emrE | Erythromycin, acriflavine, ethidium bromide, benzalkonium chloride, |
| | tetracycline, TTP, crystal violet, streptomycin, tobramycin (and additional |
| | aminoglycosides), sulfadiazine |
| mdtK | Chloramphenicol, ciprofloxacin, norfloxacin, trimethoprim, acriflavine, |
| | ethidium bromide, doxorubicin, benzalkonium chloride, fosfomycin, |
| | novobiocin, |
| mexCD | Tetracycline, chloramphenicol, novobiocin, macrolides, quinolones, |
| | meropenem, acriflavine, ethidium bromide |

TABLE 13A
Minimum inhibitory concentrations (MICs) of various compounds against the wild-type K-12,
ΔtolC, EKO-35, and the efflux-integrated EKO-35 strains, which were used to calculate
fold-change. Strains were assessed in technical duplicate and values that showed a 4-fold
or greater increase from the EKO-35 MIC are bolded. STDC: Sodium taurodeoxycholate.

| | Minimum Inhibitory Concentration (ug/mL) | | | | | | |
|---|---|---|---|---|---|---|---|
| | EKO-35 araC::gene | | | | | | |
| Compound | K-12 | ΔtolC | EKO-35 | acrB | acrD | acrEF | mdtEF |
| Rifampicin | 12.5 | 6.25 | 6.25 | 6.25 | 6.25 | 6.25 | 6.25 |
| Vancomycin | 200 | 200 | 200 | 200 | 200 | 200 | 200 |
| Fosfomycin | 6.25 | 6.25 | 3.125 | 1.563 | 1.563 | 1.563 | 1.563 |
| Ampicillin | 100 | 25 | 25 | 100 | 100 | 50 | 25 |
| Oxacillin | 160 | 0.625 | 0.625 | 160 | 160 | 160 | 20 |
| Chloramphenicol | 6.25 | 1.563 | 0.781 | 6.25 | 1.563 | 1.563 | 0.781 |
| Puromycin | 50 | 3.125 | 1.563 | 100 | 1.563 | 6.25 | 1.563 |
| Azithromycin | 6.25 | 0.781 | 0.781 | 12.5 | 0.781 | 6.25 | 6.25 |
| Erythromycin | 100 | 3.125 | 3.125 | 100 | 3.125 | 50 | 50 |
| Spectinomycin | 25 | 6.25 | 25 | 25 | 25 | 25 | 25 |
| Tetracycline | 1.563 | 0.391 | 0.195 | 0.781 | 0.195 | 0.195 | 0.195 |
| Linezolid | 500 | 7.813 | 7.813 | 250 | 7.813 | 15.625 | 3.906 |
| Kanamycin | 3.125 | 1.563 | 3.125 | 3.125 | 3.125 | 3.125 | 3.125 |
| Streptomycin | 6.25 | 3.125 | 3.125 | 6.25 | 6.25 | 3.125 | 6.25 |
| Minocycline | 0.781 | 0.098 | 0.049 | 0.391 | 0.098 | 0.098 | 0.049 |
| Fusidic acid | 400 | 3.125 | 3.125 | 400 | 100 | 50 | 50 |
| Ciprofloxacin | 0.010 | 0.005 | 0.005 | 0.020 | 0.005 | 0.020 | 0.005 |
| Norfloxacin | 0.078 | 0.020 | 0.020 | 0.078 | 0.020 | 0.078 | 0.020 |
| Nalidixic acid | 10 | 1.25 | 1.25 | 10 | 2.5 | 5 | 1.25 |
| Novobiocin | 200 | 0.781 | 3.125 | 400 | 200 | 400 | 50 |
| Trimethoprim | 0.391 | 0.098 | 0.195 | 1.563 | 0.391 | 0.781 | 0.391 |
| Doxorubicin | 200 | 1.563 | 1.563 | 200 | 3.125 | 100 | 100 |
| Daunorubicin | 200 | 1.563 | 3.125 | 200 | 6.25 | 200 | 200 |
| Ethidium bromide | 100 | 3.125 | 0.195 | 12.5 | 0.391 | 3.125 | 1.563 |
| Bicyclomycin | 200 | 200 | 200 | 200 | 200 | 200 | 200 |
| Sulfathiazole | 5 | 5 | 10 | 10 | 10 | 10 | 10 |
| Acriflavine | 50 | 3.125 | 0.098 | 3.125 | 0.195 | 0.781 | 0.195 |
| SDS | 1000 | 15.625 | 31.25 | 1000 | 1000 | 1000 | 1000 |
| Benzalkonium | 12.5 | 0.391 | 0.391 | 12.5 | 0.391 | 1.563 | 1.563 |
| chloride | | | | | | | |
| Deoxycholate | 1500 | 187.5 | 375 | 1500 | 1500 | 1500 | 1500 |
| STDC | 1000 | 250 | 500 | 1000 | 1000 | 1000 | 1000 |
| Chlorhexidine | 0.781 | 0.391 | 0.391 | 0.391 | 0.391 | 0.391 | 0.391 |
| CCCP | 25 | 0.781 | 12.5 | 12.5 | 6.25 | 12.5 | 12.5 |
| Spermine | 2000 | 2000 | 2000 | 2000 | 2000 | 2000 | 2000 |
| Compound 1 | 320 | 5 | 320 | 320 | 320 | 320 | 320 |
| Compound 2 | 320 | 1.25 | 320 | 320 | 320 | 320 | 320 |
| Compound 3 | 320 | 2.5 | 320 | 320 | 320 | 320 | 320 |
| Compound 4 | 320 | 10 | 20 | 320 | 320 | 320 | 320 |
| Compound 5 | 320 | 10 | 20 | 320 | 40 | 320 | 320 |
| Compound 6 | 320 | 0.625 | 320 | 320 | 320 | 320 | 320 |
| Compound 7 | 320 | 0.313 | 320 | 320 | 320 | 320 | 320 |
| Compound 8 | 640 | 1.25 | 320 | 640 | 160 | 320 | 320 |
| Compound 10 | 160 | 10 | 160 | 160 | 160 | 160 | 160 |
| Compound 11 | 320 | 0.156 | 0.078 | 320 | 0.625 | 320 | 10 |
| Compound 12 | 320 | 1.25 | 320 | 320 | 320 | 320 | 320 |
| Compound 13 | 320 | 1.25 | 40 | 320 | 320 | 320 | 320 |
| Compound 14 | 320 | 2.5 | 320 | 320 | 320 | 320 | 320 |
| Compound 15 | 320 | 1.25 | 320 | 320 | 320 | 320 | 320 |
| Compound 16 | 320 | 10 | 10 | 320 | 10 | 320 | 160 |
| Compound 17 | 320 | 1.25 | 320 | 320 | 320 | 320 | 320 |
| Compound 18 | 320 | 5 | 5 | 320 | 320 | 320 | 320 |
| Compound 19 | 320 | 2.5 | 320 | 320 | 320 | 320 | 320 |

TABLE 13B
Minimum inhibitory concentrations (MICs) of various compounds against
the wild-type K-12, ΔtolC, EKO-35, and the efflux-integrated EKO-35
strains, which were used to calculate fold-change. Strains were assessed
in technical duplicate and values that showed a 4-fold or greater increase
from the EKO-35 MIC are bolded. STDC: Sodium taurodeoxycholate.

| | Minimum Inhibitory Concentration (ug/mL) | | | | | |
|---|---|---|---|---|---|---|
| | EKO-35 araC::gene | | | | | |
| Compound | mdtBC | macAB | emrKY | emrAB | mexCD | acrB_{D408A} |
| Rifampicin | 6.25 | 6.25 | 6.25 | 6.25 | 3.125 | 3.125 |
| Vancomycin | 200 | 200 | 200 | 200 | 200 | 200 |
| Fosfomycin | 1.563 | 3.125 | 1.563 | 3.125 | 1.563 | 3.125 |
| Ampicillin | 25 | 12.5 | 25 | 25 | 25 | 25 |
| Oxacillin | 0.625 | 0.625 | 0.625 | 1.25 | 20 | 1.25 |
| Chloramphenicol | 0.781 | 0.781 | 0.391 | 0.781 | 0.781 | 0.781 |
| Puromycin | 1.563 | 1.563 | 1.563 | 1.563 | 6.25 | 1.563 |
| Azithromycin | 0.781 | 3.125 | 0.391 | 0.781 | 3.125 | 0.781 |
| Erythromycin | 3.125 | 12.5 | 3.125 | 3.125 | 25 | 3.125 |
| Spectinomycin | 25 | 25 | 25 | 25 | 25 | 25 |
| Tetracycline | 0.195 | 0.195 | 0.195 | 0.195 | 0.195 | 0.195 |
| Linezolid | 3.906 | 3.906 | 3.906 | 3.906 | 15.625 | 7.813 |
| Kanamycin | 3.125 | 3.125 | 1.563 | 3.125 | 1.563 | 1.563 |
| Streptomycin | 6.25 | 3.125 | 3.125 | 3.125 | 3.125 | 3.125 |
| Minocycline | 0.049 | 0.049 | 0.049 | 0.049 | 0.049 | 0.049 |
| Fusidic acid | 6.25 | 3.125 | 3.125 | 6.25 | 12.5 | 3.125 |
| Ciprofloxacin | 0.005 | 0.005 | 0.005 | 0.005 | 0.010 | 0.005 |
| Norfloxacin | 0.020 | 0.020 | 0.010 | 0.020 | 0.039 | 0.020 |
| Nalidixic acid | 1.25 | 1.25 | 1.25 | 10 | 2.5 | 1.25 |
| Novobiocin | 12.5 | 1.563 | 3.125 | 25 | 25 | 3.125 |
| Trimethoprim | 0.195 | 0.195 | 0.195 | 0.195 | 0.391 | 0.098 |
| Doxorubicin | 1.563 | 1.563 | 1.563 | 1.563 | 25 | 1.563 |
| Daunorubicin | 1.563 | 6.25 | 3.125 | 3.125 | 100 | 3.125 |
| Ethidium | 0.195 | 0.195 | 0.195 | 0.195 | 1.563 | 0.391 |
| bromide | | | | | | |
| Bicyclomycin | 200 | 200 | 200 | 200 | 200 | 200 |
| Sulfathiazole | 10 | 5 | 10 | 10 | 10 | 10 |
| Acriflavine | 0.098 | 0.098 | 0.098 | 0.098 | 0.781 | 0.098 |
| SDS | 62.5 | 31.25 | 62.5 | 125 | 1000 | 62.5 |
| Benzalkonium | 0.391 | 0.391 | 0.391 | 0.391 | 0.781 | 0.391 |
| chloride | | | | | | |
| Deoxycholate | 1500 | 187.5 | 375 | 1500 | 1500 | 375 |
| STDC | 1000 | 250 | 500 | 1000 | 1000 | 1000 |
| Chlorhexidine | 0.391 | 0.391 | 0.391 | 0.391 | 0.391 | 0.391 |
| CCCP | 12.5 | 12.5 | 12.5 | 50 | 12.5 | 12.5 |
| Spermine | 2000 | 2000 | 2000 | 2000 | 2000 | 2000 |
| Compound 1 | 320 | 320 | 320 | 320 | 320 | 320 |
| Compound 2 | 320 | 320 | 320 | 320 | 320 | 320 |
| Compound 3 | 320 | 320 | 320 | 320 | 320 | 320 |
| Compound 4 | 20 | 20 | 20 | 320 | 320 | 20 |
| Compound 5 | 10 | 20 | 20 | 320 | 320 | 20 |
| Compound 6 | 320 | 320 | 320 | 320 | 320 | 320 |
| Compound 7 | 320 | 320 | 320 | 320 | 320 | 320 |
| Compound 8 | 320 | 320 | 320 | 320 | 320 | 320 |
| Compound 10 | 160 | 160 | 160 | 160 | 160 | 160 |
| Compound 11 | 0.078 | 0.078 | 0.078 | 40 | 1.25 | 0.078 |
| Compound 12 | 320 | 320 | 320 | 320 | 320 | 320 |
| Compound 13 | 40 | 40 | 40 | 320 | 320 | 40 |
| Compound 14 | 320 | 320 | 320 | 320 | 320 | 320 |
| Compound 15 | 320 | 320 | 320 | 320 | 320 | 320 |
| Compound 16 | 5 | 10 | 5 | 160 | 160 | 10 |
| Compound 17 | 320 | 320 | 320 | 320 | 320 | 320 |
| Compound 18 | 10 | 2.5 | 2.5 | 320 | 320 | 5 |
| Compound 19 | 320 | 320 | 320 | 320 | 320 | 320 |

TABLE 14A
Minimum inhibitory concentrations (MICs) of various compounds against the wild-type (WT)-Pore,
ΔtolC-Pore, EKO-35-Pore, and the efflux-integrated EKO-35-Pore strains, which were used
to calculate fold-change. Strains were assessed in technical duplicate and values that showed
a 4-fold or greater increase from the EKO-35 MIC are bolded. STDC: Sodium taurodeoxycholate.

| | Minimum inhibitory concentration (μg/mL) | | | | | | |
|---|---|---|---|---|---|---|---|
| | EKO-35 araC::gene Pore | | | | | | |
| Compound | K-12 | ΔtolC | EKO-35 | acrB | acrD | acrEF | mdtEF |
| Rifampicin | 0.781 | 0.391 | 0.391 | 1.563 | 0.391 | 0.391 | 0.391 |
| Vancomycin | 3.125 | 6.25 | 6.25 | 3.125 | 3.125 | 3.125 | 6.25 |
| Fosfomycin | 3.125 | 3.125 | 0.781 | 0.781 | 0.781 | 1.563 | 0.781 |
| Ampicillin | | 1.563 | 1.563 | 12.5 | 25 | 3.125 | 1.563 |
| Oxacillin | 40 | 0.313 | 0.313 | 20 | 20 | 5 | 1.25 |
| Chloramphenicol | 3.125 | 0.781 | 0.781 | 3.125 | 0.781 | 0.781 | 0.781 |
| Puromycin | 50 | 1.563 | 0.781 | 50 | 0.781 | 3.125 | 1.563 |
| Azithromycin | 0.391 | 0.049 | 0.049 | 0.195 | 0.024 | 0.195 | 0.195 |
| Erythromycin | 6.25 | 0.195 | 0.195 | 6.25 | 0.195 | 1.563 | 1.563 |
| Spectinomycin | 12.5 | 6.25 | 12.5 | 25 | 25 | 12.5 | 12.5 |
| Tetracycline | 0.781 | 0.391 | 0.195 | 0.391 | 0.195 | 0.195 | 0.195 |
| Linezolid | 125 | 3.906 | 3.906 | 62.5 | 7.813 | 7.813 | 7.813 |
| Kanamycin | 3.125 | 1.563 | 1.563 | 3.125 | 1.563 | 1.563 | 1.563 |
| Streptomycin | 12.5 | 1.563 | 3.125 | 6.25 | 3.125 | 3.125 | 3.125 |
| Minocycline | 0.781 | 0.098 | 0.049 | 0.391 | 0.098 | 0.098 | 0.098 |
| Fusidic acid | 100 | 0.391 | 0.391 | 100 | 12.5 | 12.5 | 3.125 |
| Ciprofloxacin | 0.005 | 0.002 | 0.002 | 0.009 | 0.002 | 0.010 | 0.002 |
| Norfloxacin | 0.078 | 0.010 | 0.010 | 0.078 | 0.020 | 0.039 | 0.020 |
| Nalidixic acid | 5 | 1.25 | 1.25 | 10 | 1.25 | 2.5 | 1.25 |
| Novobiocin | 12.5 | 0.391 | 0.781 | 400 | 25 | 25 | 12.5 |
| Trimethoprim | 0.391 | 0.049 | 0.195 | 0.781 | 0.195 | 0.195 | 0.391 |
| Doxorubicin | 50 | 0.781 | 0.781 | 50 | 0.781 | 12.5 | 25 |
| Daunorubicin | 100 | 0.781 | 0.781 | 100 | 1.563 | 25 | 50 |
| Ethidium bromide | 100 | 3.125 | 0.098 | 6.25 | 0.195 | 1.563 | 0.781 |
| Bicyclomycin | 200 | 200 | 200 | 200 | 200 | 200 | 200 |
| Sulfathiazole | 5 | 2.5 | 5 | 10 | 5 | 5 | 10 |
| Acriflavine | 50 | 3.125 | 0.098 | 1.563 | 0.195 | 0.781 | 0.195 |
| SDS | 1000 | 15.625 | 31.25 | 1000 | 1000 | 1000 | 1000 |
| Benzalkonium | 6.25 | 0.391 | 0.391 | 3.125 | 0.391 | 0.781 | 1.563 |
| chloride | | | | | | | |
| Deoxycholate | 1500 | 46.875 | 187.5 | 1500 | 1500 | 1500 | 1500 |
| STDC | 1000 | 250 | 250 | 1000 | 1000 | 1000 | 1000 |
| Chlorhexidine | 0.781 | 0.391 | 0.391 | 0.391 | 0.391 | 0.391 | 0.391 |
| CCCP | 12.5 | 0.391 | 6.25 | 6.25 | 6.25 | 3.125 | 6.25 |
| Spermine | 2000 | 2000 | 2000 | 2000 | 2000 | 2000 | 2000 |
| Compound 1 | 320 | 10 | 320 | 320 | 320 | 320 | 320 |
| Compound 2 | 320 | 1.25 | 320 | 320 | 320 | 320 | 320 |
| Compound 3 | 320 | 1.25 | 320 | 320 | 320 | 320 | 320 |
| Compound 4 | 320 | 1.25 | 2.5 | 320 | 10 | 320 | 320 |
| Compound 5 | 320 | 2.5 | 20 | 320 | 10 | 320 | 320 |
| Compound 6 | 320 | 0.625 | 320 | 320 | 320 | 320 | 320 |
| Compound 7 | 320 | 0.313 | 320 | 320 | 320 | 320 | 320 |
| Compound 8 | 160 | 1.25 | 80 | 160 | 40 | 40 | 80 |
| Compound 10 | 160 | 5 | 10 | 160 | 160 | 160 | 160 |
| Compound 11 | 80 | 0.156 | 0.078 | 80 | 0.313 | 80 | 5 |
| Compound 12 | 320 | 0.625 | 160 | 320 | 160 | 320 | 320 |
| Compound 13 | 320 | 1.25 | 5 | 320 | 10 | 20 | 40 |
| Compound 14 | 320 | 1.25 | 320 | 320 | 320 | 320 | 320 |
| Compound 15 | 320 | 0.625 | 320 | 320 | 320 | 320 | 320 |
| Compound 16 | 160 | 5 | 5 | 160 | 5 | 80 | 80 |
| Compound 17 | 320 | 0.313 | 320 | 320 | 320 | 320 | 320 |
| Compound 18 | 320 | 1.25 | 1.25 | 320 | 10 | 320 | 320 |
| Compound 19 | 320 | 0.625 | 320 | 320 | 320 | 320 | 320 |

TABLE 14B
Minimum inhibitory concentrations (MICs) of various compounds against the
wild-type (WT)-Pore, ΔtolC-Pore, EKO-35-Pore, and the efflux-integrated
EKO-35-Pore strains, which were used to calculate fold-change. Strains were
assessed in technical duplicate and values that showed a 4-fold or greater
increase from the EKO-35 MIC are bolded. STDC: Sodium taurodeoxycholate.

| | Minimum inhibitory concentration (μg/mL) | | | | | |
|---|---|---|---|---|---|---|
| | EKO-35 araC::gene Pore | | | | | |
| Compound | mdtBC | macAB | emrKY | emrAB | mexCD | acrB_{D408A} |
| Rifampicin | 0.781 | 0.391 | 0.391 | 0.195 | 0.391 | 0.195 |
| Vancomycin | 1.563 | 6.25 | 3.125 | 3.125 | 6.25 | 6.25 |
| Fosfomycin | 0.781 | 0.781 | 0.781 | 1.563 | 1.563 | 0.781 |
| Ampicillin | 0.781 | 1.563 | 3.125 | 0.781 | 1.563 | 0.781 |
| Oxacillin | 0.313 | 0.313 | 0.313 | 0.313 | 2.5 | 0.156 |
| Chloramphenicol | 0.781 | 0.781 | 0.391 | 0.781 | 0.781 | 0.781 |
| Puromycin | 0.781 | 0.781 | 1.563 | 0.781 | 1.563 | 0.781 |
| Azithromycin | 0.049 | 0.098 | 0.024 | 0.049 | 0.098 | 0.098 |
| Erythromycin | 0.195 | 0.781 | 0.195 | 0.195 | 1.563 | 0.098 |
| Spectinomycin | 12.5 | 12.5 | 12.5 | 12.5 | 12.5 | 12.5 |
| Tetracycline | 0.195 | 0.195 | 0.195 | 0.195 | 0.195 | 0.195 |
| Linezolid | 3.906 | 3.906 | 3.906 | 7.813 | 7.813 | 3.906 |
| Kanamycin | 1.563 | 3.125 | 1.563 | 1.563 | 1.563 | 0.781 |
| Streptomycin | 3.125 | 3.125 | 3.125 | 3.125 | 3.125 | 1.563 |
| Minocycline | 0.049 | 0.049 | 0.049 | 0.049 | 0.049 | 0.049 |
| Fusidic acid | 0.391 | 0.391 | 0.391 | 1.563 | 1.563 | 0.391 |
| Ciprofloxacin | 0.002 | 0.002 | 0.002 | 0.005 | 0.005 | 0.002 |
| Norfloxacin | 0.020 | 0.020 | 0.010 | 0.020 | 0.039 | 0.020 |
| Nalidixic acid | 1.25 | 1.25 | 1.25 | 10 | 2.5 | 1.25 |
| Novobiocin | 3.125 | 0.781 | 0.781 | 6.25 | 3.125 | 0.391 |
| Trimethoprim | 0.195 | 0.195 | 0.391 | 0.391 | 0.195 | 0.195 |
| Doxorubicin | 3.125 | 0.781 | 0.781 | 0.781 | 3.125 | 0.781 |
| Daunorubicin | 3.125 | 1.563 | 0.781 | 1.563 | 12.5 | 0.781 |
| Ethidium bromide | 0.098 | 0.098 | 0.098 | 0.098 | 0.781 | 0.098 |
| Bicyclomycin | 200 | 200 | 200 | 200 | 200 | 200 |
| Sulfathiazole | 5 | 10 | 5 | 10 | 5 | 1.25 |
| Acriflavine | 0.098 | 0.195 | 0.098 | 0.195 | 0.391 | 0.098 |
| SDS | 31.25 | 31.25 | 31.25 | 125 | 125 | 31.25 |
| Benzalkonium | 0.195 | 0.391 | 0.195 | 0.391 | 0.391 | 0.391 |
| chloride | | | | | | |
| Deoxycholate | 750 | 187.5 | 187.5 | 750 | 375 | 93.75 |
| STDC | 1000 | 250 | 250 | 500 | 1000 | 250 |
| Chlorhexidine | 0.391 | 0.391 | 0.391 | 0.391 | 0.391 | 0.391 |
| CCCP | 12.5 | 6.25 | 6.25 | 12.5 | 6.25 | 6.25 |
| Spermine | 2000 | 2000 | 2000 | 2000 | 2000 | 500 |
| Compound 1 | 320 | 320 | 320 | 320 | 320 | 320 |
| Compound 2 | 320 | 320 | 320 | 320 | 320 | 320 |
| Compound 3 | 320 | 320 | 320 | 320 | 320 | 320 |
| Compound 4 | 2.5 | 5 | 1.25 | 20 | 40 | 2.5 |
| Compound 5 | 10 | 10 | 10 | 320 | 320 | 20 |
| Compound 6 | 320 | 320 | 320 | 320 | 320 | 320 |
| Compound 7 | 320 | 320 | 320 | 320 | 320 | 320 |
| Compound 8 | 80 | 80 | 80 | 40 | 80 | 80 |
| Compound 10 | 10 | 10 | 10 | 160 | 160 | 10 |
| Compound 11 | 0.078 | 0.078 | 0.078 | 1.25 | 0.625 | 0.078 |
| Compound 12 | 320 | 320 | 320 | 320 | 320 | 320 |
| Compound 13 | 5 | 5 | 2.5 | 10 | 5 | 5 |
| Compound 14 | 320 | 320 | 320 | 320 | 320 | 320 |
| Compound 15 | 320 | 320 | 320 | 320 | 320 | 320 |
| Compound 16 | 5 | 5 | 2.5 | 80 | 80 | 5 |
| Compound 17 | 320 | 320 | 320 | 320 | 320 | 320 |
| Compound 18 | 1.25 | 1.25 | 1.25 | 320 | 20 | 1.25 |
| Compound 19 | 320 | 320 | 320 | 320 | 320 | 320 |

TABLE 15A
Fold change in the MIC of profiled compounds against EKO-35
efflux pump-integrated strains compared to EKO-35. Strains
were assessed in technical duplicate and values that showed
a 4-fold or greater increase in resistance compared to
EKO-35 are bolded. Related to FIGS. 4A-4F.

| | Fold change of MICs | | | | | |
|---|---|---|---|---|---|---|
| | EKO-35 araC::gene | | | | | |
| Compound | EKO-35 | acrB | acrD | acrEF | mdtEF | mdtBC |
| Rifampicin | 1 | 1 | 1 | 1 | 1 | 1 |
| Vancomycin | 1 | 1 | 1 | 1 | 1 | 1 |
| Fosfomycin | 1 | 1 | 1 | 1 | 1 | 1 |
| Ampicillin | 1 | 4 | 4 | 2 | 1 | 1 |
| Oxacillin | 1 | 256 | 256 | 256 | 32 | 1 |
| Chloramphenicol | 1 | 8 | 2 | 2 | 1 | 1 |
| Puromycin | 1 | 64 | 1 | 4 | 1 | 1 |
| Azithromycin | 1 | 16 | 1 | 8 | 8 | 1 |
| Erythromycin | 1 | 32 | 1 | 16 | 16 | 1 |
| Spectinomycin | 1 | 1 | 1 | 1 | 1 | 1 |
| Tetracycline | 1 | 4 | 1 | 1 | 1 | 1 |
| Linezolid | 1 | 32 | 1 | 2 | 1 | 1 |
| Kanamycin | 1 | 1 | 1 | 1 | 1 | 1 |
| Streptomycin | 1 | 2 | 2 | 1 | 2 | 2 |
| Minocycline | 1 | 8 | 2 | 2 | 1 | 1 |
| Fusidic acid | 1 | 128 | 32 | 16 | 16 | 2 |
| Ciprofloxacin | 1 | 4 | 1 | 4 | 1 | 1 |
| Norfloxacin | 1 | 4 | 1 | 4 | 1 | 1 |
| Nalidixic acid | 1 | 8 | 2 | 4 | 1 | 1 |
| Novobiocin | 1 | 128 | 64 | 128 | 16 | 4 |
| Trimethoprim | 1 | 8 | 2 | 4 | 2 | 1 |
| Doxorubicin | 1 | 128 | 2 | 64 | 64 | 1 |
| Daunorubicin | 1 | 64 | 2 | 64 | 64 | 1 |
| Ethidium bromide | 1 | 64 | 2 | 16 | 8 | 1 |
| Bicyclomycin | 1 | 1 | 1 | 1 | 1 | 1 |
| Sulfathiazole | 1 | 1 | 1 | 1 | 1 | 1 |
| Acriflavine | 1 | 32 | 2 | 8 | 2 | 1 |
| SDS | 1 | 32 | 32 | 32 | 32 | 2 |
| Benzalkonium | 1 | 32 | 1 | 4 | 4 | 1 |
| chloride | | | | | | |
| Deoxycholate | 1 | 4 | 4 | 4 | 4 | 4 |
| Sodium | 1 | 2 | 2 | 2 | 2 | 2 |
| taurodeoxycholate | | | | | | |
| Chlorhexidine | 1 | 1 | 1 | 1 | 1 | 1 |
| CCCP | 1 | 1 | 1 | 1 | 1 | 1 |
| Spermine | 1 | 1 | 1 | 1 | 1 | 1 |
| Compound 1 | 1 | 1 | 1 | 1 | 1 | 1 |
| Compound 2 | 1 | 1 | 1 | 1 | 1 | 1 |
| Compound 3 | 1 | 1 | 1 | 1 | 1 | 1 |
| Compound 4 | 1 | 16 | 16 | 16 | 16 | 1 |
| Compound 5 | 1 | 16 | 2 | 16 | 16 | 1 |
| Compound 6 | 1 | 1 | 1 | 1 | 1 | 1 |
| Compound 7 | 1 | 1 | 1 | 1 | 1 | 1 |
| Compound 8 | 1 | 2 | 1 | 1 | 1 | 1 |
| Compound 10 | 1 | 1 | 1 | 1 | 1 | 1 |
| Compound 11 | 1 | 4096 | 8 | 4096 | 128 | 1 |
| Compound 12 | 1 | 1 | 1 | 1 | 1 | 1 |
| Compound 13 | 1 | 8 | 8 | 8 | 8 | 1 |
| Compound 14 | 1 | 1 | 1 | 1 | 1 | 1 |
| Compound 15 | 1 | 1 | 1 | 1 | 1 | 1 |
| Compound 16 | 1 | 32 | 1 | 32 | 16 | 1 |
| Compound 17 | 1 | 1 | 1 | 1 | 1 | 1 |
| Compound 18 | 1 | 64 | 64 | 64 | 64 | 2 |
| Compound 19 | 1 | 1 | 1 | 1 | 1 | 1 |

TABLE 15B
Fold change in the MIC of profiled compounds against EKO-35
efflux pump-integrated strains compared to EKO-35. Strains
were assessed in technical duplicate and values that showed
a 4-fold or greater increase in resistance compared to
EKO-35 are bolded. Related to FIGS. 4A-4F.

| | Fold change of MICs | | | | |
|---|---|---|---|---|---|
| | EKO-35 araC::gene | | | | |
| Compound | macAB | emrKY | emrAB | mexCD | acrBD408A |
| Rifampicin | 1 | 1 | 1 | 1 | 1 |
| Vancomycin | 1 | 1 | 1 | 1 | 1 |
| Fosfomycin | 1 | 1 | 1 | 1 | 1 |
| Ampicillin | 1 | 1 | 1 | 1 | 1 |
| Oxacillin | 1 | 1 | 2 | 32 | 2 |
| Chloramphenicol | 1 | 1 | 1 | 1 | 1 |
| Puromycin | 1 | 1 | 1 | 4 | 1 |
| Azithromycin | 4 | 1 | 1 | 4 | 1 |
| Erythromycin | 4 | 1 | 1 | 8 | 1 |
| Spectinomycin | 1 | 1 | 1 | 1 | 1 |
| Tetracycline | 1 | 1 | 1 | 1 | 1 |
| Linezolid | 1 | 1 | 1 | 2 | 1 |
| Kanamycin | 1 | 1 | 1 | 1 | 1 |
| Streptomycin | 1 | 1 | 1 | 1 | 1 |
| Minocycline | 1 | 1 | 1 | 1 | 1 |
| Fusidic acid | 1 | 1 | 2 | 4 | 1 |
| Ciprofloxacin | 1 | 1 | 1 | 2 | 1 |
| Norfloxacin | 1 | 0 | 1 | 2 | 1 |
| Nalidixic acid | 1 | 1 | 8 | 2 | 1 |
| Novobiocin | 1 | 1 | 8 | 8 | 1 |
| Trimethoprim | 1 | 1 | 1 | 2 | 0 |
| Doxorubicin | 1 | 1 | 1 | 16 | 1 |
| Daunorubicin | 2 | 1 | 1 | 32 | 1 |
| Ethidium bromide | 1 | 1 | 1 | 8 | 2 |
| Bicyclomycin | 1 | 1 | 1 | 1 | 1 |
| Sulfathiazole | 1 | 1 | 1 | 1 | 1 |
| Acriflavine | 1 | 1 | 1 | 8 | 1 |
| SDS | 1 | 2 | 4 | 32 | 2 |
| Benzalkonium | 1 | 1 | 1 | 2 | 1 |
| chloride | | | | | |
| Deoxycholate | 1 | 1 | 4 | 4 | 1 |
| Sodium | 1 | 1 | 2 | 2 | 2 |
| taurodeoxycholate | | | | | |
| Chlorhexidine | 1 | 1 | 1 | 1 | 1 |
| CCCP | 1 | 1 | 4 | 1 | 1 |
| Spermine | 1 | 1 | 1 | 1 | 1 |
| Compound 1 | 1 | 1 | 1 | 1 | 1 |
| Compound 2 | 1 | 1 | 1 | 1 | 1 |
| Compound 3 | 1 | 1 | 1 | 1 | 1 |
| Compound 4 | 1 | 1 | 16 | 16 | 1 |
| Compound 5 | 1 | 1 | 16 | 16 | 1 |
| Compound 6 | 1 | 1 | 1 | 1 | 1 |
| Compound 7 | 1 | 1 | 1 | 1 | 1 |
| Compound 8 | 1 | 1 | 1 | 1 | 1 |
| Compound 10 | 1 | 1 | 1 | 1 | 1 |
| Compound 11 | 1 | 1 | 512 | 16 | 1 |
| Compound 12 | 1 | 1 | 1 | 1 | 1 |
| Compound 13 | 1 | 1 | 8 | 8 | 1 |
| Compound 14 | 1 | 1 | 1 | 1 | 1 |
| Compound 15 | 1 | 1 | 1 | 1 | 1 |
| Compound 16 | 1 | 1 | 16 | 16 | 1 |
| Compound 17 | 1 | 1 | 1 | 1 | 1 |
| Compound 18 | 1 | 1 | 64 | 64 | 1 |
| Compound 19 | 1 | 1 | 1 | 1 | 1 |

TABLE 16A
Fold change in the MIC of profiled compounds against EKO-35
efflux pump-integrated pore strains compared to EKO-35-Pore.
Strains were assessed in technical duplicate and values
that showed a 4-fold or greater increase in resistance compared
to EKO-35 are bolded. Related to FIGS. 4A-4F.

| | Fold changes of MICs | | | | |
|---|---|---|---|---|---|
| | EKO-35 araC::gene Pore | | | | |
| Compound | EKO-35 | acr-B | acrD | acrEF | mdtEF |
| Rifampicin | 1 | 4 | 1 | 1 | 1 |
| Vancomycin | 1 | 1 | 1 | 1 | 1 |
| Fosfomycin | 1 | 1 | 1 | 2 | 1 |
| Ampicillin | 1 | 8 | 16 | 2 | 1 |
| Oxacillin | 1 | 64 | 64 | 16 | 4 |
| Chloramphenicol | 1 | 4 | 1 | 1 | 1 |
| Puromycin | 1 | 64 | 1 | 4 | 2 |
| Azithromycin | 1 | 4 | 1 | 4 | 4 |
| Erythromycin | 1 | 32 | 1 | 8 | 8 |
| Spectinomycin | 1 | 2 | 2 | 1 | 1 |
| Tetracycline | 1 | 2 | 1 | 1 | 1 |
| Linezolid | 1 | 16 | 2 | 2 | 2 |
| Kanamycin | 1 | 2 | 1 | 1 | 1 |
| Streptomycin | 1 | 2 | 1 | 1 | 1 |
| Minocycline | 1 | 8 | 2 | 2 | 2 |
| Fusidic acid | 1 | 256 | 32 | 32 | 8 |
| Ciprofloxacin | 1 | 4 | 1 | 4 | 1 |
| Norfloxacin | 1 | 8 | 2 | 4 | 2 |
| Nalidixic acid | 1 | 8 | 1 | 2 | 1 |
| Novobiocin | 1 | 512 | 32 | 32 | 16 |
| Trimethoprim | 1 | 4 | 1 | 1 | 2 |
| Doxorubicin | 1 | 64 | 1 | 16 | 32 |
| Daunorubicin | 1 | 128 | 2 | 32 | 64 |
| Ethidium bromide | 1 | 64 | 2 | 16 | 8 |
| Bicyclomycin | 1 | 1 | 1 | 1 | 1 |
| Sulfathiazole | 1 | 2 | 1 | 1 | 2 |
| Acriflavine | 1 | 16 | 2 | 8 | 2 |
| SDS | 1 | 32 | 32 | 32 | 32 |
| Benzalkonium | 1 | 8 | 1 | 2 | 4 |
| chloride | | | | | |
| Deoxycholate | 1 | 8 | 8 | 8 | 8 |
| Sodium | 1 | 4 | 4 | 4 | 4 |
| taurodeoxycholate | | | | | |
| Chlorhexidine | 1 | 1 | 1 | 1 | 1 |
| CCCP | 1 | 1 | 1 | 1 | 1 |
| Spermine | 1 | 1 | 1 | 1 | 1 |
| Compound 1 | 1 | 1 | 1 | 1 | 1 |
| Compound 2 | 1 | 1 | 1 | 1 | 1 |
| Compound 3 | 1 | 1 | 1 | 1 | 1 |
| Compound 4 | 1 | 128 | 4 | 128 | 128 |
| Compound 5 | 1 | 16 | 1 | 16 | 16 |
| Compound 6 | 1 | 1 | 1 | 1 | 1 |
| Compound 7 | 1 | 1 | 1 | 1 | 1 |
| Compound 8 | 1 | 2 | 1 | 1 | 1 |
| Compound 10 | 1 | 16 | 16 | 16 | 16 |
| Compound 11 | 1 | 1024 | 4 | 1024 | 64 |
| Compound 12 | 1 | 2 | 1 | 2 | 2 |
| Compound 13 | 1 | 64 | 2 | 4 | 8 |
| Compound 14 | 1 | 1 | 1 | 1 | 1 |
| Compound 15 | 1 | 1 | 1 | 1 | 1 |
| Compound 16 | 1 | 32 | 1 | 16 | 16 |
| Compound 17 | 1 | 1 | 1 | 1 | 1 |
| Compound 18 | 1 | 256 | 8 | 256 | 256 |
| Compound 19 | 1 | 1 | 1 | 1 | 1 |

TABLE 16B
Fold change in the MIC of profiled compounds against EKO-35 efflux pump-
integrated pore strains compared to EKO-35-Pore. Strains were assessed in
technical duplicate and values that showed a 4-fold or greater increase
in resistance compared to EKO-35 are bolded. Related to FIGS. 4A-4F.

| | Fold changes of MICs | | | | | |
|---|---|---|---|---|---|---|
| | EKO-35 araC::gene Pore | | | | | |
| Compound | mdtBC | macAB | emrKY | emrAB | mexCD | acrB_{D408A} |
| Rifampicin | 2 | 1 | 1 | 1 | 1 | 1 |
| Vancomycin | 0 | 1 | 1 | 1 | 1 | 1 |
| Fosfomycin | 1 | 1 | 1 | 2 | 2 | 1 |
| Ampicillin | 1 | 1 | 2 | 1 | 1 | 1 |
| Oxacillin | 1 | 1 | 1 | 1 | 8 | 1 |
| Chloramphenicol | 1 | 1 | 1 | 1 | 1 | 1 |
| Puromycin | 1 | 1 | 2 | 1 | 2 | 1 |
| Azithromycin | 1 | 2 | 1 | 1 | 2 | 2 |
| Erythromycin | 1 | 4 | 1 | 1 | 8 | 0 |
| Spectinomycin | 1 | 1 | 1 | 1 | 1 | 1 |
| Tetracycline | 1 | 1 | 1 | 1 | 1 | 1 |
| Linezolid | 1 | 1 | 1 | 2 | 2 | 1 |
| Kanamycin | 1 | 2 | 1 | 1 | 1 | 1 |
| Streptomycin | 1 | 1 | 1 | 1 | 1 | 1 |
| Minocycline | 1 | 1 | 1 | 1 | 1 | 1 |
| Fusidic acid | 1 | 1 | 1 | 4 | 4 | 1 |
| Ciprofloxacin | 1 | 1 | 1 | 2 | 2 | 1 |
| Norfloxacin | 2 | 2 | 1 | 2 | 4 | 2 |
| Nalidixic acid | 1 | 1 | 1 | 8 | 2 | 1 |
| Novobiocin | 4 | 1 | 1 | 8 | 4 | 1 |
| Trimethoprim | 1 | 1 | 2 | 2 | 1 | 1 |
| Doxorubicin | 4 | 1 | 1 | 1 | 4 | 1 |
| Daunorubicin | 4 | 2 | 1 | 2 | 16 | 1 |
| Ethidium bromide | 1 | 1 | 1 | 1 | 8 | 1 |
| Bicyclomycin | 1 | 1 | 1 | 1 | 1 | 1 |
| Sulfathiazole | 1 | 2 | 1 | 2 | 1 | 0 |
| Acriflavine | 1 | 2 | 1 | 2 | 4 | 1 |
| SDS | 1 | 1 | 1 | 4 | 4 | 1 |
| Benzalkonium | 1 | 1 | 1 | 1 | 1 | 1 |
| chloride | | | | | | |
| Deoxycholate | 4 | 1 | 1 | 4 | 2 | 1 |
| Sodium | 4 | 1 | 1 | 2 | 4 | 1 |
| taurodeoxycholate | | | | | | |
| Chlorhexidine | 1 | 1 | 1 | 1 | 1 | 1 |
| CCCP | 2 | 1 | 1 | 2 | 1 | 1 |
| Spermine | 1 | 1 | 1 | 1 | 1 | 0 |
| Compound 1 | 1 | 1 | 1 | 1 | 1 | 1 |
| Compound 2 | 1 | 1 | 1 | 1 | 1 | 1 |
| Compound 3 | 1 | 1 | 1 | 1 | 1 | 1 |
| Compound 4 | 1 | 2 | 1 | 8 | 16 | 1 |
| Compound 5 | 1 | 1 | 1 | 16 | 16 | 1 |
| Compound 6 | 1 | 1 | 1 | 1 | 1 | 1 |
| Compound 7 | 1 | 1 | 1 | 1 | 1 | 1 |
| Compound 8 | 1 | 1 | 1 | 1 | 1 | 1 |
| Compound 10 | 1 | 1 | 1 | 16 | 16 | 1 |
| Compound 11 | 1 | 1 | 1 | 16 | 8 | 1 |
| Compound 12 | 2 | 2 | 2 | 2 | 2 | 2 |
| Compound 13 | 1 | 1 | 1 | 2 | 1 | 1 |
| Compound 14 | 1 | 1 | 1 | 1 | 1 | 1 |
| Compound 15 | 1 | 1 | 1 | 1 | 1 | 1 |
| Compound 16 | 1 | 1 | 1 | 16 | 16 | 1 |
| Compound 17 | 1 | 1 | 1 | 1 | 1 | 1 |
| Compound 18 | 1 | 1 | 1 | 256 | 16 | 1 |
| Compound 19 | 1 | 1 | 1 | 1 | 1 | |

TABLE 17
The physicochemical substrate parameters of drug efflux pumps assessed using EKO-35
and EKO-35-Pore strains. Molecular properties were summarized using the SMILES
chemical notation for each compound and DataWarrior (Version 5.5.0). Median values
are indicated in parentheses. (Related to FIG. 4A-4F and Table 7).

| | | | Molecular | | | Polar |
|---|---|---|---|---|---|---|
| | | Compounds | Weight | | | Surface |
| Gene | Pore | Effluxed | (g/mol) | logP | logS | Area |
| AcrB | − | 28/33 | 227.778 to | −1.986 to | −7.266 to | 0 to |
| | | | 748.992 | 5.823 | −1.265 | 206.070 |
| | | | (393.447) | (1.081) | (−3.641) | (104.785) |
| | + | 30/33 | 227.778 to | −1.986 to | −7.266 to | 0 to 220.15 |
| | | | 822.95 | 5.823 | −1.435 | (107.66) |
| | | | (393.447) | (1.664) | (−4.027) | |
| AcrEF | − | 23/33 | 227.778 to | −1.986 to | −7.266 to | 0 to |
| | | | 748.992 | 5.823 | −1.705 | 206.070 |
| | | | (394.315) | (1.672) | (−4.507) | (90.240) |
| | + | 22/33 | 285.215 to | −1.986 to | −7.266 to | 54.79 to |
| | | | 748.992 | 5.823 | −2.031 | 206.070 |
| | | | (414.846) | (2.095) | (−4.807) | (106.935) |
| MdtEF | − | 17/33 | 227.778 to | −0.340 to | −7.266 to | 0 to |
| | | | 748.992 | 5.823 | −1.705 | 206.070 |
| | | | (401.442) | (2.129) | (−4.879) | (104.060) |
| | + | 19/33 | 227.778 to | −0.340 to | −7.266 to | 0 to |
| | | | 748.992 | 5.823 | −1.705 | 206.070 |
| | | | (401.442) | (2.129) | (−4.879) | (109.810) |
| AcrD | − | 10/33 | 285.215 to | −1.653 to | −7.266 to | 65.200 to |
| | | | 612.63 | 5.823 | −1.565 | 196.100 |
| | | | (397.010) | (3.125) | (−5.076) | (114.145) |
| | + | 11/33 | 285.215 to | −1.653 to | −7.266 to | 71.98 to |
| | | | 612.63 | 5.823 | −1.565 | 196.100 |
| | | | (401.442) | (2.980) | (−5.272) | (124.230) |
| EmrAB | − | 11/33 | 204.620 to | 0.536 to | −7.266 to | 54.790 to |
| | | | 612.63 | 4.077 | −2.031 | 196.100 |
| | | | (341.676) | (3.270) | (−4.879) | (77.76) |
| | + | 11/33 | 232.238 to | 0.536 to | −7.266 to | 54.790 to |
| | | | 612.63 | 5.823 | −2.031 | 196.100 |
| | | | (341.676) | (3.369) | (−5.363) | (90.240) |
| MacAB | − | 2/33 | 733.933 to | 1.657 to | −3.645 to | 180.080 to |
| | | | 748.992 | 1.672 | −3.094 | 193.910 |
| | | | (741.463) | (1.664) | (−3.370) | (186.995) |
| | + | 1/33 | 733.933 | 1.672 | −3.645 | 193.91 |
| MdtBC | − | 2/33 | 392.578 to | 3.27 to | −5.272 to | 77.760 to |
| | | | 612.63 | 4.032 | −4.879 | 196.100 |
| | | | (502.604) | (3.651) | (−5.076) | (136.930) |
| | + | 5/33 | 392.578 to | 0.167 to | −5.272 to | 77.76 to |
| | | | 612.63 | 4.032 | −4.395 | 206.70 |
| | | | (527.524) | (2.091) | (−4.879) | (185.84) |
| EmrKY | − | 0/33 | — | — | — | — |
| | + | 0/33 | — | — | — | — |
| MexCD | − | 18/33 | 285.215 to | −1.986 to | −7.266 to | 54.790 to |
| | | | 748.992 | 5.823 | −2.031 | 206.070 |
| | | | (443.887) | (2.113) | (−4.871) | (114.145) |
| | + | 17/33 | 285.215 to | −1.986 to | −7.266 to | 54.790 to |
| | | | 733.933 | 5.823 | −2.031 | 206.070 |
| | | | (428.25) | (2.098) | (−5.014) | (109.810) |

TABLE 18A
Defining the Fractional Inhibitory Concentration Index (FICI)
for PABN in combination with different antibiotics using
EKO-35 and the efflux platform. The FICI represents
the ΣFIC of each drug. The FIC for each drug was
determined by dividing the MIC of each drug in combination
by the MIC of each drug alone. ΣFIC = FIC_{A} + FIC_{B} =
(C_{A}/MIC_{A}) + (C_{B}/MIC_{B}). MIC_{A} and MIC_{B} are the MICs of drugs
A (PAβN) and B (antibiotic) alone, respectively. CA and CB
are the MICs of the drugs in combination. Synergy (FICI ≤0.5),
indifferent (FICI >1.0;), and additive (FICI >0.5-1.0).
Related to FIGS. 5A-5J.

| | | PABN | PABN | | |
|---|---|---|---|---|---|
| | | MICₐ | MICₐ | | |
| | | Alone | Combined | | |
| Strain | Compound | (μg/mL) | (μg/mL) | FICI | Effect |
| K-12 | Ciprofloxacin | 256 | 64 | 1.250 | — |
| ΔtolC | Ciprofloxacin | 64 | 32 | 1.000 | — |
| EKO-35 | Ciprofloxacin | 64 | 4 | 0.563 | Additive |
| EKO-35 | Ciprofloxacin | 64 | 8 | 0.625 | Additive |
| acrB_{D408A} | | | | | |
| EKO-35 | Ciprofloxacin | 32 | 16 | 1.000 | — |
| acrEF | | | | | |
| EKO-35 | Ciprofloxacin | 64 | 8 | 0.375 | Synergistic |
| mexCD | | | | | |
| K-12 | Ciprofloxacin | 256 | 256 | 2.000 | — |
| Pore | | | | | |
| ΔtolC | Ciprofloxacin | 128 | 64 | 0.551 | Additive |
| Pore | | | | | |
| EKO-35 | Ciprofloxacin | 64 | 2 | 0.531 | Additive |
| Pore | | | | | |
| EKO-35 | Ciprofloxacin | 32 | 8 | 0.750 | Additive |
| acrB_{D408A} | | | | | |
| Pore | | | | | |
| EKO-35 | Ciprofloxacin | 64 | 16 | 0.750 | Additive |
| acrEF | | | | | |
| Pore | | | | | |
| EKO-35 | Ciprofloxacin | 64 | 16 | 0.506 | Additive |
| mexCD | | | | | |
| Pore | | | | | |
| K-12 | Erythromycin | 256 | 16 | 0.094 | Synergistic |
| ΔtolC | Erythromycin | 64 | 8 | 0.188 | Synergistic |
| EKO-35 | Erythromycin | 128 | 8 | 0.093 | Synergistic |
| EKO-35 | Erythromycin | 64 | 8 | 0.190 | Synergistic |
| acrB_{D408A} | | | | | |
| EKO-35 | Erythromycin | 256 | 16 | 0.078 | Synergistic |
| acrB | | | | | |
| EKO-35 | Erythromycin | 128 | 16 | 0.188 | Synergistic |
| acrEF | | | | | |
| EKO-35 | Erythromycin | 256 | 16 | 0.094 | Synergistic |
| mdtEF | | | | | |
| EKO-35 | Erythromycin | 64 | 8 | 0.188 | Synergistic |
| mexCD | | | | | |
| K-12 | Erythromycin | 256 | 64 | 0.313 | Synergistic |
| Pore | | | | | |
| ΔtolC | Erythromycin | 128 | 16 | 0.188 | Synergistic |
| Pore | | | | | |
| EKO-35 | Erythromycin | 64 | 8 | 0.610 | Additive |
| Pore | | | | | |
| EKO-35 | Erythromycin | 64 | 16 | 0.735 | Additive |
| acrB_{D408A} | | | | | |
| Pore | | | | | |
| EKO-35 | Erythromycin | 256 | 16 | 0.078 | Synergistic |
| acrB Pore | | | | | |
| EKO-35 | Erythromycin | 128 | 16 | 0.385 | Synergistic |
| acrEF | | | | | |
| Pore | | | | | |
| EKO-35 | Erythromycin | 256 | 32 | 0.185 | Synergistic |
| mdtEF | | | | | |
| Pore | | | | | |
| EKO-35 | Erythromycin | 64 | 8 | 0.385 | Synergistic |
| mexCD | | | | | |
| Pore | | | | | |
| K-12 | Fusidic Acid | 256 | 32 | 0.156 | Synergistic |
| ΔtolC | Fusidic Acid | 64 | 16 | 0.375 | Synergistic |
| EKO-35 | Fusidic Acid | 128 | 16 | 0.158 | Synergistic |
| EKO-35 | Fusidic Acid | 64 | 8 | 0.250 | Synergistic |
| acrB_{D408A} | | | | | |
| EKO-35 | Fusidic Acid | 256 | 32 | 0.129 | Synergistic |
| acrB | | | | | |
| EKO-35 | Fusidic Acid | 64 | 8 | 0.141 | Synergistic |
| acrD | | | | | |
| EKO-35 | Fusidic Acid | 64 | 8 | 0.375 | Synergistic |
| acrEF | | | | | |
| EKO-35 | Fusidic Acid | 64 | 8 | 0.250 | Synergistic |
| mdtEF | | | | | |
| K-12 | Fusidic Acid | 256 | 32 | 0.188 | Synergistic |
| Pore | | | | | |
| ΔtolC | Fusidic Acid | 128 | 32 | 0.375 | Synergistic |
| Pore | | | | | |
| EKO-35 | Fusidic Acid | 64 | 16 | 0.310 | Synergistic |
| Pore | | | | | |
| EKO-35 | Fusidic Acid | 64 | 8 | 0.255 | Synergistic |
| acrB_{D408A} | | | | | |
| Pore | | | | | |
| EKO-35 | Fusidic Acid | 256 | 32 | 0.129 | Synergistic |
| acrB Pore | | | | | |
| EKO-35 | Fusidic Acid | 64 | 8 | 0.250 | Synergistic |
| acrD | | | | | |
| Pore | | | | | |
| EKO-35 | Fusidic Acid | 128 | 8 | 0.313 | Synergistic |
| acrEF | | | | | |
| Pore | | | | | |
| EKO-35 | Fusidic Acid | 64 | 8 | 0.250 | Synergistic |
| mdtEF Pore | | | | | |
| K-12 | Linezolid | 256 | 16 | 0.125 | Synergistic |
| ΔtolC | Linezolid | 128 | 64 | 0.750 | Additive |
| EKO-35 | Linezolid | 64 | 32 | 0.750 | Additive |
| EKO-35 | Linezolid | 64 | 32 | 0.750 | Additive |
| acrB_{D408A} | | | | | |
| EKO35 | Linezolid | 256 | 16 | 0.125 | Synergistic |
| araC::acrB | | | | | |
| K-12 | Linezolid | 256 | 32 | 0.250 | Synergistic |
| Pore | | | | | |
| ΔtolC | Linezolid | 128 | 64 | 0.563 | Additive |
| Pore | | | | | |
| EKO-35 | Linezolid | 64 | 32 | 1.000 | — |
| Pore | | | | | |
| EKO-35 | Linezolid | 64 | 32 | 1.000 | — |
| acrB_{D408A} | | | | | |
| Pore | | | | | |
| EKO-35 | Linezolid | 256 | 16 | 0.125 | Synergistic |
| acrB Pore | | | | | |
| K-12 | Novobiocin | 256 | 32 | 0.188 | Synergistic |
| ΔtolC | Novobiocin | 64 | 16 | 0.500 | Synergistic |
| EKO-35 | Novobiocin | 64 | 8 | 0.375 | Synergistic |
| EKO-35 | Novobiocin | 64 | 8 | 0.375 | Synergistic |
| acrB_{D408A} | | | | | |
| EKO-35 | Novobiocin | 256 | 32 | 0.156 | Synergistic |
| acrB | | | | | |
| EKO-35 | Novobiocin | 64 | 16 | 0.313 | Synergistic |
| acrD | | | | | |
| K-12 | Novobiocin | 256 | 2 | 0.508 | Additive |
| Pore | | | | | |
| ΔtolC | Novobiocin | 64 | 32 | 0.740 | Additive |
| Pore | | | | | |
| EKO-35 | Novobiocin | 32 | 16 | 0.630 | Additive |
| Pore | | | | | |
| EKO-35 | Novobiocin | 16 | 8 | 0.750 | Additive |
| acrB_{D408A} | | | | | |
| Pore | | | | | |
| EKO-35 | Novobiocin | 256 | 16 | 0.094 | Synergistic |
| acrB Pore | | | | | |
| EKO-35 | Novobiocin | 32 | 16 | 0.563 | Additive |
| acrD Pore | | | | | |
| K-12 | Oxacillin | 256 | 32 | 0.188 | Synergistic |
| ΔtolC | Oxacillin | 128 | 8 | 0.313 | Synergistic |
| EKO-35 | Oxacillin | 64 | 4 | 0.323 | Synergistic |
| EKO-35 | Oxacillin | 128 | 16 | 0.185 | Synergistic |
| acrB_{D408A} | | | | | |
| EKO-35 | Oxacillin | 256 | 16 | 0.125 | Synergistic |
| acrB | | | | | |
| EKO-35 | Oxacillin | 128 | 4 | 0.156 | Synergistic |
| acrD | | | | | |
| EKO-35 | Oxacillin | 128 | 16 | 0.133 | Synergistic |
| acrEF | | | | | |
| K-12 | Oxacillin | 256 | 64 | 0.750 | Additive |
| Pore | | | | | |
| ΔtolC | Oxacillin | 128 | 64 | 0.740 | Additive |
| Pore | | | | | |
| EKO-35 | Oxacillin | 64 | 32 | 1.020 | — |
| Pore | | | | | |
| EKO-35 | Oxacillin | 64 | 32 | 1.020 | — |
| acrB_{D408A} | | | | | |
| Pore | | | | | |
| EKO-35 | Oxacillin | 256 | 16 | 0.188 | Synergistic |
| acrB Pore | | | | | |
| EKO-35 | Oxacillin | 64 | 16 | 0.500 | Synergistic |
| acrD Pore | | | | | |
| EKO-35 | Oxacillin | 128 | 32 | 0.500 | Synergistic |
| acrEF Pore | | | | | |

TABLE 18B
The FICI represents the ΣFIC of each drug. The FIC for each drug
was determined by dividing the MIC of each drug in combination by the MIC of each
drug alone. ΣFIC = FIC_{A} + FIC_{B} = (C_{A}/MIC_{A}) + (C_{B}/MIC_{B}).
MIC_{A} and MIC_{B} are the MICs of drugs A (PAβN) and B (antibiotic) alone,
respectively. CA and CB are the MICs of the drugs in combination. Synergy
(FICI ≤0.5), indifferent (FICI >1.0), and additive (FICI >0.5-
1.0). Related to FIG. 5A-5J.

| | | MIC_{b} | MIC_{b} | | |
|---|---|---|---|---|---|
| | | Alone | Combined | | |
| Strain | Compound | (μg/mL) | (μg/mL) | FICI | Effect |
| K-12 | Ciprofloxacin | 0.0156 | 0.0156 | 1.250 | — |
| ΔtolC | Ciprofloxacin | 0.0078 | 0.0039 | 1.000 | — |
| EKO-35 | Ciprofloxacin | 0.0078 | 0.0039 | 0.563 | Additive |
| EKO-35 | Ciprofloxacin | 0.0078 | 0.0039 | 0.625 | Additive |
| acrB_{D408A} | | | | | |
| EKO-35 acrEF | Ciprofloxacin | 0.0156 | 0.0078 | 1.000 | — |
| EKO-35 | Ciprofloxacin | 0.0156 | 0.0039 | 0.375 | Synergistic |
| mexCD | | | | | |
| K-12 Pore | Ciprofloxacin | 0.0313 | 0.0313 | 2.000 | |
| ΔtolC Pore | Ciprofloxacin | 0.0039 | 0.0002 | 0.551 | Additive |
| EKO-35 Pore | Ciprofloxacin | 0.0078 | 0.0039 | 0.531 | Additive |
| EKO-35 | Ciprofloxacin | 0.0078 | 0.0039 | 0.750 | Additive |
| acrB_{D408A} Pore | | | | | |
| EKO-35 acrEF | Ciprofloxacin | 0.0078 | 0.0039 | 0.750 | Additive |
| Pore | | | | | |
| EKO-35 | Ciprofloxacin | 0.0078 | 0.002 | 0.506 | Additive |
| mexCD Pore | | | | | |
| K-12 | Erythromycin | 64 | 2 | 0.094 | Synergistic |
| ΔtolC | Erythromycin | 4 | 0.25 | 0.188 | Synergistic |
| EKO-35 | Erythromycin | 2 | 0.06 | 0.093 | Synergistic |
| EKO-35 | Erythromycin | 2 | 0.13 | 0.190 | Synergistic |
| acrB_{D408A} | | | | | |
| EKO-35 acrB | Erythromycin | 64 | 1 | 0.078 | Synergistic |
| EKO-35 acrEF | Erythromycin | 8 | 0.5 | 0.188 | Synergistic |
| EKO-35 | Erythromycin | 16 | 0.5 | 0.094 | Synergistic |
| mdtEF | | | | | |
| EKO-35 | Erythromycin | 8 | 0.5 | 0.188 | Synergistic |
| mexCD | | | | | |
| K-12 Pore | Erythromycin | 4 | 0.25 | 0.313 | Synergistic |
| δtolC Pore | Erythromycin | 1 | 0.063 | 0.188 | Synergistic |
| EKO-35 Pore | Erythromycin | 0.13 | 0.063 | 0.610 | Additive |
| EKO-35 | Erythromycin | 0.13 | 0.063 | 0.735 | Additive |
| acrB_{D408A} Pore | | | | | |
| EKO-35 acrB | Erythromycin | 32 | 0.5 | 0.078 | Synergistic |
| Pore | | | | | |
| EKO-35 acrEF | Erythromycin | 0.5 | 0.13 | 0.385 | Synergistic |
| Pore | | | | | |
| EKO-35 | Erythromycin | 1 | 0.06 | 0.185 | Synergistic |
| mdtEF Pore | | | | | |
| EKO-35 | Erythromycin | 0.5 | 0.13 | 0.385 | Synergistic |
| mexCD Pore | | | | | |
| K-12 | Fusidic Acid | 1024 | 32 | 0.156 | Synergistic |
| ΔtolC | Fusidic Acid | 2 | 0.25 | 0.375 | Synergistic |
| EKO-35 | Fusidic Acid | 4 | 0.13 | 0.158 | Synergistic |
| EKO-35 | Fusidic Acid | 4 | 0.5 | 0.250 | Synergistic |
| acrB_{D408A} | | | | | |
| EKO-35 acrB | Fusidic Acid | 1024 | 4 | 0.129 | Synergistic |
| EKO-35 acrD | Fusidic Acid | 256 | 4 | 0.141 | Synergistic |
| EKO-35 acrEF | Fusidic Acid | 32 | 8 | 0.375 | Synergistic |
| EKO-35 | Fusidic Acid | 64 | 8 | 0.250 | Synergistic |
| mdtEF | | | | | |
| K-12 Pore | Fusidic Acid | 512 | 32 | 0.188 | Synergistic |
| δtolC Pore | Fusidic Acid | 2 | 0.25 | 0.375 | Synergistic |
| EKO-35 Pore | Fusidic Acid | 1 | 0.06 | 0.310 | Synergistic |
| EKO-35 | Fusidic Acid | 1 | 0.13 | 0.255 | Synergistic |
| acrB_{D408A} Pore | | | | | |
| EKO-35 acrB | Fusidic Acid | 512 | 2 | 0.129 | Synergistic |
| Pore | | | | | |
| EKO-35 acrD | Fusidic Acid | 64 | 8 | 0.250 | Synergistic |
| Pore | | | | | |
| EKO-35 acrEF | Fusidic Acid | 4 | 1 | 0.313 | Synergistic |
| Pore | | | | | |
| EKO-35 | Fusidic Acid | 16 | 2 | 0.250 | Synergistic |
| mdtEF Pore | | | | | |
| K-12 | Linezolid | 256 | 16 | 0.125 | Synergistic |
| ΔtolC | Linezolid | 8 | 2 | 0.750 | Additive |
| EKO-35 | Linezolid | 16 | 4 | 0.750 | Additive |
| EKO-35 | Linezolid | 16 | 4 | 0.750 | Additive |
| acrB_{D408A} | | | | | |
| EKO35 | Linezolid | 256 | 16 | 0.125 | Synergistic |
| araC::acrB | | | | | |
| K-12 Pore | Linezolid | 128 | 16 | 0.250 | Synergistic |
| ΔtolC Pore | Linezolid | 8 | 0.5 | 0.563 | Additive |
| EKO-35 Pore | Linezolid | 8 | 4 | 1.000 | — |
| EKO-35 | Linezolid | 8 | 4 | 1.000 | — |
| acrB_{D408A} Pore | | | | | |
| EKO-35 acrB | Linezolid | 128 | 8 | 0.125 | Synergistic |
| Pore | | | | | |
| K-12 | Novobiocin | 128 | 8 | 0.188 | Synergistic |
| ΔtolC | Novobiocin | 2 | 0.5 | 0.500 | Synergistic |
| EKO-35 | Novobiocin | 4 | 1 | 0.375 | Synergistic |
| EKO-35 | Novobiocin | 8 | 2 | 0.375 | Synergistic |
| acrB_{D408A} | | | | | |
| EKO-35 acrB | Novobiocin | 1024 | 32 | 0.156 | Synergistic |
| EKO-35 acrD | Novobiocin | 512 | 32 | 0.313 | Synergistic |
| K-12 Pore | Novobiocin | 16 | 8 | 0.508 | Additive |
| ΔtolC Pore | Novobiocin | 0.25 | 0.06 | 0.740 | Additive |
| EKO-35 Pore | Novobiocin | 1 | 0.13 | 0.630 | Additive |
| EKO-35 | Novobiocin | 1 | 0.25 | 0.750 | Additive |
| acrB_{D408A} Pore | | | | | |
| EKO-35 acrB | Novobiocin | 512 | 16 | 0.094 | Synergistic |
| Pore | | | | | |
| EKO-35 acrD | Novobiocin | 16 | 1 | 0.563 | Additive |
| Pore | | | | | |
| K-12 | Oxacillin | 512 | 32 | 0.188 | Synergistic |
| ΔtolC | Oxacillin | 1 | 0.25 | 0.313 | Synergistic |
| EKO-35 | Oxacillin | 0.5 | 0.13 | 0.323 | Synergistic |
| EKO-35 | Oxacillin | 1 | 0.06 | 0.185 | Synergistic |
| acrB_{D408A} | | | | | |
| EKO-35 acrB | Oxacillin | 1024 | 64 | 0.125 | Synergistic |
| EKO-35 acrD | Oxacillin | 128 | 16 | 0.156 | Synergistic |
| EKO-35 acrEF | Oxacillin | 256 | 2 | 0.133 | Synergistic |
| K-12 Pore | Oxacillin | 32 | 16 | 0.750 | Additive |
| ΔtolC Pore | Oxacillin | 0.25 | 0.06 | 0.740 | Additive |
| EKO-35 Pore | Oxacillin | 0.25 | 0.13 | 1.020 | — |
| EKO-35 | Oxacillin | 0.25 | 0.13 | 1.020 | — |
| acrB_{D408A} Pore | | | | | |
| EKO-35 acrB | Oxacillin | 128 | 16 | 0.188 | Synergistic |
| Pore | | | | | |
| EKO-35 acrD | Oxacillin | 4 | 1 | 0.500 | Synergistic |
| Pore | | | | | |
| EKO-35 acrEF | Oxacillin | 4 | 1 | 0.500 | Synergistic |
| Pore | | | | | |

TABLE 19A
Defining the Fractional Inhibitory Concentration Index (FICI) for NMP in combination
with different antibiotics using EKO-35 and the efflux platform. The FICI
represents the ΣFIC of each drug. The FIC for each drug was determined by
dividing the MIC of each drug in combination by the MIC of each drug alone. ΣFIC =
FIC_{A} + FIC_{B} = (C_{A}/MIC_{A}) + (C_{B}/MIC_{B}). MIC_{A} and MIC_{B} are the MICs of
drugs A (NMP) and B (antibiotic) alone, respectively. C_{A} and C_{B} are the MICs
of the drugs in combination. Synergy (FICI ≤0.5), indifferent (FICI >1.0),
and additive (FICI >0.5-1.0). Related to FIGS. 5A-5J.

| | | NMP | NMP | | |
|---|---|---|---|---|---|
| | | MICₐ | MICₐ | | |
| | | Alone | Combined | | |
| Strain | Compound | (μg/mL) | (μg/mL) | FICI | Effect |
| K-12 | Ciprofloxacin | 512 | 128 | 0.748 | Additive |
| ΔtolC | Ciprofloxacin | 512 | 512 | 2.000 | — |
| EKO-35 | Ciprofloxacin | 512 | 512 | 2.000 | — |
| EKO-35 | Ciprofloxacin | 512 | 64 | 0.625 | Additive |
| acrB_{D408A} | | | | | |
| EKO-35 acrEF | Ciprofloxacin | 256 | 64 | 0.748 | Additive |
| EKO-35 | Ciprofloxacin | 512 | 128 | 0.750 | Additive |
| mexCD | | | | | |
| K-12 Pore | Ciprofloxacin | 512 | 512 | 2.000 | — |
| δtolC Pore | Ciprofloxacin | 512 | 256 | 0.750 | Additive |
| EKO-35 Pore | Ciprofloxacin | 512 | 512 | 2.000 | — |
| EKO-35 | Ciprofloxacin | 512 | 4 | 0.508 | Additive |
| acrB_{D408A} Pore | | | | | |
| EKO-35 acrEF | Ciprofloxacin | 512 | 256 | 0.526 | Additive |
| Pore | | | | | |
| EKO-35 | Ciprofloxacin | 512 | 256 | 1.000 | — |
| mexCD Pore | | | | | |
| K-12 | Erythromycin | 256 | 128 | 0.625 | Additive |
| ΔtolC | Erythromycin | 256 | 128 | 1.000 | — |
| EKO-35 | Erythromycin | 1024 | 256 | 0.375 | Synergistic |
| EKO-35 | Erythromycin | 1024 | 256 | 0.500 | Synergistic |
| acrB_{D408A} | | | | | |
| EKO-35 acrB | Erythromycin | 256 | 8 | 0.531 | Additive |
| EKO-35 acrEF | Erythromycin | 256 | 256 | 2.000 | — |
| EKO-35 mdtEF | Erythromycin | 512 | 64 | 0.250 | Synergistic |
| EKO-35 | Erythromycin | 64 | 16 | 0.375 | Synergistic |
| mexCD | | | | | |
| K-12 Pore | Erythromycin | 512 | 256 | 1.000 | — |
| ΔtolC Pore | Erythromycin | 512 | 256 | 0.625 | Synergistic |
| EKO-35 Pore | Erythromycin | 512 | 128 | 0.770 | Additive |
| EKO-35 | Erythromycin | 512 | 128 | 0.735 | Additive |
| acrB_{D408A} Pore | | | | | |
| EKO-35 acrB | Erythromycin | 512 | 256 | 0.563 | Additive |
| Pore | | | | | |
| EKO-35 acrEF | Erythromycin | 512 | 128 | 0.750 | Additive |
| Pore | | | | | |
| EKO-35 mdtEF | Erythromycin | 256 | 128 | 0.625 | Additive |
| Pore | | | | | |
| EKO-35 | Erythromycin | 128 | 64 | 0.565 | Additive |
| mexCD Pore | | | | | |
| K-12 | Ethidium | 512 | 64 | 0.375 | Synergistic |
| | Bromide | | | | |
| ΔtolC | Ethidium | 1024 | 32 | 0.281 | Synergistic |
| | Bromide | | | | |
| EKO-35 | Ethidium | 512 | 512 | 2.000 | — |
| | Bromide | | | | |
| EKO-35 | Ethidium | 1024 | 256 | 0.750 | Additive |
| acrB_{D408A} | Bromide | | | | |
| EKO-35 acrB | Ethidium | 1024 | 128 | 0.253 | Synergistic |
| | Bromide | | | | |
| EKO-35 acrEF | Ethidium | 1024 | 256 | 0.500 | Synergistic |
| | Bromide | | | | |
| K-12 Pore | Ethidium | 512 | 128 | 0.375 | Synergistic |
| | Bromide | | | | |
| ΔtolC Pore | Ethidium | 512 | 128 | 0.500 | Additive |
| | Bromide | | | | |
| EKO-35 Pore | Ethidium | 512 | 4 | 0.508 | Additive |
| | Bromide | | | | |
| EKO-35 | Ethidium | 512 | 256 | 1.000 | — |
| acrB_{D408A} Pore | Bromide | | | | |
| EKO-35 acrB | Ethidium | 512 | 32 | 0.191 | Synergistic |
| Pore | Bromide | | | | |
| EKO-35 acrEF | Ethidium | 512 | 128 | 0.500 | Synergistic |
| Pore | Bromide | | | | |
| K-12 | Fusidic Acid | 1024 | 256 | 0.375 | Synergistic |
| ΔtolC | Fusidic Acid | 512 | 256 | 0.625 | Additive |
| EKO-35 | Fusidic Acid | 512 | 256 | 0.625 | Additive |
| EKO-35 | Fusidic Acid | 1024 | 256 | 0.500 | Synergistic |
| acrB_{D408A} | | | | | |
| EKO-35 acrB | Fusidic Acid | 512 | 128 | 0.500 | Synergistic |
| EKO-35 acrD | Fusidic Acid | 512 | 256 | 0.563 | Additive |
| EKO-35 acrEF | Fusidic Acid | 512 | 128 | 0.750 | Additive |
| EKO-35 mdtEF | Fusidic Acid | 512 | 128 | 0.375 | Synergistic |
| K-12 Pore | Fusidic Acid | 512 | 128 | 0.500 | Synergistic |
| δtolC Pore | Fusidic Acid | 512 | 128 | 0.750 | Additive |
| EKO-35 Pore | Fusidic Acid | 512 | 256 | 0.620 | Additive |
| EKO-35 | Fusidic Acid | 512 | 128 | 0.750 | Additive |
| acrB_{D408A} Pore | | | | | |
| EKO-35 acrB | Fusidic Acid | 512 | 128 | 0.500 | Synergistic |
| Pore | | | | | |
| EKO-35 acrD | Fusidic Acid | 256 | 128 | 1.000 | — |
| Pore | | | | | |
| EKO-35 acrEF | Fusidic Acid | 512 | 64 | 0.625 | Additive |
| Pore | | | | | |
| EKO-35 mdtEF | Fusidic Acid | 256 | 128 | 0.750 | Additive |
| Pore | | | | | |
| K-12 | Linezolid | 512 | 128 | 0.375 | Synergistic |
| ΔtolC | Linezolid | 512 | 512 | 2.000 | — |
| EKO-35 | Linezolid | 512 | 16 | 0.531 | Additive |
| EKO-35 | Linezolid | 512 | 8 | 0.516 | Additive |
| acrB_{D408A} | | | | | |
| EKO-35 acrB | Linezolid | 256 | 64 | 0.281 | Synergistic |
| K-12 Pore | Linezolid | 512 | 64 | 0.375 | Synergistic |
| ΔtolC Pore | Linezolid | 512 | 256 | 1.000 | — |
| EKO-35 Pore | Linezolid | 512 | 512 | 2.000 | — |
| EKO-35 | Linezolid | 512 | 512 | 2.000 | — |
| acrB_{D408A} Pore | | | | | |
| EKO-35 acrB | Linezolid | 512 | 64 | 0.250 | Synergistic |
| Pore | | | | | |
| K-12 | Oxacillin | 512 | 64 | 0.375 | Synergistic |
| ΔtolC | Oxacillin | 256 | 8 | 0.531 | Additive |
| EKO-35 | Oxacillin | 1024 | 256 | 0.510 | Additive |
| EKO-35 | Oxacillin | 1024 | 1024 | 2.000 | — |
| acrB_{D408A} | | | | | |
| EKO-35 acrB | Oxacillin | 512 | 256 | 0.531 | Additive |
| EKO-35 acrD | Oxacillin | 512 | 256 | 0.750 | Additive |
| EKO-35 acrEF | Oxacillin | 512 | 64 | 0.375 | Synergistic |
| K-12 Pore | Oxacillin | 512 | 8 | 0.266 | Synergistic |
| δtolC Pore | Oxacillin | 512 | 16 | 0.531 | Additive |
| EKO-35 Pore | Oxacillin | 512 | 128 | 0.750 | Additive |
| EKO-35 | Oxacillin | 512 | 512 | 2.000 | — |
| acrB_{D408A} Pore | | | | | |
| EKO-35 acrB | Oxacillin | 512 | 128 | 0.500 | Synergistic |
| Pore | | | | | |
| EKO-35 acrD | Oxacillin | 512 | 256 | 0.750 | Additive |
| Pore | | | | | |
| EKO-35 acrEF | Oxacillin | 512 | 256 | 1.000 | — |
| Pore | | | | | |

TABLE 19B
Defining the Fractional Inhibitory Concentration Index (FICI) for NMP in combination
with different antibiotics using EKO-35 and the efflux platform. The FICI
represents the ΣFIC of each drug. The FIC for each drug was determined by
dividing the MIC of each drug in combination by the MIC of each drug alone. ΣFIC =
FIC_{A} + FIC_{B} = (C_{A}/MIC_{A}) + (C_{B}/MIC_{B}). MIC_{A} and MIC_{B} are the MICs of
drugs A (NMP) and B (antibiotic) alone, respectively. C_{A} and C_{B} are the MICs
of the drugs in combination. Synergy (FICI ≤0.5), indifferent (FICI >1.0),
and additive (FICI >0.5-1.0). Related to FIGS. 5A-5J.

| | | MIC_{b} | MIC_{b} | | |
|---|---|---|---|---|---|
| | | Alone | Combined | | |
| Strain | Compound | (μg/mL) | (μg/mL) | FICI | Effect |
| K-12 | Ciprofloxacin | 0.0313 | 0.0156 | 0.748 | Additive |
| ΔtolC | Ciprofloxacin | 0.0078 | 0.0078 | 2.000 | — |
| EKO-35 | Ciprofloxacin | 0.0078 | 0.0078 | 2.000 | — |
| EKO-35 | Ciprofloxacin | 0.0156 | 0.0078 | 0.625 | Additive |
| acrB_{D408A} | | | | | |
| EKO-35 acrEF | Ciprofloxacin | 0.0313 | 0.0156 | 0.748 | Additive |
| EKO-35 | Ciprofloxacin | 0.0156 | 0.0078 | 0.750 | Additive |
| mexCD | | | | | |
| K-12 Pore | Ciprofloxacin | 0.0156 | 0.0156 | 2.000 | — |
| ΔtolC Pore | Ciprofloxacin | 0.0156 | 0.0039 | 0.750 | Additive |
| EKO-35 Pore | Ciprofloxacin | 0.0078 | 0.0078 | 2.000 | — |
| EKO-35 | Ciprofloxacin | 0.0156 | 0.0078 | 0.508 | Additive |
| acrB_{D408A} Pore | | | | | |
| EKO-35 acrEF | Ciprofloxacin | 0.0078 | 0.0002 | 0.526 | Additive |
| Pore | | | | | |
| EKO-35 | Ciprofloxacin | 0.0078 | 0.0039 | 1.000 | — |
| mexCD Pore | | | | | |
| K-12 | Erythromycin | 128 | 16 | 0.625 | Additive |
| ΔtolC | Erythromycin | 4 | 2 | 1.000 | — |
| EKO-35 | Erythromycin | 2 | 0.25 | 0.375 | Synergistic |
| EKO-35 | Erythromycin | 2 | 0.5 | 0.500 | Synergistic |
| acrB_{D408A} | | | | | |
| EKO-35 acrB | Erythromycin | 128 | 64 | 0.531 | Additive |
| EKO-35 acrEF | Erythromycin | 8 | 8 | 2.000 | — |
| EKO-35 mdtEF | Erythromycin | 64 | 8 | 0.250 | Synergistic |
| EKO-35 | Erythromycin | 16 | 2 | 0.375 | Synergistic |
| mexCD | | | | | |
| K-12 Pore | Erythromycin | 16 | 8 | 1.000 | — |
| ΔtolC Pore | Erythromycin | 4 | 0.5 | 0.625 | Synergistic |
| EKO-35 Pore | Erythromycin | 0.25 | 0.13 | 0.770 | Additive |
| EKO-35 | Erythromycin | 0.13 | 0.063 | 0.735 | Additive |
| acrB_{D408A} Pore | | | | | |
| EKO-35 acrB | Erythromycin | 8 | 0.5 | 0.563 | Additive |
| Pore | | | | | |
| EKO-35 acrEF | Erythromycin | 1 | 0.5 | 0.750 | Additive |
| Pore | | | | | |
| EKO-35 mdtEF | Erythromycin | 8 | 1 | 0.625 | Additive |
| Pore | | | | | |
| EKO-35 | Erythromycin | 2 | 0.13 | 0.565 | Additive |
| mexCD Pore | | | | | |
| K-12 | Ethidium Bromide | 500 | 125 | 0.375 | Synergistic |
| ΔtolC | Ethidium Bromide | 7.81 | 1.95 | 0.281 | Synergistic |
| EKO-35 | Ethidium Bromide | 0.244 | 0.244 | 2.000 | — |
| EKO-35 | Ethidium Bromide | 0.488 | 0.244 | 0.750 | Additive |
| acrB_{D408A} | | | | | |
| EKO-35 acrB | Ethidium Bromide | 15.6 | 2 | 0.253 | Synergistic |
| EKO-35 acrEF | Ethidium Bromide | 2 | 0.5 | 0.500 | Synergistic |
| K-12 Pore | Ethidium Bromide | 250 | 31.3 | 0.375 | Synergistic |
| ΔtolC Pore | Ethidium Bromide | 1.95 | 0.488 | 0.500 | Additive |
| EKO-35 Pore | Ethidium Bromide | 0.488 | 0.244 | 0.508 | Additive |
| EKO-35 | Ethidium Bromide | 0.244 | 0.122 | 1.000 | — |
| acrB_{D408A} Pore | | | | | |
| EKO-35 acrB | Ethidium Bromide | 15.6 | 2 | 0.191 | Synergistic |
| Pore | | | | | |
| EKO-35 acrEF | Ethidium Bromide | 2 | 0.5 | 0.500 | Synergistic |
| Pore | | | | | |
| K-12 | Fusidic Acid | 1024 | 128 | 0.375 | Synergistic |
| ΔtolC | Fusidic Acid | 4 | 0.5 | 0.625 | Additive |
| EKO-35 | Fusidic Acid | 4 | 0.5 | 0.625 | Additive |
| EKO-35 | Fusidic Acid | 4 | 1 | 0.500 | Synergistic |
| acrB_{D408A} | | | | | |
| EKO-35 acrB | Fusidic Acid | 1024 | 256 | 0.500 | Synergistic |
| EKO-35 acrD | Fusidic Acid | 256 | 16 | 0.563 | Additive |
| EKO-35 acrEF | Fusidic Acid | 32 | 16 | 0.750 | Additive |
| EKO-35 mdtEF | Fusidic Acid | 64 | 8 | 0.375 | Synergistic |
| K-12 Pore | Fusidic Acid | 512 | 128 | 0.500 | Synergistic |
| ΔtolC Pore | Fusidic Acid | 2 | 1 | 0.750 | Additive |
| EKO-35 Pore | Fusidic Acid | 0.5 | 0.06 | 0.620 | Additive |
| EKO-35 | Fusidic Acid | 0.5 | 0.25 | 0.750 | Additive |
| acrB_{D408A} Pore | | | | | |
| EKO-35 acrB | Fusidic Acid | 512 | 128 | 0.500 | Synergistic |
| Pore | | | | | |
| EKO-35 acrD | Fusidic Acid | 64 | 32 | 1.000 | — |
| Pore | | | | | |
| EKO-35 acrEF | Fusidic Acid | 2 | 1 | 0.625 | Additive |
| Pore | | | | | |
| EKO-35 mdtEF | Fusidic Acid | 8 | 2 | 0.750 | Additive |
| Pore | | | | | |
| K-12 | Linezolid | 256 | 32 | 0.375 | Synergistic |
| ΔtolC | Linezolid | 8 | 8 | 2.000 | — |
| EKO-35 | Linezolid | 16 | 8 | 0.531 | Additive |
| EKO-35 | Linezolid | 16 | 8 | 0.516 | Additive |
| acrB_{D408A} | | | | | |
| EKO-35 acrB | Linezolid | 256 | 8 | 0.281 | Synergistic |
| K-12 Pore | Linezolid | 128 | 32 | 0.375 | Synergistic |
| δtolC Pore | Linezolid | 8 | 4 | 1.000 | — |
| EKO-35 Pore | Linezolid | 8 | 8 | 2.000 | — |
| EKO-35 | Linezolid | 8 | 8 | 2.000 | — |
| acrB_{D408A} Pore | | | | | |
| EKO-35 acrB | Linezolid | 128 | 16 | 0.250 | Synergistic |
| Pore | | | | | |
| K-12 | Oxacillin | 512 | 128 | 0.375 | Synergistic |
| ΔtolC | Oxacillin | 1 | 0.5 | 0.531 | Additive |
| EKO-35 | Oxacillin | 0.5 | 0.13 | 0.510 | Additive |
| EKO-35 | Oxacillin | 0.13 | 0.13 | 2.000 | — |
| acrB_{D408A} | | | | | |
| EKO-35 acrB | Oxacillin | 512 | 16 | 0.531 | Additive |
| EKO-35 acrD | Oxacillin | 128 | 32 | 0.750 | Additive |
| EKO-35 acrEF | Oxacillin | 256 | 64 | 0.375 | Synergistic |
| K-12 Pore | Oxacillin | 64 | 16 | 0.266 | Synergistic |
| ΔtolC Pore | Oxacillin | 1 | 0.5 | 0.531 | Additive |
| EKO-35 Pore | Oxacillin | 0.5 | 0.25 | 0.750 | Additive |
| EKO-35 | Oxacillin | 0.13 | 0.13 | 2.000 | — |
| acrB_{D408A} Pore | | | | | |
| EKO-35 acrB | Oxacillin | 64 | 16 | 0.500 | Synergistic |
| Pore | | | | | |
| EKO-35 acrD | Oxacillin | 2 | 0.5 | 0.750 | Additive |
| Pore | | | | | |
| EKO-35 acrEF | Oxacillin | 2 | 1 | 1.000 | — |
| Pore | | | | | |

TABLE 20
Assessing efflux pump interplay using EKO-35 and the efflux platform. MIC values
were calculated by averaging the OD_{600 nm} values of three biological replicates
and identifying the highest concentration of drug for which the OD_{600 nm} value
>0.100. The fold changes between EKO-35 and each efflux pump expressing
strain were calculated by dividing the MIC value of each strain by that of
EKO-35. Fold increases in resistance 4-fold and above are bolded.

| | | | | Fold | | |
|---|---|---|---|---|---|---|
| | | | | Change | Interplay | |
| Compound | Strain | Pore | MIC | from EKO-35 | effect | P-Value |
| Acriflavine | EKO-35 | − | 0.098 | − | − | 1.75 × 10⁻⁶ |
| | EKO-35 | | 0.781 | 8 | | |
| | pEmrE | | | | | |
| | EKO-35 | + | 0.049 | − | − | |
| | EKO-35 | | 0.781 | 16 | | 7.20 × 10⁻⁶⁴ |
| | pEmrE | | | | | |
| | acrB | − | 1.563 | 16 | Multiplicative | |
| | acrB | | 25 | 256 | | 1.75 × 10⁻⁶ |
| | pEmrE | | | | | |
| | acrB | + | 0.781 | 16 | Multiplicative | |
| | acrB | | 12.5 | 256 | | 2.01 × 10⁻² |
| | pEmrE | | | | | |
| | acrEF | − | 0.781 | 8 | Multiplicative | |
| | acrEF | | 12.5 | 128 | | 2.23 × 10⁻² |
| | pEmrE | | | | | |
| | acrEF | + | 0.781 | 16 | Multiplicative | |
| | acrEF | | 6.25 | 128 | | 9.79 × 10⁻⁶⁴ |
| | pEmrE | | | | | |
| | acrD | − | 0.098 | 1 | Multiplicative | |
| | acrD | | 1.563 | 16 | | 1.60 × 10⁻⁶ |
| | pEmrE | | | | | |
| | acrD | + | 0.098 | 2 | − | |
| | acrD | | 0.781 | 16 | | 9.79 × 10⁻⁶⁴ |
| | pEmrE | | | | | |
| | mdtEF | − | 0.098 | 1 | Multiplicative | |
| | mdtEF | | 1.563 | 16 | | 7.26 × 10⁻⁶⁴ |
| | pEmrE | | | | | |
| | mdtEF | + | 0.195 | 4 | Multiplicative | |
| | mdtEF | | 1.563 | 32 | | 1.60 × 10⁻⁶ |
| | pEmrE | | | | | |
| Ethidium | EKO-35 | − | 0.122 | − | − | |
| Bromide | EKO-35 | | 0.488 | 4 | | 4.52 × 10⁻⁵ |
| | pEmrE | | | | | |
| | EKO-35 | + | 0.061 | − | − | |
| | EKO-35 | | 0.244 | 4 | | 2.45 × 10⁻³¹ |
| | pEmrE | | | | | |
| | acrB | − | 7.813 | 64 | Multiplicative | |
| | acrB | | 31.25 | 256 | | 3.14 × 10⁻² |
| | pEmrE | | | | | |
| | acrB | + | 3.91 | 64 | Multiplicative | |
| | acrB | | 15.6 | 256 | | 4.52 × 10⁻⁵ |
| | pEmrE | | | | | |
| | acrEF | − | 1.953 | 16 | Multiplicative | |
| | acrEF | | 7.813 | 64 | | 4.52 × 10⁻⁵ |
| | pEmrE | | | | | |
| | acrEF | + | 0.977 | 16 | Multiplicative | |
| | acrEF | | 3.91 | 64 | | 1.32 × 10⁻³ |
| | pEmrE | | | | | |
| | acrD | − | 0.244 | 2 | Multiplicative | |
| | acrD | | 1.953 | 16 | | 2.86 × 10⁻² |
| | pEmrE | | | | | |
| | acrD | + | 0.061 | 1 | Multiplicative | |
| | acrD | | 0.488 | 8 | | 3.69 × 10⁻⁵ |
| | pEmrE | | | | | |
| | mdtEF | − | 0.977 | 8 | Multiplicative | |
| | mdtEF | | 3.906 | 32 | | 3.69 × 10⁻⁵ |
| | pEmrE | | | | | |
| | mdtEF | + | 0.488 | 8 | Multiplicative | |
| | mdtEF | | 1.95 | 32 | | 3.14 × 10⁻² |
| | pEmrE | | | | | |
| Novobiocin | EKO-35 | − | 0.781 | − | − | |
| | EKO-35 | | 0.781 | 1 | | 1.00 × 10⁰ |
| | pEmrE | | | | | |
| | EKO-35 | + | 0.391 | − | − | |
| | EKO-35 | | 0.391 | 1 | | 1.00 × 10⁰ |
| | pEmrE | | | | | |
| | acrB | − | 400 | 512 | − | |
| | acrB | | 400 | 512 | | 7.25 × 10⁻¹ |
| | pEmrE | | | | | |
| | acrB | + | 100 | 256 | | |
| | acrB | | 100 | 256 | | 1.32 × 10⁻¹ |
| | pEmrE | | | | | |
| | acrEF | − | 100 | 128 | − | |
| | acrEF | | 50 | 64 | | 1.16 × 10⁻¹ |
| | pEmrE | | | | | |
| | acrEF | + | 12.5 | 32 | − | |
| | acrEF | | 12.5 | 32 | | 1.00 × 10⁰ |
| | pEmrE | | | | | |
| | acrD | − | 100 | 128 | − | |
| | acrD | | 50 | 64 | | 5.50 × 10⁻² |
| | pEmrE | | | | | |
| | acrD | + | 25 | 64 | − | |
| | acrD | | 6.25 | 16 | | 5.50 × 10⁻² |
| | pEmrE | | | | | |
| | mdtEF | − | 12.5 | 16 | − | |
| | mdtEF | | 6.25 | 8 | | 3.74 × 10⁻¹ |
| | pEmrE | | | | | |
| | mdtEF | + | 3.13 | 8 | − | |
| | mdtEF | | 1.56 | 4 | | 1.48 × 10⁻¹ |
| | pEmrE | | | | | |
| Minocycline | EKO-35 | − | 1.25 | − | − | |
| | EKO-35 | | 1.25 | 1 | | 1.00 × 10⁰ |
| | pEmrE | | | | | |
| | EKO-35 | + | 0.63 | − | | |
| | EKO-35 | | 0.63 | 1 | | 3.72 × 10⁻¹ |
| | pEmrE | | | | | |
| | acrB | − | 5 | 4 | − | |
| | acrB | | 5 | 4 | | 1.00 × 10⁰ |
| | pEmrE | | | | | |
| | acrB | + | 2.5 | 4 | − | |
| | acrB | | 2.5 | 4 | | 3.74 × 10⁻¹ |
| | pEmrE | | | | | |
| | acrEF | − | 2.5 | 2 | − | |
| | acrEF | | 5.0 | 4 | | 3.74 × 10⁻¹ |
| | pEmrE | | | | | |
| | acrEF | + | 1.25 | 2 | − | |
| | acrEF | | 1.25 | 2 | | 1.00 × 10⁰ |
| | pEmrE | | | | | |
| | acrD | − | 2.5 | 2 | − | |
| | acrD | | 2.5 | 2 | | 1.00 × 10⁰ |
| | pEmrE | | | | | |
| | acrD | + | 1.25 | 2 | − | |
| | acrD | | 1.25 | 2 | | 3.98 × 10⁻¹ |
| | pEmrE | | | | | |
| | mdtEF | − | 2.5 | 2 | − | |
| | mdtEF | | 2.5 | 2 | | 1.00 × 10⁰ |
| | pEmrE | | | | | |
| | mdtEF | + | 1.25 | 2 | − | |
| | mdtEF | | 1.25 | 2 | | 1.28 × 10⁻¹ |
| | pEmrE | | | | | |

### Example 2. Development and Utilization of EKO-35v2

### Materials and Methods

### Strains, Plasmids, and Growth Conditions

Bacterial strains and plasmids used in this Example are provided in Table 21. E. coli K-12 str. BW25113, the parental strain of the Keio Collection (Baba, T. et al., 2006) was used as the background for generation of EKO-35v2. E. coli TOP10 or E. coli DH5a strains were used as routine cloning hosts. Plasmids for CRISPR-Cas9 mediated counterselection, pCas and pTargetF, were purchased from Addgene (Jiang, Y. et al., 2015). Strains were routinely grown in Lysogeny broth (LB) (Bioshop) at 37° C. or 30° C. For optimal aeration, broth cultures were grown with aeration at 220 rpm. For growth profiling, microtiter plates were incubated at 37° C. with continuous linear shaking at 600 rpm. Ampicillin (100 μg/mL) (Bioshop), kanamycin (50 μg/mL) (Sigma-Aldrich), spectinomycin (50 μg/mL) (Bioshop), and gentamicin (10 μg/mL) (BioBasic) were used at the listed concentrations for selection of resistance markers.

TABLE 21
Strains and plasmids used in this Example.

| | |
|---|---|
| Genotype or Description | Source |

Strains

| | | |
|---|---|---|
| E. coli K-12 str. | The parental strain of the KEIO collection | Baba, T. et |
| BW25113 | | al. (2006) |
| E. coli TOP10 | Cloning host, mcrA deficient for increased | Thermo |
| | efficiency in foreign DNA uptake | Fisher |
| | | Scientific |
| E. coli DH5α | Cloning host, endA deficient for high quality | Thermo |
| | DNA preparations | Fisher |
| | | Scientific |
| E. coli EKO-35v2 | BW25113 efflux deficient derivative (AacrB; | This |
| | acrD; acrF; mdtF; macB; emrB; mdtL; mdtK; | disclosure |
| | bcr; ydeA; mdtM; yddA; yebQ; emrE; mdtD; | |
| | sugE; ynfM; emrD; ydeF; mdtJ; ydiM; mdtB; | |
| | mdIA; emrY; mdfA; fsr; mdtG; mdtH; yieO; | |
| | mdlB, mdtO, yojI, yajR, ydhC; cusA) | |

Plasmids

| | | |
|---|---|---|
| pCas | Kan^{r}, temperature sensitive permissive (30° C.), | Jiang, Y. et |
| | non-permissive (37° C.), arabinose-induced | al. (2015) |
| | expression of the λ-Red recombinase for | |
| | homologous recombination. Constitutive | |
| | expression of Cas-9 | |
| pTargetF | Spec^{r}, modifiable by PCR to contain N20 | Jiang, Y. et |
| | sequence recognizable by Cas-9 | al. (2015) |
| pTargetF-mdtD- | Spec^{r}, modified by PCR to contain an N20 | |
| sugE | sequence recognizable by Cas-9 to target a | |
| | PAM site within mdtD and sugE | |
| pTargetF-ynfM- | Spec^{r}, modified by PCR to contain an N20 | |
| emrD-ydeF | sequence recognizable by Cas-9 to target a | |
| | PAM site within ynfM, emrD, and ydeF | |
| pTargetF-mdIA- | Spec^{r}, modified by PCR to contain an N20 | |
| emrY | sequence recognizable by Cas-9 to target a | |
| | PAM site within mdlA and emrY | |
| pTargetF-bcr-mdtK | Spec^{r}, modified by PCR to contain an N20 | |
| | sequence recognizable by Cas-9 to target a | |
| | PAM site within bcr and mdtK | |
| pTargetF-mdIB- | Spec^{r}, modified by PCR to contain an N20 | |
| mdtH | sequence recognizable by Cas-9 to target a | |
| | PAM site within mdlB and mdtH | |
| pTargetF-macB- | Spec^{r}, modified by PCR to contain an N20 | |
| yddA | sequence recognizable by Cas-9 to target a | |
| | PAM site within macB and yddA | |
| pTargetF-emrE | Spec^{r}, modified by PCR to contain an N20 | This |
| | sequence recognizable by Cas-9 to target a | disclosure |
| | PAM site within emrE | |
| pTargetF-mdtJ | Spec^{r}, modified by PCR to contain an N20 | This |
| | sequence recognizable by Cas-9 to target a | disclosure |
| | PAM site within mdtIJ | |
| pTargetF-ydiM | Spec^{r}, modified by PCR to contain an N20 | This |
| | sequence recognizable by Cas-9 to target a | disclosure |
| | PAM site within ydiM | |
| pTargetF-mdtB | Spec^{r}, modified by PCR to contain an N20 | This |
| | sequence recognizable by Cas-9 to target a | disclosure |
| | PAM site within mdtB | |
| pTargetF-mdfA | Spec^{r}, modified by PCR to contain an N20 | This |
| | sequence recognizable by Cas-9 to target a | disclosure |
| | PAM site within mdfA | |
| pTargetF-fsr | Spec^{r}, modified by PCR to contain an N20 | This |
| | sequence recognizable by Cas-9 to target a | disclosure |
| | PAM site within fsr | |
| pTargetF-mdtG | Spec^{r}, modified by PCR to contain an N20 | This |
| | sequence recognizable by Cas-9 to target a | disclosure |
| | PAM site within mdtG | |
| pTargetF-yieO | Spec^{r}, modified by PCR to contain an N20 | This |
| | sequence recognizable by Cas-9 to target a | disclosure |
| | PAM site within yieO | |
| pTargetF-mdtO | Spec^{r}, modified by PCR to contain an N20 | This |
| | sequence recognizable by Cas-9 to target a | disclosure |
| | PAM site within mdtO | |
| pTargetF-yojI | Spec^{r}, modified by PCR to contain an N20 | This |
| | sequence recognizable by Cas-9 to target a | disclosure |
| | PAM site within yojI | |
| pTargetF-yajR | Spec^{r}, modified by PCR to contain an N20 | This |
| | sequence recognizable by Cas-9 to target a | disclosure |
| | PAM site within yajR | |
| pTargetF-ydhC | Spec^{r}, modified by PCR to contain an N20 | This |
| | sequence recognizable by Cas-9 to target a | disclosure |
| | PAM site within ydhC | |
| pTargetF-cusA | Spec^{r}, modified by PCR to contain an N20 | This |
| | sequence recognizable by Cas-9 to target a | disclosure |
| | PAM site within cusA | |
| pTargetF-acrB | Spec^{r}, modified by PCR to contain an N20 | |
| | sequence recognizable by Cas-9 to target a | |
| | PAM site within acrB | |
| pTargetF-acrD | Spec^{r}, modified by PCR to contain an N20 | |
| | sequence recognizable by Cas-9 to target a | |
| | PAM site within acrD | |
| pTargetF-acrF | Spec^{r}, modified by PCR to contain an N20 | |
| | sequence recognizable by Cas-9 to target a | |
| | PAM site within acrF | |
| pTargetF-mdtF | Spec^{r}, modified by PCR to contain an N20 | |
| | sequence recognizable by Cas-9 to target a | |
| | PAM site within mdtF | |
| pTargetF-emrB | Spec^{r}, modified by PCR to contain an N20 | |
| | sequence recognizable by Cas-9 to target a | |
| | PAM site within emrB | |
| pTargetF-ydeA | Spec^{r}, modified by PCR to contain an N20 | |
| | sequence recognizable by Cas-9 to target a | |
| | PAM site within ydeA | |
| pTargetF-mdtM | Spec^{r}, modified by PCR to contain an N20 | |
| | sequence recognizable by Cas-9 to target a | |
| | PAM site within mdtM | |
| pTargetF-yddA | Spec^{r}, modified by PCR to contain an N20 | |
| | sequence recognizable by Cas-9 to target a | |
| | PAM site within yddA | |
| pTargetF-yebQ | Spec^{r}, modified by PCR to contain an N20 | |
| | sequence recognizable by Cas-9 to target a | |
| | PAM site within yebQ | |

### Generation of an Efflux Deficient Strain

Generation of EKO-35 was achieved using a CRISPR-Cas9 counter-selection (Jiang, Y. et al., 2015). The efflux genes were inactivated in the order denoted in Table 22. All PCR reactions and restriction enzyme digests were prepared according to manufacturers' guidelines. Amplicons were purified using a GeneJET PCR purification kit (Thermo Fisher Scientific) according to manufacturer's guidelines. The 2×GB-AMP™ high-fidelity PaCeR™ polymerase Master Mix (GeneBio Systems Inc) and Taq 2× polymerase Master Mix (FroggaBio) were used according to the manufacturer's suggested guidelines.

For CRISPR-Cas9-mediated counterselection, the methodology described by Jiang et al. was modified for high-throughput screening of mutants (Jiang, Y. et al., 2015). Multiple efflux-encoding genes were targeted simultaneously by multiplexing two or three guide RNAs in the pTargetF vector. CRISPR guide software (Benchling) was employed for selection of appropriate N20 sequences. pTargetF was modified via PCR to introduce an N20 for the gene of interest (Table 23). Amplicon size (2100 bp) was verified via gel electrophoresis, and the remaining PCR product was purified. A second N20 sequence was amplified using PaCeR™ polymerase and inserted into pTargetF-gene1 vector through restriction digest with EcoR1 and XhoI and ligation with T4 ligase. A third N20 sequence was amplified using PaCeR™ polymerase and inserted into pTargetF-gene1-gene2 vector through restriction digest with XbaI and HindIII and ligation with T4 ligase. pTargetF vectors were verified using Sanger Sequencing at the Advanced Analysis Centre (AAC) University of Guelph. To enable rapid screening of positive mutants and to disrupt the target gene, ssDNA repair oligos (˜100 bp in length) were designed to contain an AseI restriction site and three tandem stop codons (Table 23). All ssDNA repair oligos were purchased through Integrated DNA Technologies (IDT). Electrocompetent cells of the mutant strain of interest were transformed with 50 ng of pCas. A broth culture of each strain was grown to the mid exponential phase (OD600 nm˜0.5) in the presence of kanamycin and 10 mM arabinose to induce recombinase expression. To recombinase induced electrocompetent cells, 100 ng of pTargetF that was modified to contain the desired N20 sequence, and 2000 ng of repair ssDNA targeting the gene of interest were electroporated (Bio-Rad MicroPulser, Ec1 setting, 1 mm electroporation cuvette (Fisher)). The cultures were recovered in LB with 1 mM arabinose at 30° C. and propagated on selective agar (LB with kanamycin and spectinomycin) to identify successful gene disruptions. For high-throughput screening of colonies, Taq polymerase was used with primers annealing to the target region of each gene (Table 23). The amplicons were digested with AseI and successfully inactivated genes were identified via gel electrophoresis by digestion relative to a wild-type negative control. Insertion of the three tandem stop codons into the gene of interest was verified using Sanger sequencing at the Advanced Analysis Centre (AAC) (University of Guelph). Genes disrupted using CRISPR-Cas9-mediated counter selection are indicated in Table 22.

TABLE 22
Gene inactivation during the generation of EKO-35v2. For genes
inactivated using CRISPR-Cas9 mediated counter selection,
the location of the inserted stop codons are noted. Genes
are listed in the order in which they were inactivated.

| Gene | Gene Size (bp) | Stop Codon Placement (bp) |
|---|---|---|
| yajR | 1365 | 47 |
| mdtO | 2052 | 75 |
| ydhC | 1212 | 90 |
| emrE | 333 | 93 |
| yojI | 1644 | 25 |
| mdtD | 1416 | 26 |
| sugE | 318 | 93 |
| ynfM | 1254 | 115 |
| emrD | 1185 | 206 |
| ydeF | 1188 | 43 |
| mdlA | 1773 | 69 |
| emrY | 1539 | 41 |
| mdtK | 1374 | 135* |
| bcr | 1191 | 48* |
| mdtG | 1227 | 276 |
| mdtH | 1209 | 136 |
| mdlB | 1782 | 72 |
| macB | 1947 | 51* |
| yddA | 1686 | 105* |
| fsr | 1221 | 242 |
| ydiM | 1215 | 124 |
| yieO | 1428 | 177 |
| mdfA | 1233 | 116 |
| mdtM | 1233 | 48* |
| mdtJ | 366 | 98 |
| emrB | 1539 | 108* |
| mdtB | 3123 | 160 |
| mdtL | 1176 | 51* |
| yebQ | 1374 | 27* |
| cusA | 3144 | 400 |
| mdtF | 3114 | 72* |
| ydeA | 1191 | 142* |
| acrF | 3105 | 42* |
| acrD | 3114 | 45* |
| acrB | 3150 | 66* |

*indicates genes previously disrupted using λ-Red recombineering in EKO-35 of Example 1 (i.e. EKO-35v1).

TABLE 23
Primers and oligonucleotides used in this Example.

| | | SEQ ID |
|---|---|---|
| Primer | Sequence (5′-3′) | NO: |
| AcrB Seq Up | CTGAAACAAGAGAACGGCAAAGGC | 1 |
| AcrB Seq Low | CTTACTGACCTGGACTTGCCCTCTCG | 2 |
| AcrB_N20_Fwd | GATCGCCATTATCATCATGTGTTTTAGAGCTAGAAATAGCAAG | 302 |
| AcrB_N20_Rvs | ACATGATGATAATGGCGATCACTAGTATTATACCTAGGACTGAG | 303 |
| AcrB SSDNA | TTG CGC CAC CGG CAG TTT GAG GAT CGC TTA TTA | 304 |
| Repair | TTA ATT TAA CAT GAT GAT AAT GGC GAT CAC CCA | |
| | CGC AAA AAT CGG GCG ATC GAT AAA GAA ATT AGG | |
| | CAT | |
| AcrD Seq Up | CGCTGACTTTTTCACAACCTTCCG | 5 |
| AcrD Seq Low | GTCCCCCGGAGGTAGTCATCGCAGC | 6 |
| AcrD_N20_Fwd | CCAGCACCCAGGCAAAAATGGTTTTAGAGCTAGAAATAGCAAG | 305 |
| AcrD_N20_Rvs | CATTTTTGCCTGGGTGCTGGACTAGTATTATACCTAGGACTGAG | 306 |
| AcrD SSDNA | TTG TTC AAC GGG CAA TGA AAA AAT CGC CAG GGT | 307 |
| Repair | ACC TGT CAG TTA TTA TTA ATT CAG CAC CCA GGC | |
| | AAA AAT TCG ATC AAT AAA GAA ATT CGC CAT | |
| AcrF Seq Up | GAGGCTGAAGCAATCCGTAGAGC | 9 |
| AcrD Seq Low | GATACTGTATCGTTAAAAAGAGCGCG | 10 |
| AcrF_N20_Fwd | TCGACGACCGATATTTGCATGTTTTAGAGCTAGAAATAGCAAG | 308 |
| AcrF_N20_Rvs | ATGCAAATATCGGTCGTCGAACTAGTATTATACCTAGGACTGAG | 309 |
| AcrF SSDNA | CTG AGC GAC GGG CAA TTG TAG GAT CGC CAG TGC | 310 |
| Repair | GCC CGC CAT CAT CAG AAT TTA TTA TTA ATT TCA | |
| | TGC AAA TAT CGG TCG TCG AAT AAA AAA GTT TGC | |
| | CAT | |
| MdtF 200 Up | GATGTCGTGCAGCTACGCGAAAT | 13 |
| MdtF 200 Low | ACGAATGGCTGGAGTGGTTTC | 14 |
| MdtF_N20_Fwd | ATTATTATGATGCTTGCAGGGTTTTAGAGCTAGAAATAGCAAG | 311 |
| MdtF_N20_Rvs | CCTGCAAGCATCATAATAATACTAGTATTATACCTAGGACTGAG | 312 |
| MdtF SSDNA | AAT CTG CGG ATA CTG CGC AAC CGG TAA GTT CAT | 313 |
| Repair | TTA TTA TTA ATT ACC TGC AAG CAT CAT AAT AAT | |
| | GGC AAG TAC CCA GGC AAA AAC CGG GCG ATC AAT | |
| | AAA ATA | |
| MacB 200 Up | ATGTGCTGACGATCCCTCTGTC | 17 |
| MacB 200 Low | TTCGCTCATTATTCCACCATTCAG | 18 |
| MacB_N20_Fwd | ATTCGTCGCAGCTATCCTGCGTTTTAGAGCTAGAAATAGCAAG | 314 |
| MacB_N20_Rvs | GCAGGATAGCTGCGACGAATACTAGTATTATACCTAGGACTGAG | 315 |
| MacB SSDNA | ACC CGC ATA AAT ATC GAG GCT GAT GCC CTT CAG | 316 |
| Repair | CAC CTC AAC TTA TTA TTA ATT GGC AGG ATA GCT | |
| | GCG ACG AAT ATC CTT TAA TTC GAG CAA AGG CGT | |
| | CAT | |
| EmrB 200 Up | CGTTCAGCGTCTGCCTGTGCG | 21 |
| EmrB 200 Low | GCGGCAATGGAAGACGTGCTG | 22 |
| EmrB_N20_Fwd | GGACTCCACCATTGCTAACGGTTTTAGAGCTAGAAATAGCAAG | 317 |
| EmrB_N20_Rvs | CGTTAGCAATGGTGGAGTCCACTAGTATTATACCTAGGACTGAG | 318 |
| EmrB SSDNA | CTG GCT GAG CGA TGA GCC CAG ATT CCC GGC GAT | 319 |
| Repair | TTA TTA TTA ATT AAC GTT AGC AAT GGT GGA GTC | |
| | CAG CAC CTG CAT GAA TGT CGC CAG TGA CAG CGC | |
| | AAT CGT | |
| MdtL 200 Up | CATTCTCTTTGGTATAACCGTG | 25 |
| MdtL 200 Low | TTTGCTCCATGCTGACCAT | 26 |
| MdtL_N20_Fwd | GGCGGGATAAAGTAAAACCAGTTTTAGAGCTAGAAATAGCAAG | 320 |
| MdtL_N20_Rvs | TGGTTTTACTTTATCCCGCCACTAGTATTATACCTAGGACTGAG | 321 |
| MdtL SSDNA | ATT GAG ATC GGC GGC GAT GCG CGG TAA ACC AAC | 322 |
| Repair | GAG GTA TTA TTA TTA ATT GGC GGG ATA AAG TAA | |
| | AAC CAG AAA ACT ACA AAT CAA AAA GCG GGA CAT | |
| MdtK 200 Up | GAAATCAGTTAAGACATTCTGTTC | 29 |
| MdtK 200 Low | CATGTGCAACTGAAAGTGAAAC | 30 |
| MdtK_N20_Fwd | CTATAGTGCCACCGACATGGGTTTTAGAGCTAGAAATAGCAAG | 323 |
| MdtK_N20_Rvs | CCATGTCGGTGGCACTATAGACTAGTATTATACCTAGGACTGAG | 324 |
| MdtK SSDNA | ACC AAA GAG GAT CGC CGG AAG CCA GAT AGA AGT | 325 |
| Repair | ACC TTA TTA TTA ATT CAT GTC GGT GGC ACT ATA | |
| | GCC GCC CGC CAT CAC GGT ATC GAC AAA ACC CAT | |
| | CGC | |
| Bcr 200 Up | CCTCTATGGCTCTGATTTAAGTA | 33 |
| Bcr 200 Low | GTTATCATCAGGTGAAACGCAT | 34 |
| Bcr_N20_Fwd | AATCGACAGCGGCATCAACAGTTTTAGAGCTAGAAATAGCAAG | 326 |
| Bcr_N20_Rvs | TGTTGATGCCGCTGTCGATTACTAGTATTATACCTAGGACTGAG | 327 |
| Bcr ssDNA Repair | CGG TAG CGC GGG CAG ATA CAT ATC AAT CGA CAG | 328 |
| | CGG CAT CAA CAT TTA TTA TTA ATT AAG GAT AAA | |
| | AAC AAT AGC AAA CGA CGA ATG CTG TCG GGT GGT | |
| | CAC | |
| YdeA 150 Fwd | CCGTGATGTTACCGACTCTC | 329 |
| YdeA 150 Rvs | GTGGCATTGAAGCGATCTCC | 330 |
| YdeA_N20_Fwd | CAACATGATGCCGACCTGAGGTTTTAGAGCTAGAAATAGCAAG | 331 |
| YdeA_N20_Rvs | CTCAGGTCGGCATCATGTTGACTAGTATTATACCTAGGACTGAG | 332 |
| YdeA SSDNA | CAT TAG CGC TAC TAC CCA TGC GTA AAT GGT CAA | 333 |
| Repair | CAT GAT GCC GAC CTG AGC TTA TTA TTA ATT TTG | |
| | CAT GTG AAA ACT TTG CGC AAT GTC AGA GAG CAG | |
| | GCC AAC AGG GAC | |
| MdtM 250 Up | CAGCGTAACGACAAAGGTAGCAG | 41 |
| MdtM 200 Low | ACCACCGCAAACCAGTC | 42 |
| MdtM_N20_Fwd | CATCGGGAAAAACAGCGTGGGTTTTAGAGCTAGAAATAGCAAG | 334 |
| MdtM_N20_Rvs | CCACGCTGTTTTTCCCGATGACTAGTATTATACCTAGGACTGAG | 335 |
| MdtM SSDNA | CTG GAT CAG ATC CGT CGA CAG ATA CGC AGC AAA | 336 |
| Repair | GTC ATA TTA TTA TTA ATT CAT CGG GAA AAA CAG | |
| | CGT GGC ATG GCG GGT AAA AAA ACG TGG CAT | |
| YddA 250 Up | TGTCGGGTGTTTCGTCAT | 45 |
| YddA 250 Low | TCGCTGATATTGCCATTC | 46 |
| YddA_N20_Fwd | CCACGCCAAGGATCATGGCGGTTTTAGAGCTAGAAATAGCAAG | 337 |
| YddA_N20_Rvs | CGCCATGATCCTTGGCGTGGACTAGTATTATACCTAGGACTGAG | 338 |
| YddA SSDNA | GTC GTT TAA CCA GAC CTG AAT TTT AAC CAC GCC | 339 |
| Repair | AAG GAT CAT GGC GAG TTA TTA TTA ATT TAA CAA | |
| | CAC TGA AGT TTT ATT ATT CTT ACG CAG CCA AAA | |
| | GGG CTT | |
| YebQ 200 Up | CACGGAAGATACAGAATCAGG | 49 |
| YebQ 200 Low | CAGCTATGAACCGCAAGAA | 50 |
| YebQ_N20_Fwd | AATATCGCACCGTATCGCTGGTTTTAGAGCTAGAAATAGCAAG | 340 |
| YebQ_N20_Rvs | CAGCGATACGGTGCGATATTACTAGTATTATACCTAGGACTGAG | 341 |
| YebQ SSDNA | AAT ACC AAT CAC AAT GGT TAA TAT CGC ACC GTA | 342 |
| Repair | TCG CTG TTA TTA TTA ATT GCC GTC GGC CTG AAC | |
| | TTT TGG CAT AGG AAT TTT ATA TCT TTG GTG AAT | |
| | AAT | |
| EmrE N20 Up | AGTTTTCAGAAGGTTTTACAGTTTTAGAGCTAGAAATAGCAAG | 51 |
| EmrE N20 Low | TGTAAAACCTTCTGAAAACTACTAGTATTATACCTAGGACTGAG | 52 |
| EmrE SSDNA | ATA ACA AAT AAT TGT ACC AAC AGA TGG CCA TAA | 53 |
| Repair | TTA TTA TTA ATT TGT AAA ACC TTC TGA AAA CTT | |
| | CAT TAA GGT TGT ACC AAT GAC CTT TAT TAT TAC | |
| | TGC | |
| EmrE 200 Up | CGGTTCGCTACCAGAGAAGAATG | 54 |
| EmrE 200 Low | CATGGTGACACCTGCTAACGTATGC | 55 |
| MdtD N20 Up | CCACAATCCACAATTGCCAAGTTTTAGAGCTAGAAATAGCAAG | 56 |
| MdtD N20 Low | TTGGCAATTGTGGATTGTGGACTAGTATTATACCTAGGACTGAG | 57 |
| MdtD SSDNA | TG TCC AGC GAC TGC ATA AAG AAG CCG AAA GCC ACA | 58 |
| Repair | ATC CAC AAT TGC CAA TTA TTA TTA ATT GGT GCT | |
| | GTC GGG AAG ATC TGT CAT TTA CTC GGT TAC CGT | |
| | TTG TTT AGG TT | |
| MdtD 200 Up | CGCCCGATTATGATGACTAC | 59 |
| MdtD 200 Low | CTGAAAGACAAAGCGATCATTG | 60 |
| SugE N20 Up | CGTCAAACGACTAAAGCCGTGTTTTAGAGCTAGAAATAGCAAG | 61 |
| SugE N20 Low | CGTCAAACGACTAAAGCCGTACTAGTATTATACCTAGGACTGAG | 62 |
| SugE SSDNA | GAC AAT CAT CGC CGT CAC AGT AAT AAC ACT CGG | 63 |
| Repair | CGT CAA ACG ACT AAA GCC GTG TTA TTA TTA ATT | |
| | ATA TTT CAG GCC AAC GGC CCA TAC CAC TTC CAG | |
| | CAG ACC AGC AAT AAC TAA GAT | |
| SugE 200 Up | CGCAGCAACGAAAGCGCA | 64 |
| YnfM N20 Up | TCCGGCAGAGAACAGCGCCAGTTTTAGAGCTAGAAATAGCAAG | 65 |
| YnfM N20 Low | TGGCGCTGTTCTCTGCCGGAACTAGTATTATACCTAGGACTGAG | 66 |
| YnfM SSDNA | CTG CAC ACA ATA GAG AAG TGC AAA TGT TGC CAG | 67 |
| Repair | TCC GGC AGA GAA CAG CGC CAG TTA TTA TTA ATT | |
| | GAC GCG CAT AAA TTG CGG CGT ACC GCG TTT AAT | |
| | AAA TTG ATT TGG CTG AGA AAT | |
| YnfM 200 Up | GTTGCGAAATATTCAGGC | 68 |
| YnfM 200 Low | AAAGCAGTAGAATAACTGC | 69 |
| EmrD N20 Up | TCACCGGTCGGCGGCCCACGGTTTTAGAGCTAGAAATAGCAAG | 70 |
| EmrD N20 Low | CGTGGGCCGCCGACCGGTGAACTAGTATTATACCTAGGACTGAG | 71 |
| EmrD SSDNA | CGT TGC CAG CAT AAA AAT GGA CAT TCC GAC GAG | 72 |
| Repair | GAT CAC CGG TCG GCG GCC CAC GCG TTA TTA TTA | |
| | ATT GGA AAT CGG GCC ATA AAA CAG CTG TGA GAC | |
| | ACC GTA AGT CAG CAG ATA AGC GCC | |
| EmrD 200 Up | CGATGCTGACGCATCTTATCCGCCC | 73 |
| EmrD 200 Low | GGTGCGGGCAGATATCAGTCGTATC | 74 |
| YdeF N20 Up | GATGGTTAATAACAACGACGGTTTTAGAGCTAGAAATAGCAAG | 75 |
| YdeF N20 Low | CGTCGTTGTTATTAACCATCACTAGTATTATACCTAGGACTGAG | 76 |
| YdeF SSDNA | AAT GGT CAT AAA TGG CAG CGT AGC GCC GCG TCC | 77 |
| Repair | GAT GGT TAA TAA CAA CGA CGA TTA TTA TTA ATT | |
| | AAG AAG GGC GCT GGT AGA GCG TCG TAG GGA TAA | |
| | GTT CAT | |
| YdeF 250 Up | CTGATGGTTAATCCATACCCCAGC | 78 |
| YdeF Check | GATGCTCTGCATTACCAACAGCGTG | 79 |
| Internal | | |
| MdtJ N20 Up | AGCGTCAGTGAGGGAAATGGGTTTTAGAGCTAGAAATAGCAAG | 80 |
| MdtJ N20 Low | CCATTTCCCTCACTGACGCTACTAGTATTATACCTAGGACTGAG | 81 |
| MdtJ SSDNA | CGA CAG AGA AAT CAT CAC CAG CAT TAA AAT AAA | 82 |
| Repair | TTA TTA TTA ATT GCC ATT TCC CTC ACT GAC GCT | |
| | CGC CCA TTT CAT TGA CAG CGT ACC GGT | |
| MdtJ 200 Up | CAATGCATAAGCGACAGACAAGTCG | 83 |
| MdtJ 200 Low | CATCCGCGATGACGAGAAGCAACAC | 84 |
| YdiM N20 Up | GATAACTATCGAGACACCCGGTTTTAGAGCTAGAAATAGCAAG | 85 |
| YdiM N20 Up | CGGGTGTCTCGATAGTTATCACTAGTATTATACCTAGGACTGAG | 86 |
| YdiM SSDNA | CAA GAC ACT TAA TCG ACC AAT GCC CAG CGA TGA | 87 |
| Repair | GAT AAC TAT CGA GAC ACC CGC TTA TTA TTA ATT | |
| | ATT AGT CTG CCA AAG TGT CTC CAG CGA GGC CAT | |
| | ATT CAG | |
| YdiM Check Up | CAAGTGTGCCATTCCTGATCGTG | 88 |
| YdiM Check Up | GAACCCACGGTGTAGATACTGAG | 89 |
| MdtB N20 Up | GTAGAGCGTGACCACCTGAAGTTTTAGAGCTAGAAATAGCAAG | 90 |
| MdtB N20 Low | TTCAGGTGGTCACGCTCTACACTAGTATTATACCTAGGACTGAG | 91 |
| MdtB SSDNA | AAC GGC AGA GGT CAT GAC ATC CGG GCT GGC ACC | 92 |
| Repair | TGG GTA GAG CGT GAC CAC CTG AAT TTA TTA TTA | |
| | ATT CGG ATA GTC CAC TTC CGG CAG CGC CGA AAC | |
| | GGG CAG | |
| MdtB 200 Up | GTCAGAAAGTGGTGATCCGTGCAG | 93 |
| MdtB Check Low | GATCGCTCGGCAACAAGTTGGTCG | 94 |
| MdlA N20 Up | CATCGCGATAATGACAAGCAGTTTTAGAGCTAGAAATAGCAAG | 95 |
| MdlA N20 Low | GCTTGTCATTATCGCGATGACTAGTATTATACCTAGGACTGAGT | 96 |
| MdlA SSDNA | ACC AAC CAC TTT TGG CGG AAC CAG TTG CAG CAT | 97 |
| Repair | CGC GAT AAT GAC AAG CAA TTA TTA TTA ATT GAC | |
| | AGC CCC GAG ATA GCG ACG CCA TTC CCG ACG GAA | |
| | ATA CCA GCT | |
| MdlA 300 Up | GTCACGGTGGTTACCGAAATGCCAG | 98 |
| MdlA Check | GCGTTAAACTGCCCTGCACCAC | 99 |
| Internal | | |
| EmrY N20 Up | ACTCCGGCACCATTAACCGGGTTTTAGAGCTAGAAATAGCAAG | 100 |
| EmrY N20 Low | CCGGTTAATGGTGCCGGAGTACTAGTATTATACCTAGGACTGAG | 101 |
| EmrY SSDNA | TTG CAT AAA TGT CGC TAA TGA CAA TGC AAT AGT | 102 |
| Repair | GAC GCA CCA TAA CGT TTA TTA TTA ATT ACC GGT | |
| | TAA TGG TGC CGG AGT TGA TTT AGT GAT TGC CAT | |
| EmrY 200 Up | AGCGCAGAACAACTGCGTAATA | 103 |
| EmrY 200 Low | GTACGGGTTGAAGTTTCTCTTG | 104 |
| MdfA N20 Up | GGCAACGATATGATTCAACCGTTTTAGAGCTAGAAATAGCAAG | 105 |
| MdfA N20 Low | GGTTGAATCATATCGTTGCCACTAGTATTATACCTAGGACTGAG | 106 |
| MdfA SSDNA | AAT GCC CGC CTG ATA TTG TTC CAC CAC GGC CAA | 107 |
| Repair | CAT TTA TTA TTA ATT GGG TTG AAT CAT ATC GTT | |
| | GCC GAT ATA GGT TGA AAA TTC GTA AAG CAC CAG | |
| | ACA | |
| MdfA_200_Up | ATCGTCTTATTTCCCTCAAGC | 108 |
| MdfA_200_Low | ATGTGCCGAGTGGATACAAAGT | 109 |
| Fsr N20 Up | TCGCTACTGCAACCAGTGGTGTTTTAGAGCTAGAAATAGCAAG | 110 |
| Fsr N20 Up | ACCACTGGTTGCAGTAGCGAACTAGTATTATACCTAGGACTGAG | 111 |
| Fsr ssDNA Repair | CGA CCA TGG CAT CGG ATA TTT ATC GGT CCA GTA | 112 |
| | TTA TTA TTA ATT GAC CAC TGG TTG CAG TAG CGA | |
| | AGA GGC GAG CTG GAA GGT GAG GGT TAT CAT GCC | |
| | AAT CTG | |
| Fsr Check Up | GGTTAACAGCGCTAACGCCACG | 113 |
| Fsr Check Low | GTGGCGTGATGCATTCCGTCTC | 114 |
| MdtG N20 Up | ATGCTATTACGCTCTGCCCTGTTTTAGAGCTAGAAATAGCAAG | 115 |
| MdtG N20 Low | AGGGCAGAGCGTAATAGCATACTAGTATTATACCTAGGACTGAG | 116 |
| MdtG SSDNA | GAT ATT TTG TGC CAG CCC CAT CAA CAC CAT CAC | 117 |
| Repair | GAT GCC CAT TTA TTA TTA ATT GAG GGC AGA GCG | |
| | TAA TAG CAT GAG TTT TCG GCC TTT ACG GTC GGC | |
| | GAG TCC ACC CCA AAA CGG | |
| MdtG Check Up | GGCATTGAACTGTTGCACATTCGC | 118 |
| MdtG Check Low | CATGATGGCACCAGAGCAGTATATG | 119 |
| MdtH N20 Up | GAGAGCAATACCGACCATGAGTTTTAGAGCTAGAAATAGCAAG | 120 |
| MdtH N20 Low | TCATGGTCGGTATTGCTCTCACTAGTATTATACCTAGGACTGAG | 121 |
| MdtH SSDNA | GAA AAT ACC CAG ACC TTG CTG AAT AAA TTG GCG | 122 |
| Repair | TAG ACC GAG AGC AAT ACC GAC CAT GAC TTA TTA | |
| | TTA ATT GGC CCA GCC CAT TTG ATC AAC GAA GCG | |
| | GAT AGA | |
| MdtH Check Up | GCGTCGTCGTTGAGCAGAACATG | 123 |
| MdtH Check Low | GTCGGTCTGTGGTTAAGCGCAC | 124 |
| YieO N20 Up | CATCAGTTATACGCTGACGGGTTTTAGAGCTAGAAATAGCAAG | 125 |
| YieO N20 Low | CCGTCAGCGTATAACTGATGACTAGTATTATACCTAGGACTGAG | 126 |
| YieO SSDNA | GCG ATC GGC TAG CCA TCC GCT TAC CGG AAT AAG | 127 |
| Repair | CAT TTA TTA TTA ATT CAC CGT CAG CGT ATA ACT | |
| | GAT GAT GGC TGA TTG CAT CGC GAG AGG AGA ACG | |
| | ATT AAG | |
| YieO Check Up | CGTCAATTACCAGCGACACAGTG | 128 |
| YieO Check | CGTGCATGGAGAATATAGAGAAGC | 129 |
| Internal | | |
| MdlB N20 Up | ATCAGGACCGCAATCCCCAGGTTTTAGAGCTAGAAATAGCAAG | 130 |
| MdlB N20 Low | CTGGGGATTGCGGTCCTGATACTAGTATTATACCTAGGACTGAG | 131 |
| MdlB SSDNA | ACT GAC TTC TGC CGC CGC CGC AAC CCA CAT CAT | 132 |
| Repair | CAG GAC CGC AAT CCC CAG TTA TTA TTA ATT TTT | |
| | ACG CCA CGG CGA ACC GTA CGC TAA CAG GCG CTT | |
| | GAG AGT | |
| MdlB Check Up | CTTGATGATGCGCTTTCGGCGGTG | 133 |
| MdlB Check | CGCCATATAGTGTGAACGACTGGCC | 134 |
| Internal | | |
| MdtO N20 Up | TTCATGAAGAGTTAAGCGAGGTTTTAGAGCTAGAAATAGCAAG | 135 |
| MdtO N20 Low | CTCGCTTAACTCTTCATGAAACTAGTATTATACCTAGGACTGAG | 136 |
| MdtO SSDNA | GAG TTG CAC GGT CTG CGG CAC GCG ACC TGG TCG | 137 |
| Repair | TTA TTA TTA ATT CTC GCT TAA CTC TTC ATG AAA | |
| | GAA CGC CAG CAG CCT GAC CAC CGG TAA TGG CAG | |
| | GGA GTT | |
| MdtO Check | CGTAGCGCATATAGTCTGGATTGG | 138 |
| Internal | | |
| MdtO Check Up | GAGGGTAAAGTGGATTCGATTGGC | 139 |
| YojI N20 Up | AACTTCTTGTACTTGTCTGGGTTTTAGAGCTAGAAATAGCAAG | 145 |
| YojI N20 Low | GCCAGACAAGTACAAGAAGTTACTAGTATTATACCTAGGACTGAG | 146 |
| YojI SSDNA | TAG CGC CAT CAC ACT GAT AAA TGG CCA GCG ATA | 147 |
| Repair | CTG TTA TTA TTA ATT CCA GAC AAG TAC AAG AAG | |
| | TTC CAT GCA GAA AAC CCG GAC AAT GAA TTA CAG | |
| | CCC GCA GTT | |
| YojI Check | GTCGCGGCAACGTTGGTATCAG | 148 |
| Internal | | |
| YojI Check Up | GCTGCATCAGGATAAAGACGAACCG | 149 |
| YajR N20 Up | GGTGAGAGGCGCGCGACCTGGTTTTAGAGCTAGAAATAGCAAG | 150 |
| YajR N20 Low | CAGGTCGCGCGCCTCTCACCACTAGTATTATACCTAGGACTGAG | 151 |
| YajR SSDNA | GCC CAG CAT GCG CAA CGA GAA TAC GGT CCC TTA | 152 |
| Repair | TTA TTA ATT CCA GGT CGC GCG CCT CTC ACC TGG | |
| | CGT CAT TTT ATA ATC GTT cat TAC CAC CTC TGT | |
| | TTT AAA TTC | |
| YajR Check Up | GTTGCTGATGACAGAATCTGGGCGC | 153 |
| YajR Check | CCATTCCGGACTCACGATTAAGTACG | 154 |
| Internal | | |
| YdhC N20 Up | TTGCAGGTCGGCCTGTATGGGTTTTAGAGCTAGAAATAGCAAG | 155 |
| YdhC N20 Low | CCATACAGGCCGACCTGCAAACTAGTATTATACCTAGGACTGAG | 156 |
| YdhC SSDNA | AAG GAA CAG ACT AAG GCT GGC ACT GAC AGC AGA | 157 |
| Repair | CGC AGG CGT TTG CAG GTC GGC CTG TAT GGC TTA | |
| | TTA TTA ATT GAA AGC AGG CAG ATA CAT ATC GGT | |
| | TGC CAG AAA | |
| YdhC Check Up | CACATCACGGTGCCGTCGTTCAAAG | 158 |
| YdhC Check | CCGGTTTACGACCATAACGGTCGG | 159 |
| Internal | | |
| CusA N20 Up | ATAGATCCAGCCAACACCCGGTTTTAGAGCTAGAAATAGCAAG | 160 |
| CusA N20 Low | CGGGTGTTGGCTGGATCTATACTAGTATTATACCTAGGACTGAG | 161 |
| CusA SSDNA | CAG ATC GTG CTT ACC GCT GCG ATC CAC CAG TGC | 162 |
| Repair | ATA TTC ATA GAT CCA GCC AAC ACC CGT TTA TTA | |
| | TTA ATT ATC TGG CCC CAG CTC GGC GCT GAC TCC | |
| CusA 200 Up | GGTGATTACCGTTGATGCCGAC | 163 |
| CusA Check | GAGAAACCAGTCCTGTAATGAGCG | 164 |
| Internal | | |
| YhaM_N20_Fwd | GTTGGTATGCCCGCCGACGAGTTTTAGAGCTAGAAATAGCAAG | 343 |
| YhaM_N20_Rvs | TCGTCGGCGGGCATACCAACACTAGTATTATACCTAGGACTGAG | 344 |
| YhaM_Check_Fwd | CAACATTAACGAATTAAACAACCCG | 345 |
| YhaM_Check_Rvs | GTTGCTGTGTGTTTCTCCGTTC | 346 |
| YhaM SSDNA | CAC ACC ATC GTG CGT CTC GAT ATG CAC AAT GTT | 347 |
| Repair | GGT ATG CCC GCC GAC GAT TGT GAC ACA CGC CCA | |
| | CTT CTC ACC GTT CCA GAC TTT GGC TCG AGA GAA | |
| | GAG GAT TTC ATC | |

### Whole Genome Sequencing of EKO-35v2

Genomic DNA was extracted using the Purelink Genomic DNA Mini Kit (Invitrogen), according to the manufacturer's guidelines. Quality of the extracted gDNA was assessed using gel electrophoresis. Illumina DNA library preparation was performed using an Illumina Nextera kit by the Microbial Genome Sequencing Center (Pennsylvania, USA), which was followed by Illumina sequencing on a NextSeq 2000 platform. Analysis of the raw reads was performed using Geneious Prime 2021.0.2 (Kearse, M. et al., 2012). Low quality reads were trimmed using an in-suite BBDuk plug-in. Raw wild-type reads were assembled to an NCBI reference genome (Accession No. CP009273.1) with bowtie2. The resulting assembly was used as a reference to assemble the EKO-35v2 mutant reads. Differences between the wild-type BW25113 and EKO-35v2 strains were identified by searching for single nucleotide polymorphisms (SNPs) and deletions using the following thresholds: minimum variant frequency of 0.75, maximum variant P-value of 10⁻⁶, and minimum variant P-value of 10⁻⁵. The results were confirmed using the breseq (v 0.35.6) pipeline. Three intergenic mutations and three secondary mutations were identified, as summarize in Table 24. The nonsynonymous mutation in yhaM was repaired using CRISPR-Cas9-mediated counterselection, which introduced three intentional silent mutations (yhaM A390T, C393A, C432A) to remove the adjacent PAM site and introduce XhoI-guided screening.

TABLE 24
EKO-35v2 genomic mutations. Single nucleotide polymorphisms were
identified relative to the parent E. coli K-12 genome.
Secondary Mutations

| | Base Pair | | |
|---|---|---|---|
| Gene | Mutation | Effect | Gene Function |
| xylG | A116C | Missense | D-xylose ABC |
| | | | transporter |
| ydgK | C231T | Silent | Putative inner |
| | | | membrane protein |

Intergenic Mutations

| 5′ Flanking | 3′ Flanking | Position relative to | |
|---|---|---|---|
| gene | Gene | flanking genes | Mutation |
| ecpR | ykgL | −144/−632 | (T)₈ → (T)₇ |
| cysZ | cysK | +21/−164 | A→ T |
| kduI | yqeF | −123/+164 | T→ C |

### Phenotypic Profiling of EKO-35

For growth profiling in nutrient-rich conditions, strains were propagated on LB agar for 18 h at 37° C. Single colonies were inoculated into LB and grown at 37° C. until the mid-exponential phase (OD600 nm˜0.6) was reached. All strains were assessed with at least three biological replicates. The cultures were standardized to an OD600 nm˜0.1 in sterile 0.85% saline (w/v). Standardized cultures were diluted 1/200 into LB and 100 μL of the resulting dilution were applied to round-bottom 96-well microtiter plates (VWR). To prevent evaporation, the microtiter plates were sealed (labeling tape, Fisher Scientific). The OD600 nm was measured every 15 minutes over the course of 24 h using a BioTek Synergy H1 microplate reader. Growth was assessed at both 37° C. and 25° C.

### Results

Generation of EKO-35: Inactivation of E. coli Drug Efflux Pumps

Inventors' first goal was to generate a simplified genetic background to overcome the challenges associated with the complexities of intact drug efflux networks. To generate the first-generation strain, inventors started with an ΔacrB mutant from the Keio Collection and removed a further 13 pumps using the λ-Red system and 22 additional pumps using CRISPR Cas9-mediated counterselection. As such, EKO-35 of Example 1 (i.e. EKO-35v1) contains 13 flippase recognition target (FRT) sites and 11 secondary mutations. To circumvent genomic mobility due to FRT site-mediated DNA translocation, the inventors created the second-generation strain, EKO-35v2, using only CRISPR Cas9-mediated counterselection. EKO-35v2 is a scarless genetic background with significantly fewer secondary mutations, representing a stable isogenic background devoid of 35 efflux-encoding genes (Table 24).

The efflux genes were inactivated in the following order: ΔyajR; mdtO; ydhC; emrE; yojI; mdtD, sugE; ynfM, emrD, ydeF; mdlA, emrY; mdtK, bcr; mdtG; mdtH, mdlB, macB, yddA; fsr; ydiM; yieO; mdfA; mdtM; mdtJ; emrB, mdtB, mdtL; yebQ; cusA; mdtF; ydeA; acrF; acrB. While generating the strain, inventors identified a missense mutation (K₁₃₉T) within the gene encoding a putative L-cysteine desulfidase (YhaM). In effort to create a scarless genetic background devoid of secondary mutations, the inventors repaired this mutation. However, an additional missense mutation (N39H) arose within a gene encoding a xylose ABC transporter (XlyG).

Since many of the efflux pump-encoding genes have predicted start codons and are poorly characterized, it is possible that alternative start codons located downstream from the inserted tandem stop codons (Table 26A) could be utilized for a subset of the CRISPR-inactivated pumps. To investigate, inventors profiled the genome of the efflux-deficient strain, which included the inserted stop codons, using the Prodigal prokaryotic gene recognition and translation initiation site identifier (Hyatt et al, 2010). Overall, the program did not predict production of any potentially functional efflux pumps in EKO-35v1 and EKO-35v2, which supports the notion that the tandem stop codons were sufficient to prematurely terminate translation, and that alternate start codons would not be utilized (Tables 26A and 26B). Indeed, Prodigal analysis of the wild-type strain's genome predicted production of all pumps (Table 26B). In addition, inventors also took advantage of new developments in protein structure predictions, and carefully analyzed AlphaFold-generated models of each efflux pump to ascertain whether the predicted start codons were correct based on the structural features of the proteins. Such an analysis indicated the inserted stop codons were sufficient to inactivate the different genes. This strain was subsequently designated Efflux KnockOut-35 version 2 (EKO-35v2), and was used for phenotypic characterization and construction of an efflux platform, as described below. The EKO-35v2 genome sequence (SEQ ID NO: 255) confirmed successful disruption of the 35 efflux-encoding genes, including successful repair of yhaM, and also revealed two additional secondary mutations, one of which encoded missense mutations and one silent substitutions (Table 24).

### Characterizing EKO-35: Phenotypic Analysis

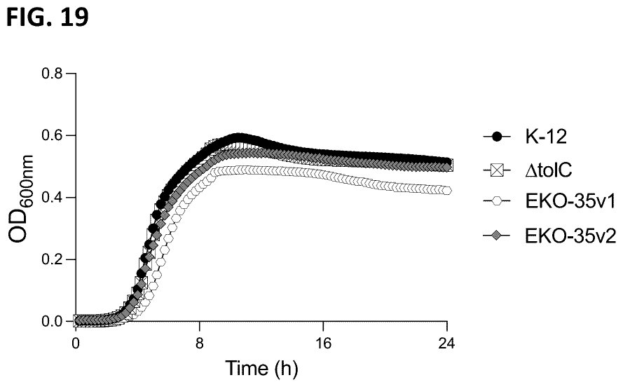

With the intent of using EKO-35 as a simplified genetic background to study the functions of drug efflux pumps, inventors' second goal was to fully characterize EKO-35v1 and v2 prior to further investigations. Other efflux-deficient backgrounds (e.g., tolC mutants) display pleiotropic phenotypes, including severe growth defects in minimal M9 medium with glucose supplied as the carbon source. Indeed, tolC inactivation causes periplasmic accumulation of enterobactin in this low-iron growth medium, which underlies this conditionally essential phenotype. In addition, tolC mutants exhibit membrane stress and depletion of essential metabolites, resulting in ‘metabolic shutdown’ in minimal glucose medium. Phenotypic analysis of EKO-35v1 growth kinetics under standard laboratory conditions, nutrient-rich Lysogeny broth, revealed EKO-35v1 exhibited a 1 h extended lag phase. To assess the impact of the secondary mutations and genomic mobility on the growth kinetics of EKO-35v1, the growth kinetics of EKO-35v2 were also profiled. Compared to the wild-type strain, EKO-35v2 shows no significant difference in the lag phase of growth or doubling time (FIG. 19, Table 25). As such, inventors conclude the previously observed phenotypic differences between the parent strain and EKO-35v1 were due to genomic limitations of the strain, rather than loss of efflux-encoding genes. Therefore, both EKO-35v1 and EKO-35v2 are useful platforms for investigating drug efflux.

TABLE 25
Measurement of growth kinetics revealed statistically significant
differences between EKO-35v2 and the wild-type strain. Generation
time and the duration of the lag phase for each strain are
shown in minutes. The measurements represent the mean ±
standard deviation for three biological replicates.

| | | | |
|---|---|---|---|
| | Generation | | |
| Strain | time (min) | Lag phase (min) | Final OD_{600 nm} |

Nutrient-rich medium at 37° C.

| | | | |
|---|---|---|---|
| K-12 | 29.66 ± 1.067 | 207.85 ± 0.663 | 0.501 ± 0.010 |
| EKO-35v1 | 32.04 ± 1.531 | 256.15 ± 0.946 | 0.415 ± 0.008 |
| EKO-35v2 | 30.50 ± 0.7512 | 209.09 ± 1.234 | 0.496 ± 0.021 |
| P-value_{EKO-35v1} | 5.54 × 10⁻³ | 1.99 × 10⁻¹⁹ | 5.29 × 10⁻¹⁰ |
| P-value_{EKO-35v2} | 1.15 × 10⁻¹* | 3.82 × 10⁻² | 5.29 × 10⁻¹* |

*non-significant P-values.
Statistical significance was assessed using a two-tailed Student's t-test (P-value ≤ 0.05).

TABLE 26A
Prediction of protein-coding genes using Prodigal prokaryotic gene
recognition and translation initiation site identifier. A consensus sequence (60%
threshold) of the efflux-deficient strain genome assembly, containing the tandem stop
codons, was utilized as the query fasta. As a control, the wild-type E. coli K-12
genome, without any CRISPR modifications, was also used (GenBank: CP009273.1).
The Prodigal results were searched for efflux-encoding genes using BLASTP (cutoff
threshold E < 1.0 e⁻⁵⁰). Predicted peptide size in EKO-35 is denoted as the length of
the truncated peptide as a fraction of the total protein size.
EKO-35 Genome and EKO-35v2 Genome

| | | Predicted | Predicted | | SEQ |
|---|---|---|---|---|---|
| | | Translation Start | Peptide | Predicted | ID |
| Gene | Predicted RBS | Site | Size | Stop Site | NO: |
| acrB | — | — | — | | |
| acrD | — | — | — | | |
| acrF | — | — | — | | |
| mdtF | — | — | — | | |
| macB | — | — | — | | |
| emrB | — | — | — | | |
| mdtL | — | — | — | | |
| mdtK | — | — | — | | |
| bcr | — | — | — | | |
| ydeA | — | — | — | | |
| mdtM | — | — | — | | |
| yddA | — | — | — | | |
| yebQ | — | — | — | | |
| emrE | AGGA/GGAG/ | MNPYIYLG [...] | 233 | GFTN* | 244 |
| | GAGG | | | | |
| mdtD | — | — | | | |
| sugE | — | — | — | | |
| ynfM | — | — | — | | |
| emrD | Unknown | MKRQRNVN [...] | 234 | PISN* | 245 |
| ydeF | — | — | | | |
| mdtJ | AGGAG | MYIYWILL [...] | 235 | GNGN* | 245 |
| ydiM | AGxAG | MKNPYFPT [...] | 236 | QTNN* | 246 |
| mdtB | AGGAG | MQVLPPSS [...] | 237 | DYPN* | 247 |
| mdlA | — | — | | | |
| emrY | — | — | | | |
| mdfA | — | — | | | |
| fsr | Unknown | MAMSEQPQ [...] | 238 | PVVN* | 249 |
| mdtG | GGA/GAG/AGG | MSPCENDT [...] | 239 | SALN* | 250 |
| mdtH | GGAGG | MSRVSQAR [...] | 240 | GWAN* | 251 |
| yieO | AGGA | MSDKKKRS [...] | 241 | LTVN* | 252 |
| mdlB | — | — | | | |
| mdtO | — | — | | | |
| yojI | — | — | | | |
| ydhC | GGA/GAG/AGG | MQPGKRFL [...] | 242 | PAFN* | 253 |
| cusA | GGAG/GAGG | MIEWIIRR [...] | 243 | GPDN* | 254 |

TABLE 26B
Prediction of protein-coding genes using Prodigal prokaryotic gene
recognition and translation initiation site identifier. A consensus sequence
(60% threshold) of the efflux-deficient strain genome assembly, containing
the tandem stop codons, was utilized as the query fasta. As a control, the
wild-type E. coli K-12 genome, without any CRISPR modifications, was also
used (GenBank: CP009273.1). The Prodigal results were searched for efflux-
encoding genes using BLASTP (cutoff threshold E < 1.0 e⁻⁵⁰). Predicted peptide
size in EKO-35 is denoted as the length of the truncated peptide as a
fraction of the total protein size.
K-12 Genome

| | | Predicted Translation | |
|---|---|---|---|
| Gene | Predicted RBS | Start Site | SEQ ID NO: |
| acrB | AGGAG | MPNFFIDR [....] | 199 |
| acrD | GGAG/GAGG | MANFFIDR [....] | 200 |
| acrF | GGA/GAG/AGG | MANFFIRR [....] | 201 |
| mdtF | GGA/GAG/AGG | MANYFIDR [....] | 202 |
| macB | AGGAG | MTPLLELK [....] | 203 |
| emrB | GGAG/GAGG | MQQQKPLE [....] | 204 |
| mdtL | AGGA/GGAG/GAGG | MSRFLICS [....] | 205 |
| mdtK | GGA/GAG/AGG | MQKYISEA [....] | 206 |
| bcr | AGGAG | MTTRQHSS [....] | 207 |
| ydeA | Unknown | MTTNTVSR [....] | 208 |
| mdtM | AGGAG | MPRFFTRH [....] | 209 |
| yddA | Unknown | MITIPITL [....] | 210 |
| yebQ | Unknown | MPKVQADG [....] | 211 |
| emrE | AGGA/GGAG/GAGG | MNPYIYLG [....] | 212 |
| mdtD | GGAG/GAGG | MTDLPDST [....] | 213 |
| sugE | GGAG/GAGG | MSWIILVI [....] | 214 |
| ynfM | AGGA | MSRTTTVD [...] | 215 |
| emrD | Unknown | MKRQRNVN [...] | 216 |
| ydeF | GGA/GAG/AGG | MNLSLRRS [...] | 217 |
| mdtJ | AGGAG | MYIYWILL [...] | 218 |
| ydiM | AGxAG | MKNPYFPT [...] | 219 |
| mdtB | AGGAG | MQVLPPSS [...] | 220 |
| mdlA | AGGA | MRLFAQLS [...] | 221 |
| emrY | GGA/GAG/AGG | MAITKSTP [...] | 222 |
| mdfA | Unknown | MQNKLASG [...] | 223 |
| fsr | Unknown | MAMSEQPQ [...] | 224 |
| mdtG | GGA/GAG/AGG | MSPCENDT [...] | 225 |
| mdtH | GGAGG | MSRVSQAR [...] | 226 |
| yieO | AGGA | MSDKKKRS [...] | 227 |
| mdlB | AGGAG | MRSFSQLW [...] | 228 |
| mdtO | GGA/GAG/AGG | MSALNSLP [...] | 229 |
| yojI | Unknown | MELLVLVW [...] | 230 |
| ydhC | GGA/GAG/AGG | MQPGKRFL [...] | 231 |
| cusA | GGAG/GAGG | MIEWIIRR [...] | 232 |

While the present disclosure has been described with reference to what are presently considered to be the preferred example, it is to be understood that the disclosure is not limited to the disclosed example. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Baba, T. et al. Construction of Escherichia coli K-12 in-frame, single-gene knockout mutants: the Keio collection. Mol. Syst. Biol. 2, (2006).
Cox, J. & Mann, M. MaxQuant enables high peptide identification rates, individualized p.p.b.-range mass accuracies and proteome-wide protein quantification. Nat. Biotechnol. 26, 1367-1372 (2008).
Cox, J. & Mann, M. 1D and 2D annotation enrichment: a statistical method integrating quantitative proteomics with complementary high-throughput data. BMC Bioinformatics 13 Suppl 16, S12 (2012).
Cox, J. et al. Accurate proteome-wide label-free quantification by delayed normalization and maximal peptide ratio extraction, termed MaxLFQ. Mol. Cell. Proteomics 13, 2513-2526 (2014).
Hazel, A. J. et al. Conformational dynamics of AcrA govern multidrug efflux pump assembly. ACS Infect. Dis. 5, 1926-1935 (2019).
Johnson, J. W. et al. Antibacterial Activity of Metergoline Analogues: Revisiting the Ergot Alkaloid Scaffold for Antibiotic Discovery. ACS Med. Chem. Lett. 13, 284-291 (2022).
Rappsilber, J., Mann, M. & Ishihama, Y. Protocol for micro-purification, enrichment, pre-fractionation and storage of peptides for proteomics using StageTips. Nat. Protoc. 2, 1896-1906 (2007).
Stover, C. K. et al. Complete genome sequence of Pseudomonas aeruginosa PAO1, an opportunistic pathogen. Nature 406, 959-964 (2000).
Datsenko, K. A. & Wanner, B. L. One-step inactivation of chromosomal genes in Escherichia coli K-12 using PCR products. Proc. Natl. Acad. Sci. U.S.A. 97, 6640-6645 (2000).
Cherepanov, P. P. & Wackernagel, W. Gene disruption in Escherichia coli: TcR and KmR cassettes with the option of Flp-catalyzed excision of the antibiotic-resistance determinant. Gene 158, 9-14 (1995).
Jiang, Y. et al. Multigene editing in the Escherichia coli genome via the CRISPR-Cas9 system. Appl. Environ. Microbiol. 81, 2506-2514 (2015).
Cox, G. et al. A Common Platform for Antibiotic Dereplication and Adjuvant Discovery. Cell Chem Biol 24, 98-109 (2017).
Kearse, M. et al. Geneious Basic: an integrated and extendable desktop software platform for the organization and analysis of sequence data. Bioinformatics 28, 1647-1649 (2012).
Kitagawa, M. et al. Complete set of ORF clones of Escherichia coli ASKA library (a complete set of E. coli K-12 ORF archive): unique resources for biological research. DNA Res. 12, 291-299 (2005).
Lewinson, O. et al. Alkalitolerance: a biological function for a multidrug transporter in pH homeostasis. Proc. Natl. Acad. Sci. U.S.A. 101, 14073-14078 (2004).
Patel J. B., Cockerill R. F., Bradford A. P., Eliopoulos M. G., Hindler A. J., Jenkins G. S., Lewis S. J., Limbago B., Miller A. L., Nicolau P. D., Pwell M., Swenson M. J., Traczewski M. M., Turnidge J. D. WPMZLB. M07-A10: Methods for Dilution Antimicrobial Susceptibility Tests for Bacteria That Grow Aerobically; Approved Standard—Tenth Edition. CLSI (Clinical Lab Stand Institute) (2015).
Tyanova, S. et al. The Perseus computational platform for comprehensive analysis of (prote)omics data. Nat. Methods 13, 731-740 (2016).
Grenier, F. et al. Complete genome sequence of Escherichia coli BW25113. Genome announcements 2.5, e01038-14 (2014).

1. An Escherichia coli strain comprising at least 20 of inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA.

2. The Escherichia coli strain of claim 1, comprising at least 34 of the inactivated genes.

3. The Escherichia coli strain of claim 1, comprising all 35 inactivated genes.

4. The Escherichia coli strain of claim 1, wherein the Escherichia coli strain is deposited under IDAC accession number 310522-01 or IDAC accession number 070623-01, or an Escherichia coli comprising a nucleic acid having the sequence as shown in SEQ ID NO: 255.

5. The Escherichia coli strain of claim 1, further comprising an open variant of outer membrane ferric siderophore transporter FhuA.

6. The Escherichia coli strain of claim 3, wherein at least one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, is reactivated, optionally under the control of a constitutive promoter.

7. The Escherichia coli strain of claim 6, wherein one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, is reactivated.

8. The Escherichia coli strain of claim 5, wherein at least one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, is reactivated.

9. The Escherichia coli strain of claim 8, wherein one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, is reactivated.

10. The Escherichia coli strain of claim 1, wherein inactivation comprises deletion of the gene, or introducing a mutation into the gene to ablate expression of the gene or eliminate efflux pump activity of the protein expressed by the gene.

11. A method for identifying a compound that is an antibacterial agent, comprising
(a) i) contacting the compound with an Escherichia coli strain of comprising at least 20 of inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA (Strain A) and with wild-type Escherichia coli; and/or
ii) contacting the compound with Strain A and an Escherichia coli strain comprising inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, wherein at least one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, is reactivated (Strain B); and/or
iii) contacting the compound with Strain A and an Escherichia coli strain comprising inactivated genes acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, wherein one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, is reactivated (Strain C); and
(b) detecting viability of each of the Escherichia coli;
wherein the compound is identified as an antibacterial agent if the compound decreases viability of wild-type less than Strain A;
optionally wherein the compound is identified as an antibacterial agent if the compound decreases viability of Strain B less than Strain A;
optionally wherein the compound is identified as an antibacterial agent if the wild-type Escherichia coli or Strain B is resistant to the compound, and the compound decreases the viability of Strain A.

12. The method of claim 11, wherein the decrease in viability of wild-type or Strain B after contacting the compound is at most 10%, 20%, 30%, 40%, 50%, 60%, 70%, or 80%, and the viability of Strain A is at least 20%, 30%, 40%, 50%, 60%, 70%, 80%, 90%, or 100%.

13. The method of claim 11, wherein the compound is identified as an antibacterial agent if the compound decreases the viability of Strain A at a faster rate than the decrease in viability of wild-type or Strain B.

14. The method of claim 11, wherein the compound decreases the viability of Strain C less than Strain A, thereby identifying specificity of the compound for an efflux pump encoded by the reactivated gene, optionally the compound has a lower minimum inhibitory concentration (MIC) in Strain A than Strain C.

15. The method of claim 11, wherein the contacting comprises incubating the Escherichia coli in a suitable culturing media for optimal Escherichia coli growth.

16. The method of claim 11, wherein the contacting comprises incubating the Escherichia coli in nutrient-limiting culturing media.

17. The method of claim 15, wherein the contacting comprises incubating the Escherichia coli for at least 24 h, 48 h, 72 h, or 96 h.

18. The method of claim 11, wherein the culturing media is a media having a pH of about 2, about 3, about 4, or about 5.

19. A method for creating an Escherichia coli strain producing one or more efflux pump, comprising reactivating one of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA in the Escherichia coli strain of claim 3.

20. The method of claim 19, wherein the reactivation comprises introducing one or more of acrB, acrD, acrF, mdtF, macB, emrB, mdtL, mdtK, bcr, ydeA, mdtM, yddA, yebQ, emrE, mdtD, sugE, ynfM, emrD, ydeF, mdtJ, ydiM, mdtB, mdlA, emrY, mdfA, fsr, mdtG, mdtH, yieO, mdlB, mdtO, yojI, yajR, ydhC, and cusA, optionally by reversing the inactivation, optionally by re-introducing the gene back in genome with the same promoter or a different promoter, at the same locus or a different locus, optionally by introducing a knock down-resistant version of the gene.
